(19) United States
(12) Patent Application Publication
(10) Pub. No.: US 2024/0355418 A1
(43) Pub. Date: Oct. 24, 2024

(54) MULTIPLEX, TEMPORALLY RESOLVED MOLECULAR SIGNAL RECORDER AND RELATED METHODS

(71) Applicant: University of Washington, Seattle, WA (US)

(72) Inventors: Wei Chen, Seattle, WA (US); Jay Shendure, Seattle, WA (US); Junhong Choi, Seattle, WA (US)

(73) Assignee: University of Washington, Seattle, WA (US)

(21) Appl. No.: 18/688,244

(22) PCT Filed: Sep. 1, 2022
(86) PCT No.: PCT/US2022/075857

§ 371 Date: Feb. 29, 2024

Related U.S. Application Data
(60) Provisional application No. 63/240,143, filed on Sep. 2, 2021.

(51) Int. Cl. G16B 25/10; G06N 3/12; G16B 30/00

(52) U.S. Cl. CPC G16B 25/10; G06N 3/12; G16B 30/00

(57) ABSTRACT

Embodiments of the present disclosure provide composition and methods for recording an iterative nucleic acid editing event. The compositions and methods described herein comprise a first active target domain, comprising an editable recording sequence configured to hybridize with a first prime editing guide RNA (pegRNA) and one or more inactive truncated target domains comprising a non-editable sequence configured to not hybridize with the pegRNA, wherein the first pegRNA edits the first active target domain, wherein the pegRNA edit shifts the position of the recoding sequence from the editable sequence to the non-editable sequence, thereby changing the editable sequence to a non-editable sequence and the inactive truncated target domain to a second active target domain comprising a second recoding sequence configured to hybridize with a second pegRNA.

### CROSS-REFERENCE(S) TO RELATED APPLICATION(S)

This application claims the benefit of U.S. Provisional Application No. 63/240,143 filed on Sep. 2, 2021.

### STATEMENT OF GOVERNMENT LICENSE RIGHTS

This invention was made with Government support under Grant No. HG011586 awarded by the National Institutes of Health. The Government has certain rights in the invention.

### STATEMENT REGARDING SEQUENCE LISTING

The sequence listing XML associated with this application is provided in XML format and is hereby incorporated by references into the specification. The name of the XML file containing sequence the listing is 3915-P1216WOUW_Seq_List_20200830.xml. The text file is 132 KB; was created on Aug. 30, 2022; and is being submitted via Patent Center with the filing of the specification.

### BACKGROUND

There are current methodologies available to learn the order of molecular events in living systems. For example, a first approach is direct observation, e.g., live cell fluorescence microscopy to quantify the interactions in real time. A second approach is time-series experiments, e.g., destructively sampling and transcriptionally profiling a system at different timepoints. A third approach is epistatic analysis, e.g., ordering the actions of genes by comparing the phenotypes of single and double mutants. Although these and other approaches have important strengths, they are also limited in key ways. Specifically, live imaging is largely restricted to in vitro models. For time series experiments, resolution and accuracy are constrained by the frequency of sampling and the reproducibility of the biological process under investigation. Epistatic analysis is confounded by pleiotropy, particularly in multicellular organisms.

Another approach, theoretically promising but methodologically underdeveloped relative to the aforementioned alternatives is a DNA memory device, which is defined as an engineered system for recording molecular events through permanent changes to a cell's genome that can be read out post factum. To date, several proof-of-concept DNA memory devices have been described that leverage diverse approaches for the “write” operation, including site-specific recombinases (SSRs), CRISPR/Cas9 genome editing, CRISPR integrases, terminal deoxynucleotidyl transferases, base-pair misincorporation, base editing, and others.

The nature of the write operation in such DNA memory devices shapes their performance in terms of channel capacity for encoding and decoding signals, temporal resolution, interpretability, and portability. For example, SSRs record molecular signals with high efficiency, but the number of distinct signals that can be concurrently recorded is limited by the number of available SSRs. DNA memory devices relying on CRISPR/Cas9 can potentially overcome this limitation, e.g., if each signal of interest were coupled to the expression of a different guide RNA (gRNA), but in that case each signal would also require its own target(s). Furthermore, the CRISPR/Cas9 molecular recorders described to date rely on double-stranded breaks (DSBs) and nonhomologous end-joining (NHEJ) to “scar” target sites. In addition to being toxic, frequent DSBs often excise or corrupt consecutively located target sites, the molecular equivalent of accidental data deletion.

A further handicap of nearly all DNA memory devices described to date is that while recordings might stochastically accumulate at unordered target sites, the order in which they occurred is not explicitly captured. CRISPR integrase systems, which rely on the signal-induced, unidirectional incorporation of DNA spacers or transcript-derived tags to an expanding CRISPR array, overcome this limitation. However, at least to date, their reliance on accessory integration host factors has restricted such recorders to prokaryotic systems. Another approach, CHYRON, enables directional writing of information to DNA by combining self-targeting CRISPR gRNAs with the expression of terminal deoxynucleotidyl transferase (TdT), whose presence shifts the most likely outcome of NHEJ from short deletions to short insertions. While this approach unidirectionally inserts nucleotides in a signal-responsive manner, it continues to rely on NHEJ-mediated repair of DSBs. Furthermore, because each gRNA/target yields a homogenous signal (TdT-mediated insertions of variable length), it is not clear how it could be used to explicitly record the precise order of more than a handful of distinct signals. Finally, at least two groups have independently developed “logic-circuit architectures” that use sequential base editing to record the order and identity of biological signals both in bacterial and mammalian cells (DOMINO and CAMERA). However, because base editors are currently limited to writing single base substitutions to predefined targets, the order of signals can only be recorded via pre-programmed circuits, rendering multiplex recording challenging.

Many of the limitations described above for understanding the order of molecule events are also true for understanding gene expression because biological systems are complex and dynamic. During development, a modest number of core signaling pathways and gene regulatory modules are leveraged to program a precise spatiotemporal unfolding of programs of cell differentiation, proliferation, morphogenesis, and tissue patterning. Across species, differences in how these conserved pathways and modules are used underlie an incredible diversity of organismal form and function. Within species, genetic differences and environmental effects are presumed to influence these core modules in specific developmental or homeostatic contexts, giving rise to both natural phenotypic variation as well as myriad disease states.

Measurements of gene expression and signal transduction activity are conventionally performed with methods that require either the destruction or live imaging of a biological sample. These include RNA sequencing (RNA-seq), which measures the global transcriptional state of a system; massively parallel reporter assays (MPRAs), which use sequencing to measure the relative ability of members of a library of DNA fragments to act as enhancers of transcriptional activity in a controlled context; and fluorescent probes and reporters, which track the dynamics of specific signaling pathways in living systems.

These classes of methods are remarkably useful and yet limited in keyways. For example, with RNA-seq, individual samples provide only static snapshots of cell state, such that the temporal dynamics of gene expression must be pieced together by inference with a resolution that is limited by sampling density. Sequencing-based reporter assays are also destructive and static. Although time-series MPRAs can successfully define the temporal dynamics of enhancer activity, such studies are similarly limited by inference and sampling density. Fluorescent probes and reporters are better positioned to capture temporal dynamics, but require that the biological system be physically transparent, at least for live imaging, and are limited in terms of multiplexibility. Overall, there remains a need for a means of capturing signaling and gene regulatory activity that is at once quantitative, reproducible, non-destructive, multiplexable, applicable to physically opaque biological systems and capable of integrating large numbers of signals.

Although pioneering, the systems described above for DNA recording are also fundamentally limited with respect to multiplexibility—that is, the number of independent signals that can be recorded at once. In examples that have been demonstrated to date, enhancers are used to selectively drive the enzyme that mediates an alteration in DNA sequence or limited transcription repression elements are used to drive the gRNA expression in response to signals. In this framing, each signal requires its own enzyme or repression element, and it is difficult to imagine how more than a handful of independent signals could be concurrently recorded within the same cell or population of cells, let alone how extensive, concurrent recording of large numbers of biological signals could be achieved throughout the development of a multicellular organism.

Another challenge for the current recording systems is reading out. In current Cas9/Base editor-based recording systems, single guide RNAs (sgRNAs) program the location of editing but not the edit itself. As such, each sgRNA would require its own target, making it difficult to read out all targets at once. This challenge has been partially solved with paired sgRNA-target or homing gRNA (hgRNA) or self-targeting gRNA (stgRNA), but still has limited compatibility with recent development of RNA-seq. (See summary in Table 1). An ideal recorder should be able to simultaneously record multiple signals and read them out with either DNA amplicon or RNA sequencing.

In view of the limitations of the present art, a need remains for a highly multiplexed DNA-based memory device capable of recording biological signals, including transcriptional activity to DNA in an iterative and unidirectional manner. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect of the invention, the disclosure provides a nucleic acid construct for recording an iterative nucleic acid editing event. The construct can comprise a first active target domain, comprising an editable recording sequence configured to hybridize with a first prime editing guide RNA (pegRNA) and one or more inactive truncated target domains comprising a non-editable sequence configured to not hybridize with the pegRNA, wherein the first pegRNA edits the first active target domain, wherein the pegRNA edit shifts the position of the recoding sequence from the editable sequence to the non-editable sequence, thereby changing the editable sequence to a non-editable sequence and the inactive truncated target domain to a second active target domain comprising a second recoding sequence configured to hybridize with a second pegRNA.

In another aspect, the disclosure provides a vector comprising a nucleic acid sequence encoding the nucleic construct as described above coupled to a promoter and/or a transcribed form of an RNA molecule.

In another aspect, the disclosure provides a system for recording iterative nucleic acid editing events, the system comprising: the nucleic acid construct above, or a nucleic acid encoding the nucleic acid construct; one or more pegRNAs or one or more nucleic acids encoding the one or more pegRNAs configured to hybridize to a first active target domain; a prime editing enzyme, or a nucleic acid encoding the prime editing enzyme; wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

In another aspect, the disclosure provides a method of iteratively recording editing events, the method comprising: contacting the nucleic acid construct as described above with one or more pegRNAs and a prime editing enzyme; wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

In another aspect, the disclosure provides a method for multiplexed transcription recording, the method comprising: contacting the nucleic acid above with a prime editing guide RNA (pegRNA) expression cassette, a prime editing enzyme, and an endonuclease, wherein the expression cassette comprises a promoter, an endonuclease system comprising a first endonuclease target 5′ to the pegRNA and a second endonuclease target 3′ to the pegRNA, an optional nucleic acid construct encoding a functional GFP and/or an endonuclease, wherein the transcribed region of the nucleic acid construct comprises one or more pegRNAs and expression of one or more pegRNAs is driven by activation of the promoter releasing the one or more pegRNA by cleavage of the endonuclease target by an endonuclease; hybridizing the one or more pegRNAs to a target domain; and editing the target domain by inserting a barcode tag sequence.

In another aspect, the disclosure provides an expression cassette comprising a cis-regulatory-element (CRE) coupled promoter sequence and a nucleic acid sequence encoding from 5′ to 3′ a first endonuclease target, one or more prime editing guide RNAs (pegRNA), and a second endonuclease target, wherein the nucleic acid sequence is operably linked to the CRE coupled promoter sequence, and wherein cleavage of the first endonuclease target and the second endonuclease target releases the one or more pegRNAs causing the one or more pegRNAs to hybridize to a nucleic acid target and edit the nucleic acid target by inserting a barcode tag sequence.

In another aspect, the disclosure provides a method for multiplex transcriptional recording, the method comprising: coupling a cis-regulatory element (CRE) coupled promoter sequence to a nucleic acid sequence encoding from 5′ to 3′ a first endonuclease target, one or more prime editing guide RNAs (pegRNAs), and a second endonuclease target, releasing the one or more pegRNAs from a transcript by the addition of an endonuclease; and editing of a target nucleic acid sequence by inserting a barcode tag sequence.

In another aspect, the disclosure provides a method for screening transcriptional activity in response to external stimuli, the method comprising using any of the methods described above to record transcription activity of a plurality of DNA sequences in both the absence and presence of external stimuli and comparing the difference between transcriptional activity in both the absence and presence of external stimuli, wherein the difference in transcription activity in the presence of external stimuli can be used as a screening method for regulating therapeutic treatments.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1G. Sequential genome editing with DNA Typewriter.

(A). Schematic of two successive editing events at the “type-guide,” which shifts in position with each editing event. The DNA Tape consists of a tandem array of CRISPR-Cas9 target sites (grey boxes), all but the first of which are truncated at their 5′ ends, and therefore inactive. The 5-bp insertion includes a 2-bp pegRNA-specific barcode as well as a 3-bp key that activates the next monomer. Because genome editing is sequential in this scheme, the temporal order of recorded events can simply be read out by their physical order along the array.

(B). Schematic of prime editing with DNA Typewriter. Prime editing recognizes a CRISPR-Cas9 target and modifies it with the edit specified by the pegRNA. With DNA Typewriter, an insertional editing event generates a new prime editing target at the subsequent monomer.

(C). Schematic of ordered recording via DNA Typewriter. Individual pegRNAs are potentially event-driven or constitutively expressed, together with the PE2 enzyme.

(D-F). Specificity of genome editing on versions of TAPE-1 with two (D), three (E), or five (F) monomers. Cells bearing stably integrated TAPE-1 target arrays were transfected with a pool of plasmids expressing pegRNAs and PE2. Each class of outcomes is inclusive of all possible NNGGA insertions, and collectively the classes shown include (2ⁿ-1) possible outcomes, where n is the number of monomers. We observe that editing of any given target site is highly dependent on the preceding sites in the array having already been edited.

(G). Edit score of 16 barcodes used in the experiment with 5×TAPE-1. Edit scores for each insertion are calculated as log 2 of the ratio between insertion frequencies and the abundances of pegRNAs in the plasmid pool, averaged over n=3 transfection replicates.

FIGS. 2A through 2H. Transfection programs for 16 sequential epochs.

(A). Schematic of five transfection programs over 8 or 16 epochs. For Programs 1 and 2, pegRNAs with single barcodes were introduced in each epoch for 16 epochs. The specific orders aimed to maximize (Program-1) or minimize (Program-2) the edit distances between temporally adjacent transfections. For Program-3, pegRNAs with two different barcodes were introduced at a 1:1 ratio for 16 epochs, with one barcode always shared between adjacent epochs (and between epoch 1 and 16). For Programs 4 and 5, pegRNAs with two different barcodes were introduced either at constant ratio (1:3) or at varying ratios in each epoch (1:1, 1:2, 1:4, or 1:8) for 8 epochs, respectively.

(B). Barcode frequencies across 5 insertion sites in 5×TAPE-1 in Programs 1 and 2 following epoch 16. Barcodes introduced in early epochs are more frequently observed at the first site.

(C-G). Bigram transition matrix for Programs 1 (C), 2 (D), 3 (E), 4 (F), and 5 (G). Barcodes are ordered from early (left/top) to late (right/bottom).

(H). Calculated vs. intended relative frequencies between Programs 4 and 5. Program ratios are calculated by combining sequencing reads from n=3 independent transfection experiments.

FIGS. 3A through 3E. Recording and decoding short digital text messages with DNA Typewriter.

(A). Base64 binary-to-text was modified to assign 64 NNNGGA barcodes for TAPE-1 to 64 text characters.

(B). Illustration of the encoding strategy for “WHAT HATH GOD WROUGHT?,” which has 22 characters including whitespaces. The message is grouped into sets of 4 characters, converted to NNN barcodes according to the TAPE64 encoding table, and plasmids corresponding to each set mixed at a ratio of 7:5:3:1 for transfection. To encode 22 characters, we sequentially transfected 5 sets of 4 characters and 1 set of 2 characters 3 days apart to PE2(+) 5×TAPE-1(+) HEK293T cells.

(C-E). Decoding of 3 messages based on sequencing of 5×TAPE-1 arrays: (C) “WHAT HATH GOD WROUGHT?”, (D) “MR. WATSON, COME HERE!”, (E) “BOUND FOREVER, DNA”. For each message, the full set of NNNGGA insertions was first identified, and then co-transfected sets of characters identified from the bigram transition matrix (left). Within each set of characters inferred to have been co-transfected, ordering was based on corrected unigram counts (middle), resulting in the final decoded message (right). Both 2D histogram and corrected read counts are calculated by combining sequencing reads over n=3 independent transfection experiments. Read counts are corrected using the edit score for each insertion barcode.

FIGS. 4A through 4F. Reconstruction of a monophyletic cell lineage tree using DNA Typewriter and single cell RNA-seq.

(A). Schematic of lentiviral vector used in the DNA Typewriter-based lineage tracing experiment. The integration cassette includes a 5×TAPE-1 sequence associated with an 8-bp random barcode (TargetBC) and a pegRNA expression cassette. The pegRNA targets TAPE-1 and inserts 6-bp, wherein the first 3-bp is the random barcode (InsertBC) and the last 3-bp is the key sequence of GGA for TAPE-1. Each TargetBC-5×TAPE-1 array is embedded in the 3′-UTR (untranslated region) of eGFP with an RNA capture sequence at its 3′-end, and transcribed from the eEFIA promoter.

(B). Schematic of monophyletic lineage tracing experiment. A HEK293T line expressing Dox-inducible PE2 was transfected with the lentiviral construct shown in panel (A) at a high MOI. A monoclonal line was then established and expanded in the presence of Dox. During expansion, pegRNAs expressed by TargetBC-defined integrants compete to mediate insertions at the type-guides of TAPE-1 arrays within the same cell.

(C). Cumulative editing of each site within TAPE-1. Each colored line shows the cumulative editing rate for one of 13 TargetBCs. Grey bars denote the cumulative editing of TAPE-1 sites across all 13 independent TargetBCs within the n=1 single cell experiment.

(D). Histogram of the number of edits across 59 editable sites in each cell. The dotted line denotes the average.

(E). Histogram of the number of differences across 59 editable sites between all possible pairs of the 3,257 sampled cells. The red dotted line denotes the average.

(F). Distribution of the number of pairwise differences between each cell and its “nearest neighbour” among the 3,257 sampled cells.

FIGS. 5A through 5F. The relative insertional frequencies of k-mers to DNA Tape are determined by relative pegRNA abundances as well as by insertion-dependent sequence bias.

(A). Conditional, site-specific editing efficiencies across 3 sites within the 3×TAPE-1 or 5 sites within the 5×TAPE-1, calculated as the number of reads that contain an edit in the indicated site over the total number of reads that contain an edit in the immediately preceding site, which activates the indicated site as a target for editing. The number of all 5×TAPE-1 (or 3×TAPE-1) reads were used for calculating the site-specific editing efficiency for the Site-1, which is activated by its own key sequence. The center and error bars are mean and standard deviations, respectively, from n=2 transfection replicates for the second plot from the left and n=3 transfection replicates for the other 3 plots.

(B). Pairwise scatterplots of unigram frequencies of NNGGA insertions at the initiating monomer of 5×TAPE-1 among three transfection replicates.

(C). Scatterplot of unigram frequencies, averaged across three transfection replicates, at the initiating vs. second monomer of 5×TAPE-1.

(D). Scatterplot of averaged unigram frequencies at the initiating monomer in “pre-cloning pooling” experiment vs. the abundances of NNGGA pegRNA-expressing plasmids (left). Insertional bias was corrected for with data from a separate experiment using NNGGA pegRNA-expressing plasmids that were pooled post-cloning, resulting in a better correlation with the abundances of pegRNAs in the plasmid pool (right). Corrections were done by dividing pre-cloning unigram frequencies by post-cloning unigram frequencies at the initiating monomer and multiplying by post-cloning pegRNA plasmid frequencies.

(E). Scatterplot of NNGGA edit scores calculated on the initiating monomer of the 5×TAPE-1 target edited by pegRNA-expressing plasmids pooled pre-cloning vs. post-cloning. Edit scores for each insertion are calculated as log 2 of the ratio between insertion frequencies and the abundances of pegRNAs in the plasmid pool. Spearman's p was used instead of Pearson's r.

(F). Scatterplot of averaged unigram frequencies at the initiating monomer in “post-cloning pooling” experiment vs. the abundances of NNGGA pegRNA-expressing plasmids (left). Correcting for insertional bias with pre-cloning unigram frequencies improves the correlation (right).

FIGS. 6A through 6L. Enhancements of prime editing facilitate DNA Typewriter's range and efficiency.

(A). Editing efficiencies at the first site of 5×TAPE-1 integrated in HEK293T cells. A pool of plasmids expressing TAPE-1 targeting epegRNAs were transfected with the pCMV-PEmax-P2A-hMLH1dn plasmid. Five pools with different insertion lengths ranging from 5-bp (NNGGA) to 9-bp (NNNNNNGGA or 6N+GGA) were tested separately. The center and error bars are mean and standard deviations, respectively, from n=3 transfection replicates.

(B). Scatterplot of 16 NNGGA edit scores with pegRNAs vs. epegRNAs.

(C). Edit scores for 16 NNGGA insertions with epegRNA. Edit scores for each insertion are calculated as log 2 of the ratio between insertion frequencies and the abundances of pegRNAs in the plasmid pool.

(D). Scatterplot of 64 NNNGGA edit scores with pegRNAs vs. epegRNAs.

(E). Edit scores for 64 NNNGGA insertions with epegRNAs.

(F). Knee plot of read-counts for 4,096 possible 6N+GGA insertions, across three replicates. A minimum threshold of requiring at least 20 reads for a given insertion in each of the three transfection replicates was determined based on this plot.

(G). Knee plot of read-counts for 4,096 possible 6N+GGA-inserting pegRNAs from the pool of plasmids. A minimum threshold of 30 reads for each insertion plasmid was determined based on this plot.

(H). Edit scores for 1,908 6N+GGA insertions. Only insertions that appeared more than 20 reads in each of three transfection replicates and more than 30 reads in the sequencing of the plasmid pool were considered. Edit scores for each insertion are calculated as log 2 of the ratio between insertion frequencies and the abundances of pegRNAs in the plasmid pool.

(I). Top 25 edit scores for 6N+GGA insertions.

(J). Editing efficiencies at the first site of 5×TAPE-1 integrated in the mouse embryonic fibroblasts (MEFs) or mouse embryonic stem cells (mESCs). For mESCs, up to two sequential transfections of a pool of epegRNA-expressing plasmids were tested. The error bars are standard deviations from n=3 transfection replicates.

(K, L). Scatterplot of 16 NNGGA (k) and 64 NNNGGA (1) edit scores with epegRNAs in mESCs vs. HEK293T cells. Edit scores were calculated after one transfection (left) or two serial transfections (right) of the same pool of pCMV-PEmax-P2A-hMLH1dn/U6-epegRNA plasmids. The edit score calculated with two serial transfections showed higher correlations (Spearman's p) with the edit score measured in HEK293Ts, probably due to better coverage of the insertion pools. Edit scores shown in this figure are calculated by combining sequencing data across n=3 transfection replicate experiments.

FIGS. 7A through 7E. Characterising diverse DNA Tape designs for efficiency and directional accuracy.

(A). Deriving 48 TAPE designs from the eight basal CRISPR spacer sequences that previously demonstrated reasonable prime editing efficiencies via six distinct sequence shuffling procedures. Sequence identification numbers: HEK3 (SEQ ID NO: 1); TAPE-1 (SEQ ID NO: 2); TAPE-2 (SEQ ID NO: 3); TAPE-3 (SEQ ID NO: 4); TAPE-4 (SEQ ID NO: 5); TAPE-5 (SEQ ID NO: 6); TAPE-6 (SEQ ID NO: 7); K14 (SEQ ID NO: 8); TAPE-7 (SEQ ID NO: 9); TAPE-8 (SEQ ID NO: 10); TAPE-9 (SEQ ID NO: 11); TAPE-10 (SEQ ID NO: 12); TAPE-11 (SEQ ID NO: 13); TAPE-12 (SEQ ID NO: 14); k21 (SEQ ID NO: 15); TAPE-13 (SEQ ID NO: 16); TAPE-14 (SEQ ID NO: 17); TAPE-15 (SEQ ID NO: 18); TAPE-16 (SEQ ID NO: 19); TAPE-17 (SEQ ID NO: 20); TAPE-18 (SEQ ID NO: 21); k22 (SEQ ID NO: 22); TAPE-19 (SEQ ID NO: 23); TAPE-20 (SEQ ID NO: 24); TAPE-21 (SEQ ID NO: 25); TAPE-22 (SEQ ID NO: 26); TAPE-23 (SEQ ID NO: 27); TAPE-24 (SEQ ID NO: 28); FANCF (SEQ ID NO: 29); TAPE-25 (SEQ ID NO: 30); TAPE-26 (SEQ ID NO: 31); TAPE-27 (SEQ ID NO: 32); TAPE-28 (SEQ ID NO: 33); TAPE-29 (SEQ ID NO: 34); TAPE-30 (SEQ ID NO: 35); PD1 (SEQ ID NO: 36); TAPE-31 (SEQ ID NO: 37); TAPE-32 (SEQ ID NO: 38); TAPE-33 (SEQ ID NO: 39); TAPE-34 (SEQ ID NO: 40); TAPE-35 (SEQ ID NO: 41); TAPE-36 (SEQ ID NO: 42); PD2 (SEQ ID NO: 43); TAPE-37 (SEQ ID NO: 44); TAPE-38 (SEQ ID NO: 45); TAPE-39 (SEQ ID NO: 46); TAPE-40 (SEQ ID NO: 47); TAPE-41 (SEQ ID NO: 48); TAPE-42 (SEQ ID NO: 49); PD3 (SEQ ID NO: 50); TAPE-43 (SEQ ID NO: 51); TAPE-44 (SEQ ID NO: 52); TAPE-45 (SEQ ID NO: 53); TAPE-46 (SEQ ID NO: 54); TAPE-47 (SEQ ID NO: 55); TAPE-48 (SEQ ID NO: 56).

(B). Efficiency (fraction of edited reads out of all reads) vs. sequential error rate (fraction of edited reads inconsistent with sequential, directional editing out of all edited reads) for 48 3×TAPE constructs on episomal DNA (left) and piggyBAC transposon integrated DNA (right). Both horizontal and vertical error bars are standard deviations from n=3 transfection replicates.

(C). Boxplots of the efficiencies and sequential error rates of 3×TAPE constructs derived from 8 basal sequences for each of 6 design procedures. Each data point is either mean efficiencies or mean sequential error rates over n=3 independent transfection experiments with 8 basal sequences in each experiment. In general, a longer key sequence was associated with a lower error rate, while a longer insertion did not appreciably impact efficiency (e.g., NNGGAC with Design-6 vs. NNGA with Design-5).

(D). Boxplots of sequential error rates (left) and efficiencies (right) of 3×TAPE constructs grouped by their basal CRISPR target sequences. Each data point is either mean efficiencies or mean sequential error rates over n=3 independent transfection experiments with 6 design procedures in each experiment. Boxplot elements in (C, D) represent: Thick horizontal lines, median; upper and lower box edges, first and third quartiles, respectively; whiskers, 1.5 times the interquartile range; circles, outliers.

(E). Correlation between the sequential error rate (left) and editing efficiency (right) of each 3×TAPE construct either in the context of episomal DNA vs. integrated DNA. Each data point is both mean efficiencies and mean sequential error rates over n=3 independent transfection experiments with 48 designs in each experiment.

FIGS. 8A through 8F. Inferred event order and magnitude from sequential transfections.

(A). Sequential editing efficiency and sum of sequential errors from five sites in 5×TAPE-1 across 16 transfection epochs of Program-1.

(B). Repeat-length change of 5×TAPE-1 array sampled over 16 transfection epochs.

(C). For each of the five transfection programs, the event orders are inferred using “Unigram” (top) and “Bigram” (bottom) information.

(D). Undersampling analysis of Program-1. From the original 277,397 sequencing reads used for Program-1, it was undersampled to 10,000, 2,500, 2,000, 1,500, or 1,000 reads. For each sampling point, the bigram transition matrix (top) was plotted, and order of events (bottom) were inferred using bigram information. In (C), (D), sequencing reads from n=3 independent transfection experiments are combined.

(E, F). For Program-4 (E) and Program-5 (F), the absolute barcode read counts (left) are corrected based on the edit score of 16 NNGGA barcodes (middle) and used to calculate the relative magnitude of two co-transfected barcodes (right). The expected barcode ratios are marked with a “X” mark in each epoch. The center and error bars in panels (A), (B), (E), and (F) are mean and standard deviations, respectively, from n=3 transfection replicates.

FIGS. 9A and 9B. Inferring the barcode overlap in each message.

(A). Hierarchical clustering analyses of identified unigram barcodes based on the bigram matrices. For each message, the normalised bigram matrix was converted to a distance matrix using the Euclidean distance measure. The resulting distance matrix was then used for clustering 3-mer barcodes using the complete-linkage clustering method, resulting in a cluster dendrogram for each message. Based on these dendrograms, groups of 2 to 4 barcodes were manually grouped as putative co-transfection sets and ordered within the set based on unigram frequencies. Sets were ordered relative to one another using the normalised bigram matrix, following the sorting algorithm described in the text.

(B). Undersampling analysis of the short text “WHAT HATH GOD WROUGHT?.” From the original 1,256,996 sequencing reads, it was undersampled to 4 sampling points: 1,000,000, 100,000, 10,000, and 5,000 reads. For each sampling point, the bigram transition matrix (top), the corrected unigram counts (middle), and the hierarchical clustering (bottom) were plotted. From these, the original short text was inferred at the end. Both 2D histogram and corrected read counts are calculated by summing the sequencing reads over n=3 independent transfection experiments. Read counts are corrected using the edit score for each insertion barcode.

FIGS. 10A through 10E. Characterising the monoclonal lineage tracing experiment.

(A). Cell doubling times measured for HEK293T and the monoclonal lineage tracing cell line (iPE2(+) LT(+)), with or without Doxycycline (Dox). The presence of Dox lengthened the cell doubling time, possibly negatively affecting the cell physiology. P values were obtained using the two-tailed Student's t-test with Bonferroni correction: only *P<0.05 are shown. The center and error bars are mean and standard deviations, respectively, from n=3 independent experiments.

(B). Determining a set of valid TargetBCs based on frequencies. The Y-axis is on a log 10-scale. Recovered TargetBCs were first ranked by their read counts to estimate multiplicity of infection (MOI) (left). Any additional TargetBCs that are 1-bp Hamming distance away from the set of 19 were corrected. 3,257 cells were retained for which 13 of the most frequent TargetBCs (excluding one tape sequence with a corrupted type-guide) for lineage analysis (right) were recovered.

(C). Read counts of InsertBCs observed in TAPE-1 arrays. The Y-axis is on a log 10-scale. For the 3,257 selected cells, it was additionally required that all observed edits were amongst the 19 most frequent InsertBCs in the overall dataset, as this was presumed to be the valid set of pegRNA-defined insertional edits.

(D). Characterization of indel error rates of prime editing on TargetBC-5×TAPE-1 arrays. The Y-axis is on a log 10-scale. Correct length insertions with prime editing are >100-fold more likely than an insertion of a different length product. Furthermore, some of the apparent longer insertions are likely to correspond to a contraction of TAPE-1 monomer within 5×TAPE-1 before the integration, such as contraction of TGATGGTGAGCACG (SEQ ID NO: 57) TAPE-1 monomer to the observed TGAGCACG 8-bp sequence appearing between two TAPE-1 monomers.

(E). Characterization of substitution error rates during prime editing-mediated insertion of the GGA key sequence on TargetBC-5×TAPE-1 arrays. The X-axis is on a log 10-scale. Correct insertions are >100-fold more likely than insertions with substitution errors. The most frequent class of errors are transition errors, and these may be occurring during PCR amplification or sequencing-by-synthesis of cDNA amplicons, rather than during prime editing. Data in panel (B) to (E) is generated from n=1 monoclonal lineage experiment, followed by n=1 single-cell RNA-seq data collection.

FIGS. 11A through 11H. Editing and recovering longer TAPE arrays.

(A-B). Sanger sequencing traces for cloned (A) 12×TAPE-1 and (B) 20×TAPE-1 constructs. Each TAPE-array includes the 3-bp key sequence (GGA for TAPE-1), 12 or 20 repeats of 14-bp TAPE-1 monomer, and a 11-bp partial TAPE-1 monomer to serve as a prime-editing homology sequence for the last editing site. Grey bars in the background are proportional to quality (Phred-scale) for each base call.

(C-H). Integration, editing, and recovery of 12× and 20×TAPE-1 arrays. Each construct was integrated into PE2(+) 3N-TAPE-1-pegRNA(+) HEK293T cell line in triplicate, cultured for 40 days for prolonged editing, and recovered via PCR and long-read sequencing on the PacBio platform. Circular consensus sequencing (CCS) reads that had at least 3 NNNGGA insertions and no small indel errors were grouped based on the site of integration (using 8-bp TargetBC barcodes), and a read with the maximum number of TAPE-1 monomers (and within that set, the read with the maximum number of edits) was selected per TargetBC. (C). Histogram of the number of TAPE-1 monomers recovered from ˜12×TAPE-1 (top) and ˜20×TAPE-1 (bottom) integrants. (D). Histogram of number of edits recovered from ˜12×TAPE-1 (top) and ˜20×TAPE-1 (bottom) integrants. (E). For TargetBC groups with a given maximum number of TAPE-1 monomers (X-axis), the mean proportion were shown with the same number of monomers as the maximum (Y-axis), for both 12×TAPE-1 (dark gray) and 20×TAPE-1 (gray) integrants. It was concluded from this that shorter arrays are more stable, and that the length-dependent stability is consistent between the two experiments. (F). Similar to (E) but showing the full distribution of monomer lengths (Y-axis) for each TargetBC group with a given maximum number of TAPE-1 monomers (X-axis), for both ˜12×TAPE-1 (dark gray) and ˜20×TAPE-1 (gray) integrants. The size of dots are proportional to these proportions. Data shown in panels (C) to (F) are generated by combining sequencing reads from n=3 transfection replicate experiments. (G and H). Recovery of (G) ˜12×-TAPE-1 and (H) ˜20×-TAPE-1 arrays after prolonged editing. Edited portions of each TAPE-array are colored dark gray and overwhelmingly exhibit sequential editing. Very rarely, were instances of non-sequential editing, e.g., internal monomers that are edited observed. These are marked with asterisks below the corresponding column.

FIGS. 12A through 12H. ENhancer-driven Genomic Recording of transcriptional Activity in Multiplex (ENGRAM).

(A) Schematic of ENGRAM. Endogenous or synthetic cis-regulatory elements (CREs) drive activity-dependent transcription of a prime editing guide RNA (pegRNA) encoding a CRE-specific insertion. pegRNA is flanked by two 17 bp csy4 hairpin and can be released from pol-2 transcript by Csy4 ribonuclease. Endogenous CREs are sequences with enhancer activity measured by MPRA. Synthetic CREs are tandem repeats of TF motifs. The insertion is written to a natural or synthetic recording site within genomic DNA (“DNA Tape”). Thus, the signal is stored as a barcode in the DNA Tape for further readout.

(B) Three versions of ENGRAM 2.0 with the csy4 hairpin-flanked pegRNA embedded in the 5′ or 3′ UTR of a transcript encoding Csy4, or 3′ ENGRAM 2.0 with an additional csy4 hairpin in the 5′ UTR in order to impose auto-regulatory negative feedback on Csy4 levels.

(C) All three ENGRAM 2.0 recorders were integrated via PiggyBac into PE2-expressing cells in triplicate, each driving 1,024 5N barcodes with minP. The background editing efficiency was periodically checked over 20 days. Error bars correspond to standard deviations across 3 transfection replicates.

(D) NF-κB response element is cloned to upstream of minP in all three ENGRAM 2.0 recorders. NF-κB responsive ENGRAM recorders were integrated via PiggyBac into PE2-expressing cells. Recording activity was measured in the absence or presence of 10 ng/ml of TNFα in triplicate. Both 5′-ENGRAM and 3′-FT ENGRAM showed low background activity and strong activation in response to NF-κB activation, while 3′-ENGRAM showed high background and limited activation. Error bars correspond to standard deviations across 3 replicates. P-values were obtained using the two-tailed Student's t-test.

(E) Schematic of 5N barcode recording. pegRNA encoding degenerate 5N is cloned into 5′-ENGRAM architecture and driven by a PGK promoter.

(F) Log-scaled insertion proportions (calculated as the proportion of edited HEK3 sites with a given insertion) are highly correlated between transfection replicates.

(G) Range of editing scores (ES) for 5N insertions. ES are calculated as (genomic reads with specific insertion/total edited HEK3 reads)/(plasmid reads with specific insertion/total plasmid reads), plotted here in rank order on a log 2-scale. A total of 948 of 1024 all potential 5N barcodes were recovered after removing underrepresented barcodes. A few of the highest and lowest ranked insertions are highlighted (sequences shown are those observed in DNA Tape, which are the reverse complement of sequences in pegRNAs).

(H) A linear lasso regression model trained on these data with one-hot encoded single and dinucleotide content of the 5-mer and MFE of secondary structure as features predicts insertional efficiencies with reasonably high accuracy. Samples were split with 680 barcodes in a training set and 268 barcodes in a test set. The model was trained with 10-fold cross-validation on the training set and then used to predict the test set.

FIGS. 13A through 13E. The architecture and performance of ENGRAM recorders.

(A) Schematic of the ENGRAM 1.0 recorder. A pegRNA writing unit is flanked by csy4 hairpins and embedded within the 3′ UTR of a Pol-2-driven GFP mRNA. PE2 and Csy4 are constitutively expressed from a separate locus. Csy4 cleaves at the csy4 hairpins and releases the active pegRNA.

(B) Across three transfection replicates, the ENGRAM 1.0 recorder driven by a constitutive Pol-2 PGK promoter (PGK-CTT) exhibited comparable efficiency for inserting CTT at the HEK3 locus to a U6-driven CTT-pegRNA (U6-CTT). In the K562 cell line in which this experiment was performed, PE2 and Csy4 were constitutively expressed.

(C) A schematic of the constructs used for the two pools of ENGRAM 1.0 recorders is shown on the left, and the observed editing efficiency for each pool on the right. Briefly, a pool of 13 enhancers known to be active in this cell line, cloned upstream of minP and driving a pool of pegRNAs encoding insertion of a 5N degenerate sequence to HEK3, was 2.14-fold more active than a control construct bearing minP alone. Error bars correspond to standard deviations across 3 transfection replicates. P-values were obtained using the two-tailed Student's t-test.

(D) Schematic of the ENGRAM 2.0 recorder. A pegRNA writing unit is flanked by csy4 hairpins and embedded within the 3′ or 5′ UTR of a Pol-2-driven Csy4 mRNA. PE2 is constitutively expressed from a separate locus. Csy4 cleaves at the csy4 hairpins and releases the active pegRNA.

(E) ENGRAM 2.0 exhibits lower levels of background recording than ENGRAM 1.0. Measurements are for minP alone driving pegRNAs programming a degenerate 5N insertion to the HEK3 locus in triplicate, 3 days post-transfection. Error bars correspond to standard deviations across 3 transfection replicates. P-values were obtained using the two-tailed Student's t-test.

FIGS. 14A through 14G. The ENGRAM recorder installs barcodes with reasonable efficiency and reproducibility.

(A-C) Reproducibility of the relative proportions of 1023 5N barcodes installed by ENGRAM driven by the constitutive Pol-2 PGK promoter. Log-scaled insertion proportions (calculated as the proportion of edited HEK3 sites with a given insertion) were well correlated between pairs of transfection replicates.

(D-E) Predicted secondary structures for pegRNAs with the lowest (left) and highest (right) insertional efficiencies. Sequences shown above are those observed in DNA Tape, which are the reverse complement of sequences in pegRNAs.

(F) The rank-ordered coefficients of the linear lasso regression. Positional information of single nucleotides and dinucleotides and minimum free energy (MFE) of secondary structure were used as input features for training. In addition to MFE, which received the highest coefficient, the top 4 and bottom 4 coefficients for sequence features are annotated (e.g., 1-A and 3-TC mean A at first nucleotide or TC dinucleotide starting at position 3, respectively).

(G) MFE alone can explain 70% of the variance of the model.

FIGS. 15A through 15C. ENGRAM recording with new pegRNA and prime editor architecture.

(A) Comparison of recording efficiency between epegRNA and pegRNA. pegRNA/epegRNA encoding 5N degenerate barcode is cloned into 5′-ENGRAM architecture and is driven by a PGK promoter. These two libraries were transiently transfected into PE2+ HEK293T cells separately in triplicate. Genomic DNA was harvested three days post-transfection. Unexpectedly, pegRNA showed 30% higher recording efficiency than epegRNA. It was reasoned that the csy4 hairpin might serve a similar role as the tevoPreQ1 hairpin to protect pegRNA from degradation, additional hairpin might affect RNA folding. Error bars correspond to standard deviations across 3 transfection replicates. P-values were obtained using the two-tailed Student's t-test.

(B) Comparison of recording efficiency between PE2 and PEmax. PE2/PEmax and PGK-5N-ENGRAM were co-transfected into K562 cells in triplicate. Genomic DNA was harvested three days post-transfection. It was observed that PEmax showed 3.9-fold more efficient than PE2. It is recommended to use PEmax for all future recording assays. In this paper, PE2 was used. Error bars correspond to standard deviations across 3 transfection replicates. P-values were obtained using the two-tailed Student's t-test.

(C) tRNA processing for pegRNA release doesn't work in ENGRAM architecture. csy4 hairpin was replaced with tRNA to see if tRNA can provide an alternative approach for pegRNA releasing. Both ENGRAM pegRNA and tRNA flanked pegRNA encoding 5N degenerate insertion were driven by the NF-κB response element. Recorders were integrated into cells via PiggyBac. Recording activities were measured in the absence or presence of 10 ng/ml TNFα in triplicate. However, tRNA flanked pegRNA failed to show recording activity in both conditions.

FIGS. 16A through 16E. Recording enhancer activity with 5′ ENGRAM recorders.

(A) Schematic of enhancer recording. Enhancer library is cloned to upstream of a minP in 5′-ENGRAM recorders and integrated into PE2+ K562 cells using PiggyBac. Enhancer activity can be recorded to endogenous DNA TAPE (genomic HEK3 locus, n=2) or synthetic DNA TAPE (HEK3 locus integrated into the genome via PiggyBac, n=10-30).

(B) Benchmarking of ENGRAM with enhancers with known activities in a reporter assay. 5′-ENGRAM recorders with active and inactive enhancers upstream of a minP, together with minP-only and promoter-less constructs, were cloned, each driving expression of distinct pegRNA-encoded barcodes.

(C) Barcodes corresponding to the active enhancer showed 17.3, 18.3, and 22.5-fold more abundance than inactive enhancer, minP and promoter less control, respectively. Error bars correspond to standard deviations from 3 transfection replicates. Error bars correspond to standard deviations across 3 transfection replicates. P-values were obtained using the two-tailed Student's t-test. (D-E) Further benchmarking of ENGRAM 2.0 with 300 enhancers known to have a range of activities in a reporter assay.

(D) This library was designed such that each enhancer drove expression of a distinct pegRNA-encoded 6-mer insertional barcode.

(E) Values correspond to the proportion of each barcode read out from the HEK3 genomic locus (ENGRAM) or from the pegRNAs (MPRA), out of the total. The log-scaled proportions of ENGRAM events recorded to DNA were highly correlated with log-scaled proportions of barcodes measured directly from RNA.

FIGS. 17A through 17G. Benchmarking of ENGRAM 2.0 recorders.

(A) Recording efficiency on synthetic HEK3 locus and endogenous Hek3 locus. In the same pool of cells, endogenous and synthetic HEK3 locus show 3.08% and 1.76% overall recording efficiency, respectively. Of note, ˜15 copies of synthetic HEK3 are integrated.

(B) Log-transformed insertion proportions for 300 6-mer barcodes were highly correlated between synthetic and endogenous HEK3 locus.

(C-D) Different cell numbers were sampled (6,000, 12,000, 24,000, 48,000, 96,000 cells) on both endogenous and synthetic HEK3 locus to compare their recording efficiency and sensitivity. Overall, with 12,000 cells, most enhancers can be captured with reasonable reproducibility.

(E) Log-transformed insertion proportions for 300 6-mer barcodes were highly reproducible across transfection replicates. Each value corresponds to the proportion of barcodes read out at the DNA level from the HEK3 locus.

(F) Log-transformed RNA proportions for 300 6-mer barcodes were highly reproducible across transfection replicates. Each value corresponds to the proportion of barcodes read out at the RNA level from transcribed pegRNAs.

(G) The log-scaled proportions of ENGRAM events recorded to DNA were highly correlated with log-scaled proportions of barcodes measured directly from RNA.

FIGS. 18A through 18J. Recording the intensity and duration of signaling pathway activation or small molecule exposure.

(A) Signal-responsive regulatory elements were used to construct ENGRAM 2.0 recorders for activation by doxycycline (TetON; Tet Response Element), TNFα (a NF-κB responsive element) and CHIR99021 (a TCF-LEF responsive element, responsive to Wnt signaling).

(B-D) Upon 48 hours of stimulation with the corresponding stimulant, the TetON (B), NF-κB (C), and Wnt (D) recorders exhibited dose-dependent levels of recording. These experiments were conducted on separate, polyclonal cell lines, each of which had one recorder integrated via PiggyBac. Cells were exposed to a serial two-fold dilution series of doxycycline (B), TNFα (C) or CHIR99021 (D), with starting concentrations of 8 ng/ml, 64 ng/ml and 32 μM, respectively. For CHIR99021, more concentrations were sampled between 1 to 4 μM.

(E) Dynamic range observed in signal recording experiments. Recorders show an 11.5-fold, 19.0-fold and 22.6-fold between activation and background for the Tet, NF-κB and Wnt recorders, respectively. Error bars correspond to standard deviations from 3 stimulus replicates.

(F, G) Heatmap showing editing efficiencies resulting from matrix experiment on the NF-κB (F) and Wnt (G) recorders, in which both stimulant concentrations and durations of exposure were varied (2 recorders×8 concentrations×8 durations×3 replicates=384 conditions), illustrating the joint dependence of recording levels on the dose and duration of stimulation.

(H) Schematic of multiplex recording of signaling pathways. Similar to (A) except that all three recorders are integrated within a single population of cells and are writing to a shared DNA Tape.

(I) Cells bearing multiple recorders were exposed to all possible on/off combinations of three stimuli for 48 hours, followed by harvesting and sequencing-based quantification of the levels of signal-specific barcodes. Colored shapes as in panel (A). Concentrations used were 500 ng/ml, 10 ng/ml and 3 μM for doxycycline, TNFα and CHIR99021, respectively.

(J) Cells bearing multiple recorders were exposed to all possible combinations of high, medium, or low concentrations of three stimuli for 48 hours, followed by harvesting and sequencing-based quantification of the levels of signal-specific barcodes. For Dox, 62.5, 250 or 1000 ng/ml were used; for TNFα, 1, 4 or 16 ng/ml; and for CHIR99021, 1, 2 or 2.5 μM.

FIGS. 19A through 19E. Multiplex recording of signaling pathway activation or small molecule exposure with ENGRAM.

(A) A minimal level of background recording was observed in the absence of stimulus with the signal-responsive ENGRAM recorders. This background did not accumulate over time, consistent with the hypothesis that it primarily accumulates shortly after transfection, potentially due to ORI-driven, plasmid-mediated transcription. Plotted points correspond to three transfection replicates.

(B-C) Histograms showing editing efficiencies resulting from matrix experiment on the NF-κB (B) and Wnt (C) recorders, in which both stimulant concentrations and durations of exposure were varied (2 recorders×8 concentrations×8 durations×3 replicates=384 conditions). Error bars correspond to standard deviations from 3 stimulus replicates.

(D) Barcode composition of DNA Tape from cells treated with different combinations of stimuli. The recorders exhibit minimal crosstalk between signaling pathways (e.g., stimulating with CHIR does not lead to appreciable recording by the NF-κB recorder).

(E) Heatmap visualization of the data shown in FIG. 13J. Levels of recording are informative of each stimulant's concentration, even in the context of concurrent recording of three signals to a shared DNA Tape.

FIGS. 20A through 20C. Multiplex recording of signaling pathways or the order of signaling events with ENGRAM.

(A) Strategy for ENGRAM-based recording of the order of events A & B. In brief, each signal-responsive recorder programs the expression of two pegRNAs, one of which targets blank DNA Tape, and the other of which targets DNA Tape that has already been edited in response to the other signal.

(B) The editing outcomes (A only, B only, A-B′ and B-A′) associated with 11 transfection programs in which either both A & B were introduced simultaneously (1 program), only A or B was introduced (2 programs), or the recorders were serially transfected with varying recovery periods (A→B or B→A: 8 programs) were quantified.

(C) The different classes of transfection programs can be distinguished by the ratios of A-B′/B-A′ (y-axis) and A/B editing (x-axis) outcomes. Provided at least 24 hours of recovery between transfections, A→B programs are readily distinguished from B→A programs. Error bars correspond to standard deviations across 3 transfection replicates.

FIGS. 21A and 21B. Multiplex recording of signaling pathways or the order of signaling events with ENGRAM.

(A) Overall editing efficiencies for the eleven transfection programs represented in FIG. 19A.

(B) Bar plot representation of the same data shown in FIG. 18B. The different classes of transfection programs can be distinguished by the ratios of A-B′/B-A′ and A/B editing outcomes. Provided at least 24 hours of recovery between transfections, A→B programs are readily distinguished from B→A programs.

### DETAILED DESCRIPTION

DNA is naturally well-suited to serve as a digital medium for in vivo molecular recording. However, DNA-based memory devices described to date are constrained in terms of the number of distinct signals that can be concurrently recorded as well as by a failure to capture the precise order of recorded events. This disclosure is based on development of advanced platforms for molecular recording. As described in more detail below in Example 1, a DNA Ticker Tape platform was developed, functioning as a general system for in vivo molecular recording that largely overcomes these limitations.

Briefly, blank DNA Ticker Tape comprises a tandem array of partial CRISPR/Cas9 target sites, with all but the first site truncated at their 5′ ends, and therefore inactive. Signals of interest are coupled to the expression of specific prime editing guide RNAs. Editing events are insertional and record the identity of the guide RNA mediating the insertion while also shifting the position of the “write head” by one unit along the tandem array, i.e., iterative genome editing. In this proof-of-concept of DNA Ticker Tape, the inventors demonstrate the recording and decoding of complex event histories (e.g., the temporal order of 16 distinct signals); evaluate the performance of dozens of target array monomers; and test the encoding of short digital texts; and show how the ordered nature of DNA Ticker Tape simplifies the decoding of cell lineage histories.

Example 2 describes the development of a compatible framework for multiplexed molecular recording using prime editing and Csy4, which is termed “ENGRAM” (Enhancer-mediated genome recording of transcriptional activity). ENGRAM is shown to record events with high sensitivity and in a dose dependent manner. ENGRAM can simultaneously record multiple transcriptional events, their relative activities, and the temporal orders. ENGRAM can be widely used in measuring the temporal regulation of gene expression that is critical to understand highly dynamic biological processes, and impact diverse areas such as including functional genomics, neuroscience, and developmental biology. The ENGRAM embodiments are discussed in the context of use with Ticker Tape. However, a person of ordinary skill in the art would readily understand that it can be used independently in other aspects. Such aspects are encompassed by the present disclosure. For example, ENGRAM can be also repurposed as a screening platform to identify enhancer candidates. Traditional MPRA has identified many non-coding regions as potential enhancers. However, MPRA uses RNA as a readout, limiting its application to relatively highly expressed enhancers and constitutively active enhancers. In contrast, ENGRAM efficiently captures transcription activities, including low or transient transcription activities, and permanently records them into DNA tape. The DNA tape can be designed to include a restriction site so that the unedited DNA tape would be digested and recorded information would be enriched, reducing the cost of downstream DNA sequencing. Compared to MPRA, ENGRAM may provide higher sensitivity at a lower sequencing cost. For example, ENGRAM can be used to identify tissue specific enhancer, ligand specific enhancers, developmental enhancers.

DNA is naturally well-suited to serve as a digital medium for in vivo molecular recording. However, contemporary DNA-based memory devices are constrained in terms of the number of distinct “symbols” that can be concurrently recorded and/or by a failure to capture the order in which events occur. Here we describe DNA Typewriter, a general system for in vivo molecular recording that overcomes these and other limitations. For DNA Typewriter, blank recording media (“DNA Tape”) consists of a tandem array of partial CRISPR-Cas9 target sites, with all but the first site truncated at their 5′ ends, and therefore inactive. Short insertional edits serve as “symbols” that record the identity of the prime editing guide RNA mediating the edit while also shifting the position of the “type-guide” by one unit along the DNA Tape, i.e., sequential genome editing. In this proof-of-concept of DNA Typewriter, we demonstrate recording and decoding of thousands of symbols, complex event histories and short text messages; evaluate performance of dozens of orthogonal tapes; and construct “long tape” potentially capable of recording as many as 20 serial events. Finally, we leverage DNA Typewriter in conjunction with single cell RNA-seq to reconstruct a monophyletic lineage of 3,257 cells and find that the Poisson-like accumulation of sequential edits to multi-copy DNA Tape can be maintained across at least 20 generations and 25 days of in vitro clonal expansion.

As used herein, the term “recording”, “recording editing events”, or “sequential recording” and any similar terms refer to permanently fixing the history of a cellular event as modification of selected target DNA sequences. The modification of selected target DNA sequences can be used as a readout of (past) cellular events. As used herein “events” refers to history (i.e., cellular history or molecular history) of a change in expression of a particular gene, a change in a particular protein, a change in the level of an intracellular molecule, a change in a posttranslational modification, a change in the activity of a factor of interest, a change in microenvironment, exposure to a molecule of interest, activation of a transcription factor, deactivation of a transcriptional repressor, recruitment of a transcription factor, activation of a signal transduction pathway, cell lineage (e.g., cell development), or remodeling of chromatin.

As used herein “iterative” or “iterative recording” refers to recording events in a sequential ordered fashion. For example, these terms refer to recording at least two events in an ordered manner in which one could review the recorded history and identify and first event and when it occurred, identify the last event and when it occurred and determine the identity and ordering of all events occurring between the first and last event.

As used herein, “multiplex” refers to capturing and recording a plurality of independent signals. In some embodiments, these signals can include any biological signal or event of interest, including but not limited to, changes in gene expression and signal transduction.

The biological signal or event of interest can be any type of molecular event occurring in vivo associated with a particular gene and the event is not limited by the particular gene's structure or function. In some embodiments, the gene can be a transcription factor, enzyme, ribosomal gene, structural gene, miRNA, etc. and may be involved in any type of cellular function, such as without limitation cell signaling, cell division, etc. In other embodiments, the gene of interest is endogenous to the cell, however, embodiments of the constructs disclosed in this application can be used to record events of heterologous expressed genes or artificial genes. In some embodiments, the gene can include genes associated with a signaling biochemical pathway (e.g., a signaling biochemical pathway-associated gene or polynucleotide). In other embodiments, the genes can be a disease associated gene. As used herein, a “disease-associated” gene refers to any gene which is yielding transcription or translation products at an abnormal level or in an abnormal form in cells derived from a disease-affected tissues compared with tissues or cells of a non-disease control, such as oncogenes or tumor suppressor genes or metastasis suppressor genes. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown and may be at a normal or abnormal level. In other embodiments, molecular events associate with certain genes can be the result of measuring events associated with this gene as a result of small molecule, therapeutic agents, or any other compounds that are intended to elicit a change in cellular function to treat a disease condition.

The cell in which embodiments of the present disclosure are expressed can be any cell. In some embodiments, the cell is a prokaryotic cell. In other embodiments, the cell is a eukaryotic cell, such as without limitation an animal or plant cell. In certain embodiments, the cell is a mammalian cell.

As used herein, the term “eukaryotic cell” may refer to a cell or a plurality of cells derived from a eukaryotic organism. In some embodiments, the eukaryotic cells can be derived from an animal (e.g., primate, rodent, mouse, rat, rabbit, canine, dog, cow, bovine, sheep, ovine, goat, pig, fowl, poultry, chicken, fish, insect, or arthropod). In other embodiments, the eukaryotic cells can be derived from a rodent (e.g., mouse). In still other embodiments, the eukaryotic cells can be non-human eukaryotic cells. In other embodiments, eukaryotic cells can be primary cells or cell lines that are well known to one of ordinary skill in the art. In still other embodiments, eukaryotic cells can be dividing cells (e.g., stem cells) or partially or terminally differentiated cells. In other embodiments, eukaryotic cells may in certain embodiments be disease cells (e.g., tumor cells).

It is understood by one of ordinary skill in the art that embodiments of the constructs disclosed herein are non-naturally occurring and are engineered or exogenous to the cells. Methods for introducing embodiments of the disclosed constructs into a cell can be any method well known to one of ordinary skill in the art.

As used herein, the term “targeting” of a selected DNA sequence or a “target domain” means that a pegRNA is capable of hybridizing with a selected DNA sequence. As uses herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A sequence capable of hybridizing with a given sequence is referred to as the “complement” of the given sequence.

As used herein prime editing RNA (pegRNA) refers to a prime editing system as described in Anzalone, A. V. et. al., Search-and-replace genome editing without double-strand breaks or donor DNA. Nature 576, 149-157 (2019), the contents of which is herein incorporated by reference. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. As recognized by one of ordinary skill in the art, a prime editing system, as exemplified by PE1, PE2, and PE3 can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide.

In some embodiments, the prime editing guide molecule can specify both the target polynucleotide information (e.g., sequence) and contain new polynucleotide information that replaces target polynucleotides. Information transfer from the guide molecule to the target polynucleotide, the PE system can nick the target polynucleotide at a target side to expose a 3′ hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

In some embodiments, the prime editing system can be a PE1 system or variant thereof, a PE2 system or variant thereof, or a PE3 (e.g., PE3, PE3b) system. See e.g., Anzalone et al. 2019. Nature. 576:149-157.

In accordance with the foregoing, in one aspect of the invention, the disclosure provides a nucleic acid construct for recording an iterative nucleic acid editing event. The construct can comprise a first active target domain, comprising an editable recording sequence configured to hybridize with a first prime editing guide RNA (pegRNA) and one or more inactive truncated target domains comprising a non-editable sequence configured to not hybridize with the pegRNA, wherein the first pegRNA edits the first active target domain, wherein the pegRNA edit shifts the position of the recoding sequence from the editable sequence to the non-editable sequence, thereby changing the editable sequence to a non-editable sequence and the inactive truncated target domain to a second active target domain comprising a second recoding sequence configured to hybridize with a second pegRNA.

In another aspect, the disclosure provides a vector comprising a nucleic acid sequence encoding the nucleic construct as described above coupled to a promoter and/or a transcribed form of an RNA molecule.

In another aspect, the disclosure provides a system for recording iterative nucleic acid editing events, the system comprising: the nucleic acid construct above, or a nucleic acid encoding the nucleic acid construct; one or more pegRNAs or one or more nucleic acids encoding the one or more pegRNAs configured to hybridize to a first active target domain; a prime editing enzyme, or a nucleic acid encoding the prime editing enzyme; wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

In another aspect, the disclosure provides a method of iteratively recording editing events, the method comprising: contacting the nucleic acid construct as described above with one or more pegRNAs and a prime editing enzyme; wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

In another aspect, the disclosure provides a method for multiplexed transcription recording, the method comprising: contacting the nucleic acid above with a prime editing guide RNA (pegRNA) expression cassette, a prime editing enzyme, and an endonuclease, wherein the expression cassette comprises a promoter, an endonuclease system comprising a first endonuclease target 5′ to the pegRNA and a second endonuclease target 3′ to the pegRNA, an optional nucleic acid construct encoding a functional GFP and/or an endonuclease, wherein the transcribed region of the nucleic acid construct comprises one or more pegRNAs and expression of one or more pegRNAs is driven by activation of the promoter releasing the one or more pegRNA by cleavage of the endonuclease target by an endonuclease; hybridizing the one or more pegRNAs to a target domain; and editing the target domain by inserting a barcode tag sequence.

In another aspect, the disclosure provides a method for screening transcriptional activity in response to external stimuli, the method comprising using any of the methods described above to record transcription activity of a plurality of DNA sequences in both the absence and presence of external stimuli and comparing the difference between transcriptional activity in both the absence and presence of external stimuli, wherein the difference in transcription activity in the presence of external stimuli can be used as a screening method for regulating therapeutic treatments.

### DNA Typewriter

Embodiments of the nucleic acid construct for recording iterative nucleic acid editing events comprise a tandem array of partial CRISPR-Cas9 target sites all but the first at truncated at their 5′ ends. In some embodiments, the first full CRISPR-Cas9 target site can be the most 5′ unit, wherein the adjacent units in the 5′ to 3′ direction are truncated at their 5′ ends. In other embodiments, the first full CRISPR-Cas9 target site can be the most 3′ unit wherein the adjacent units in the 3′ to 5′ directed are truncated at their 5′ ends. In still other embodiments the tandem array (e.g., TAPE array) comprises at least one monomer. In some embodiments, the TAPE array can comprise two monomers. In other embodiments, the TAPE array can comprise three monomers. In other embodiments, the TAPE array can comprise four monomers. In other embodiments, the TAPE array can comprise five monomers. In other embodiments, the TAPE array can comprise six monomers. In other embodiments, the TAPE array can comprise seven monomers. In other embodiments, the TAPE array can comprise eight monomers. In other embodiments, the TAPE array can comprise nine monomers. In other embodiments, the TAPE array can comprise ten monomers. In still other embodiments, the TAPE array can comprise more than 10 monomers. For example, the TAPE array can comprise 15 or more monomers, 20 or more monomers, or 25 or more monomers. In still other embodiments, the TAPE array can comprise 30, 40, 50, 60, 70, 80, 90, 100 or more monomers.

In still other embodiments, each monomer can comprise one unit, wherein the one unit comprises a full length CRISPR-Cas9 target site. In still other embodiments, each monomer can comprise at least two units, wherein the most 5′ unit comprises a full length CRISPR-Cas9 target site and the second unit comprises a truncated CRISPR-Cas9 target site. In still other embodiments, each monomer can comprise at least two units, wherein the most 3′ unit comprises a full length CRISPR-Cas9 target site and the second unit comprises a truncated CRISPR-Cas9 target site. In still other embodiments, each monomer can comprise at least three units, at least four units, at least five units, at least six units, at least seven units, at least eight units, at least nine unit, or at least 10 units. In still other embodiments, each monomer can comprise 10 or more units, 15 or more units, 20 or more units, 25 or more units, or 30 units. In still other embodiments, each monomer can comprise 30 or more units, 40 or more units, 50 or more units, 60 or more units, 70 or more units, 80 or more units, 90 or more units, or 100 units. In still other embodiments, each monomer can comprise 100 or more units, 150 or more units, 200 or more units, 250 or more units, or 300 units. In still other embodiments, each monomer can comprise 300 or more units, 400 or more units, 500 or more units, 600 or more units, 700 or more units, 800 or more units, 900 or more units, or 1,000 units. In still other embodiments, each monomer can comprise 1,000 or more units.

As used herein, each monomer independent of the number of units comprises 1 full length CRISPR-Cas9 target site and the remaining units within the monomer comprise a truncated CRISPR-Cas9 target site.

As used herein the “first active target domain” or “active target domain” refers to the full length CRISPR-Cas9 target site. The full length CRISPR-Cas9 target site allows for hybridization of the prime editing RNA (pegRNA). As used herein, the “inactive truncated target domain” or “inactive target domain” does not have the full length CRISPR-Cas9 target site, and for this reason, the pegRNA cannot hybridize to the inactive truncated target domain.

In some embodiments, the active target domain is at least 5 nucleotides in length. In other embodiments, the active target domain is at least 10 nucleotides in length. In other embodiments, the active target domain is at least 15 nucleotides in length. In some embodiments, the active target domain is between 15 to 45 nucleotides in length. In some embodiments, the active target domain is 16, 17, 18, 19, or 20 nucleotides in length. In other embodiments, the active target domain is 21, 22, 23, 24, 25, 26, 27, 28, 29, or 30 nucleotides in length. In other embodiments, the active target domain is 31, 32, 33, 34, 35, 36, 37, 38, 39, or 40 nucleotides in length. In other embodiments, the active target domain is 41, 42, 43, 44, or 45 nucleotides in length. In still other embodiments, the active target domain is 45 or more nucleotides in length, 50 or more nucleotides in length, 60 or more nucleotides in length, 70 or more nucleotides in length, 80 or more nucleotides in length, 90 or more nucleotides in length, or 100 nucleotides in length.

In some embodiments, the TAPE monomer does not comprise an inactive truncated target domain. In other embodiments, the inactive truncated target domain is between 1 to 45 nucleotides in length. In other embodiments, the inactive truncated target domain is at least 2 nucleotides in length. In other embodiments, the inactive truncated target domain is at least 3 nucleotides in length. In other embodiments, the inactive truncated target domain is at least 4 nucleotides in length. In other embodiments, the inactive truncated target domain is at least 5 nucleotides in length. In other embodiments, the inactive truncated target domain is at least 6, 7, 8, 9, or 10 nucleotides in length. In still other embodiments, the inactive truncated target domain is at least 11, 12, 13, 14, 15, 16, 17, 18, 19, or 20 nucleotides in length. In some embodiments, the inactive truncated target domain is at least 21, 22, 23, 24, 25, 26, 27, 28, 29, or 30 nucleotides in length. In some embodiments, the inactive truncated target domain is at least 31, 32, 33, 34, 35, 36, 37, 38, 39, or 40 nucleotides in length. In some embodiments, the inactive truncated target domain is at least 41, 42, 43, 44, 45, 46, 47, 48, 49, or 50 nucleotides in length. In still other embodiment, the inactive truncated target domain is at least 50 or more nucleotides in length, 60 or more nucleotides in length, 70 or more nucleotides in length, 80 or more nucleotides in length, 90 or more nucleotides in length, or 100. In still other embodiments, the inactive truncated target domain is at least 100 nucleotides in length.

In some embodiments, the first active target domain comprises from 5′ to 3′ a full length CRISPR-Cas9 target site, a protospacer adjacent motif (PAM) sequence, and a homology sequence. In some embodiments, the first active target domain comprises from 3′ to 5′ 3′ a full length CRISPR-Cas9 target site, a PAM sequence, and a homology sequence. In still other embodiments, a second or subsequent (e.g., third, fourth, or fifth) active target domain comprises from 5′ to 3′ a full length CRISPR-Cas9 target site, a PAM sequence, and a homology sequence. In still other embodiments, a second or subsequent (e.g., third, fourth, or fifth) active target domain comprises from 5′ to 3′ a full length CRISPR-Cas9 target site, a PAM sequence, and a homology sequence.

In some embodiments, the first inactive truncated target domain comprises from 5′ to 3′ a truncated CRISPR-Cas9 target site, a protospacer adjacent motif (PAM) sequence, and a homology sequence, wherein the pegRNA/PE2 edit inserts 5′ to the truncated CRISPR-Cas9 target site a sequence comprising from 5′ to 3′ the barcode tag sequence and the target activation sequence. In other embodiments, the first inactive truncated target domain comprises from 3′ to 5′ a truncated CRISPR-Cas9 target site, a protospacer adjacent motif (PAM) sequence, and a homology sequence, wherein the pegRNA/PE2 edit inserts 3′ to the truncated CRISPR-Cas9 target site a sequence comprising from 5′ to 3′ the target activation sequence and the barcode tag sequence.

As used herein, “shifts” or “shifting” the position of the recoding sequence refers to the pegRNA hybridizing to the target active domain, and the pegRNA/PE2-mediated insertion of a second sequence at the target active domain. The pegRNA/PE2-mediated insertion of a second sequence at the target active domain inactivates the current target active domain by disrupting its sequence and activates the adjacent inactive domain by extending the partial (truncated) CRISPR-Cas9 target site. This iterative process (i.e., inactivating the current active target domain and activating the adjacent inactive truncated target domain) occurs in sequential order along each unit of the monomer. In some embodiments, the 5′ most active target domain is shifted in the 5′ to 3′ direction following the process described above. In other embodiments, the 3′ most active target domain is shifted in the 3′ to 5′ direction following the process described above. As described herein, following inactivation of an active target domain, a second pegRNA cannot hybridize to that target domain. For example, a pegRNA can only hybridize to the active target domain.

In some embodiments, the active target domain is shifted by one unit. In still other embodiments, the active target domain is shifted to the adjacent unit in either the 5′ to 3′ direction or in the 3′ to 5′ direction.

As used herein, the pegRNA/PE2 “edit” refers to the insertion of a sequence that comprises a target activation sequence and a barcode tag sequence. In other embodiments, the pegRNA/PE2 edit refers to the insertion of a sequence that comprises a target activation sequence. In still other embodiments, the pegRNA/PE2 edit refers to the insertion of a sequence that comprises a barcode tag sequence.

In some embodiments, the pegRNA/PE2 edit can be mediated through the same pegRNA, such that each unit within the monomer is edited by the same pegRNA. In other embodiments, the pegRNA/PE2 edit can be mediated through a different pegRNA, such that each unit within the monomer is edited by a different pegRNA. In still other embodiments, the pegRNA/PE2 edit can be mediated through two or more different pegRNAs, such that each unit within the monomer is edited in an alternating manner. For example, a first unit is edited by a first pegRNA; a second unit is edited by a second pegRNA; and a third unit is edited by the first pegRNA. The alternating pattern of edits can be determined by one of ordinary skill in the art. In still other embodiments, the pegRNA/PE2 edit can be mediated through three or more, four or more, five or more, six or more, seven or more, eight or more, nine or more, or 10 different pegRNAs. In still other embodiments, the pegRNA/PE2 edit can be mediated through 10 or more, 15 or more 20 or more 25 or more, or 30 different pegRNAs. In still other embodiments, the pegRNA/PE2 edit can be mediated through 30 or more, 40 or more, 50 or more, 60 or more, 70 or more, 80 or more, 90 or more, or 100 different pegRNAs. In still other embodiments, the pegRNA/PE2 edit can be mediated through 100 or more different pegRNAs.

In some embodiments, the pegRNA/PE2 edit inserts a sequence 5′ to the inactive truncated target domain. In other embodiments, the pegRNA/PE2 edit inserts a sequence 3′ to the inactive truncated target domain. In some embodiments, the sequence inserted by the pegRNA/PE2 comprises from 5′ to 3′ a barcode sequence tag and a target activation sequence, wherein the target activation sequence extends the 5′ portion of the inactive truncated target domain. In other embodiments, the sequence inserted by the pegRNA/PE2 comprises from 5′ to 3′ a target activation sequence and a barcode sequence tag, wherein the target activation sequence extends the 3′ portion of the inactive truncated target domain.

In still other embodiments, the pegRNA inserts a unique barcode tag sequence, wherein the unique barcode tag sequence can be used to identify each pegRNA. In other embodiments, the pegRNA inserts the same barcode tag sequence. In still other embodiments, the pegRNA can insert 2 or more different barcode tag sequences, 3 or more different barcode tag sequences, 4 or more different barcode tag sequences, 5 or more different barcode tag sequences, 6 or more different barcode tag sequences, 7 or more different barcode tag sequences, 8 or more different barcode tag sequences, 9 or more different barcode tag sequences, or 10 different barcodes in an alternating manner. In still other embodiments, the pegRNA can insert 10 or more different barcode tag sequences, 20 or more different barcode tag sequences, 30 or more different barcode tag sequences, 40 or more different barcode tag sequences, or 50 different barcode tag sequences in an alternating manner. In still other embodiments, the pegRNA can insert 50 or more different barcode tag sequences in an alternating manner.

In some embodiments, the pegRNA can insert a constant (i.e., same sequence) activation target sequence at each active target domain. In other embodiments, the pegRNA can insert a unique activation target sequence at each active target domain. In still other embodiments, the pegRNA can insert 2 or more different activation target sequences, 3 or more different activation target sequences, 4 or more different activation target sequences, 5 or more different activation target sequences, 6 or more different activation target sequences, 7 or more different activation target sequences, 8 or more different activation target sequences, 9 or more different activation target sequences, or 10 different activation target sequences in an alternating manner. In still other embodiments, the pegRNA can insert 10 or more different activation target sequences, 20 or more different activation target sequences, 30 or more different activation target sequences, 40 or more different activation target sequences, or 50 different activation target sequences in an alternating manner. In still other embodiments, the pegRNA can insert 50 or more different activation target sequences in an alternating manner.

In some embodiments, the pegRNA can additionally insert a homology sequence to correct insertion errors.

### Enhancer-Driven Genomic Recording of Transcriptional Activity in Multiplex (ENGRAM)

In another aspect, the disclosure provides an expression cassette comprising a cis-regulatory-element (CRE) coupled promoter sequence and a nucleic acid sequence encoding from 5′ to 3′ a first endonuclease target, one or more prime editing guide RNAs (pegRNA), and a second endonuclease target, wherein the nucleic acid sequence is operably linked to the CRE coupled promoter sequence, and wherein cleavage of the first endonuclease target and the second endonuclease target releases the one or more pegRNAs causing the one or more pegRNAs to hybridize to a nucleic acid target and edit the nucleic acid target by inserting a barcode tag sequence.

In another aspect, the disclosure provides a method for multiplex transcriptional recording, the method comprising: coupling a cis-regulatory element (CRE) coupled promoter sequence to a nucleic acid sequence encoding from 5′ to 3′ a first endonuclease target, one or more prime editing guide RNAs (pegRNAs), and a second endonuclease target, releasing the one or more pegRNAs from a transcript by the addition of an endonuclease; and editing of a target nucleic acid sequence by inserting a barcode tag sequence.

Embodiments of these aspects can include a DNA transcriptional recorder referred to as Enhancer-driven Genomic Recording of transcriptional Activity in Multiplex (ENGRAM). Embodiments of ENGRAM can include a construct or a method for multiplex transcriptional recording. As further described in Example 2, ENGRAM can use enzymatic release of a prime editing guide RNA (pegRNA) from a synthetic transcript driven by cis-regulatory-element (CRE) coupled Pol-II promoters, wherein each pegRNA programs insertion of a specific barcode to a genomically-encoded recording locus. In some embodiments, the genomically-encoded recording locus can be any DNA tape. In other embodiments, the genomically-encoded recording locus can be the DNA Typewriter described in Example 1.

In some embodiments, the construct comprises an enhancer positioned upstream of a minimal promoter, wherein the enhancer and minimal promoter are coupled to the expression of a library of writing units. In some embodiments, the construct comprises an enhancer positioned upstream of a minimal promoter, wherein the enhancer is coupled to the expression of a library of writing units. In still other embodiments, the construct comprises an enhancer position upstream of a minimal promoter, wherein the minimal promoter is coupled to the expression of a library of writing units.

In some embodiments, the enhancer positioned upstream of a minimal promoter is a natural enhancer. In still other embodiments, the enhancer positioned upstream of a minimal promoter is a synthetic enhancer. The term, “enhancer”, is used in a manner that is consistent with its meaning as understood by one of ordinary skill in the art. For example, an enhancer can refer to short regulatory elements of accessible that DNA that help establish the transcriptional program of cells by increasing transcription of target genes.

In some embodiments, the methods for using enhancers, can include but are not limited to enhancers and techniques of using enhancers that are well known to one of ordinary skill in the art. See e.g., (Klein, J. C. et al., A systematic evaluation of the design and context dependencies of massively parallel reporter assays. Nat. Methods 17, 1083-1091 (2020), which is herein incorporated by reference). In still other embodiments, the enhancer can be a signal-responsive regulatory element. In some embodiments, the signal-responsive regulatory element can be Tet Response Element (TRE; activated by doxycycline) (Gossen, M. et al. Transcriptional Activation by Tetracyclines in Mammalian Cells. Science vol. 268 1766-1769 (1995). In some embodiments, the signal-responsive regulatory element can be a NF-κB responsive element (activated by TNFα) (Zabel, U., Schreck, R. & Baeuerle, P. A. DNA binding of purified transcription factor NF-kappa B. Affinity, specificity, Zn2 dependence, and differential half-site recognition. Journal of Biological Chemistry vol. 266 252-260 (1991). In some embodiments, the signal-responsive regulatory element can be a TCF-LEF responsive element (Wnt signaling pathway; activated by CHIR99021) (pGL4.49 [luc2P/TCF-LEF/Hygro] Vector Protocol. Promega website).

As used herein, the term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

In some embodiments, the promoter can include any promoter well known to one of ordinary skill in the art. In other embodiments, the promoter can be a minimal promoter (minP). In still other embodiments, the promoter is a constitutive promoter. In other embodiments, the promoter is a signal specific inducible promoter. In still other embodiments, the enhancer coupled to the promoter together as a unit can function as a constitutive promoter. In some embodiments, the enhancer coupled to the promoter together as a unit can function as a signal specific inducible promoter.

As used herein the term “writing unit” refers to any gene editing technology well known to one of ordinary skill in the art. For example, a writing unit can include but is not limited to a prime editing guide RNA (pegRNA).

Embodiments of ENGRAM depend on CRE-minP-driven reporter transcripts, which are made by RNA polymerase II (Pol-2). Guide RNAs are made by RNA polymerase III (Pol-3). As described further in Example 2, embodiments of this construct use CRISPR endoribonuclease Csy4 (i.e., Cas6f), which can recognize and cut at the 3′ end of 17-bp RNA hairpins (csy4). As such, expression of Csy4, together with CRE-activity-dependent expression of csy4-pegRNA-csy4 can result in a liberated functional pegRNA. In some embodiments, the csy4-pegRNA-csy4 is embedded within the 3′ untranslated region of a GFP transcript. In some embodiments, the csy4-pegRNA-csy4 is embedded within the 5′ untranslated region of a GFP transcript. In still other embodiments, the csy4-pegRNA-csy4 is embedded within the 3′ and 5′ untranslated region of a GFP transcript.

In some embodiments, Csy4 is constitutively expressed. In other embodiments, PE2 is constitutively expressed. In still other embodiments, both Cys4 and PE2 are constitutively expressed. In other embodiments, PE2 is constitutively expressed and expression of Cys4 is driven by the promoter.

In some embodiments, the pegRNA programs insertion of a signal specific barcode tag sequence to a genomically-encoded recording locus of interest. In some embodiments, the genomically-encoded recording locus of interest can be any encoded DNA Tape. In some embodiments, the DNA Tape is DNA Typewriter as described in Example 1.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); Ran, F. A., et al., Genome engineering using the CRISPR-Cas9 system, Nature Protocols, 8:2281-2308 (2013), and Jiang, F. and Doudna, J. A., CRISPR-Cas9 Structures and Mechanisms, Annual Review of Biophysics, 46:505-529 (2017) for definitions and terms of art.

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casnl nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species.

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (me) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA.

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. Conversely, a first element is downstream of a second element in a nucleic acid molecule where the first element is positioned somewhere that is 3′ to the second element.

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpfl (a type-V CRISPR-Cas systems), C2c1 (a type V CRISPR-Cas system), C2c2 (a type VI CRISPR-Cas system) and C2c3 (a type V CRISPR-Cas system). As used herein, the “guide RNA” may also be referred to as a “traditional guide RNA” to contrast it with the modified forms of guide RNA termed “prime editing guide RNAs” (or “pegRNAs”) which have been invented for the prime editing methods and composition disclosed herein.

The term “homology arm” refers to a portion of the extension arm that encodes a portion of the resulting reverse transcriptase-encoded single strand DNA flap that is to be integrated into the target DNA site by replacing the endogenous strand. The portion of the single strand DNA flap encoded by the homology arm is complementary to the non-edited strand of the target DNA sequence, which facilitates the displacement of the endogenous strand and annealing of the single strand DNA flap in its place, thereby installing the edit. The homology arm is part of the DNA synthesis template since it is by definition encoded by the polymerase of the prime editors described herein.

As used herein, the term “polymerase” refers to an enzyme that synthesizes a nucleotide strand. The polymerase can be a “template-dependent” polymerase (i.e., a polymerase which synthesizes a nucleotide strand based on the order of nucleotide bases of a template strand). The polymerase can also be a “template-independent” polymerase (i.e., a polymerase which synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a pegRNA, wherein the extension arm comprises a strand of DNA. In such cases, the pegRNA may be referred to as a chimeric or hybrid pegRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the pegRNA is RNA, i.e., including an RNA extension.

The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a pegRNA) and will proceed toward the 5′ end of the template strand. A “DNA polymerase” catalyzes the polymerization of deoxynucleotides.

As used herein, the term “protospacer” refers to the sequence (−20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target. Protospacer adjacent motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

As used herein, the term “spacer sequence” in connection with a guide RNA or a pegRNA refers to the portion of the guide RNA or pegRNA of about 20 nucleotides which contains a nucleotide sequence that is complementary to the protospacer sequence in the target DNA sequence. The spacer sequence anneals to the protospacer sequence to form a ssRNA/ssDNA hybrid structure at the target site and a corresponding R loop ssDNA structure of the endogenous DNA strand that is complementary to the protospacer sequence.

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Embodiment 1. A nucleic acid construct for recording an iterative nucleic acid editing event, the construct comprising a first active target domain, comprising an editable recording sequence configured to hybridize with a first prime editing guide RNA (pegRNA) and one or more inactive truncated target domains comprising a non-editable sequence configured to not hybridize with the pegRNA, wherein the first pegRNA edits the first active target domain, wherein the pegRNA edit shifts the position of the recoding sequence from the editable sequence to the non-editable sequence, thereby changing the editable sequence to a non-editable sequence and the inactive truncated target domain to a second active target domain comprising a second recoding sequence configured to hybridize with a second pegRNA.

Embodiment 2. The nucleic acid construct of embodiment 1, wherein the pegRNA edit inactivates the first active domain preventing a second hybridization with a second pegRNA and extends the truncated target domain, thereby activating this domain and allowing hybridization with a second pegRNA.

Embodiment 3. The nucleic acid construct of embodiment 2, wherein the pegRNA edit comprises the insertion of a sequence comprising from 5′ to 3′ a barcode tag sequence and a target activation sequence.

Embodiment 4. The nucleic acid construct of embodiment 3, wherein the barcode tag sequence uniquely identifies each pegRNA and each active target domain is programmed by a different pegRNA, thereby each active target domain includes a different barcode tag sequence.

Embodiment 5. The nucleic acid construct of embodiment 3, wherein the barcode tag sequence is constant for each pegRNA and each active target domain is programmed by the same pegRNA, thereby each active target domain includes the same barcode tag sequence.

Embodiment 6. The nucleic acid construct of embodiment 3, wherein the barcode tag sequence is designed to allow 2, 3, or more unique pegRNAs to alternatively target each activation target domain, thereby every alternating active domain or every 2, 3, or more alternative active domains include the same barcode tag sequence.

Embodiment 7. The nucleic acid construct of embodiment 3, wherein the target activation sequence extends the inactive truncated target domain.

Embodiment 8. The nucleic acid construct of embodiments 1-7, comprising 1, 5, 10, 15, 20, 25, 30, 35, 40, 45, 50, 60, 70, 80, 90, 100, 125, 150, 175, 200, or more truncated target domains adjacent to the first active target domain.

Embodiment 9. The nucleic acid construct of embodiment 8, wherein each truncated target domain comprises 1, 5, 10, 15, 20, 25, 30, 35, 40, 45, 50, 60, 70, 80, 90, 100, 125, 150, 175, 200, or more units.

Embodiment 10. The nucleic acid construct of embodiments 1-9, wherein the pegRNA additionally inserts a homology sequence to correct insertion errors.

Embodiment 11. The nucleic acid construct of embodiments 1-10, wherein the active target domain is 15-45 nucleotides in length and the inactive truncated target domain is 0-45 nucleotides in length.

Embodiment 12. The nucleic acid construct of embodiments 1-11, wherein the first active target domain comprises from 5′ to 3′ a full length CRISPR-Cas9 target site, a protospacer adjacent motif (PAM) sequence, and a homology sequence.

Embodiment 13. The nucleic acid construct of embodiments 1-12, wherein the inactive truncated target domain comprises a truncated CRISPR-Cas9 target site, a protospacer adjacent motif (PAM) sequence, and a homology sequence, wherein the pegRNA edit inserts 5′ to the truncated CRISP-Cas9 target site a sequence comprising from 5′ to 3′ the barcode tag sequence and the target activation sequence, wherein the target activation sequence extends the truncated CRISPR-Cas9 target site.

Embodiment 14. The nucleic acid construct of embodiments 1-13, wherein the nucleic acid construct is a double stranded DNA.

Embodiment 15. A vector comprising a nucleic acid sequence encoding the nucleic acid construct of embodiments 1-14 coupled to a promoter and/or a transcribed form of a RNA molecule.

Embodiment 16. A cell comprising the nucleic acid construct of any one of embodiments 1-14 or the vector of embodiment 15.

Embodiment 17. The cell of embodiment 16, further comprising one or more nucleic acids encoding one or more pegRNAs.

Embodiment 18. The cell of embodiment 16 or embodiment 17, further comprising a nucleic acid encoding a prime editing enzyme.

Embodiment 19. The cell of embodiments 16-18, wherein the prime editing enzyme comprises a nickase enzyme operatively associated with a reverse-transcriptase enzyme.

Embodiment 20. A system for recording iterative nucleic acid editing events, the system comprising: the nucleic acid construct recited in any one of embodiments 1-14, or a nucleic acid encoding the nucleic acid construct; one or more pegRNAs or one or more nucleic acids encoding the one or more pegRNAs configured to hybridize to a first active target domain; a prime editing enzyme, or a nucleic acid encoding the prime editing enzyme; wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

Embodiment 21. The system of embodiment 20, wherein the system is a cell.

Embodiment 22. A method of iteratively recording editing events, the method comprising: contacting the nucleic acid construct recited in any one of embodiments 1-14 with one or more pegRNAs and a prime editing enzyme; wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

Embodiment 23. The method of embodiment 22, wherein the barcode tag sequence uniquely identifies each pegRNA and each active target domain is programmed by a different pegRNA, thereby each active target domain includes a different barcode tag sequence.

Embodiment 24. The method of embodiments 22 and 23, wherein the barcode tag sequence is constant for each pegRNA and each active target domain is programmed by the same pegRNA, thereby each active target domain includes the same barcode tag sequence.

Embodiment 25. The method of embodiments 22-24, wherein the barcode tag sequence is designed to allow 2, 3, or more unique pegRNAs to alternatively target each activation target domain, thereby every alternating active domain or every 2, 3, or more alternative active domains include the same barcode tag sequence.

Embodiment 26. The method of embodiments 22-25, wherein the one or more pegRNAs edit the active target domain with a sequence from 5′ to 3′ the target activation sequence and the barcode tag sequence, wherein each sequence inserts by the pegRNAs comprise the same target activation sequence and a different barcode tag sequence.

Embodiment 27. The method of embodiments 22-26, wherein the method further comprises sequencing the nucleic acid construct following iterative editing.

Embodiment 28. A method for multiplexed transcription recording, the method comprising: contacting the nucleic acid construct recited in embodiments 1-14 with a prime editing guide RNA (pegRNA) expression cassette, a prime editing enzyme, and an endonuclease, wherein the expression cassette comprises a promoter, an endonuclease system comprising a first endonuclease target 5′ to the pegRNA and a second endonuclease target 3′ to the pegRNA, an optional nucleic acid construct encoding a functional GFP and/or an endonuclease, wherein the transcribed region of the nucleic acid construct comprises one or more pegRNAs and expression of one or more pegRNAs is driven by activation of the promoter releasing the one or more pegRNA by cleavage of the endonuclease target by an endonuclease; hybridizing the one or more pegRNAs to a target domain; and editing the target domain by inserting a barcode tag sequence.

Embodiment 29. An expression cassette comprising a cis-regulatory-element (CRE) coupled promoter sequence and a nucleic acid sequence encoding from 5′ to 3′ a first endonuclease target, one or more prime editing guide RNAs (pegRNA), and a second endonuclease target, wherein the nucleic acid sequence is operably linked to the CRE coupled promoter sequence, and wherein cleavage of the first endonuclease target and the second endonuclease target releases the one or more pegRNAs causing the one or more pegRNAs to hybridize to a nucleic acid target and edit the nucleic acid target by inserting a barcode tag sequence.

Embodiment 30. A method for multiplex transcriptional recording, the method comprising coupling a cis-regulatory element (CRE) coupled promoter sequence to a nucleic acid sequence encoding from 5′ to 3′ a first endonuclease target, one or more prime editing guide RNAs (pegRNAs), and a second endonuclease target, releasing the one or more pegRNAs from a transcript by the addition of an endonuclease; and editing of a target nucleic acid sequence by inserting a barcode tag sequence.

Embodiment 31. A method for multiplexed transcriptional recording, comprising contacting a nucleic acid construct with a pegRNA expression cassette, a prime editing enzyme, and an endonuclease, or a protein with endonuclease domain and an optional nucleic construct.

Embodiment 32. The method of embodiment 31, wherein the expression cassette comprising an enhancer and/or promoter for transcription and an endonuclease system, the endonuclease system comprising a sequence specific endonuclease that has target domains flanking the pegRNA, and an endonuclease.

Embodiment 33. The method of embodiments 31 and 32, wherein the optional nucleic acid construct encodes a functional GFP and/or an endonuclease, and wherein the transcribed region of the nucleic acid construct comprises one or more pegRNAs.

Embodiment 34. The method of embodiments 31-33, wherein the 5′ and 3′ ends of the pegRNAs are attached to the sequence specific endonuclease target.

Embodiment 35. The method of embodiments 31-34, wherein the expression of one or more pegRNAs is driven by activation of the enhancer and/or promoter.

Embodiment 36. The method of embodiments 31-35, wherein the release of the one or more pegRNAs from the transcript is driven by the cleavage of the sequence specific endonuclease target, wherein the one or more pegRNAs are configured to hybridize to a DNA target domain.

Embodiments 37. The method of embodiments 31-36, where the DNA target domain comprises the nucleic acid construct recited in embodiments 1-14.

Embodiment 38. The method of embodiments 31-37, wherein the one or more pegRNAs insert a barcode tag sequence in the DNA target domain.

Embodiment 39. The method of embodiments 31-38, wherein the enhancer and/or promoter pair is a constitutive promoter or a signal specific inducible promoter.

Embodiment 40. The method of embodiments 31-39, wherein the sequence-specific endonuclease target is selected from the group comprising a cys4 hairpin sequence, a tRNA sequence, a self-cleaving ribozyme, a customized sequence for site-specific RNA endonuclease, and the like, wherein the endonuclease target sequence is placed 5′ and/or 3′ to the pegRNA sequence.

Embodiment 41. The method of embodiments 31-40, wherein the prime editing enzyme is constitutively expressed, inducibly expressed, or transiently expressed.

Embodiment 42. The method of embodiments 31-41, wherein the sequence-specific endonuclease is constitutively expressed, inducibly expressed, or transiently expressed, and wherein the endonuclease expression is coupled with all or a subset of pegRNAs.

Embodiment 43. A system for multiplexed transcriptional recording, comprising a pegRNA expression cassette, a prime editing enzyme, and an endonuclease, or a protein with endonuclease domain and an optional nucleic construct.

Embodiment 44. The system of embodiment 43, wherein the expression cassette comprising an enhancer and/or promoter for transcription and an endonuclease system, the endonuclease system comprising a sequence specific endonuclease that has target domains flanking the pegRNA, and an endonuclease.

Embodiment 45. The system of embodiments 43 and 44, wherein the optional nucleic acid construct encodes a functional GFP and/or an endonuclease, and wherein the transcribed region of the nucleic acid construct comprises one or more pegRNAs.

Embodiment 46. The system of embodiments 43-45, wherein the 5′ and 3′ ends of the pegRNAs are attached to the sequence specific endonuclease target.

Embodiment 47. The system of embodiments 43-46, wherein the expression of one or more pegRNAs is driven by activation of the enhancer and/or promoter.

Embodiment 48. The system of embodiments 43-47, wherein the release of the one or more pegRNAs from the transcript is driven by the cleavage of the sequence specific endonuclease target, wherein the one or more pegRNAs are configured to hybridize to a DNA target domain.

Embodiment 49. The system of embodiments 43-48, where the DNA target domain comprises the nucleic acid construct recited in embodiments 1-14.

Embodiment 50. The system of embodiments 43-49, wherein the one or more pegRNAs insert a barcode tag sequence in the DNA target domain.

Embodiment 51. The system of embodiments 43-50, wherein the enhancer and/or promoter pair is a constitutive promoter or a signal specific inducible promoter.

Embodiment 52. The system of embodiments 43-51, wherein the sequence-specific endonuclease target is selected from the group comprising a cys4 hairpin sequence, a tRNA sequence, a self-cleaving ribozyme, a customized sequence for site-specific RNA endonuclease, and the like, wherein the endonuclease target sequence is placed 5′ and/or 3′ to the pegRNA sequence.

Embodiment 53. The system of embodiments 43-52, wherein the prime editing enzyme is constitutively expressed, inducibly expressed, or transiently expressed.

Embodiment 54. The system of embodiments 43-53, wherein the sequence-specific endonuclease is constitutively expressed, inducibly expressed, or transiently expressed, and wherein the endonuclease expression is coupled with all or a subset of pegRNAs.

Embodiment 55. The system of embodiments 43-54, wherein the system is in a cell.

Embodiment 56. A method for iterative transcriptional recording, the method comprising contacting the nucleic acid construct recited in embodiments 1-14 with the method for multiplexed transcriptional recording recited in embodiments 31-42.

Embodiment 57. A method for screening new cis-regulatory elements (CREs), the method comprising contacting the nucleic acid construct recited in embodiments 1-14 with a pegRNA expression cassette, a prime editing enzyme, and an endonuclease, or a protein with endonuclease domain and an optional nucleic construct.

Embodiment 58. The method of embodiment 57, wherein the expression cassette comprising an enhancer and/or promoter for transcription and an endonuclease system, the endonuclease system comprising a sequence specific endonuclease that has target domains flanking the pegRNA, and an endonuclease.

Embodiment 59. The method of embodiments 57 and 58, wherein the optional nucleic acid construct encodes a functional GFP and/or an endonuclease, and wherein the transcribed region of the nucleic acid construct comprises one or more pegRNAs.

Embodiment 60. The method of embodiments 57-59, wherein the 5′ and 3′ ends of the pegRNAs are attached to the sequence specific endonuclease target.

Embodiment 61. The method of embodiments 57-60, wherein the expression of one or more pegRNAs is driven by activation of the enhancer and/or promoter.

Embodiment 62. The method of embodiments 57-61, wherein the release of the one or more pegRNAs from the transcript is driven by the cleavage of the sequence specific endonuclease target, wherein the one or more pegRNAs are configured to hybridize to a DNA target domain.

Embodiments 63. The method of embodiments 57-62, where the DNA target domain comprises the nucleic acid construct recited in embodiments 1-14.

Embodiment 64. The method of embodiments 57-63, wherein the one or more pegRNAs inserts an insertion sequence, wherein the insertion sequence activates a selection marker downstream of the target domain.

Embodiment 65. The method of embodiments 57-64, wherein the selection marker is an antibiotic resistant protein, a fluorescent protein, a cell surface protein, a functional protein that enriches the target domain with one or more nucleic acid sequence insertions.

Embodiment 66. A method for screening transcriptional activity in response to external stimuli, the method comprising using any of embodiments 1-65 to record transcription activity of a plurality of DNA sequences in both the absence and presence of external stimuli and comparing the difference between transcriptional activity in both the absence and presence of external stimuli, wherein the difference in transcription activity in the presence of external stimuli can be used as a screening method for regulating therapeutic treatments.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

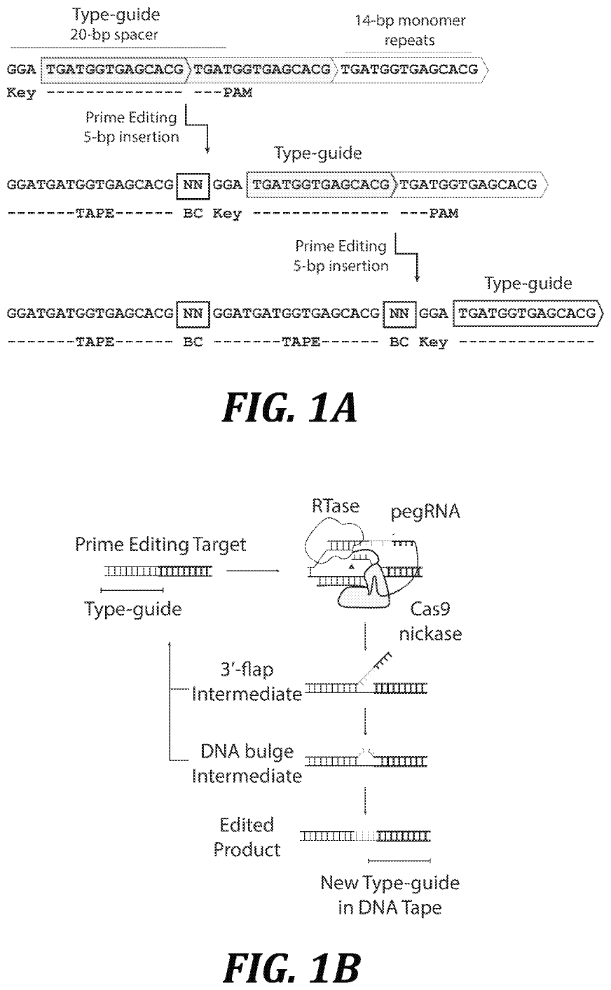

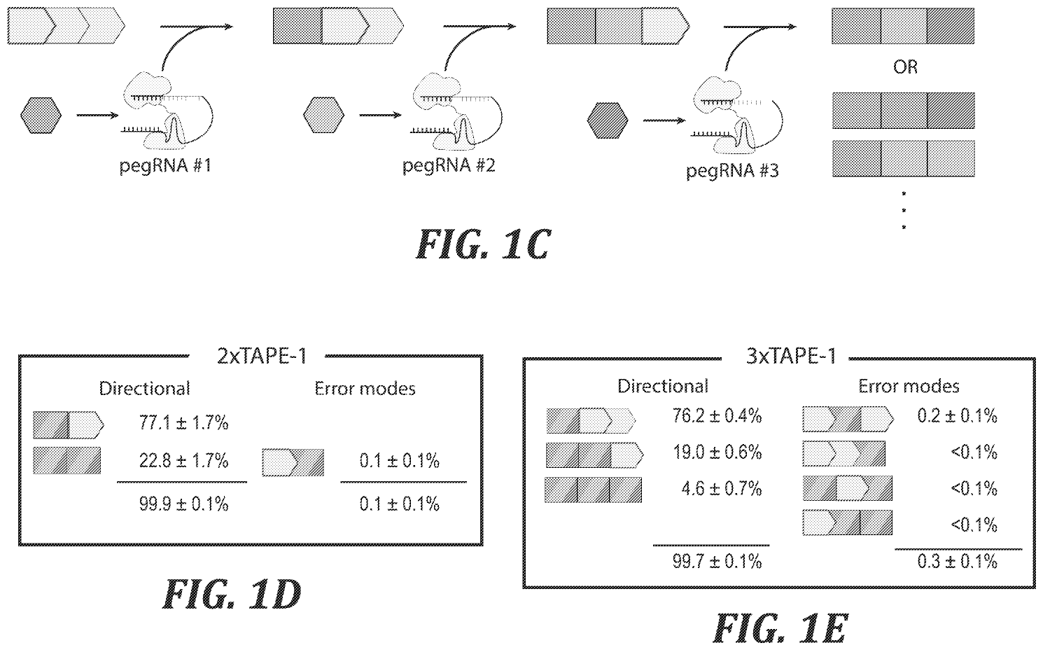

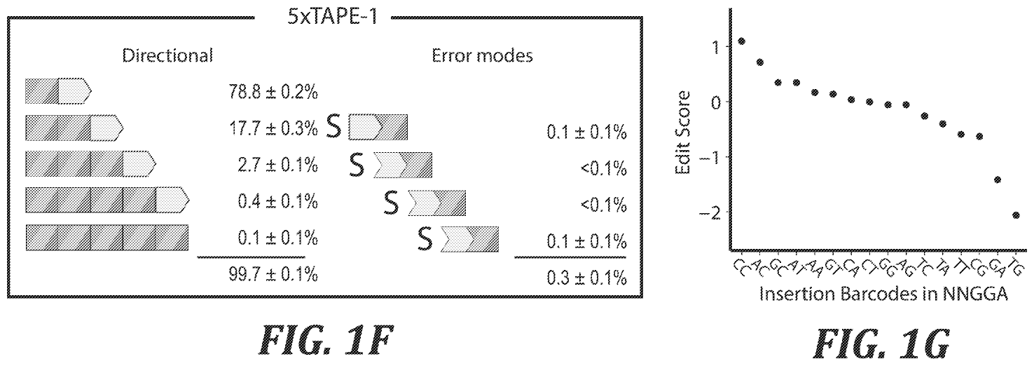

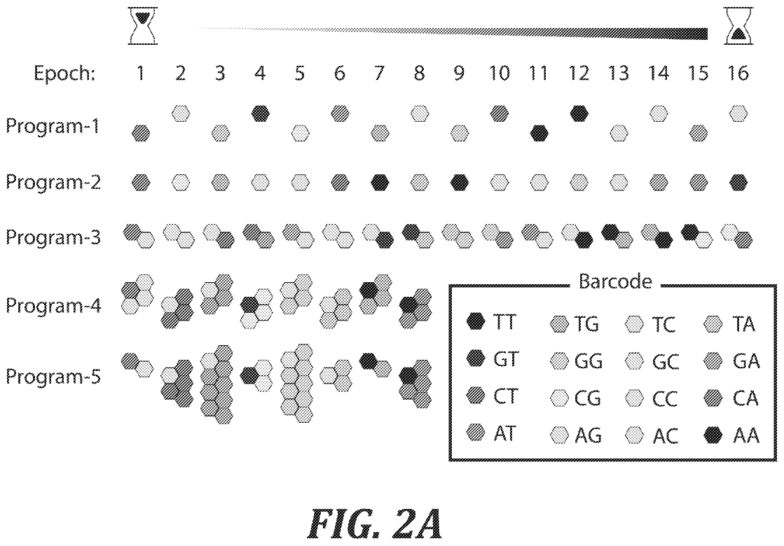

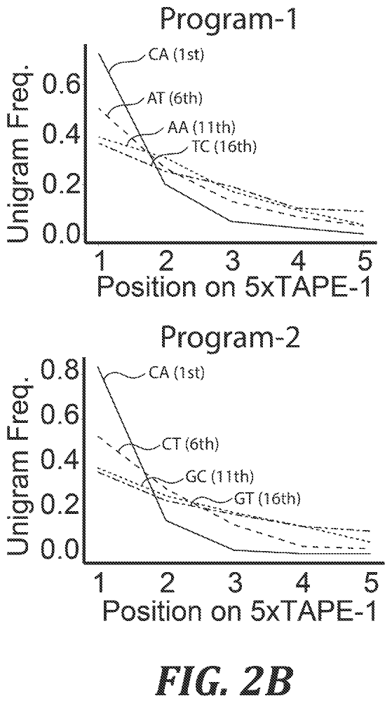

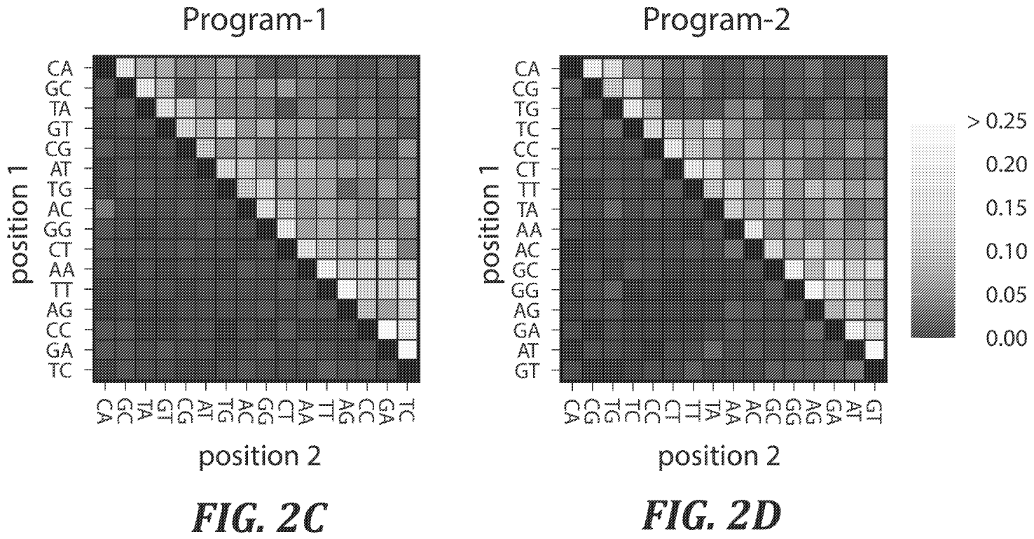

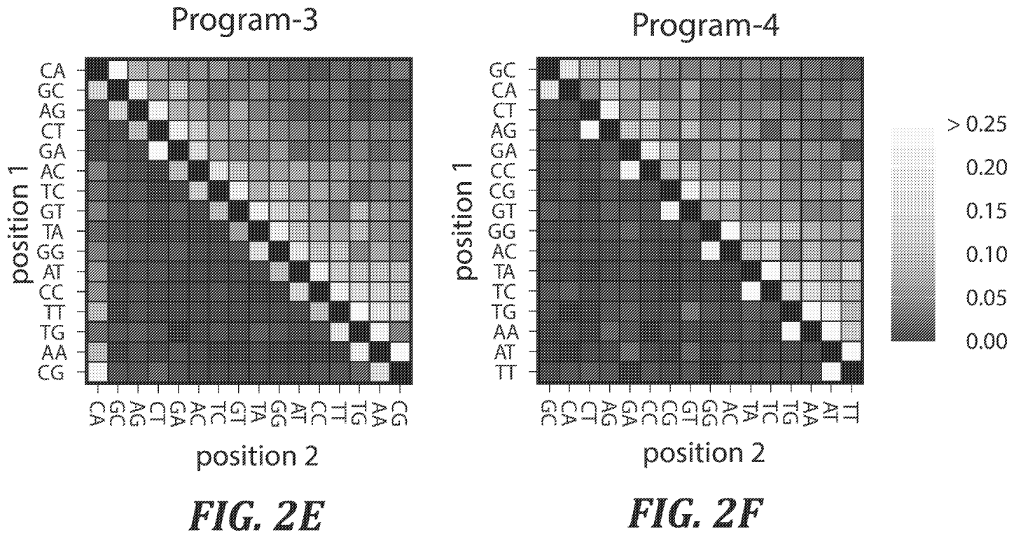

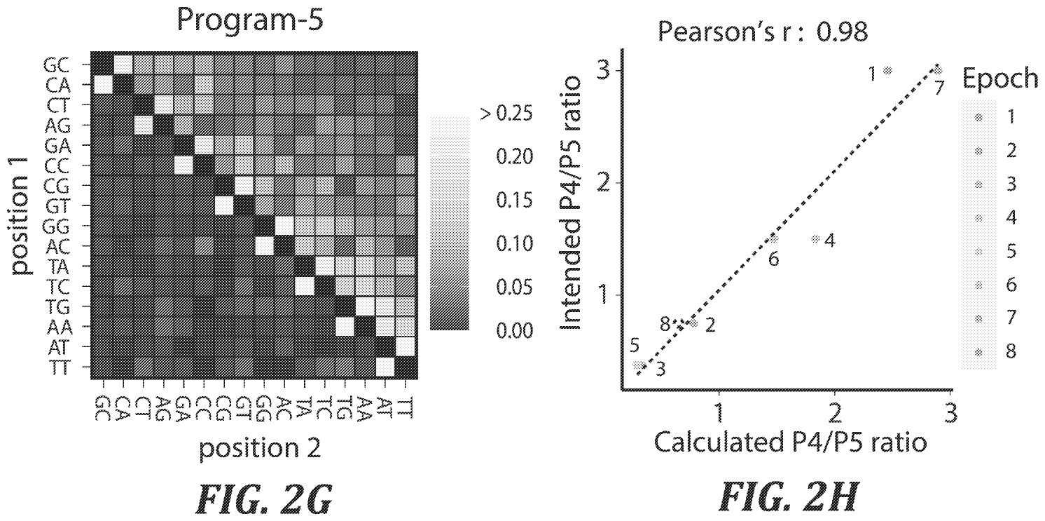

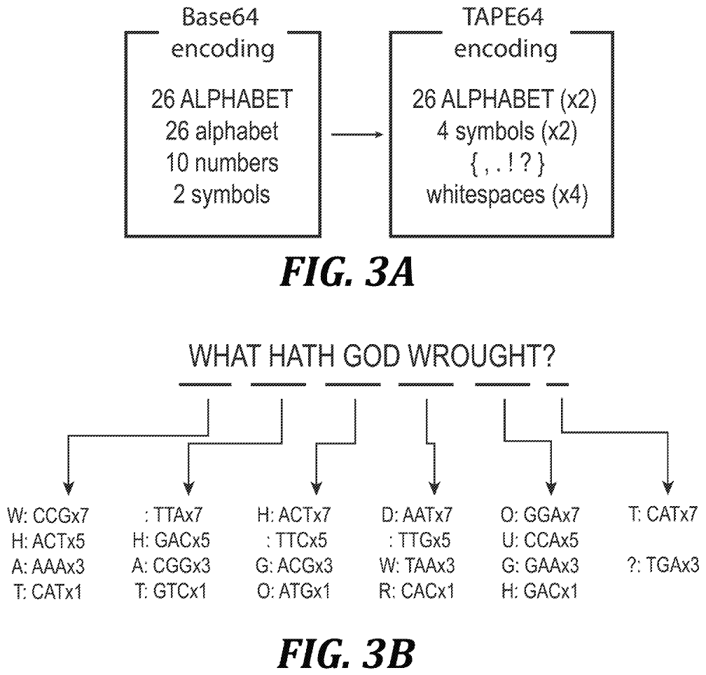

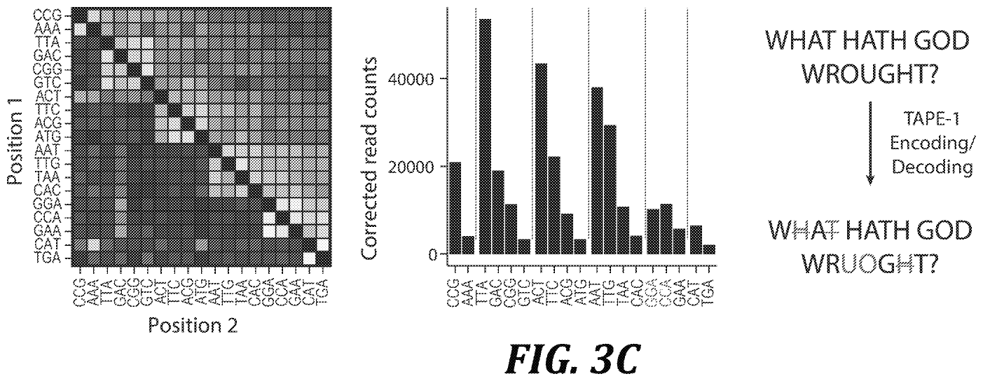

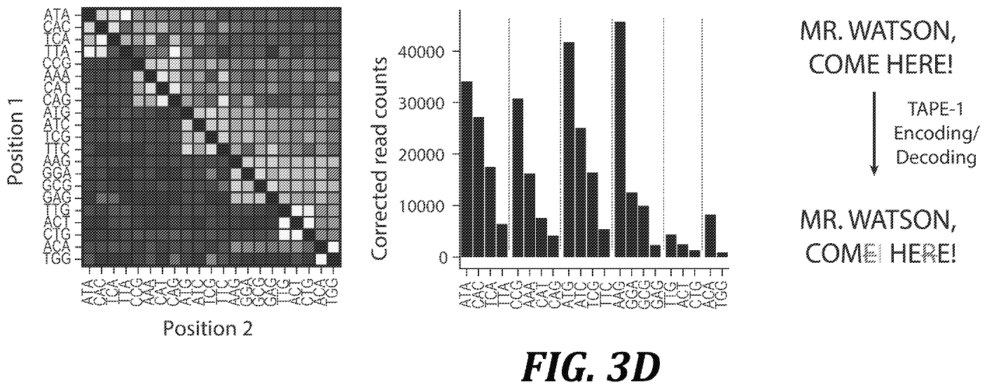

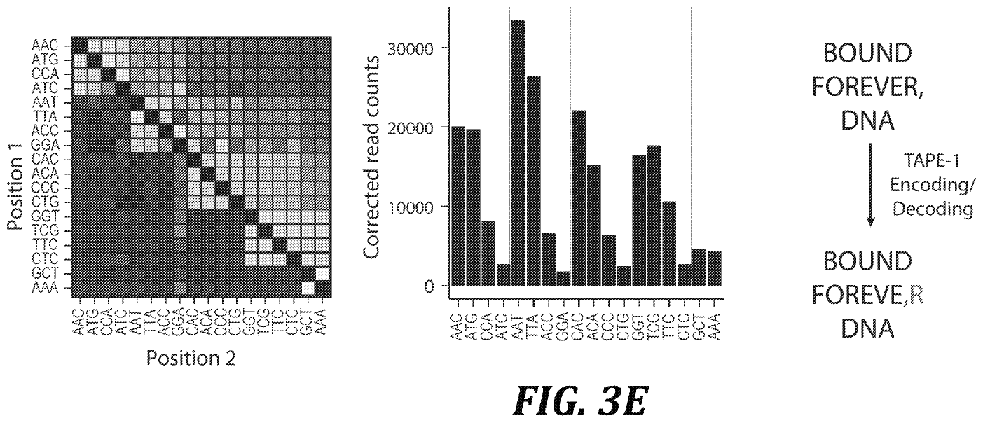

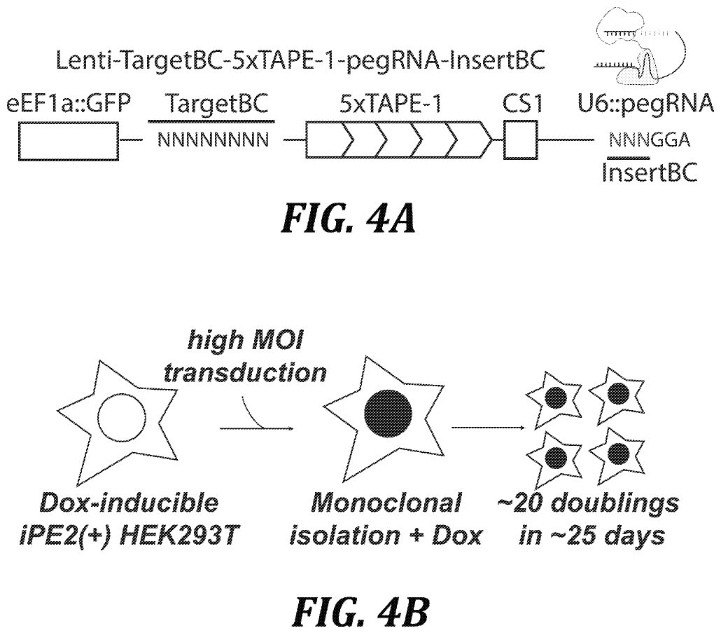

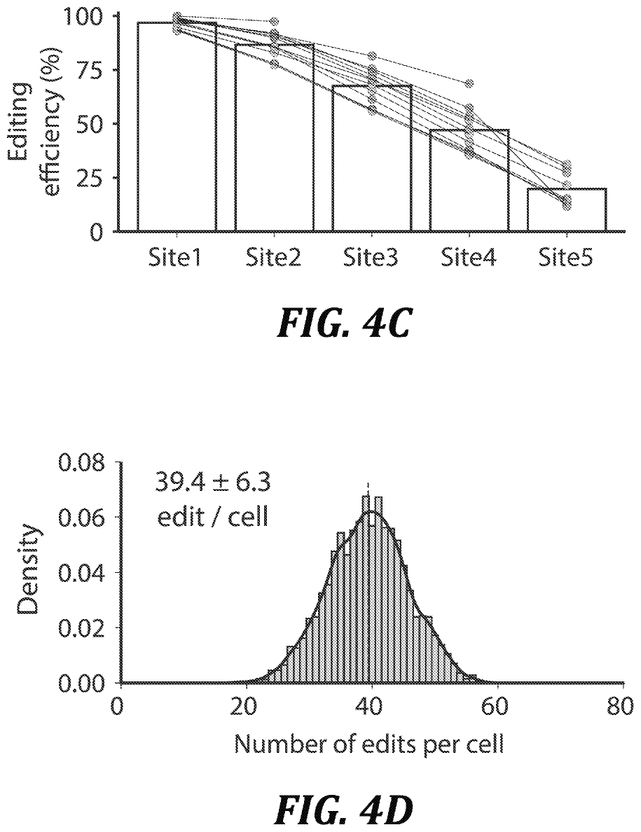

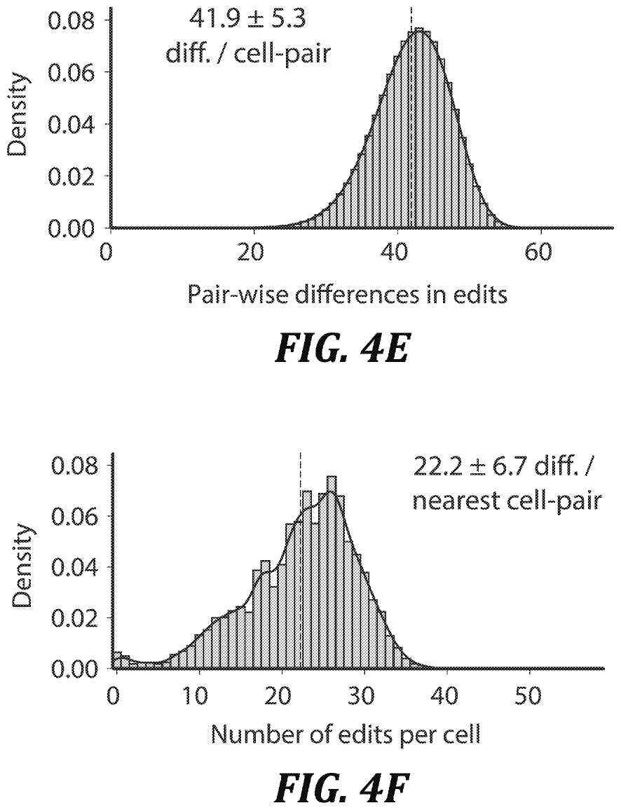

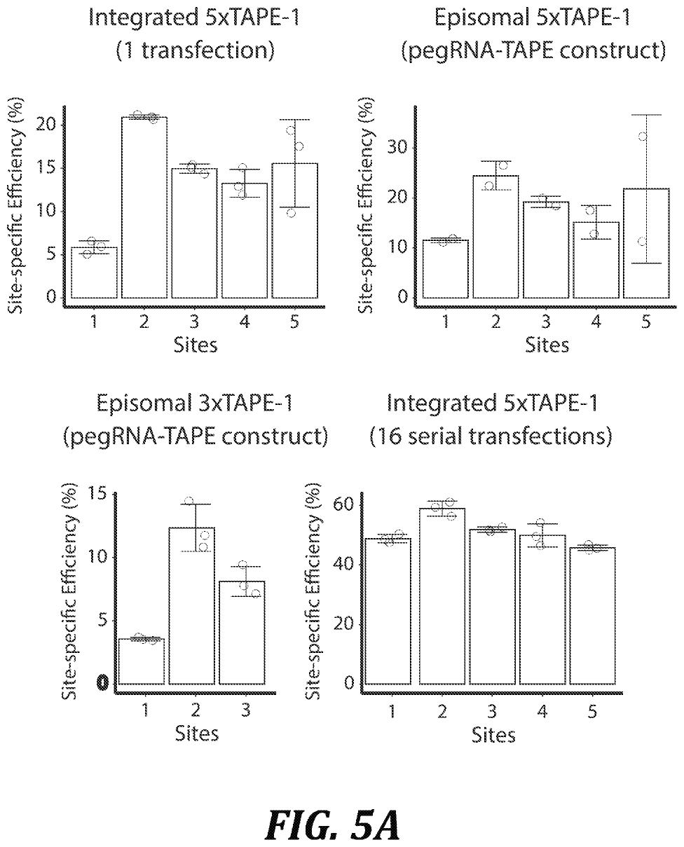

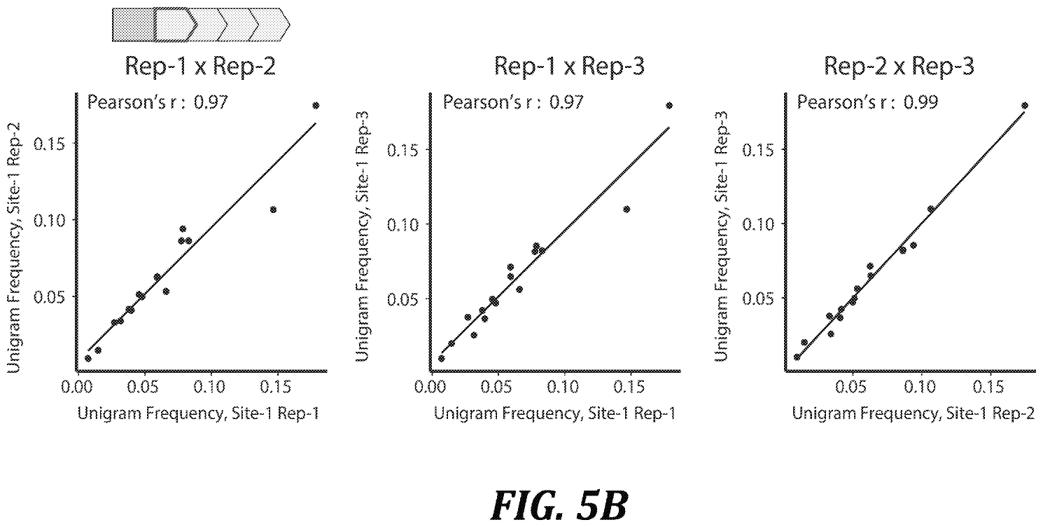

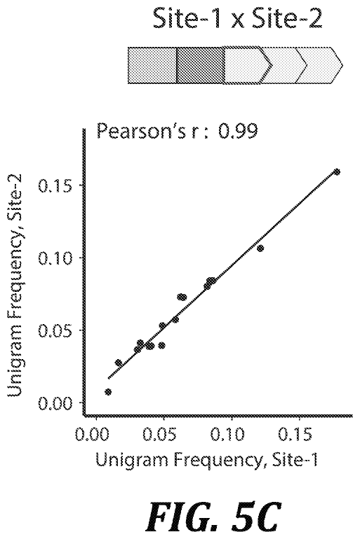

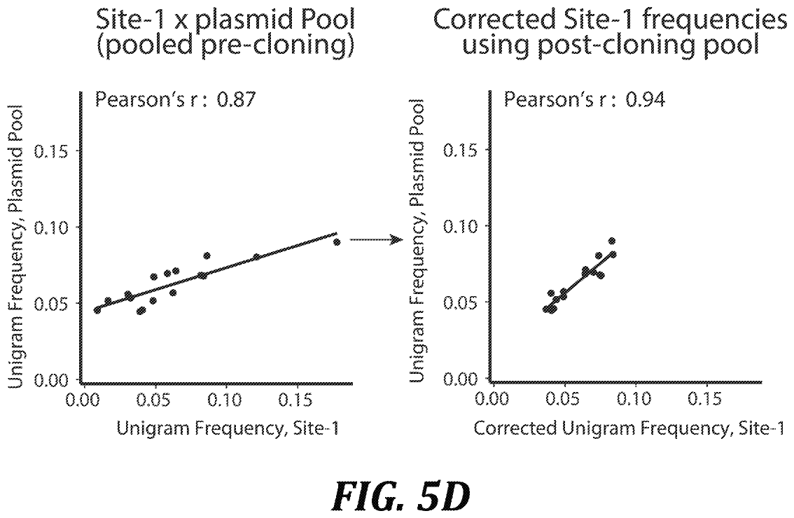

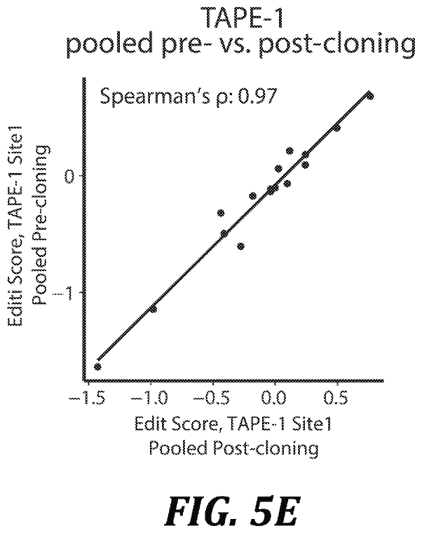

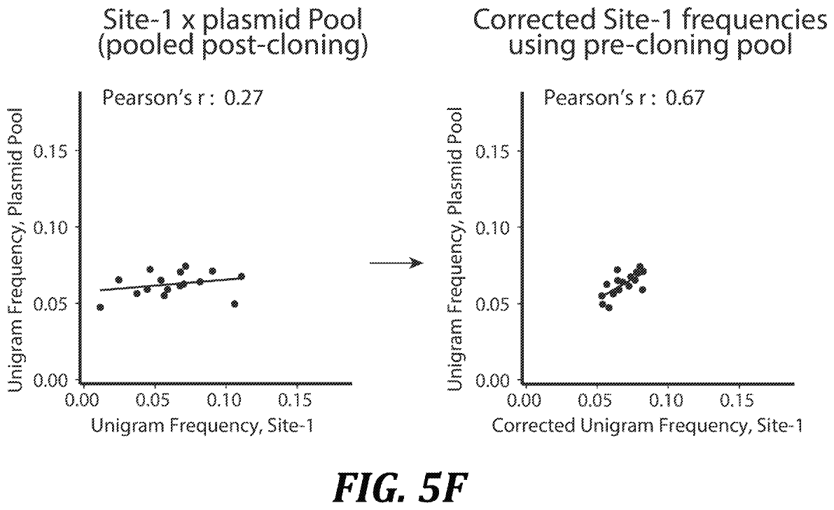

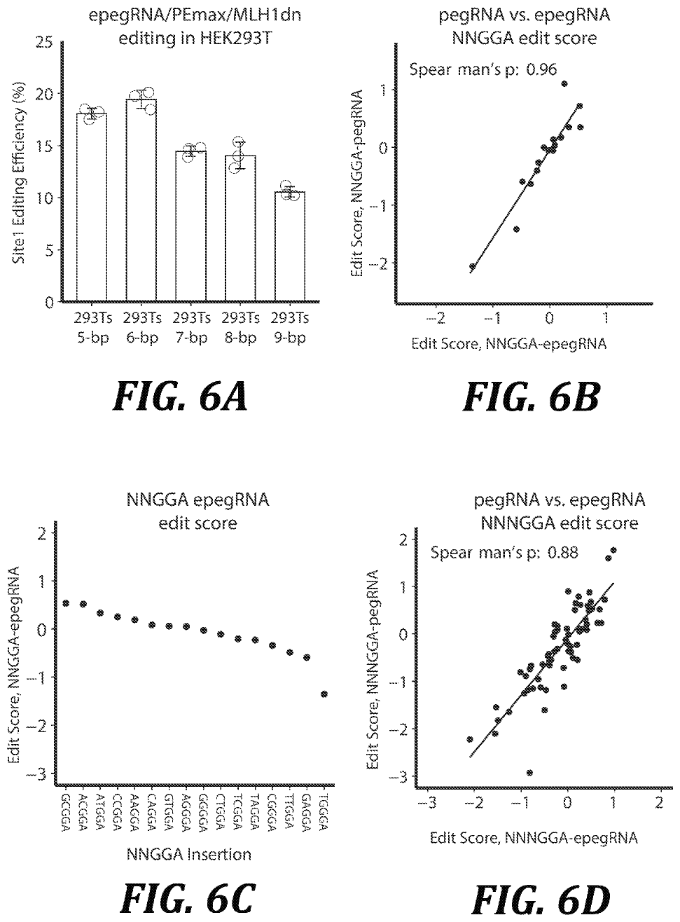

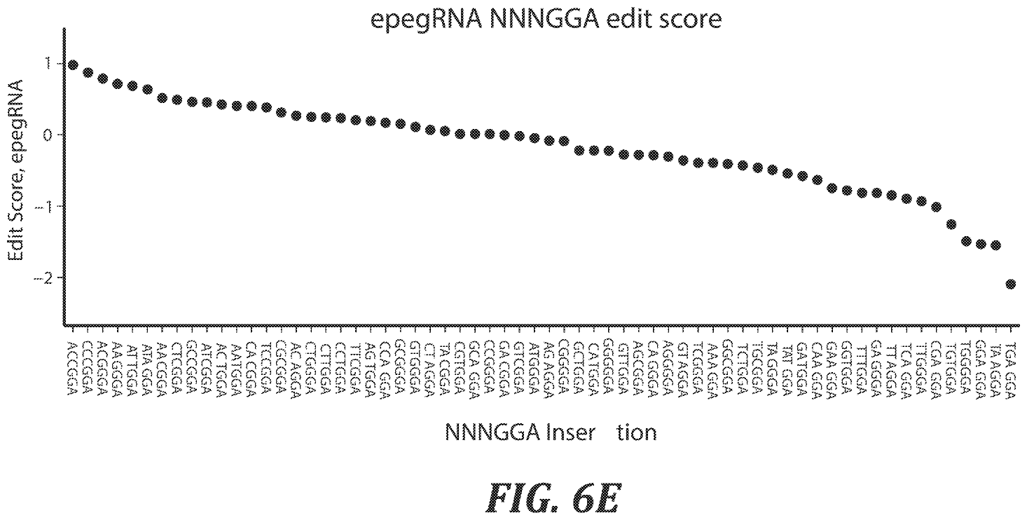

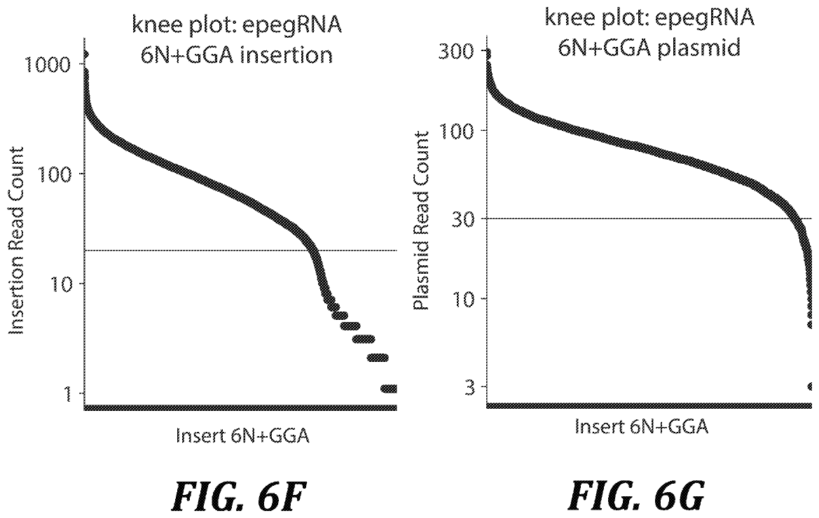

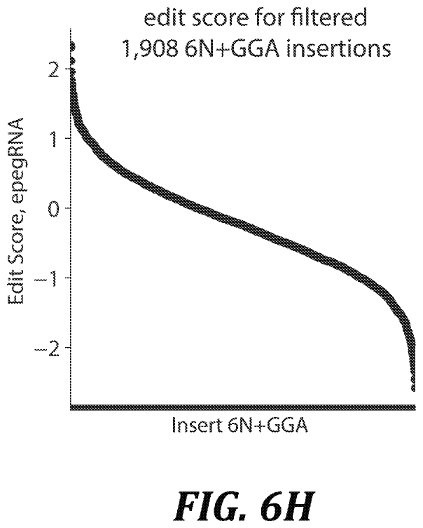

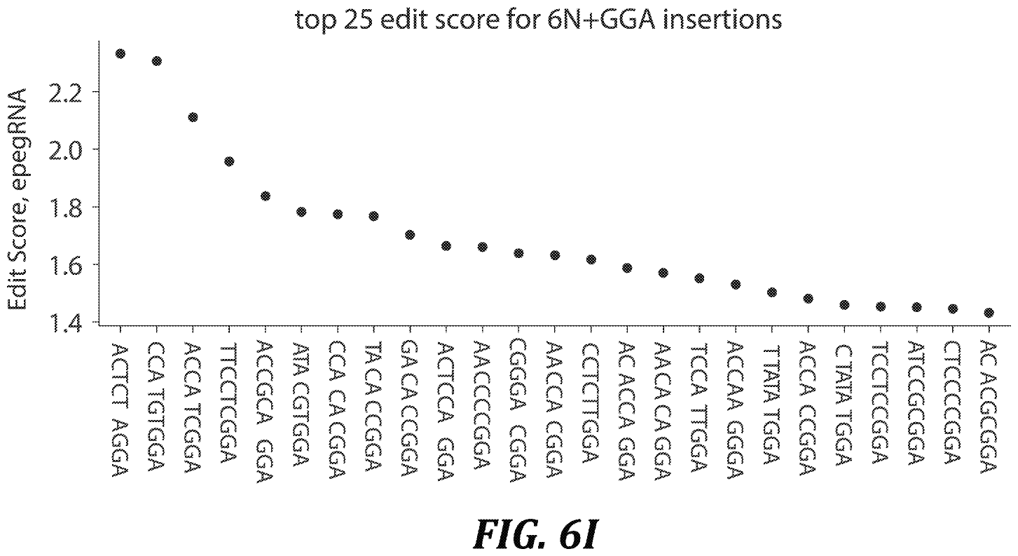

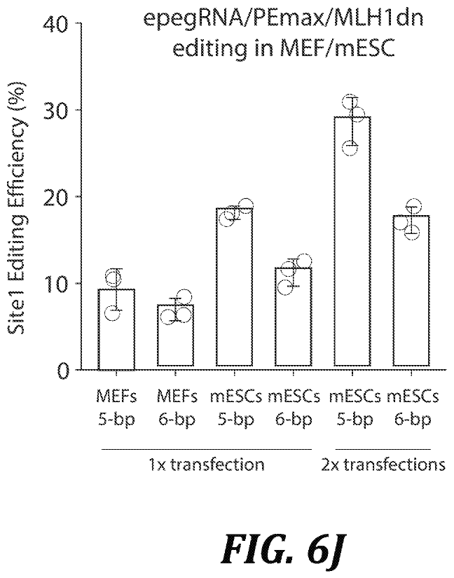

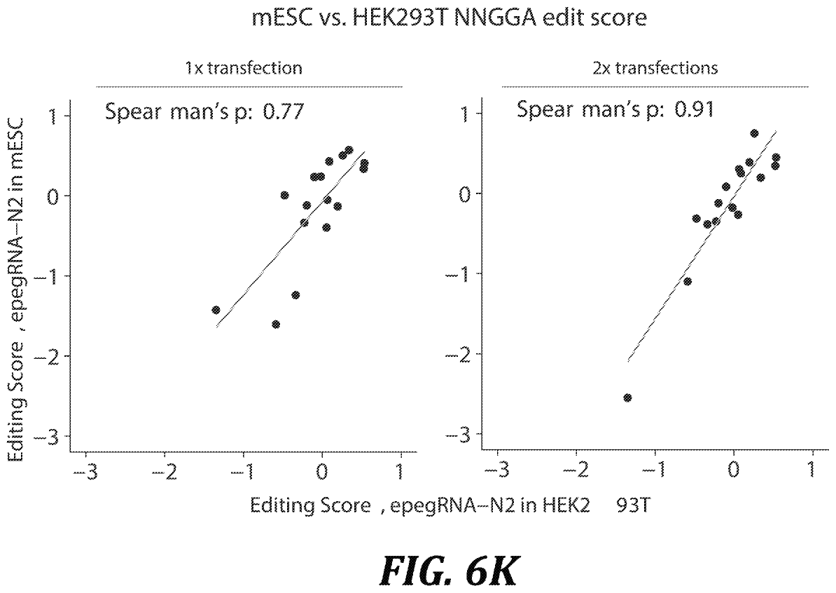

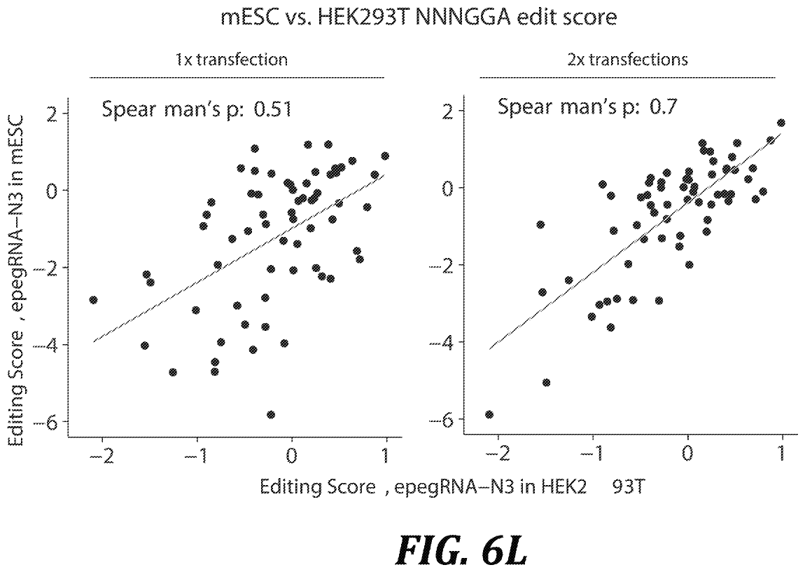

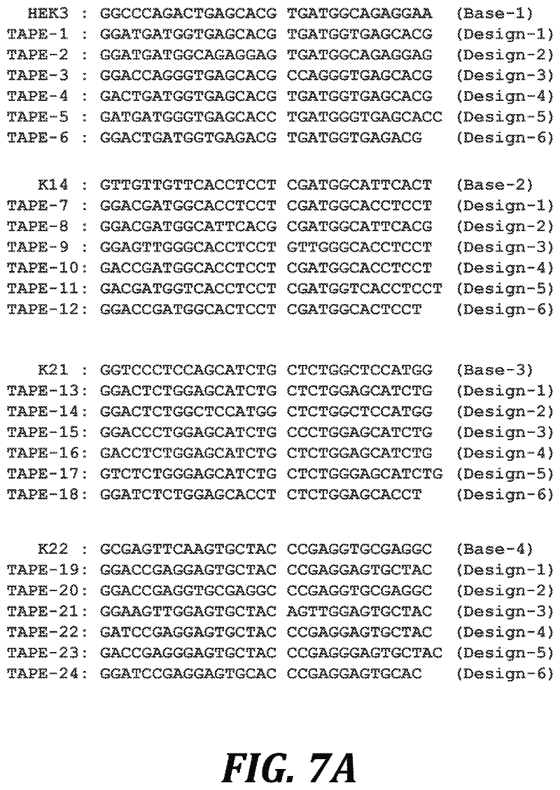

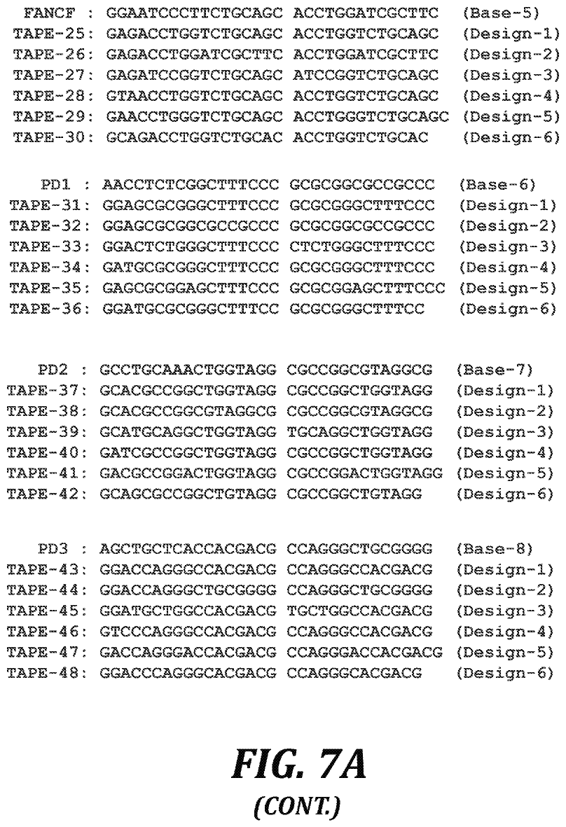

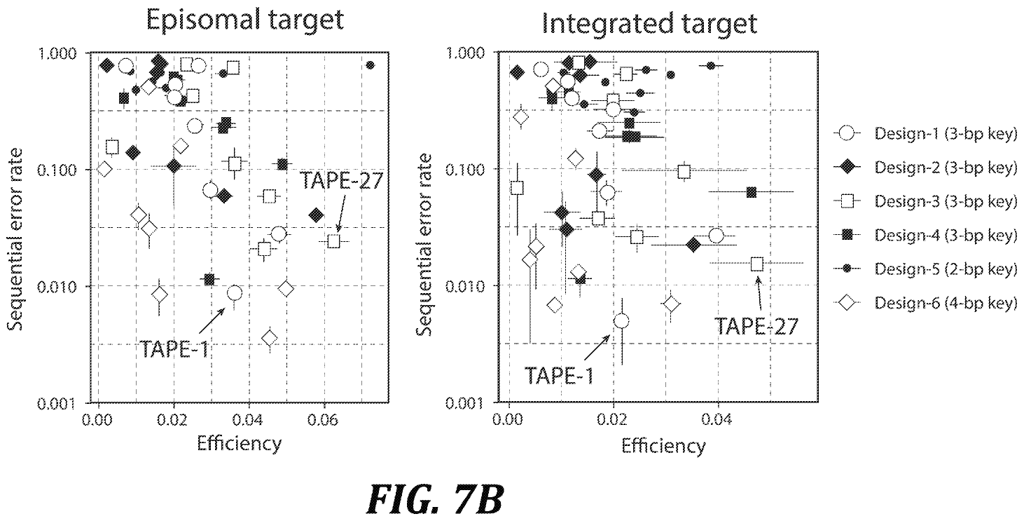

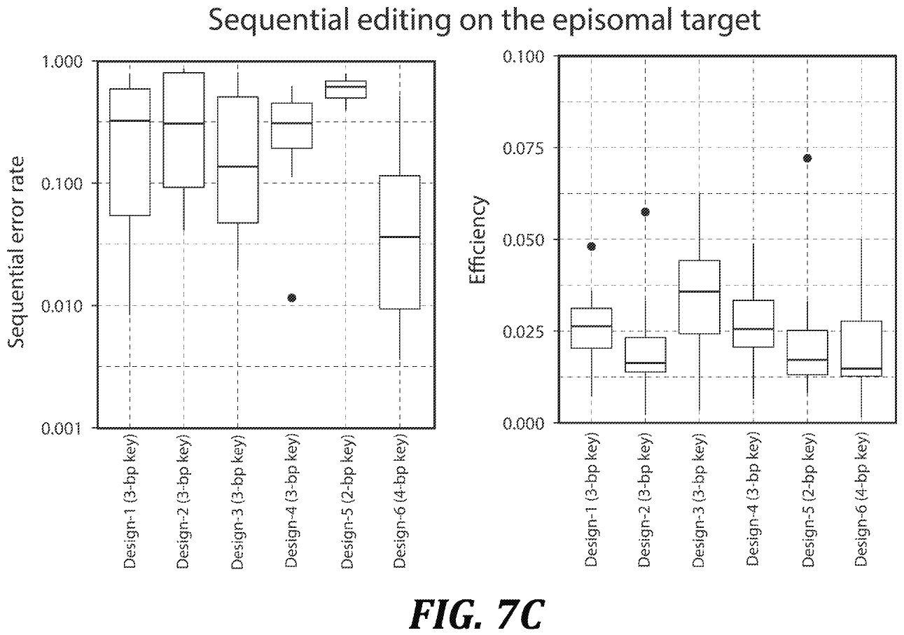

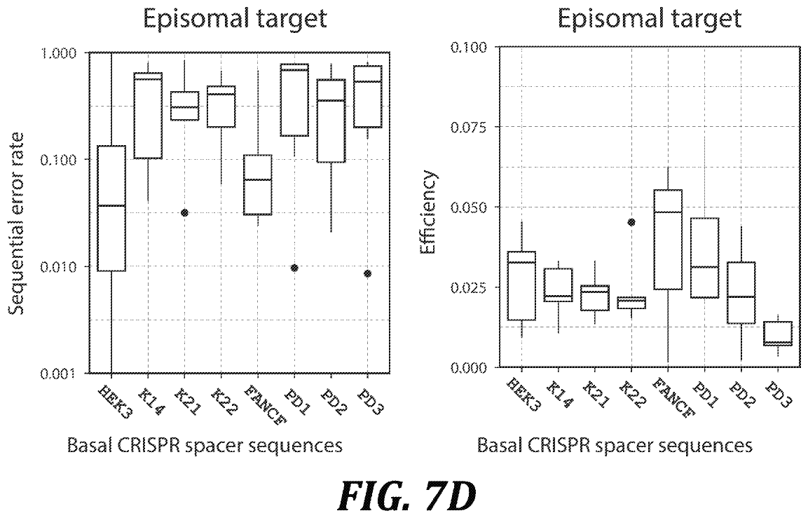

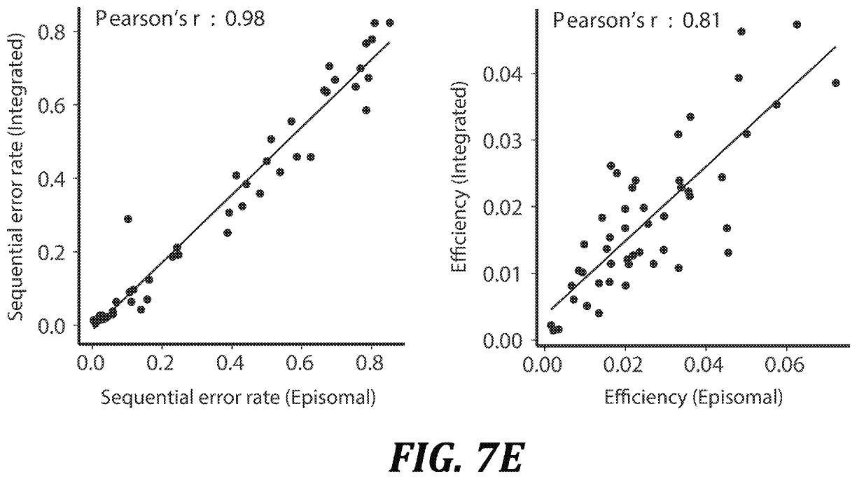

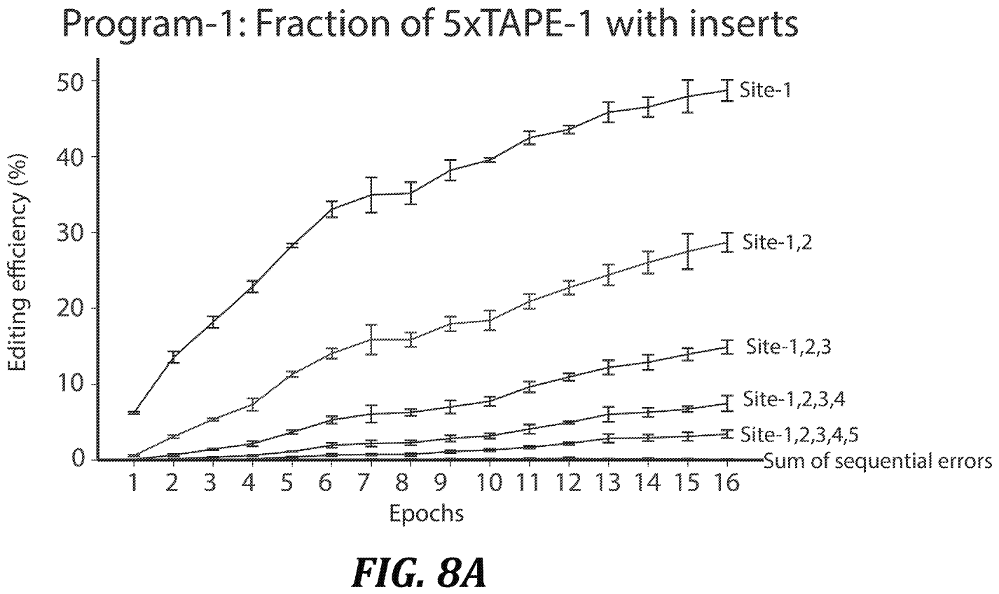

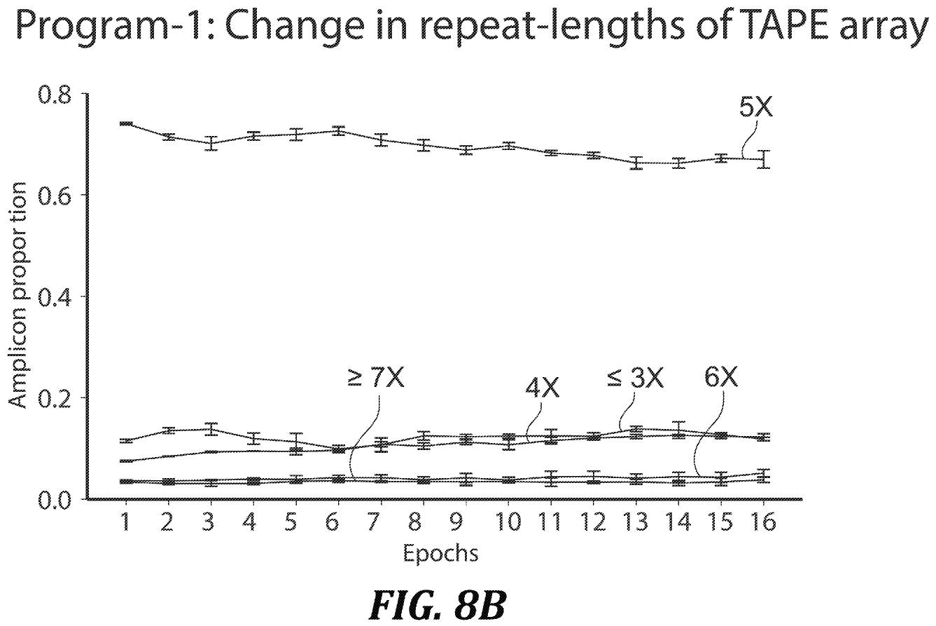

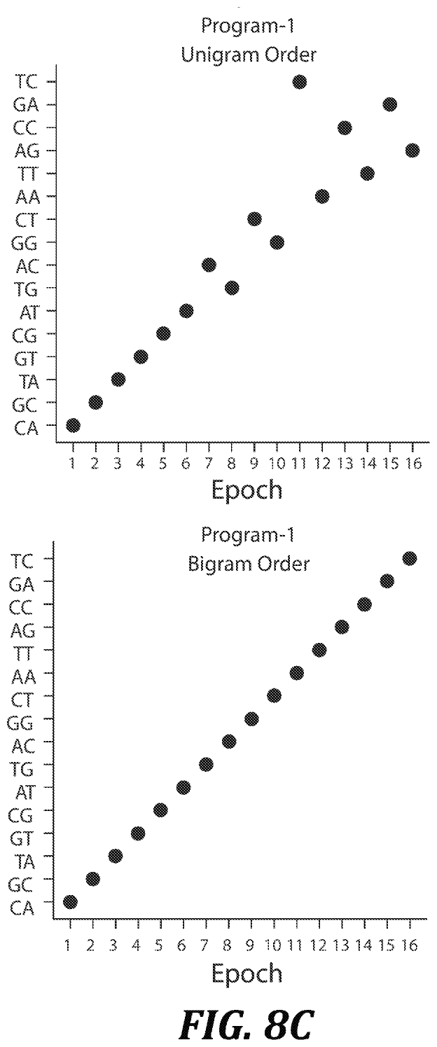

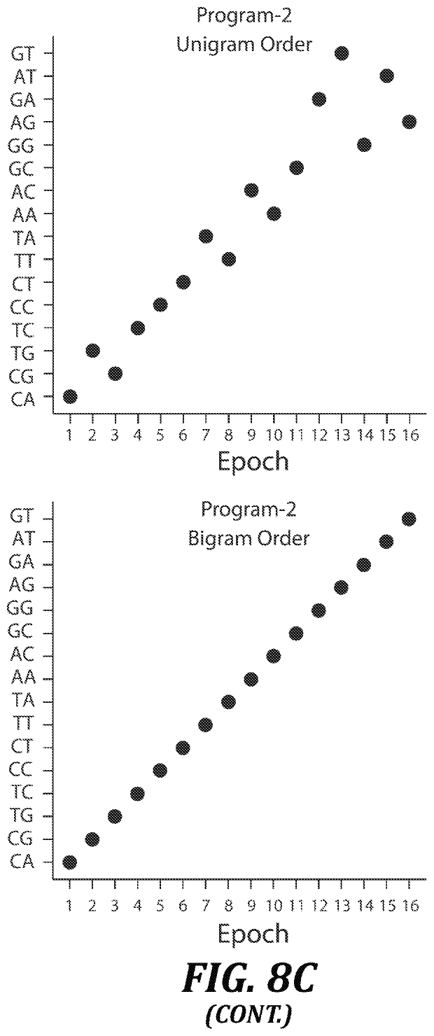

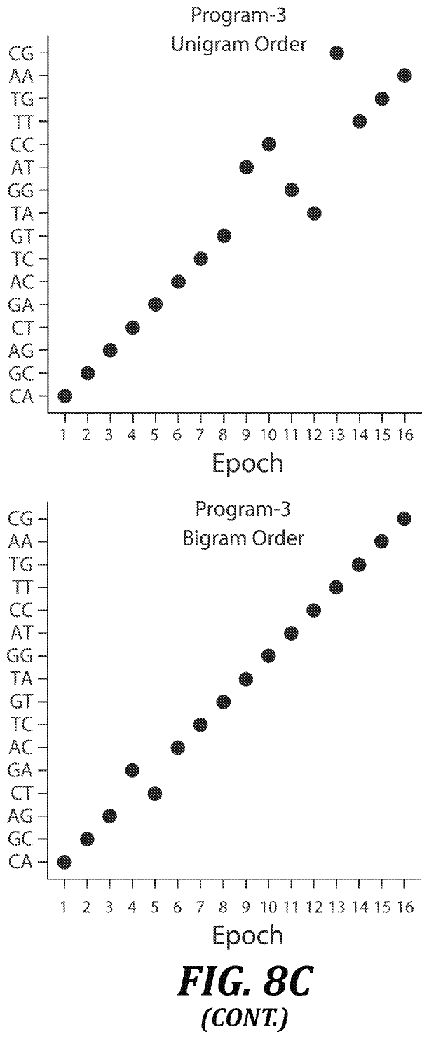

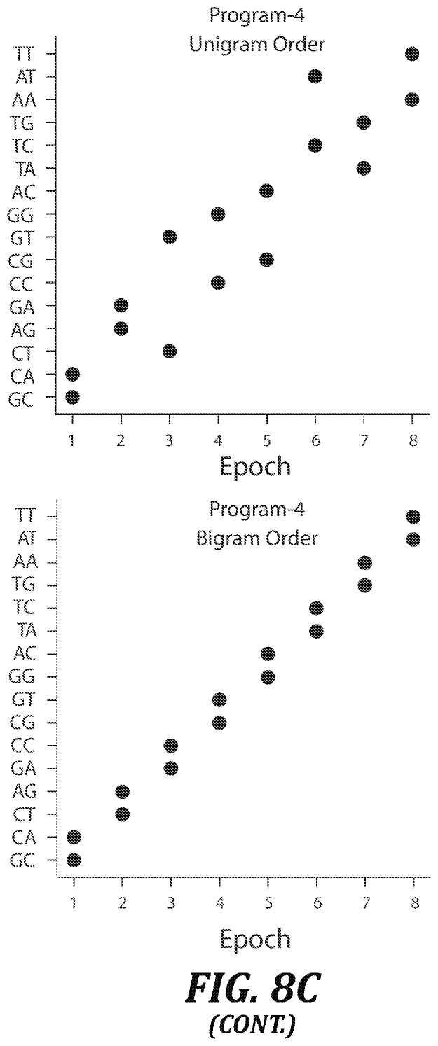

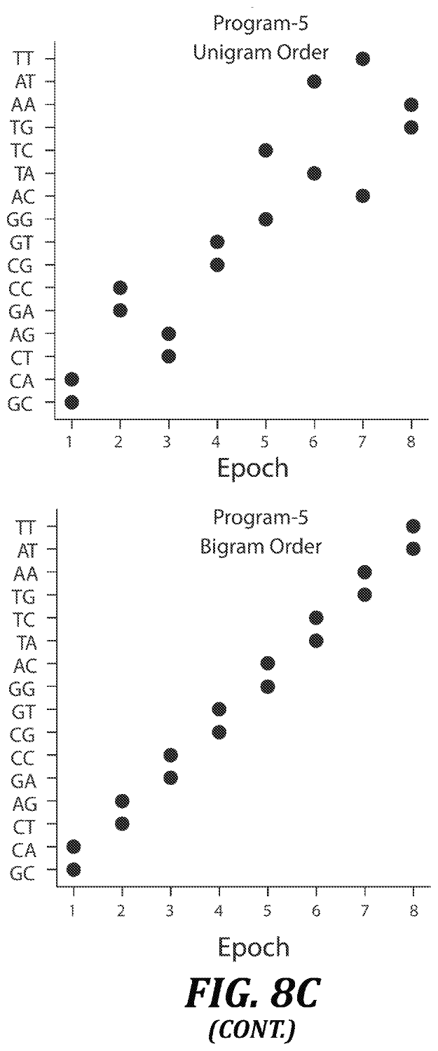

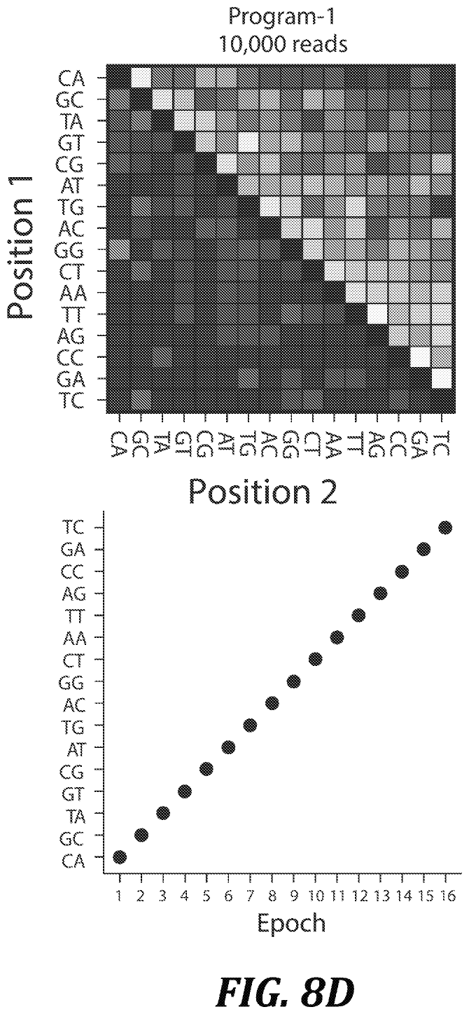

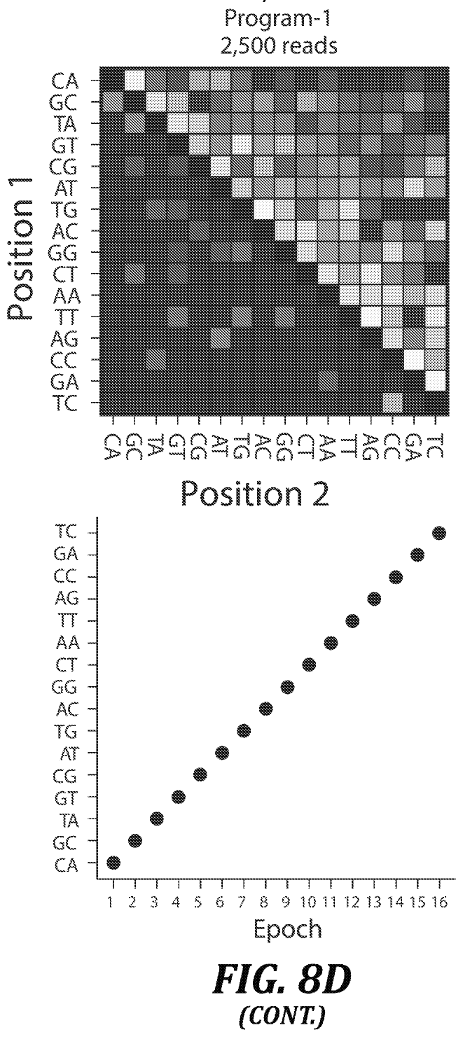

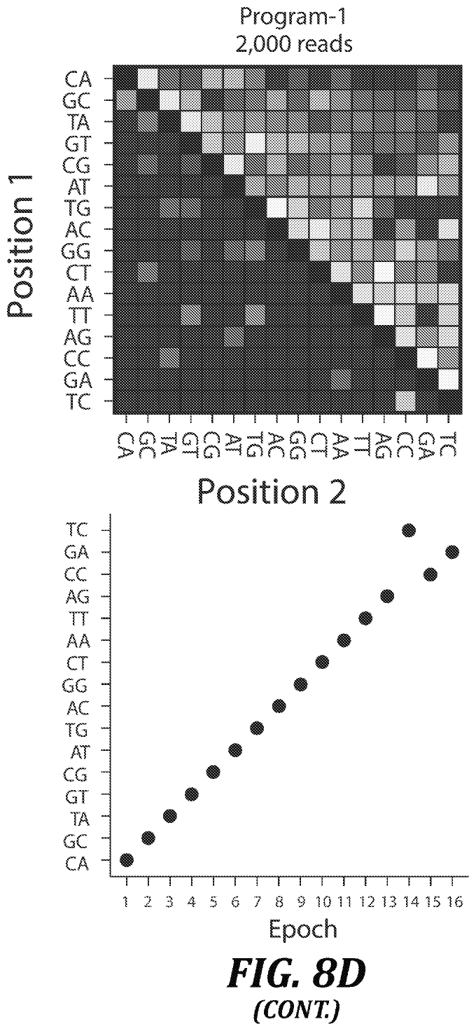

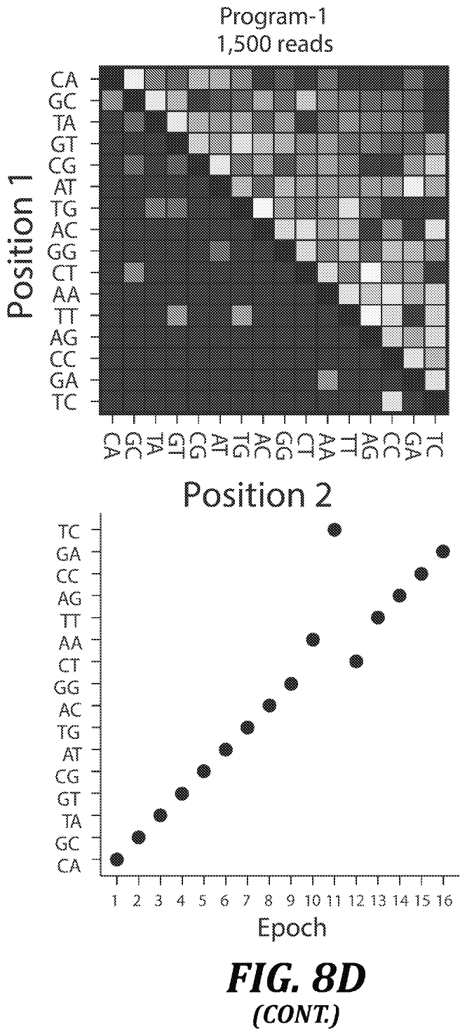

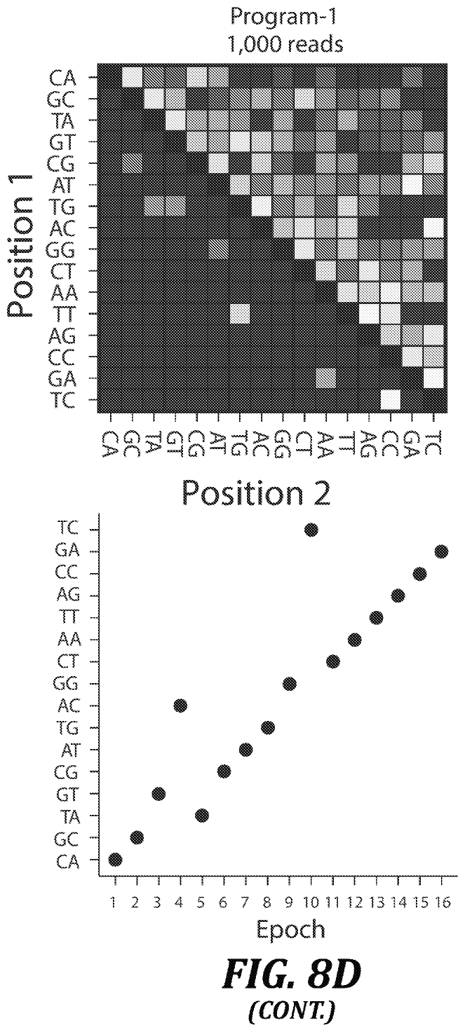

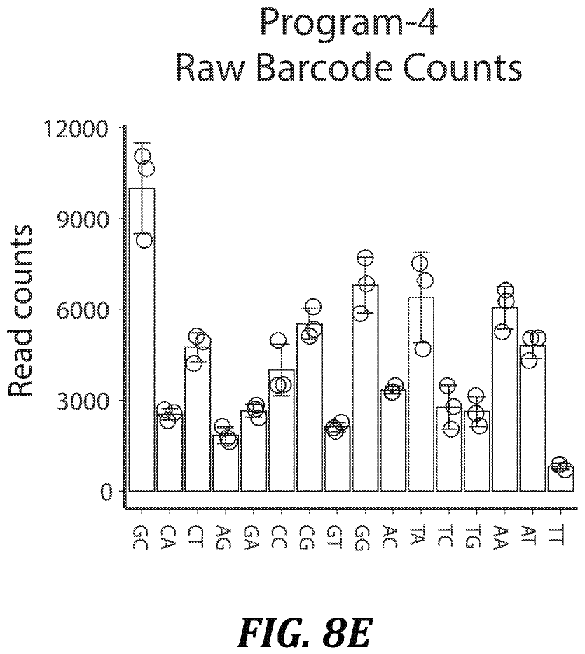

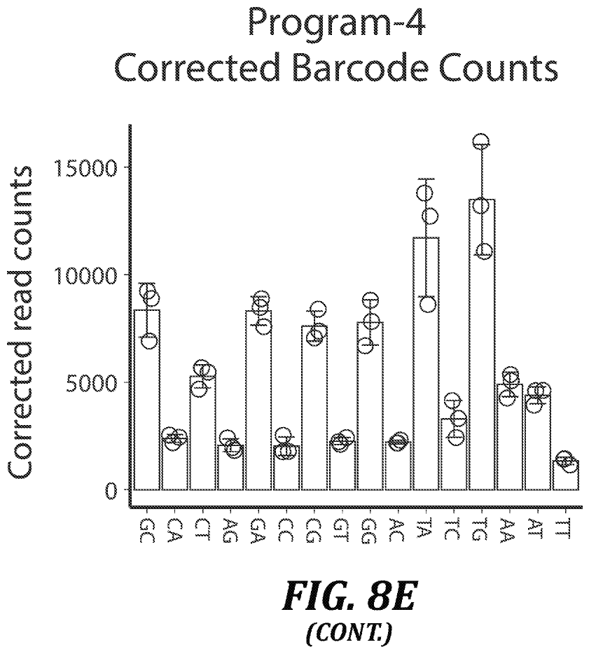

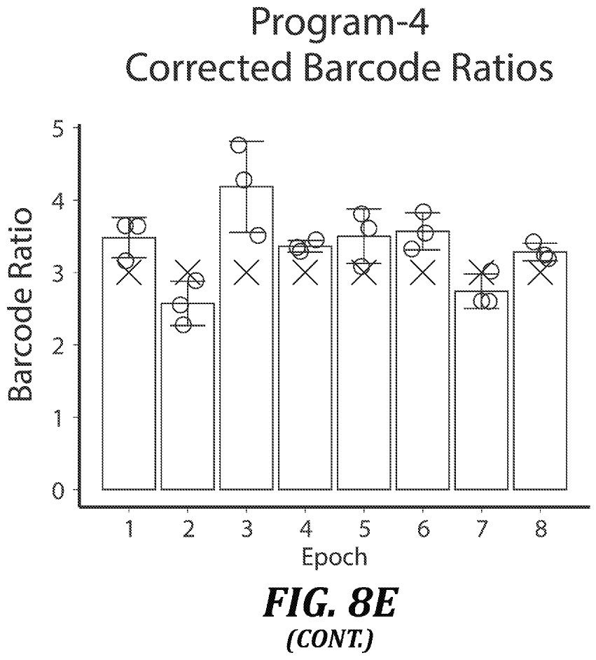

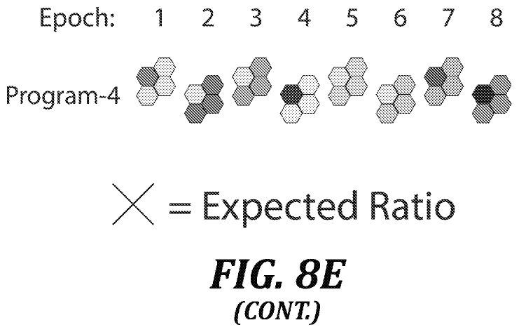

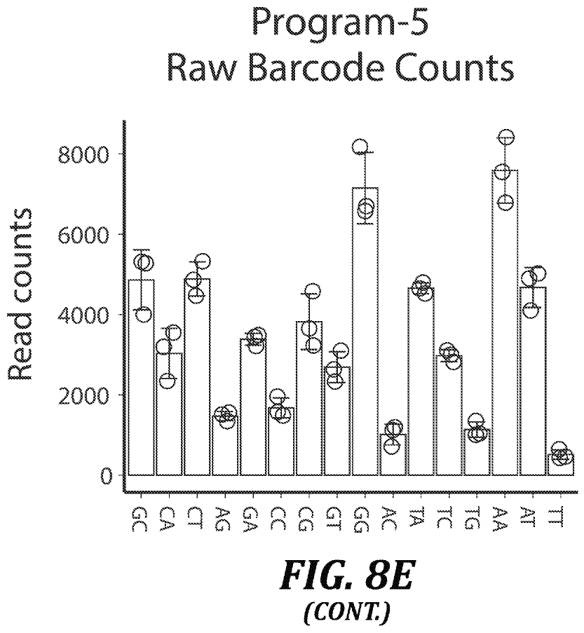

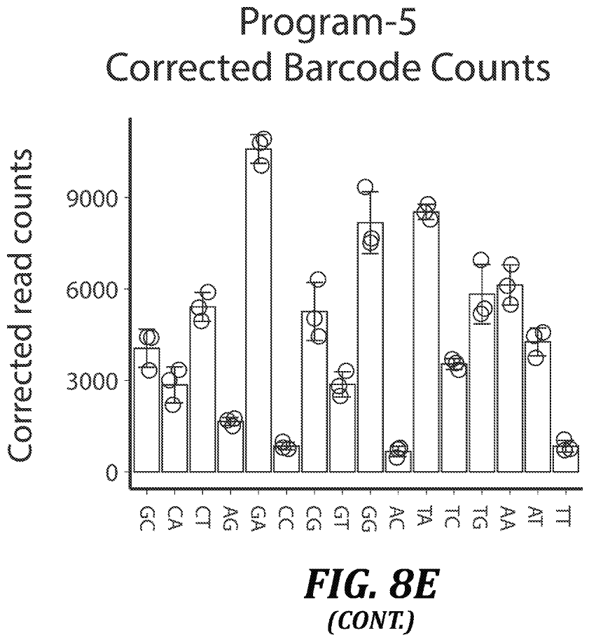

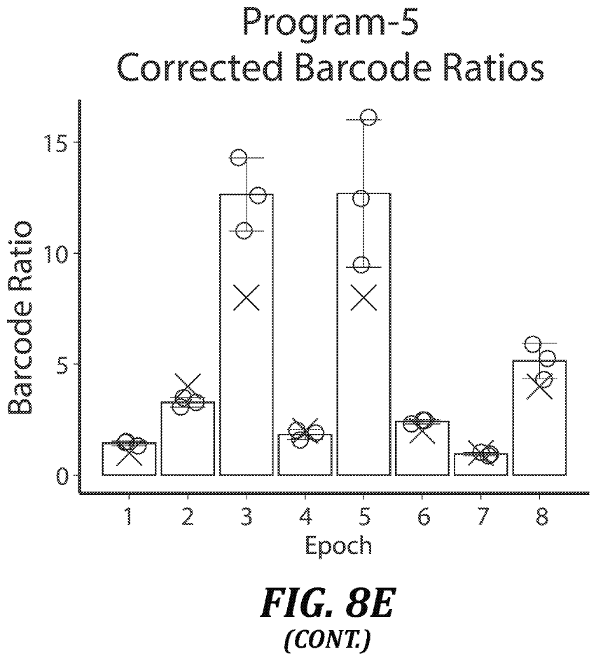

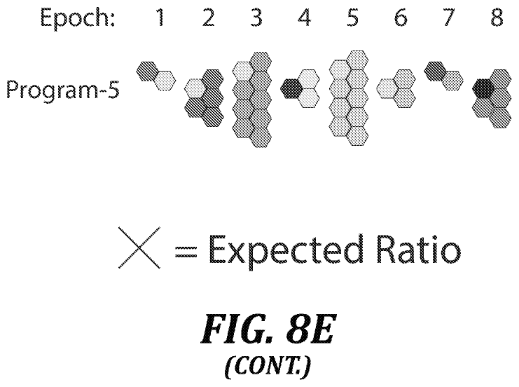

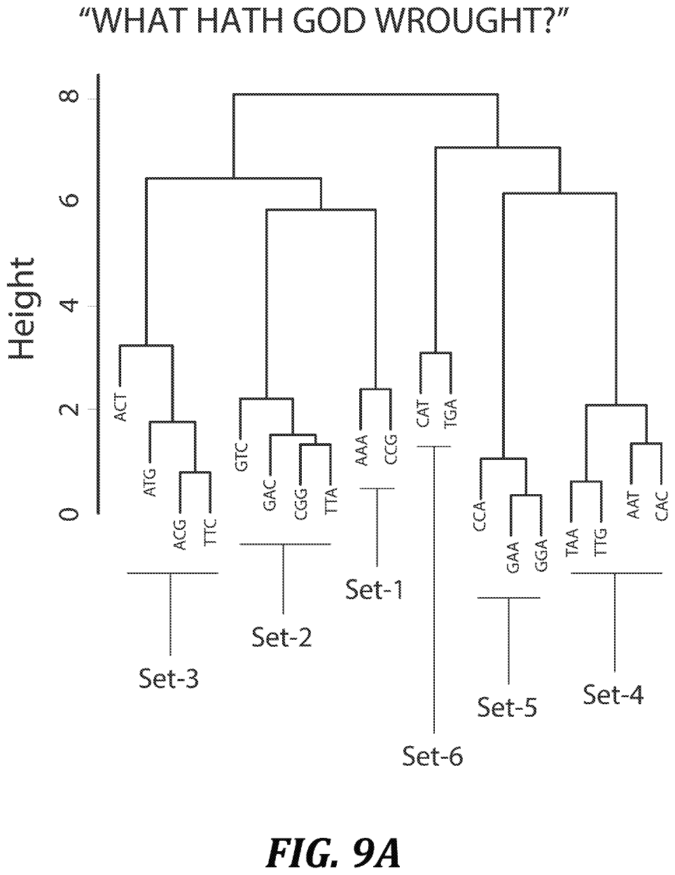

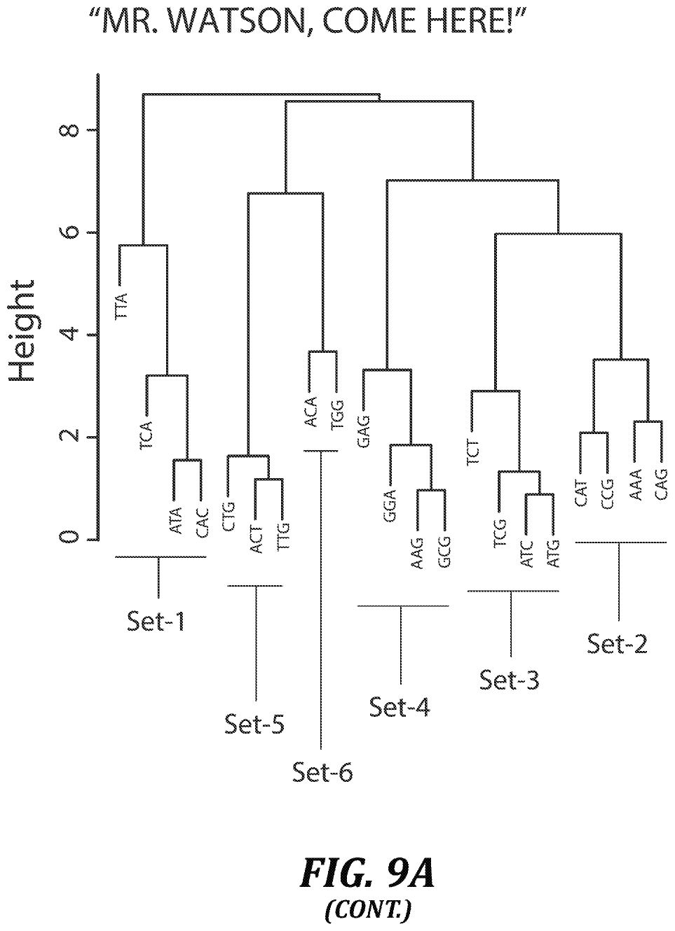

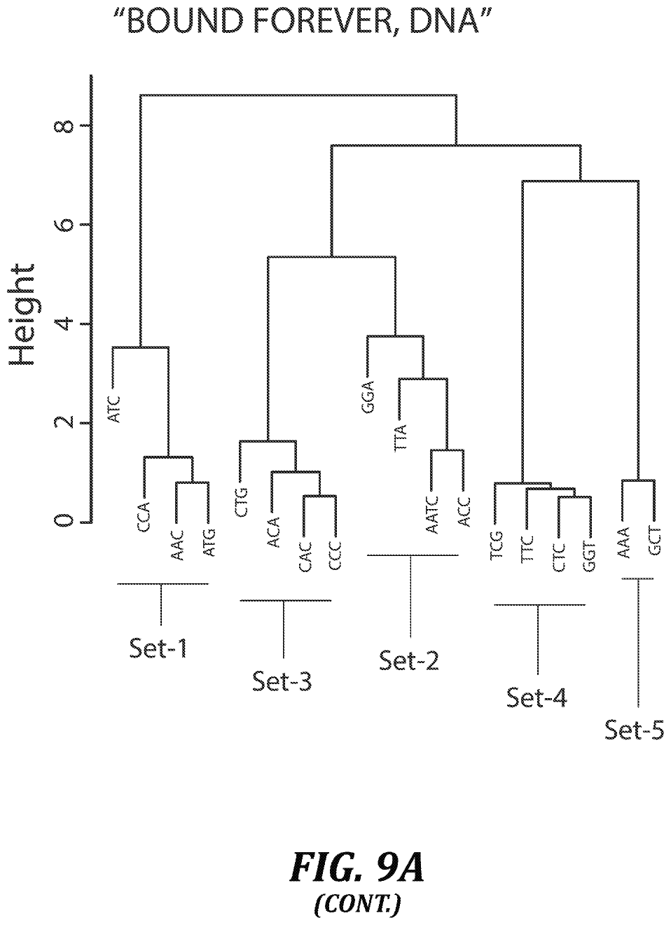

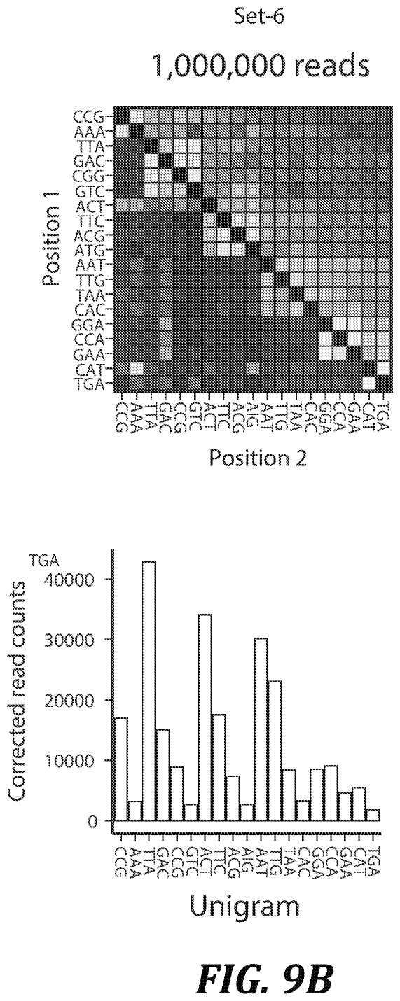

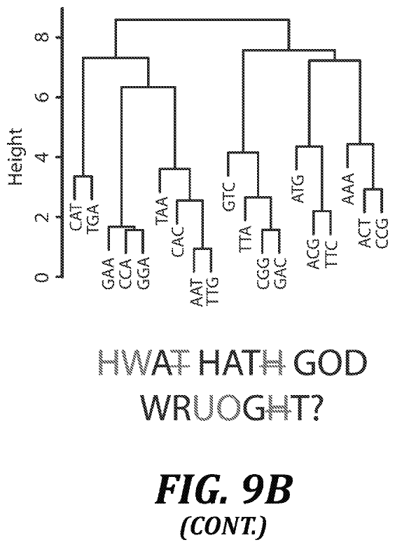

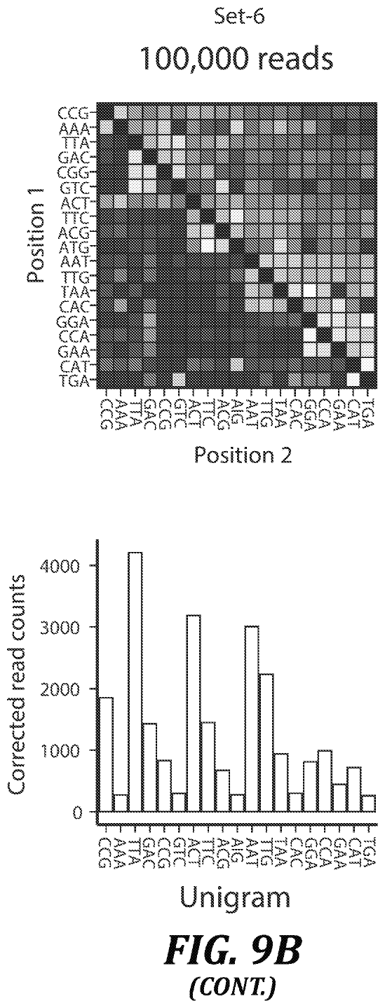

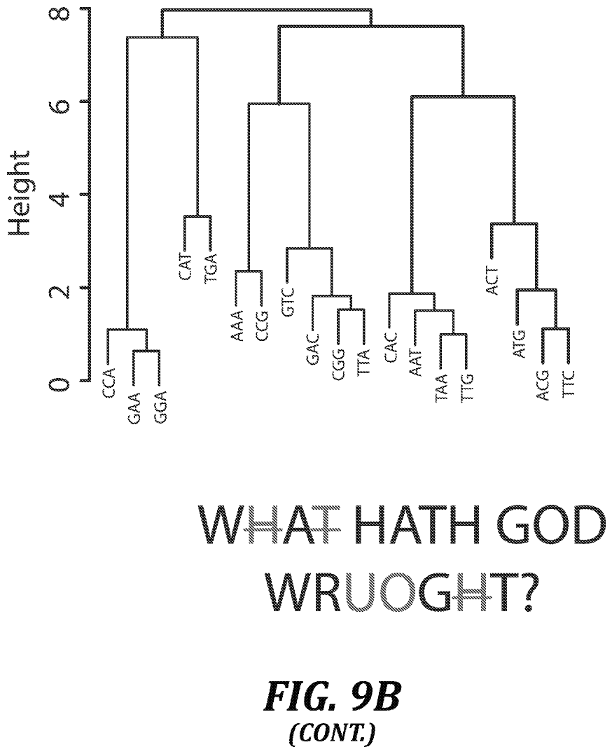

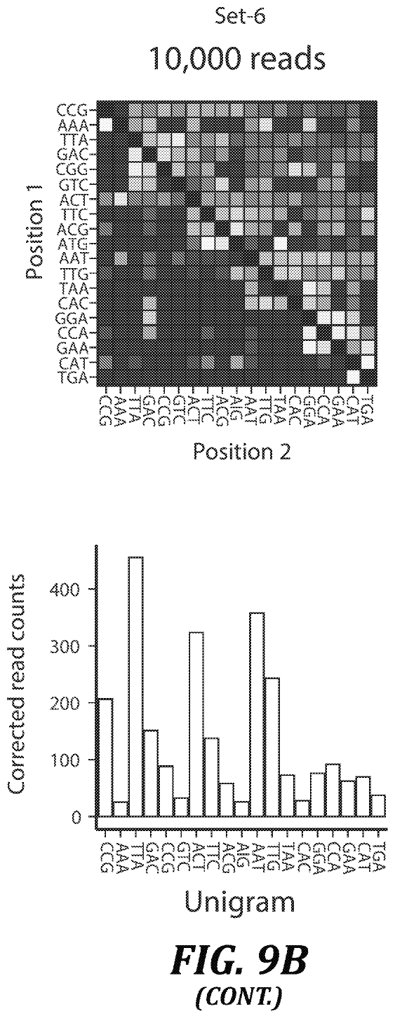

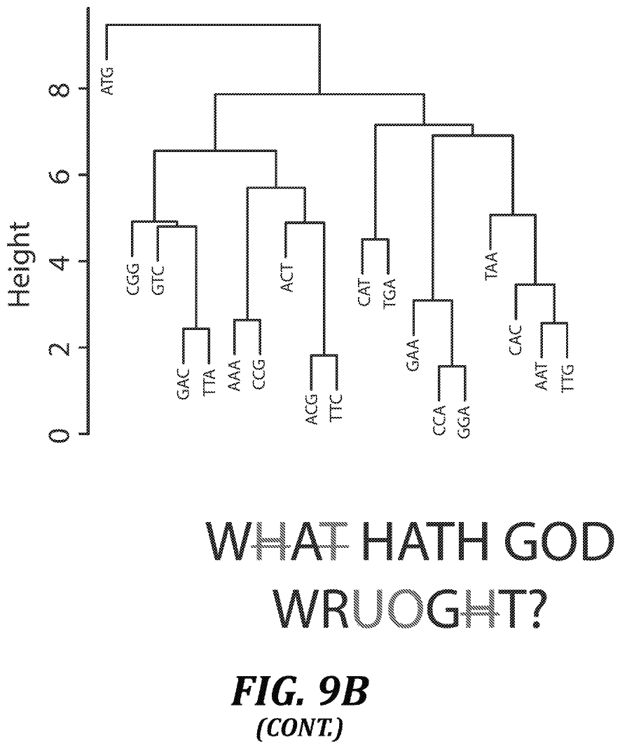

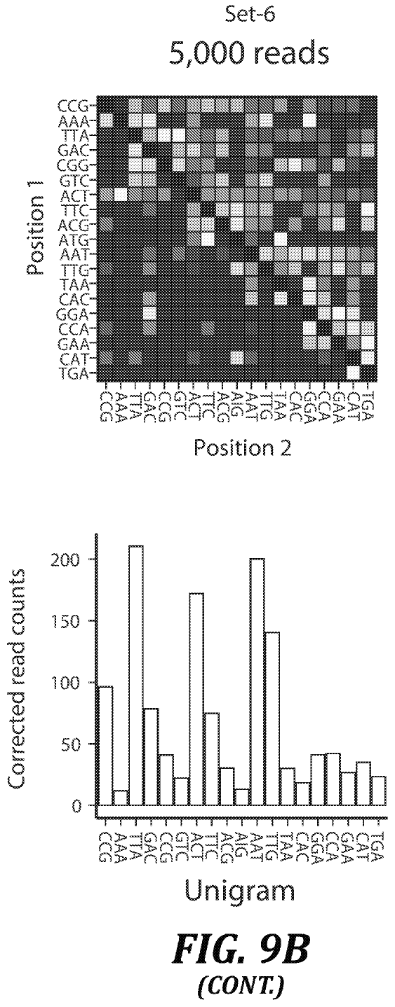

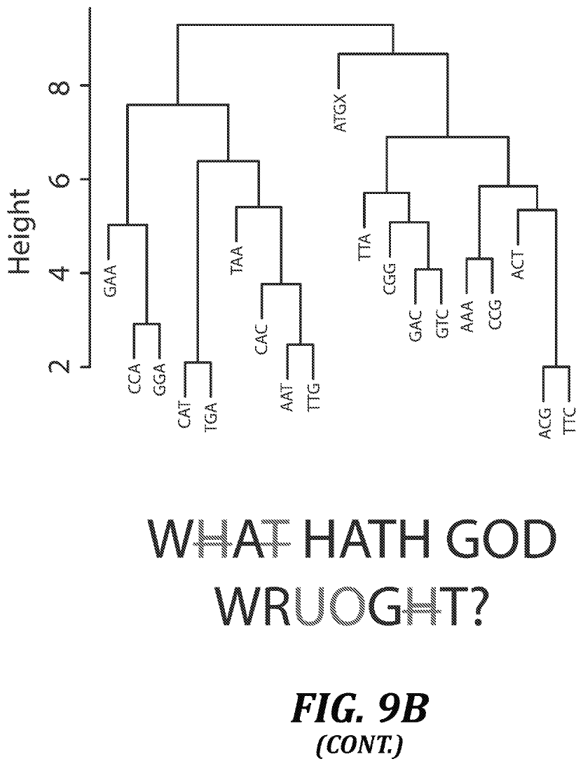

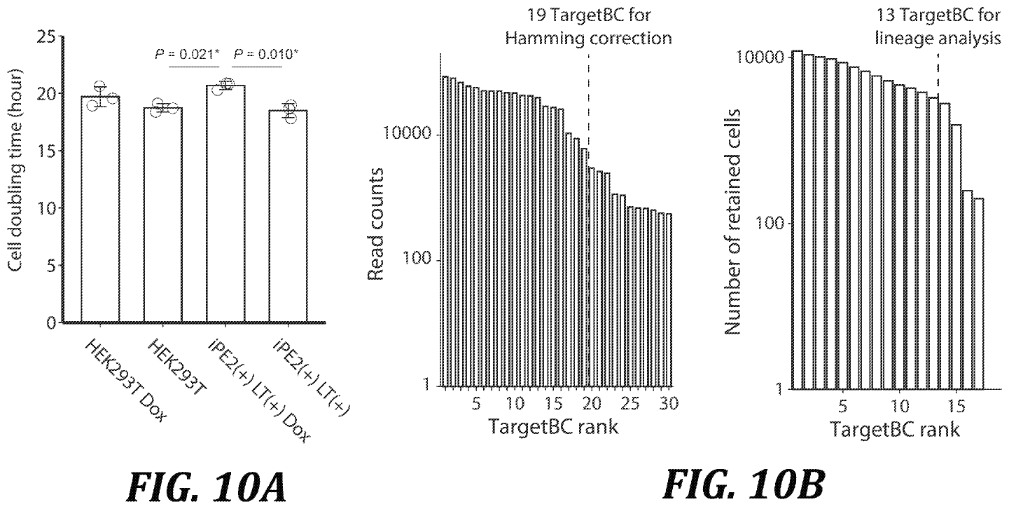

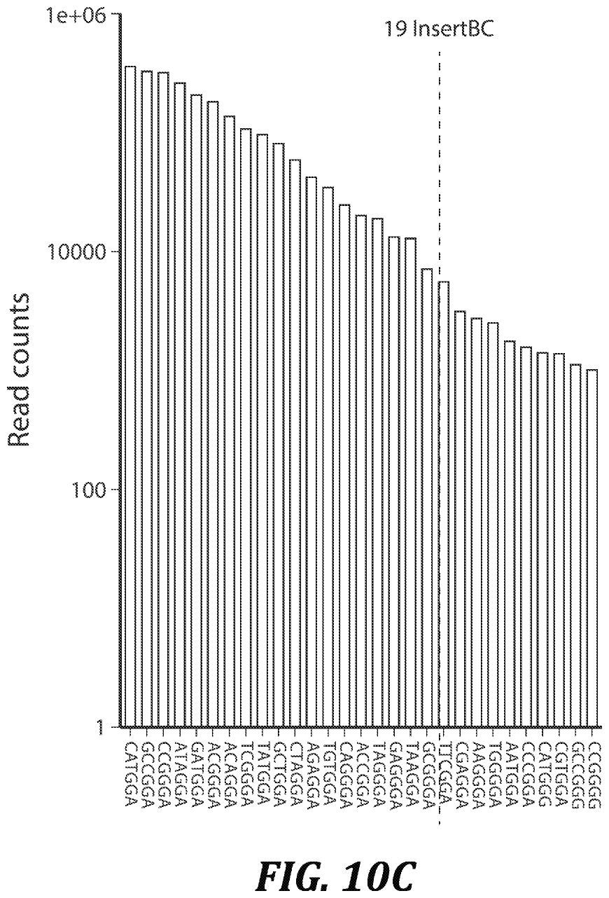

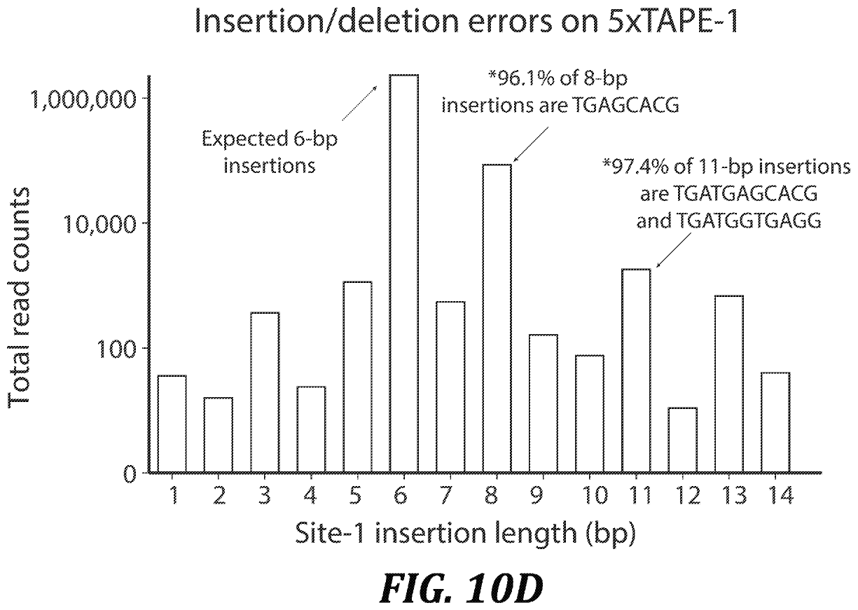

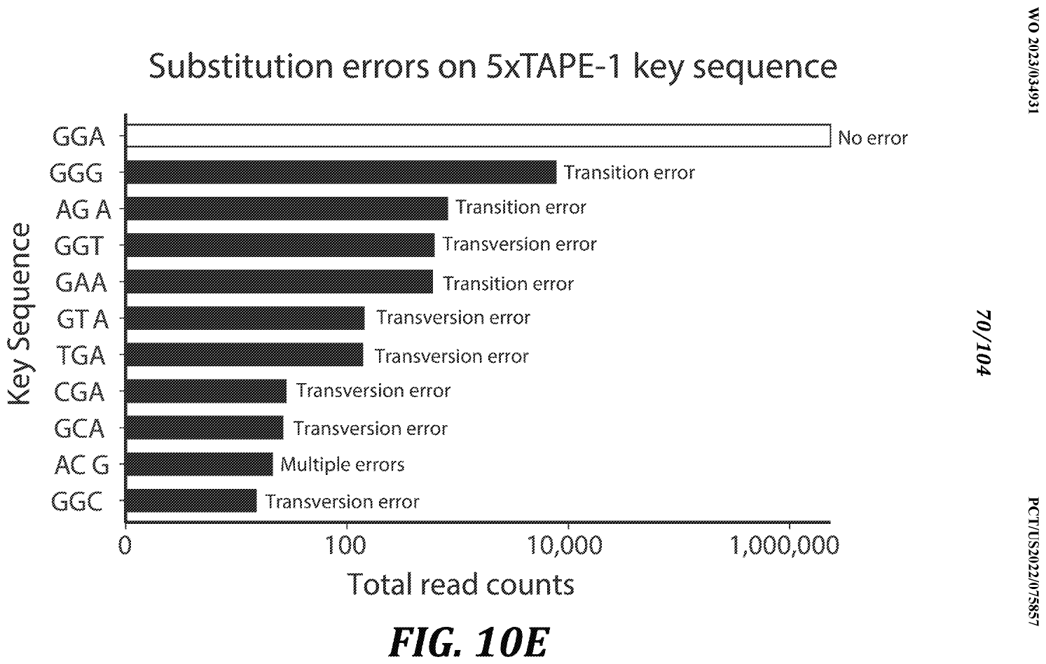

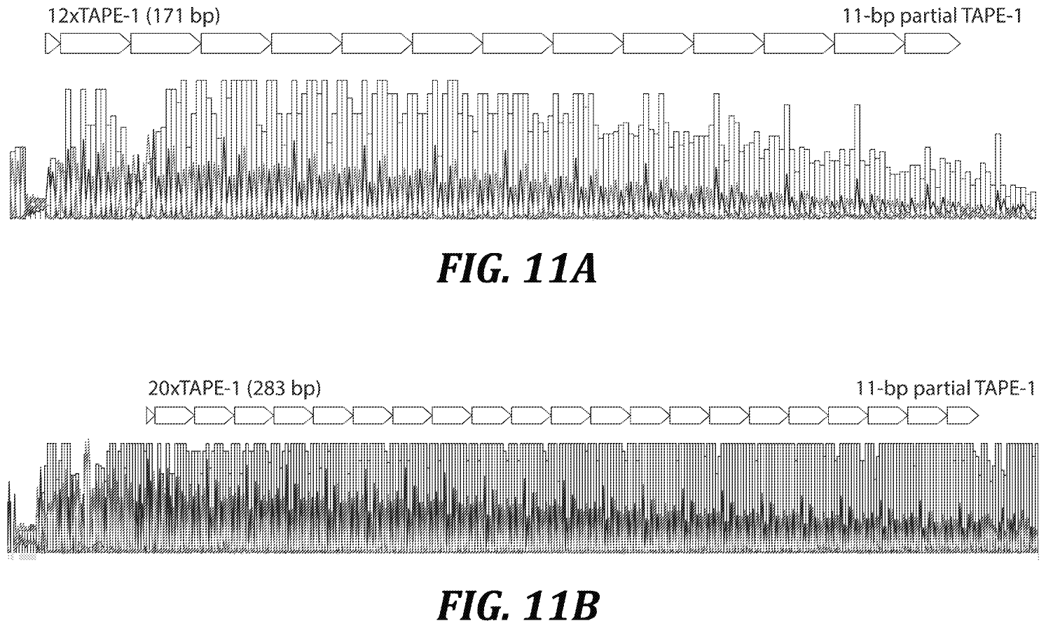

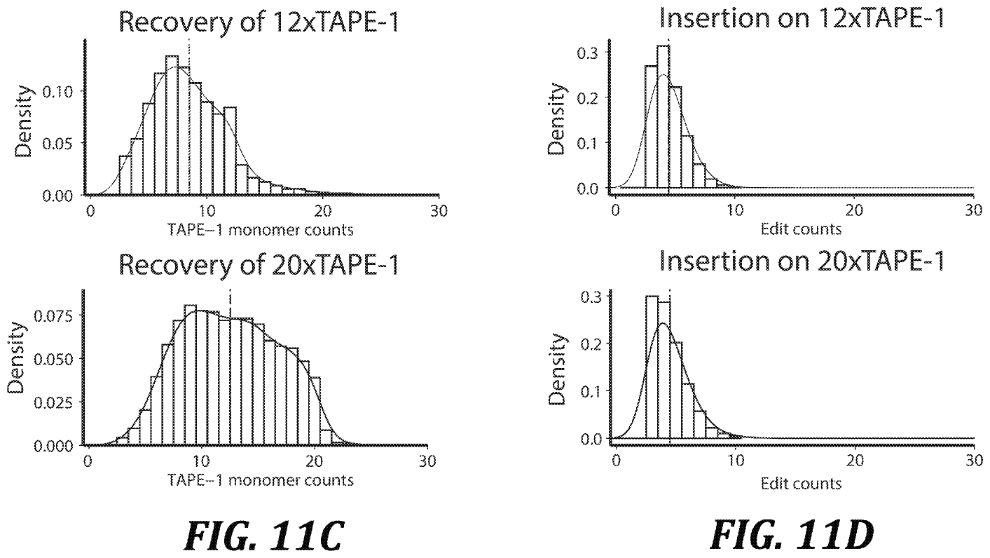

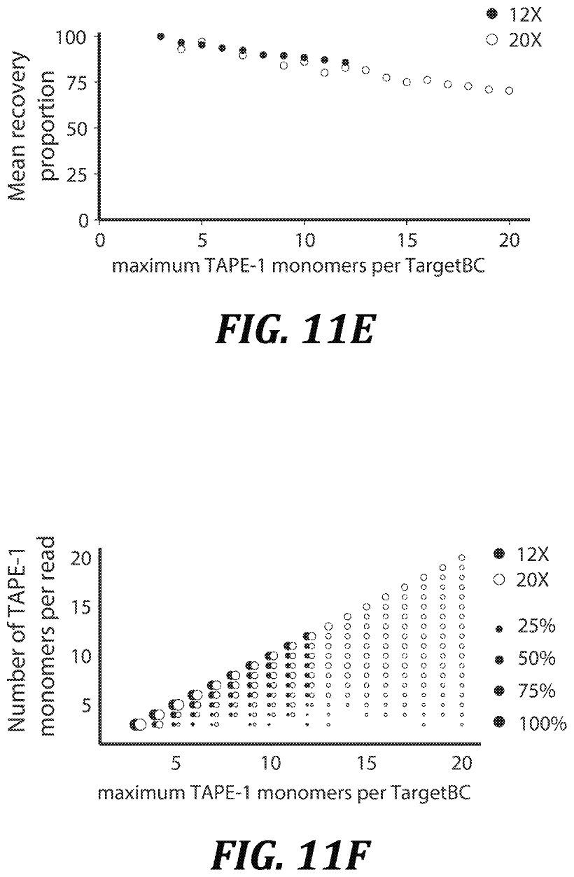

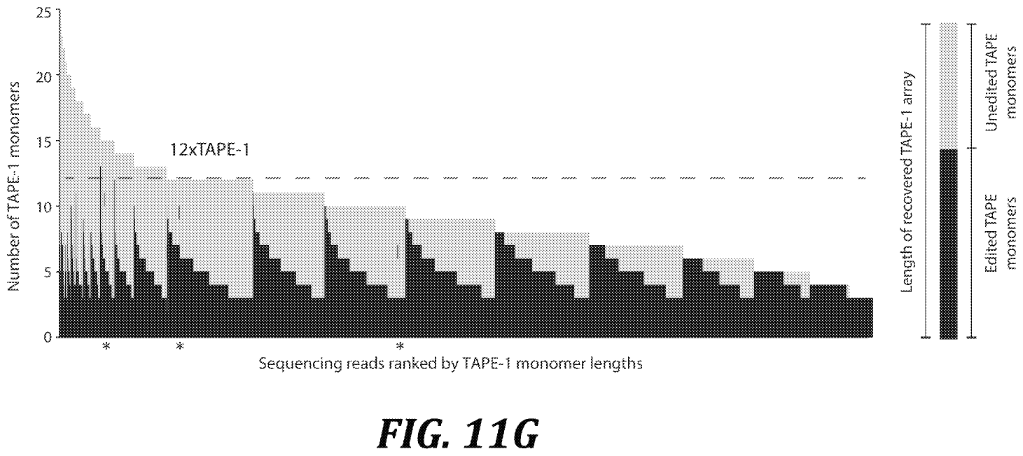

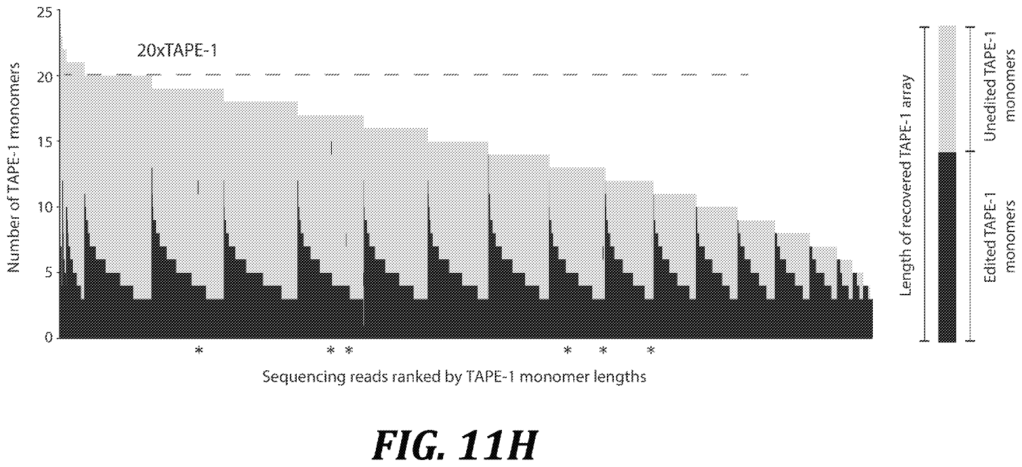

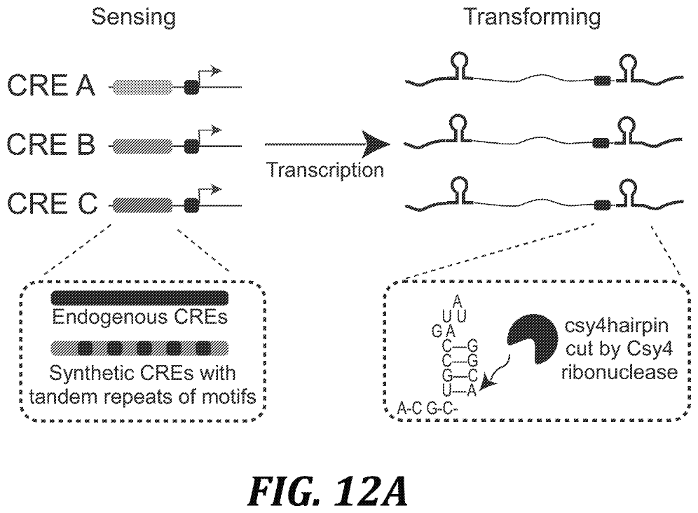

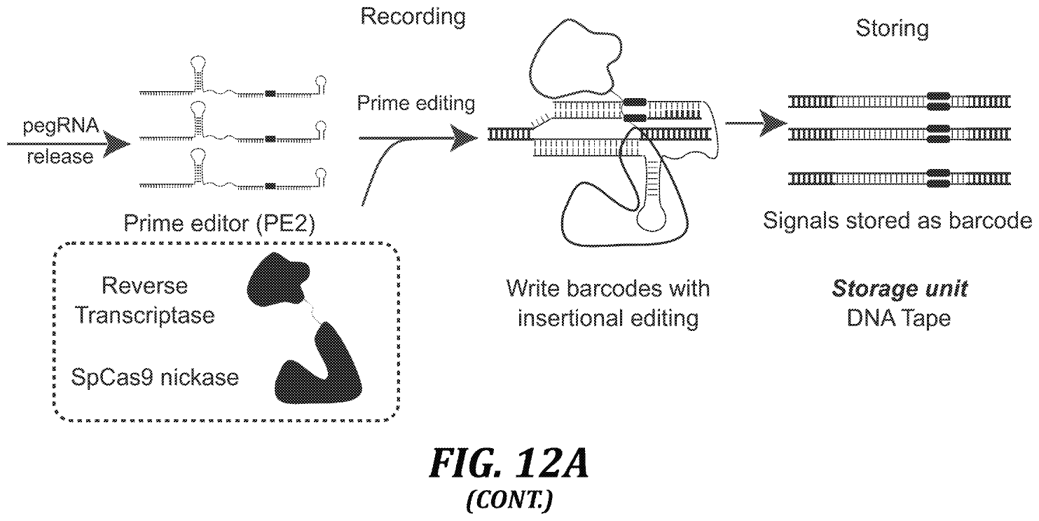

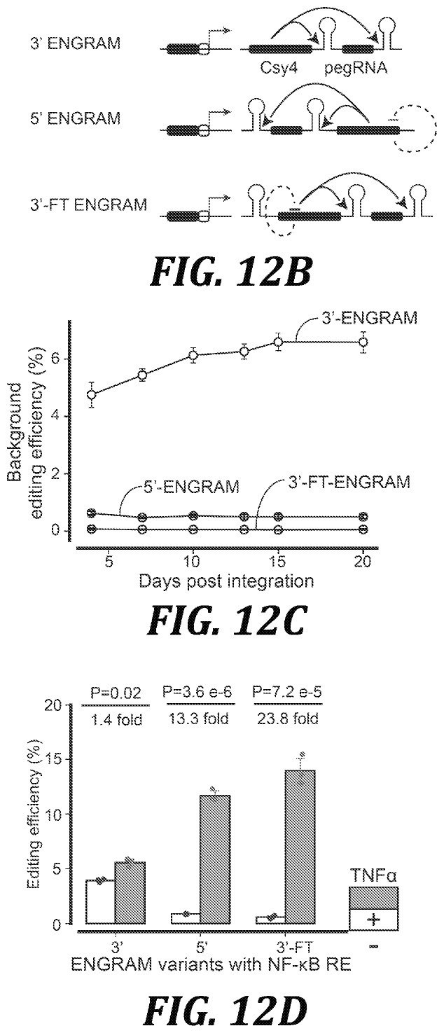

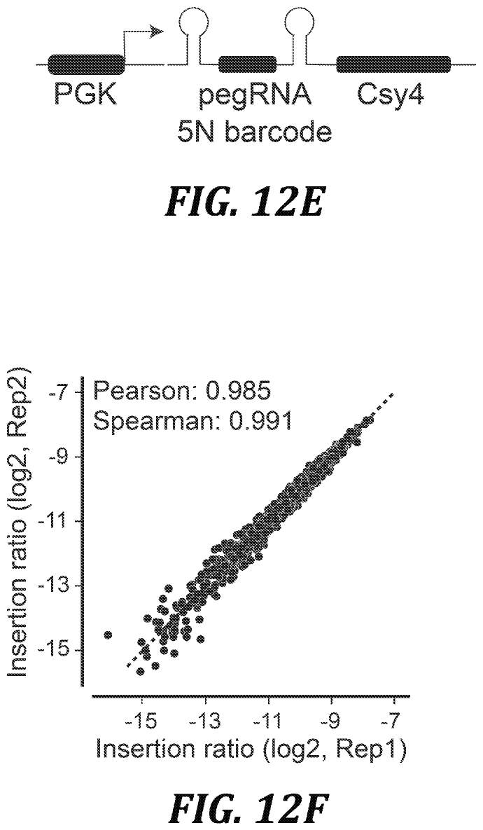

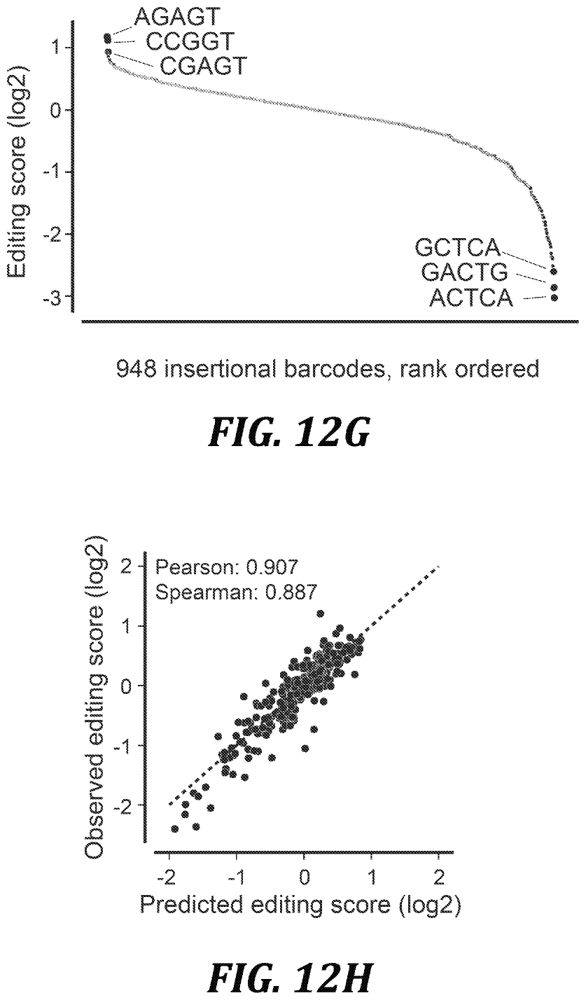

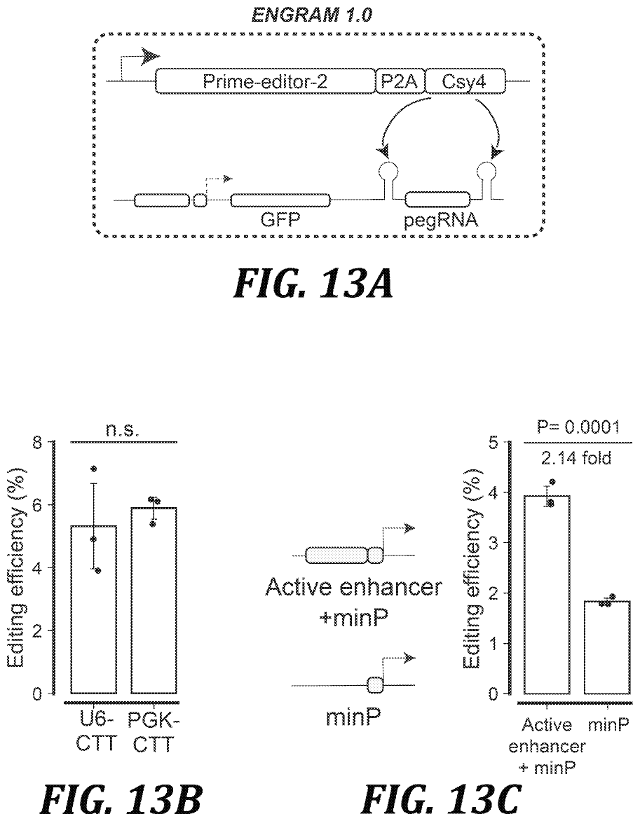

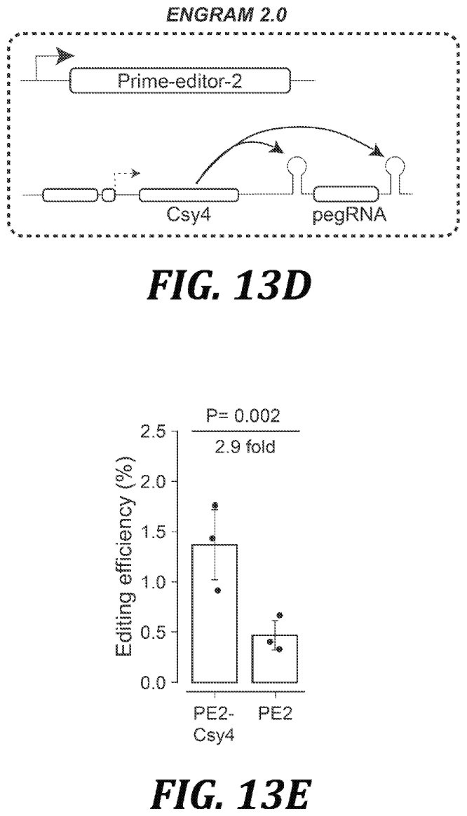

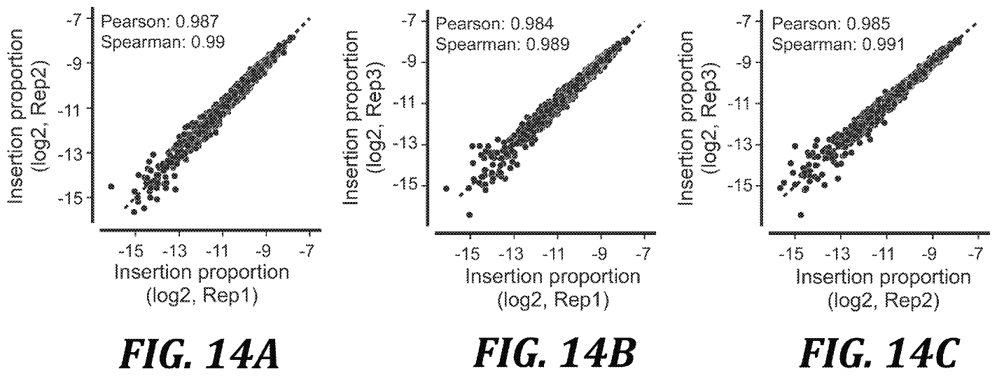

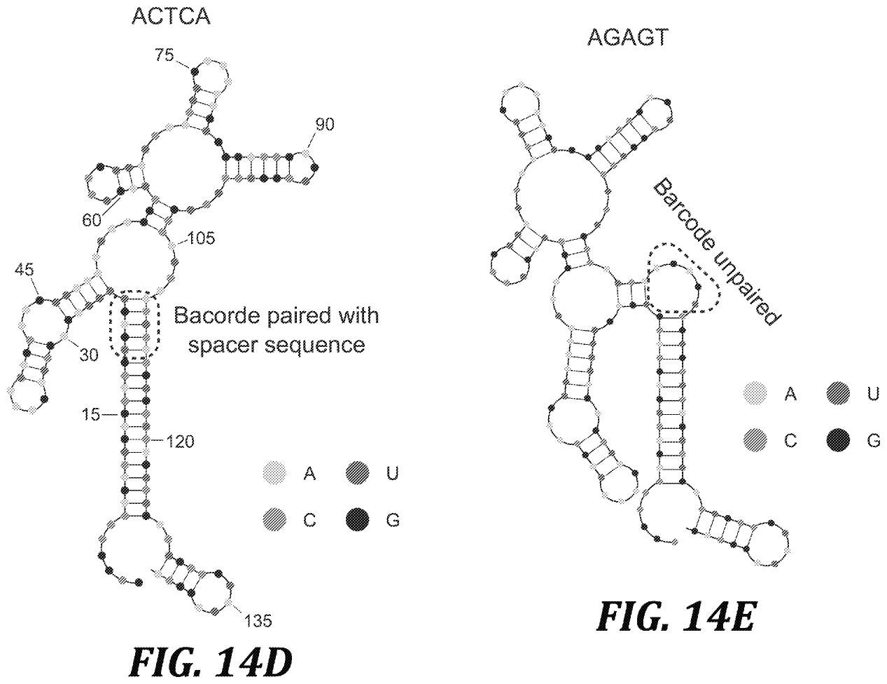

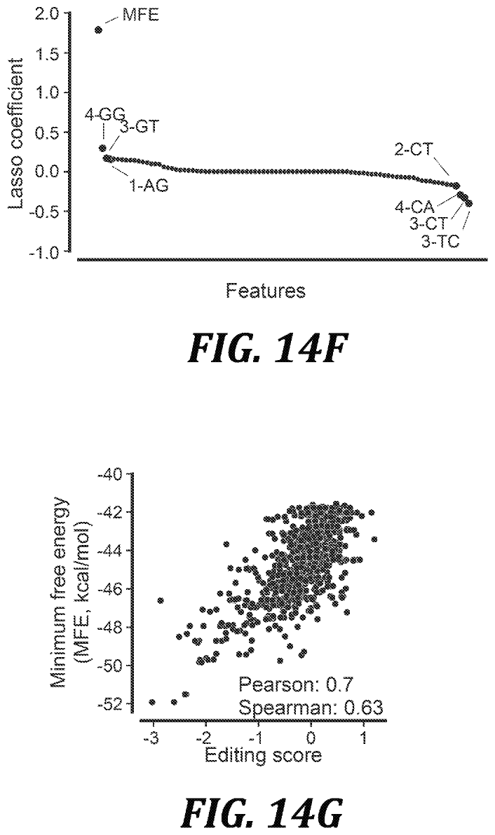

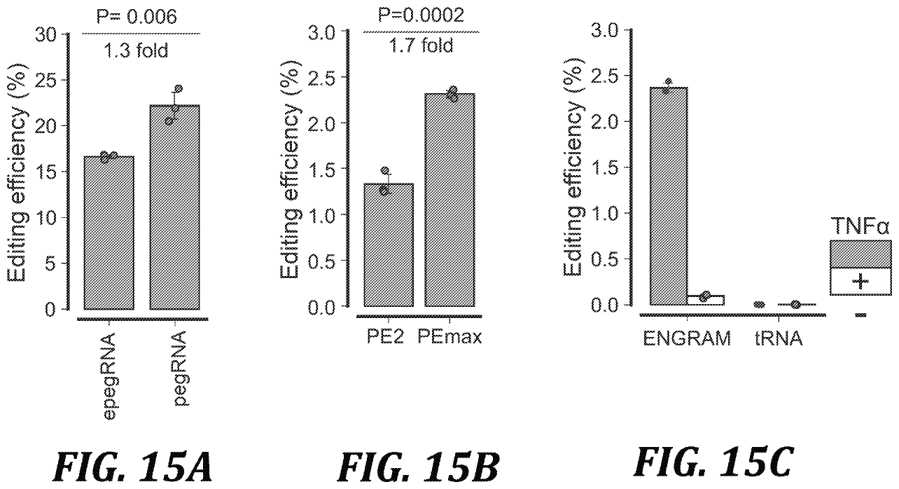

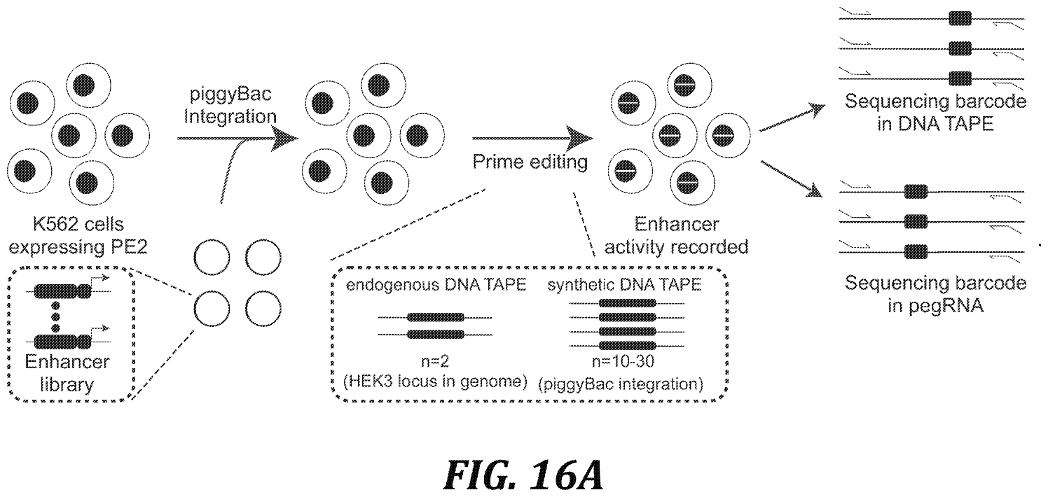

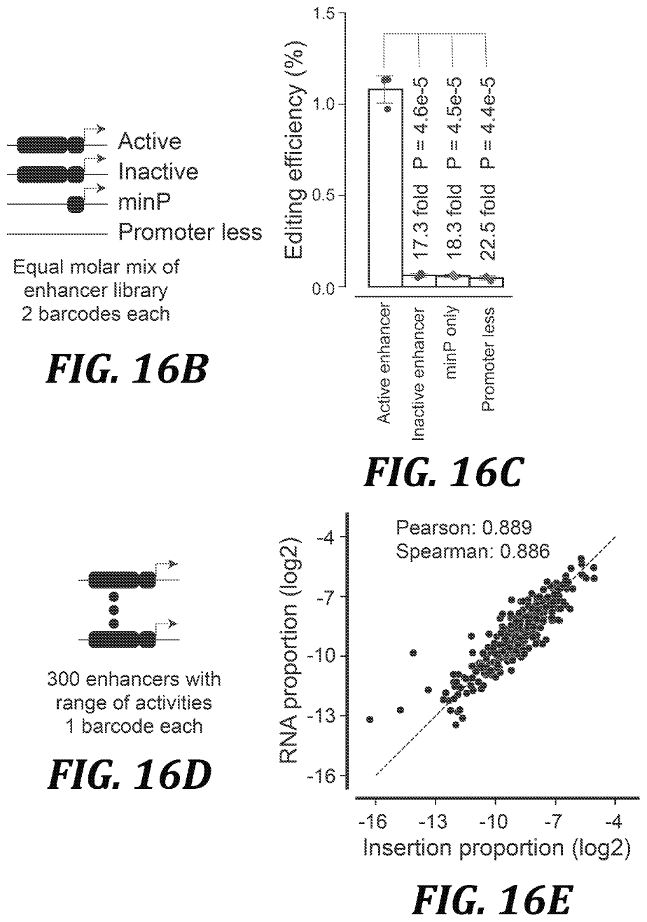

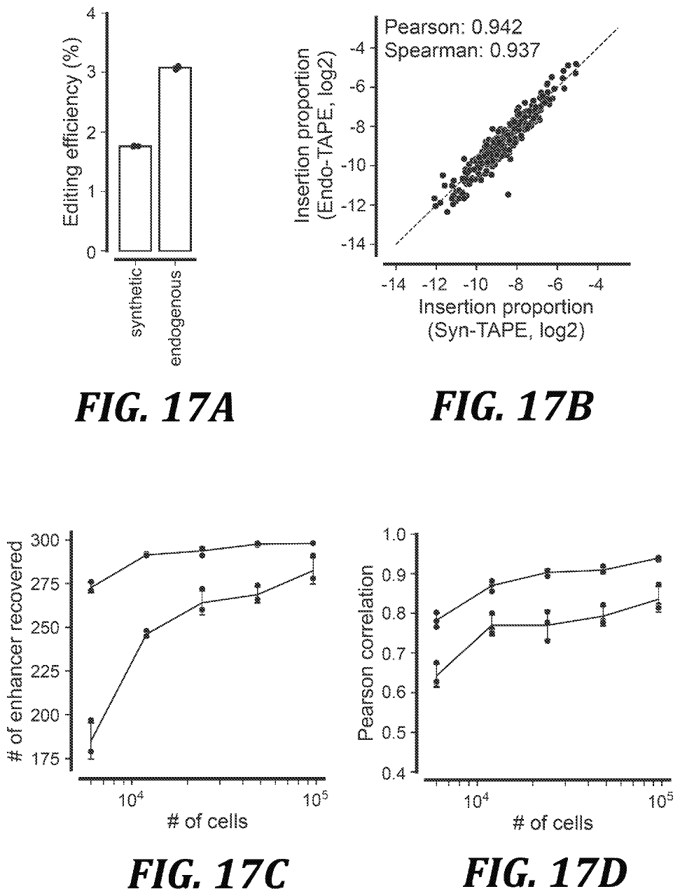

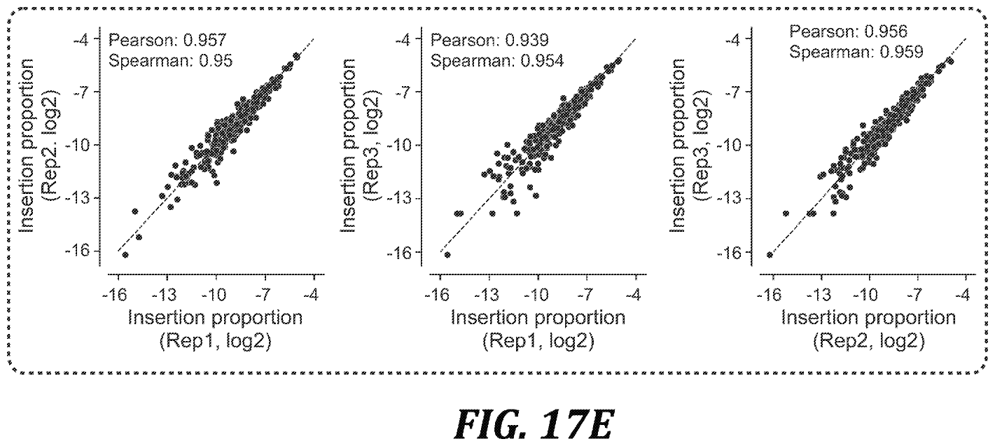

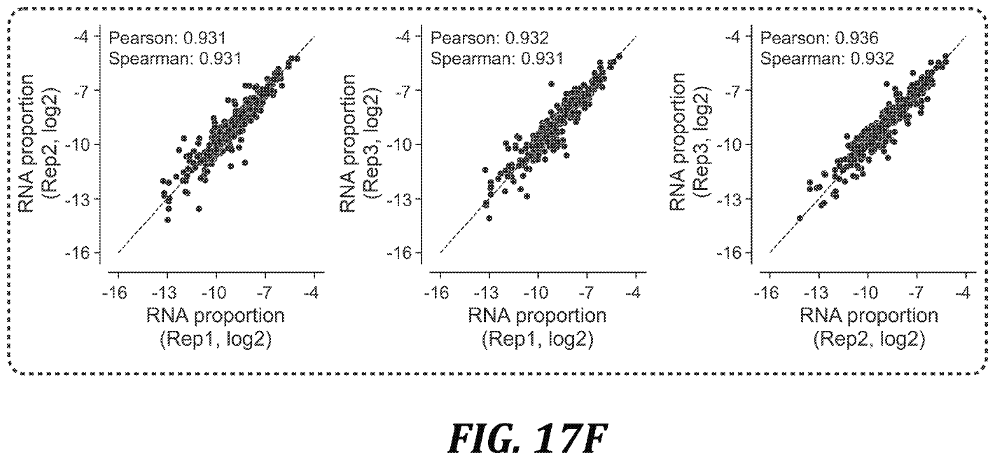

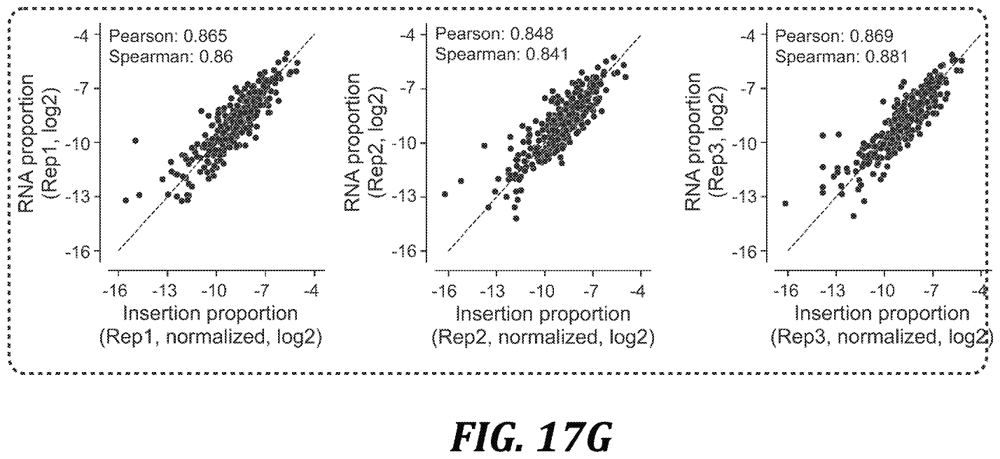

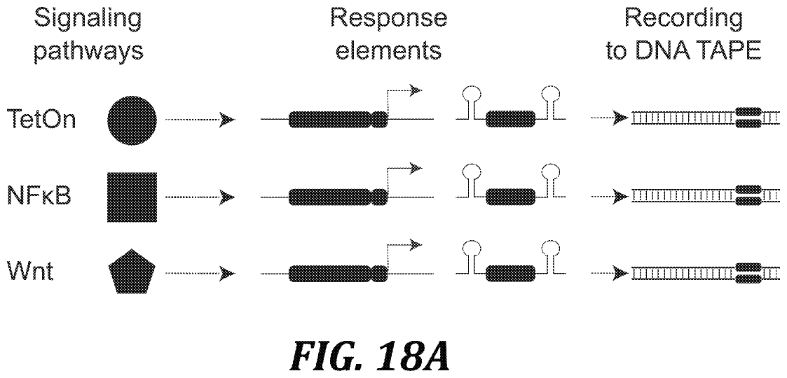

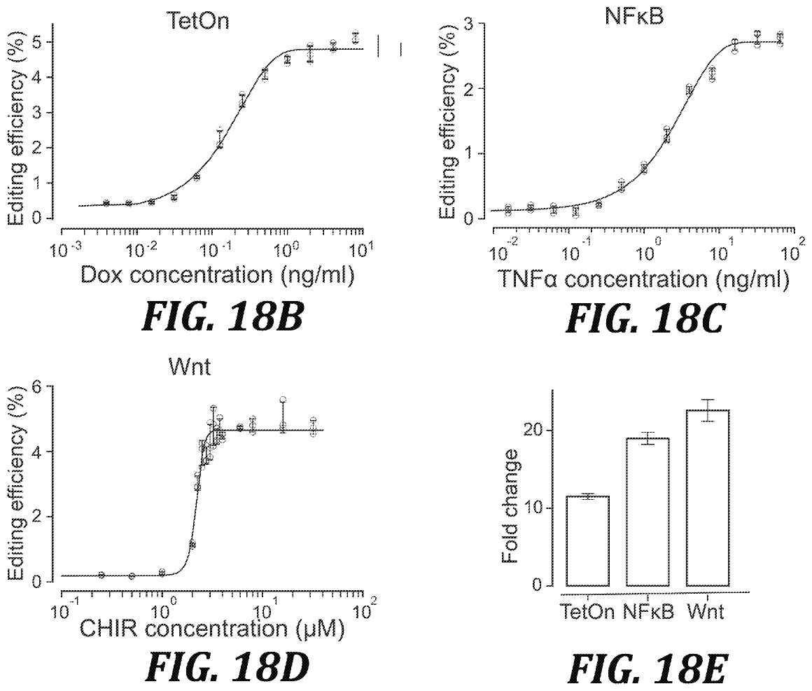

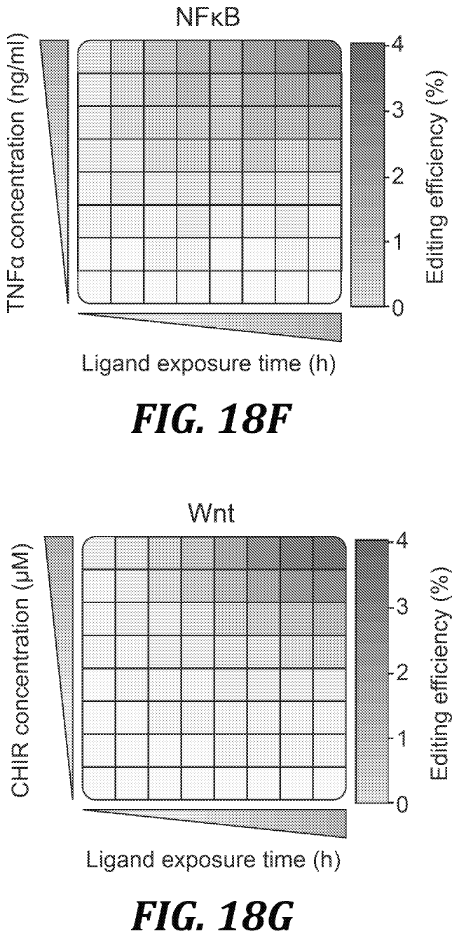

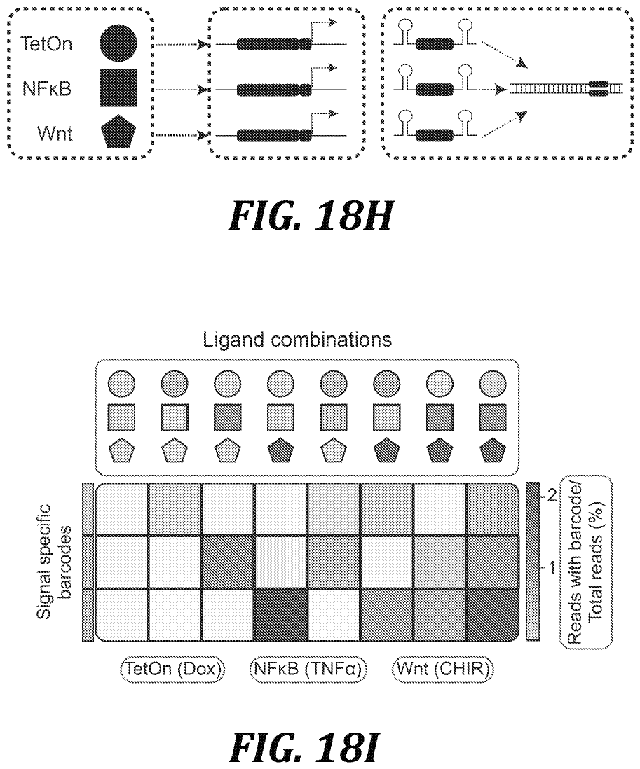

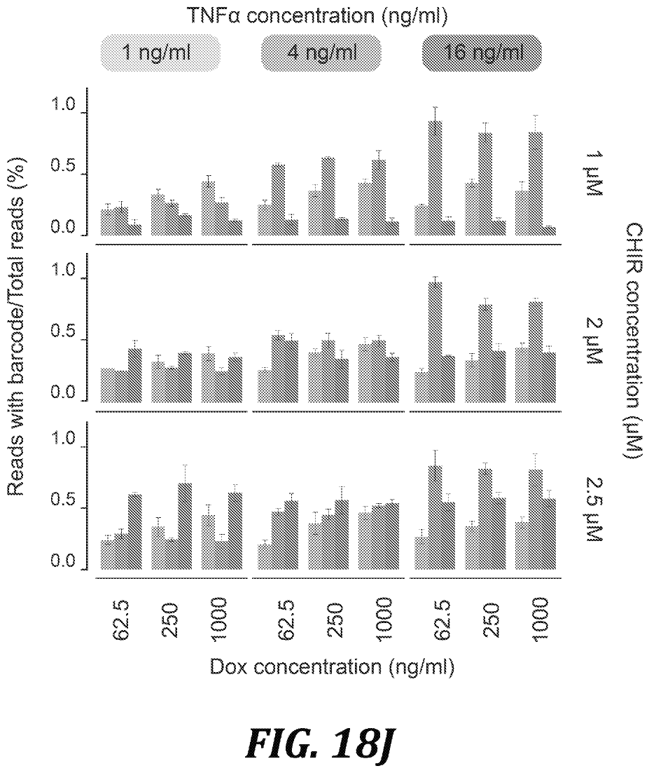

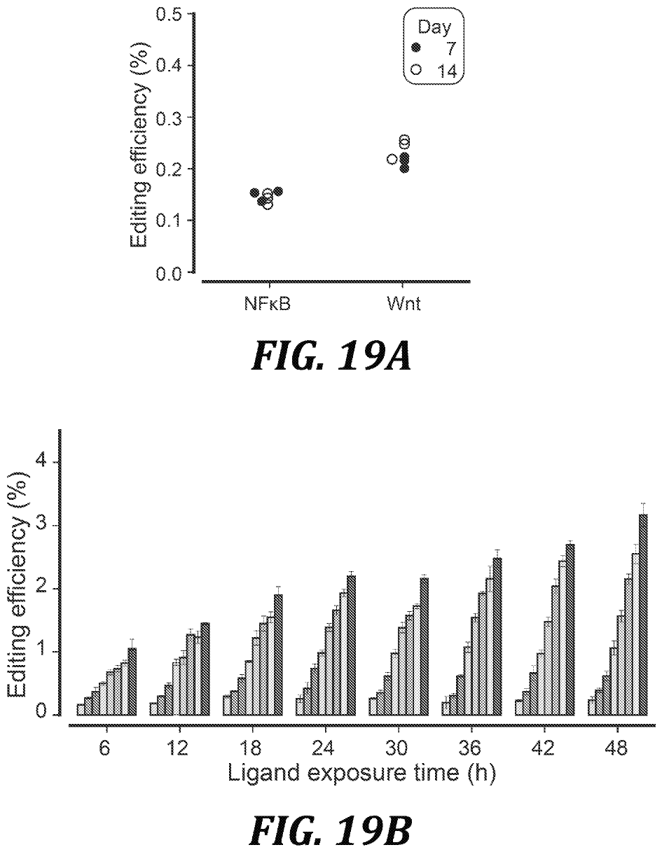

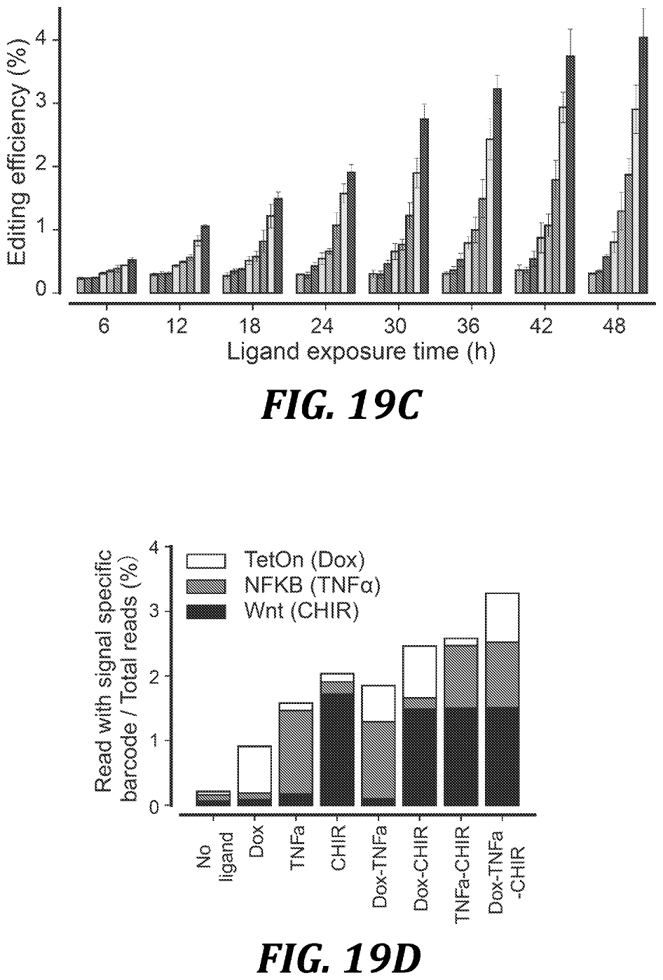

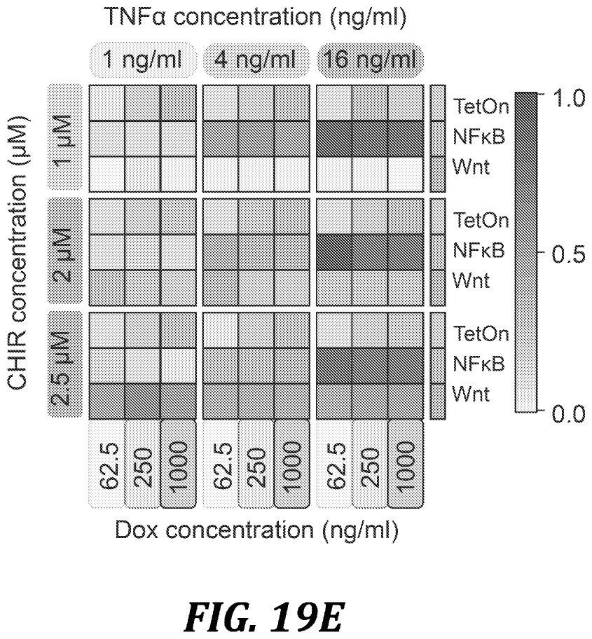

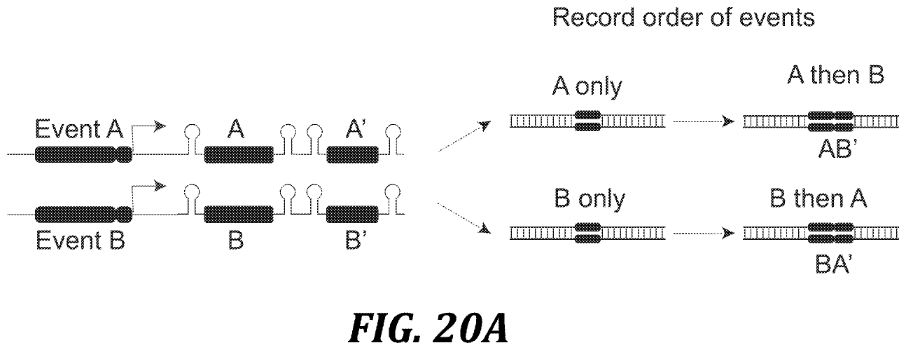

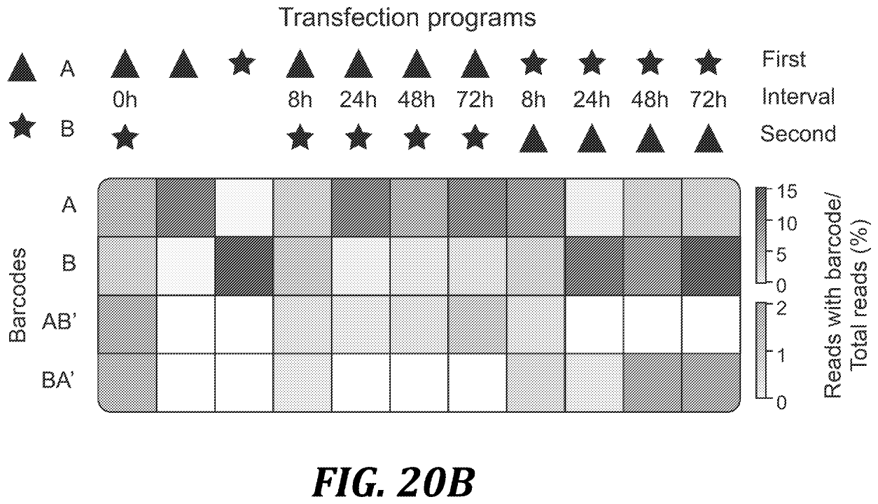

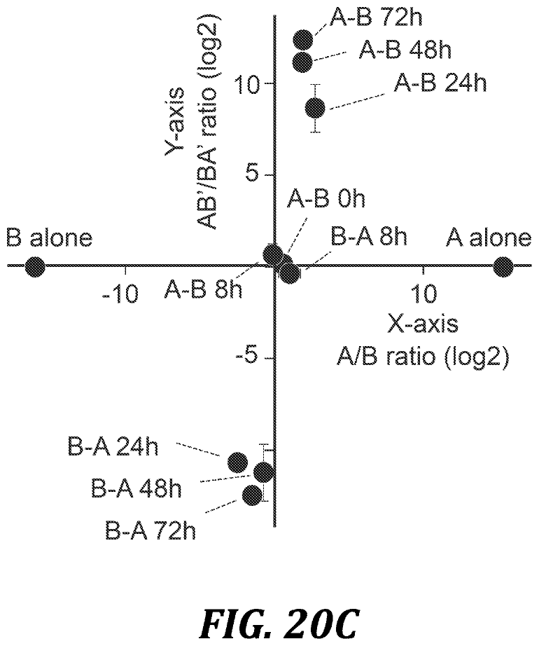

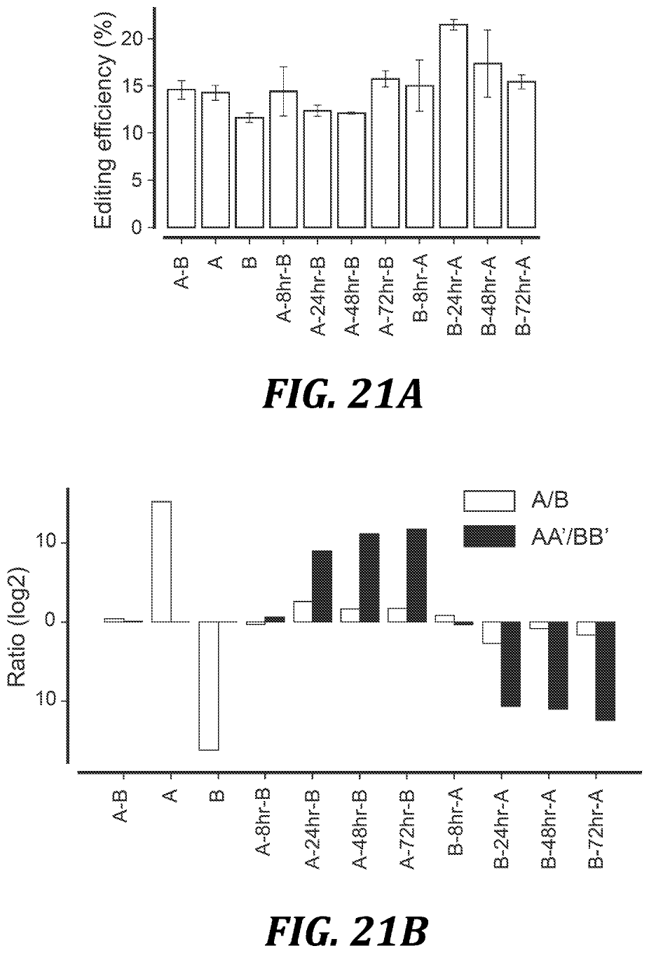

This Example describes a DNA-based memory device that is: (1) highly multiplexable, i.e., compatible with the concurrent recording of at least thousands of distinct symbols or event types; (2) sequential and unidirectional in recording events to DNA, and therefore able to explicitly capture the precise order of recorded events; and (3) active in mammalian cells. This This system, called DNA Typewriter, begins with a tandem array of partial CRISPR-Cas9 target sites (“DNA Tape”), all but the first of which are truncated at their 5′ ends, and therefore inactive (FIG. 1A-C). Each of many prime editing guide RNAs (pegRNAs), together with the prime editing enzyme, is designed to mediate the insertion of a k-mer within the sole active site of the tandem array, which is initially its 5′-most target site. In the simplest implementation, all pegRNAs target the same 20-bp spacer, but each encodes a unique “symbol” in the form of a k-mer insertion. Specifically, the 5′ portion of the k-mer insertion is the variable and encodes the identity of the pegRNA, while its 3′ portion is constant, and activates the subsequent target site in the tandem array by restoring its 5′ end. Thus, each successive edit records the identity of the pegRNA mediating the edit, while also shifting the position of the active target site by one unit along the array. At any moment, an intact spacer and PAM are present at only one location along the array, analogous to the “write-head” of a disk drive or the “type-guide” of a typewriter.

### Proof-of-Concept of DNA Typewriter

To test this idea, DNA Tape (“TAPE-1”) was designed by modifying a spacer sequence previously shown to be highly amenable to prime editing by the PE2 enzyme (HEK293 target 3 or HEK3). In TAPE-1, a 3-bp key (GGA) is followed by one tandem array of a 14-bp monomer (TGATGGTGAGCACG) (SEQ ID NO: 57) that includes the PAM sequence (TGG) at positions 4-6. At the 5′-most end of the TAPE-1 array, the key sequence, the first 14-bp monomer, and the first 6 bases of the subsequent 14-bp monomer, collectively comprise an intact 20-bp spacer and PAM (FIG. 1A). A set of 16 pegRNAs was further designed to target TAPE-1, with each pegRNA programming a distinct 5-bp insertion (FIG. 1B). The first 2-bp of the insertion is unique to each of the 16 pegRNAs. The remaining 3-bp of the insertion corresponds to the key (GGA). The inventors reasoned that when a pegRNA/PE2-mediated insertion occurred at the active TAPE-1 site, it would: (1) record the identity of the pegRNA via the 2-bp portion of the insertion; (2) inactivate the current active site by disrupting its sequence; and (3) activate the next monomer along the array, as the newly inserted GGA key, together with the subsequent 20-bp, creates an intact 20-bp spacer and PAM. In the next iteration of genome editing, a pegRNA-mediated insertion to the second monomer would be recorded while also moving the type-guide to the third monomer, and then to the fourth, the fifth, and so on (FIG. 1C).

TAPE-1 arrays were synthesized and cloned with varying numbers of monomer units (2×TAPE-1, 3×TAPE-1, 5×TAPE-1), and stably integrated these arrays into the genome of HEK293T cells via the piggyBAC system. The resulting cells were transiently transfected with a pool of plasmids designed to express PE2 (pCMV-PE2-P2A-GFP; Addgene #132776) and sixteen pegRNAs, each programmed to insert an NNGGA barcode to TAPE-1, and harvested them after four days. The TAPE-1 region was PCR-amplified from genomic DNA and sequenced.

For each TAPE-1 array, the sequencing reads were categorized into those in which: (1) no editing occurred; (2) the observed pattern was consistent with sequential, directional editing; or (3) the observed pattern was inconsistent with sequential, directional editing (FIG. 1D-F; Table 1). Overall editing rates were modest, as only 4.7±0.5%, 5.2±0.6%, and 5.9±0.8% of all reads for 2×TAPE-1, 3×TAPE-1, and 5×TAPE-1, respectively, exhibited any editing. However, within the set of reads showing edits, the data were overwhelmingly consistent with sequential, directional editing. For example, with 2×TAPE-1, the second monomer was edited in 22.8±1.7% of reads in which the first monomer was also edited (FIG. 1D). In contrast, the second monomer was only edited in 0.6% of reads in which the first monomer was not edited. This observation strongly suggests that edits of the second monomer were dependent on an edit of the first monomer having already occurred. Furthermore, it confirms that the 3-bp mismatch at the PAM-distal end of “inactive” spacers of the TAPE-1 design is sufficient to inhibit prime editing. Data obtained from 3×TAPE-1 and 5×TAPE-1 were also consistent with sequential genome editing. For example, 98.5% (3×TAPE-1) and 99.0% (5×TAPE-1) of reads that were edited at the second monomer were also edited at the first monomer, while 97.6% (3×TAPE-1) and 98.8% (5×TAPE-1) of reads that were edited at the third monomer were also edited at the first and second monomers (FIG. 1E-1F). These results were consistent across three transfection replicates (Table 1).

TABLE 1
Read counts and editing efficiencies for 2xTAPE-1, 3xTAPE-1, and 5xTAPE-1
2xTAPE-1

| Editing groups: | OO | XO | XX | OX | Total reads | 2xTAPE | Retained % |
|---|---|---|---|---|---|---|---|
| replicate 1 | 207933 | 9142 | 2445 | 8 | 225727 | 219528 | 97.3 |
| replicate 2 | 208899 | 7267 | 2349 | 20 | 224350 | 218535 | 97.4 |
| replicate 3 | 266298 | 9320 | 2771 | 13 | 286088 | 278402 | 97.3 |

| Editing efficiencies: | Site 1 | Site 2 |
|---|---|---|
| replicate 1 | 0.053 | 0.211 |
| replicate 2 | 0.044 | 0.244 |
| replicate 3 | 0.043 | 0.229 |

3xTAPE-1

| Editing | | | | | | | | Total | | Retained |
|---|---|---|---|---|---|---|---|---|---|---|
| groups: | OOO | XOO | XXO | XXX | OXO | OOX | OXX | reads | 3xTAPE | % |
| replicate 1 | 283965 | 13522 | 3480 | 714 | 28 | 1 | 8 | 303326 | 301718 | 99.5 |
| replicate 2 | 350054 | 14408 | 3516 | 818 | 54 | 3 | 0 | 370743 | 368853 | 99.5 |
| replicate 3 | 413965 | 15573 | 3827 | 1086 | 50 | 9 | 7 | 436740 | 434517 | 99.5 |

| Editing | | | |
|---|---|---|---|
| efficiencies: | Site 1 | Site 2 | Site 3 |
| replicate 1 | 0.059 | 0.237 | 0.170 |
| replicate 2 | 0.051 | 0.231 | 0.189 |
| replicate 3 | 0.047 | 0.240 | 0.221 |

5xTAPE-1

| Editing | | | | | | | | |
|---|---|---|---|---|---|---|---|---|
| groups: | OOOOO | XO | XXO | | XXXO | | XXXXO | |
| replicate 1 | 177509 | 9860 | 2221 | | 345 | | 46 | |
| replicate 2 | 136239 | 6764 | 1556 | | 230 | | 25 | |
| replicate 3 | 224153 | 9470 | 2084 | | 322 | | 47 | |
| Editing | | | | | | | | |
| efficiencies: | Site 1 | Site 2 | Site 3 | | Site 4 | | Site 5 | |
| replicate 1 | 0.066 | 0.210 | 0.151 | | 0.129 | | 0.098 | |
| replicate 2 | 0.059 | 0.212 | 0.144 | | 0.119 | | 0.194 | |
| replicate 3 | 0.051 | 0.206 | 0.154 | | 0.150 | | 0.175 | |
| Editing | | | | | | Total | 5xTAPE | Retained |
| groups: | XXXXX | OX | NOX | NNOX | NNNOX | reads | reads | % |
| replicate 1 | 5 | 28 | 5 | 3 | 8 | 194828 | 190030 | 97.5 |
| replicate 2 | 6 | 9 | 3 | 1 | 12 | 148403 | 144845 | 97.6 |
| replicate 3 | 10 | 11 | 1 | 9 | 24 | 242373 | 236131 | 97.4 |

5xTAPE-1 (6-bp ins.)

| Editing | | | | | | | | |
|---|---|---|---|---|---|---|---|---|
| groups: | OOOOO | XO | XXO | | XXXO | | XXXXO | |
| replicate 1 | 257305 | 12189 | 2458 | | 201 | | 14 | |
| replicate 2 | 202035 | 10126 | 2002 | | 159 | | 1 | |
| replicate 3 | 139753 | 6671 | 1345 | | 97 | | 35 | |
| Editing | | | | | | | | |
| efficiencies: | Site 1 | Site 2 | Site 3 | | Site 4 | | Site 5 | |
| replicate 1 | 0.055 | 0.180 | 0.083 | | 0.099 | | 0.364 | |
| replicate 2 | 0.057 | 0.177 | 0.078 | | 0.059 | | 0.900 | |
| replicate 3 | 0.055 | 0.181 | 0.089 | | 0.265 | | 0.000 | |
| Editing | | | | | | Total | 5xTAPE | Retained |
| groups: | XXXXX | OX | NOX | NNOX | NNNOX | reads | reads | % |
| replicate 1 | 8 | 13 | 7 | 3 | 14 | 279612 | 272212 | 97.4 |
| replicate 2 | 9 | 12 | 0 | 5 | 17 | 219587 | 214366 | 97.6 |
| replicate 3 | 0 | 11 | 11 | 11 | 0 | 151607 | 147934 | 97.6 |

Table 1. Read counts and editing efficiencies for 2×TAPE-1, 3×TAPE-1, and 5×TAPE-1. Sequencing reads were grouped based on the observed editing pattern. For example, reads from 2×TAPE-1 array were categorized into four groups: (A) no edit at either TAPE-1 site (‘OO’); (B) 5-bp insertion at the first TAPE-1 site only (‘XO’); (C) 5-bp insertion at the second TAPE-1 site only (‘OX’); or (D) 5-bp insertions at both TAPE-1 sites (‘XX’). For reads from 5×TAPE-1 array, editing groups were simplified by categorizing directional and iterative editing pattern (OOOOO, XO, XXO, XXXO, XXXXO, and XXXXX) and the erroneous editing patterns (OX, NOX, NNOX, and NNNOX, where N can be either O or X). Editing efficiencies at each site were calculated as the fraction of reads with an edit at the site over the total number of reads in which the site had been activated via insertion of the ‘key’ that completed the spacer sequence. 5-bp insertions were tested except for the 5×TAPE-1 array, where 6-bp insertions (random 3-bp plus 3-bp key sequence) were also tested.

An interesting phenomenon is that while the observed editing rate of the first TAPE-1 monomer was ˜6%, the editing rates of the second or third TAPE-1 monomers, conditional on the preceding monomers already being edited, were ˜20% (FIG. 5A). A simple explanation for this ˜14% greater “elongation” than “initiation” of editing is that some integrated tapes are more amenable to prime editing than others, resulting in an excess of fully unedited tapes. However, a similar pattern was also observed with episomal tapes, as well as upon multiple sequential transfections of pegRNA/PE2-expressing plasmids to edit integrated tapes (7-15% increase in the conditional editing efficiency of the second site). Factors that might contribute to the observed “pseudo-processivity” include heterogeneous susceptibility of cells to transfection, chromatin context, and cell cycle phase, but the primary explanation remains unclear. Modest reductions were also observed in the conditional editing efficacy after the second site (1-10% decreases), which might be explained simply by each site being “active” for less time than its predecessor.

The distribution of the 16 NNGGA barcode insertions were analysed focusing on 5×TAPE-1. Their frequencies were correlated across three replicates as well as between the first and second target sites (Pearson's r=0.97-0.99; FIGS. 5B and 5C). The observed variation was partly explained by the relative abundances of the individual pegRNAs in the plasmid pool (Pearson's r=0.87; FIG. 5D). To explore whether the sequence of the insertion itself influences editing efficiency, the experiment was repeated but with an equimolar pool of 16 pegRNA-expressing plasmids that had been individually cloned and purified (rather than cloned as a pool). For each of the NNGGA insertions in each experiment, “edit scores” were calculated as their log 2-scaled insertion frequencies normalised by the abundances of pegRNAs in the corresponding plasmid pools (FIG. 1G). The maximal edit score difference between the best barcode (CCGGA with an edit score of 0.98) and the worst barcode (TGGGA with an edit score of −2.38) is 3.36, i.e., a nearly 10-fold difference in editing efficiency. However, 10 of 16 barcodes exhibited efficiencies within a 2-fold range. Edit scores were well correlated between 5×TAPE-1 edited by the 16 pegRNA plasmids pooled pre-vs. post-cloning (Spearman's p=0.97; FIG. 5E), consistent with an insertion sequence-dependent bias. Indeed, when the relative efficiencies observed were used in the “post-cloning pooling” experiment to correct the TAPE-1 unigram barcode frequencies measured in the “pre-cloning pooling” experiment, their correlation with the abundances of the corresponding pegRNAs in the plasmid pool improved (Pearson's r=0.87→0.94; FIG. 5D), and vice versa (Pearson's r=0.27→0.67; FIG. 5F).

### Enhanced Prime Editing of DNA Tape

Several strategies to improve the efficiency of prime editing via modular engineering were recently reported: 1) Adding degradation-resistant secondary structure to the 3′-end of the pegRNA (Nelson, J. W. et al. Engineered pegRNAs improve prime editing efficiency. Nat. Biotechnol. (2021) doi: 10.1038/s41587-021-01039-7) (resulting in enhanced pegRNAs or epegRNAs); 2) Introducing human MLH1 dominant negative peptide (hMLH1dn) to favor the intended edit (Chen, P. J. et al. Enhanced prime editing systems by manipulating cellular determinants of editing outcomes. Cell 184, 5635-5652.e29 (2021)); and 3) Modifications to the primary sequence of the prime editing enzyme (Chen, P. J. et al., Cell 184, 5635-5652.e29 (2021)) (resulting in PEmax). Combined deployment of these strategies has been reported to improve the editing efficiency by ˜3.5-fold in HEK293T cells and ˜72-fold in Hela cells, relative to PE2 and pegRNAs (Chen, P. J. et al., Cell 184, 5635-5652.e29 (2021)).

As the initial experiments with PE2 and pegRNAs resulted in only modest editing of the first site of TAPE-1 (˜6%), the inventors sought to incorporate these new strategies. A pool of U6-driven epegRNAs were cloned, each programmed to insert an NNGGA barcode to TAPE-1, and transfected them to HEK293T cells integrated with 5×TAPE-1 (5×TAPE-1(+) HEK293T) along with a plasmid expressing PEmax and hMLH1dn (pCMV-PEmax-P2A-hMLH1dn; Addgene #174828). After 4 days, genomic DNA was harvested, and then PCR amplified and sequenced TAPE-1. The first site was edited 18.1±0.5% (FIG. 6A), a nearly 3-fold increase relative to PE2 and pegRNAs, while editing remained overwhelmingly sequential (>99.5%). Next, 4 more pools were cloned, encoding 6-bp (NNNGGA) to 9-bp (NNNNNNGGA) barcodes. The epegRNA/PEmax/hMLH1dn prime editing system achieved reasonably high efficiencies for longer insertions, (e.g., 10.6±0.5% for 9 bp insertions; FIG. 6A). Edit scores for pegRNA/PE2 vs. epegRNA/PEmax/hMLH1dn were highly correlated (Spearman's p=0.96 for NNGGA; Spearman's p=0.88 for NNNGGA; FIG. 6B-6E). The edit scores for epegRNAs were more uniform compared to standard pegRNAs, as 14 of 16 NNGGA barcodes exhibited efficiencies within a 2-fold range (FIG. 6C) and 59 of 64 NNNGGA barcodes within a 4-fold range (FIG. 6E). Edit scores were calculated for more than 1,900 barcodes in NNNNNNGGA (or 6N+GGA) TAPE-1 targeting epegRNAs in a single experiment (FIG. 6F-6I), markedly expanding the number of unique “symbols” that can be encoded and deployed to write to a shared DNA Tape by two orders of magnitude, relative to the inventors' original NNGGA experiment. 1,509 out of 1,908 6N+GGA barcodes exhibited efficiencies with edit scores between-1 and 1, i.e., a 4-fold range (FIG. 6H).

To evaluate the compatibility of DNA Typewriter with cell types other than HEK293Ts, 5×TAPE-1 target was integrated to mouse embryonic fibroblasts (MEFs) and mouse embryonic stem cells (mESCs) using the piggyBAC transposase system and transfected them with either a pool of 16 NNGGA epegRNAs or a pool of 64 NNNGGA epegRNAs with PEmax/hMLH1dn expressing plasmids via electroporation of DNA plasmids. After 4 days, genomic DNA was harvested, and then amplified and sequenced TAPE-1. After 4 days, the first site was edited 7.0-18.1% (FIG. 6J). In mESCs, where prolonged culturing was permitted compared to MEFs, a second transfection was performed with the same set of epegRNA/PEmax/hMLH1dn expressing plasmids, 4 days after the first transfection. The cumulative editing of the first site increased to 28.7±2.8% when the sample was collected another 4 days after the second transfection. Of note, the edit scores for NNGGA and NNNGGA pegRNAs in mESCs are reasonably well correlated with those measured in HEK293Ts (FIG. 6K-6L), suggesting that measurements of relative pegRNA efficiencies made in in HEK293Ts are applicable to other cell types. Collectively, these results demonstrate that the performance of DNA Typewriter can be improved using methods that enhance prime editing, and furthermore that the method can be used in primary and stem cells. Overall, the range and efficiency of DNA Typewriter will be tightly coupled to that of prime editing, which has also been demonstrated to work in human induced pluripotent stem cells (iPSCs) and primary human T cells (Chen, P. J. et al., Cell 184, 5635-5652.e29 (2021)).

### Screening Additional DNA Tape Sequences

The TAPE-1 construct exhibited sequential, directional editing, wherein the editing of any given site along the array was strongly dependent on all preceding sites having already been edited. This behaviour is consistent with the DNA Typewriter's design, as the key sequence must be inserted 5′ to any given monomer within DNA Tape in order to complete the spacer that is recognized by any of the guide RNAs used. However, performance would presumably be corrupted by non-specific editing, e.g. if a guide were able to mediate edits to a non-type-guide monomer despite several mismatches at the 5′ end of the spacer (Hsu, P. D. et al. DNA targeting specificity of RNA-guided Cas9 nucleases. Nat. Biotechnol. 31, 827-832 (2013), Kim, D. Y., Moon, S. B., Ko, J.-H., Kim, Y.-S. & Kim, D. Unbiased investigation of specificities of prime editing systems in human cells. Nucleic Acids Res. 48, 10576-10589 (2020)).

Although TAPE-1 exhibited reasonable efficiency and specificity, the inventors sought to explore whether this would be the case for other spacers. To this end, 48 TAPE constructs (TAPE-1 through TAPE-48) were designed and synthesised, each derived from one of eight basal spacers that previously demonstrated reasonable efficiency for prime editing (Anzalone, A. V. et al. Search-and-replace genome editing without double-strand breaks or donor DNA. Nature 576, 149-157 (2019), Kim, H. K. et al. Predicting the efficiency of prime editing guide RNAs in human cells. Nat. Biotechnol. 39, 198-206 (2021), Choi, J. et al. Precise genomic deletions using paired prime editing. Nat. Biotechnol. (2021) doi: 10.1038/s41587-021-01025-z) and one of six design rules that vary monomer sequence, key sequence and key/monomer length (FIG. 7A). In each of these 48 constructs, a 3×TAPE region was accompanied by a pegRNA-expressing cassette designed to target it with a 4-6 bp insertion (16 possible 2-bp barcodes followed by a 2-4 bp key sequence). HEK293T cells were transiently transfected with PE2-encoding plasmid and a pool of 48 pegRNA-by-3×TAPE constructs and harvested them after four days. The 3×TAPE region was PCR-amplified from genomic DNA and sequenced.

Two quantities were calculated for each 3×TAPE array: (1) efficiency, calculated by summing all edited reads and dividing by the total number of reads; and (2) sequential error rate, calculated by summing all edited reads inconsistent with sequential, directional editing and dividing by the total number of edited reads (FIG. 7B). Of note, the initial TAPE-1 construct had one of the lowest sequential error rates among the 48 tested tapes. The only construct that had a lower sequential error rate than TAPE-1 was TAPE-6, which was derived from the same basal spacer (HEK3) but had a 4-bp rather than 3-bp key sequence. Indeed, across the full experiment, a longer key sequence was associated with a lower sequential error rate (FIG. 7C). Performance differences between basal spacers were modest, with TAPEs based on the HEK3 and FANCF spacers exhibiting the best combination of efficiency and specificity (FIG. 7D). Among FANCF-based spacers, TAPE-27 exhibited over 50% greater efficiency than TAPE-1, but also a 2-fold greater sequential error rate (FIG. 7B). Performance characteristics were highly consistent when the experiment was repeated with integration rather than transient transfection of the constructs (FIG. 7E).

Overall, these results show considerable variation in efficiencies and sequential error rates, specific to particular 13- to 15-bp TAPE sequences. Although a single well-performing monomer such as either TAPE-1 or TAPE-27 is sufficient to construct a generic substrate to which thousands of distinct symbols can be written, additional screening might yield monomers with even better performance characteristics, and would also facilitating modelling of the sequence determinants of monomer performance (Hsu, P. D. et al. DNA targeting specificity of RNA-guided Cas9 nucleases. Nat. Biotechnol. 31, 827-832 (2013), Kim, D. Y., Moon, S. B., Ko, J.-H., Kim, Y.-S. & Kim, D. Unbiased investigation of specificities of prime editing systems in human cells. Nucleic Acids Res. 48, 10576-10589 (2020), Kim, H. K. et al. Predicting the efficiency of prime editing guide RNAs in human cells. Nat. Biotechnol. 39, 198-206 (2021), Doench, J. G. et al. Optimized sgRNA design to maximize activity and minimize off-target effects of CRISPR-Cas9. Nat. Biotechnol. 34, 184-191 (2016)).

### Recording Complex Event Histories

Next, experiments were performed to determine whether DNA Typewriter can be applied to record, recover, and decode complex event histories. A set of synthetic signals was prepared by individually cloning 16 individual pegRNA-expressing plasmids, each encoding a unique 2-bp barcode insertion to TAPE-1. A polyclonal population of HEK293T cells was prepared with integrated 5×TAPE-1 to serve as the substrate for recording. Finally, a set of five “transfection programs”-complex event histories that could be recorded and then subsequently decode-were designed (FIG. 2A).

At the beginning of each epoch of each transfection program, one or more pegRNA plasmids were introduced to a population of HEK293T cells with integrated 5×TAPE-1 (5×TAPE-1(+) HEK293T) via transient transfection of plasmids expressing the corresponding pegRNA(s) and PE2. After each transfection, cells were passaged the next day into a new plate and excess cells were harvested for genomic DNA. 5×TAPE-1 from each epoch of each program was amplified and sequenced. Successive epochs occurred at 3-day intervals.

Programs 1 and 2 each consisted of a distinct, non-repeating sequence of transfection of the 16 pegRNAs, i.e., one per epoch. The specific orders aimed to maximise (Program-1) or minimise (Program-2) the edit distances between temporally adjacent signals. Based on sequencing of 5×TAPE-1 after Epoch-16, it was observed that barcodes introduced in the early epochs were more frequent at the first target site (Site-1) than barcodes introduced at late epochs (FIG. 2B). This is expected, as each editing round shifts more of the type-guides to Site-2 (and subsequently to Site-3 to Site-5) (FIG. 8A), with minimal effects on the integrity of the 5×TAPE-1 array (FIG. 8B). A trivial decoding approach would be to simply arrange barcodes in the order of decreasing Site-1 unigram frequencies, but for both Programs 1 and 2, this results in an incorrect order (FIG. 8C).

However, the inference can be improved by leveraging the sequential aspect of DNA Typewriter, for instance by analysing bigram frequencies or pair-wise appearance of events as used in inferring orders from CRISPR-Cas spacer acquisition process (Cas1-Cas2 system used in bacteria) (Shipman, S. L., Nivala, J., Macklis, J. D. & Church, G. M. Molecular recordings by directed CRISPR spacer acquisition. Science 353, aaf1175 (2016), Shipman, S. L., Nivala, J., Macklis, J. D. & Church, G. M. CRISPR-Cas encoding of a digital movie into the genomes of a population of living bacteria. Nature 547, 345-349 (2017)). For example, if signal B preceded signal A, then we expect many more B-A bigrams than A-B bigrams at adjacent, edited sites within 5×TAPE-1. In FIG. 2C-2D, heatmaps are shown of bigram frequencies measured from all 4 pairs of adjacent editing sites on 5×TAPE-1, arranged by the true order in which the signals were introduced for Programs 1 and 2. Indeed, the bigram frequencies appear to capture event order information, evidenced by the gross excess of observations immediately above vs. immediately below the diagonal (e.g. in Program-1, CA-GC>>GC-CA). One way to leverage this information is by enumerating “ordering rules” among all events for possible permutations and then checking which the observed data matches best (Shipman, S. L., Nivala, J., Macklis, J. D. & Church, G. M. Molecular recordings by directed CRISPR spacer acquisition. Science 353, aaf1175 (2016), Shipman, S. L., Nivala, J., Macklis, J. D. & Church, G. M. CRISPR-Cas encoding of a digital movie into the genomes of a population of living bacteria. Nature 547, 345-349 (2017)). However, the number of ordering rules for n events increases to the order of n² (for ordering 16 events, there are 136 ordering rules, or (n²+n)/2 in general), while the number of possible permutations increases to n factorial. As a more computationally efficient approach, the following algorithm was implemented: (1) initialise with the event order inferred from the Site-1 unigram frequencies; (2) iterate through adjacent epochs from beginning to end, and swap signals A and B if the bigram frequency of B-A is greater than A-B; (3) repeat step 2 until no additional swaps are necessary. For both Programs 1 and 2, this algorithm resulted in the correct ordering of the 16 signals, out of 16 factorial or 21 trillion possibilities (FIG. 8C). This inference was robust to the sequencing depth, as the correct order could be reconstructed from as few as 2500 reads of the 5×TAPE-1 amplicon (FIG. 8D).

The dearth of bigrams inconsistent with the true order, illustrated by the lack of signal below the diagonal in the Program-1 and Program-2 heatmaps (FIG. 2C-2D), indicates minimal interference between adjacent epochs, i.e., transfected pegRNAs from adjacent epochs, did not overlap in their activities. To evaluate performance in the presence of such overlap, Program-3 was designed, in which two barcodes are introduced at each epoch, but adjacent epochs always share one barcode (FIG. 2A). The concurrent transfection of two pegRNAs with distinct barcodes is evident in the resulting bigram frequency matrix, specifically by the signal both immediately above and below the diagonal (FIG. 2E). The aforementioned decoding algorithm performs slightly worse on Program-3, with a single swap between epochs 4 and 5 required to revise the inferred order to the correct order (FIG. 8C).

Finally, it was determined whether the relative strength of signals could be inferred from symbols recorded to DNA Tape. For this, Programs 4 and 5 were designed, which share the same order of barcodes—a pair in each epoch—but with each pair at different ratios in the two programs. In Program-4, pegRNAs encoding each pair of barcodes were always mixed at a 1:3 ratio, whereas in Program-5, the same pairs for each epoch were mixed at a 1:1, 1:2, 1:4 or 1:8 ratio (FIG. 2A). For both programs, the resulting bigram frequency matrix is consistent with expectation, and the order of events was accurately inferred (FIG. 2F-2G; FIG. 8C). However, in addition, the relative ratios at which each pair of barcodes was introduced was compared within each epoch between Programs 4 and 5 and found these to be well correlated with expectation (FIG. 2H; FIG. 8E-8F). Taken together, these results show that DNA Typewriter can record, recover, and decode complex event histories including the order, overlap, and relative strength of signals.

### Recording and Recovering Short Texts

Next, a strategy was designed to record and decode short text messages to populations of cells with DNA Typewriter. In brief, the Base64 binary-to-text encoding scheme was modified by assigning each of the 64 possible 3-mers to 6-bit binaries. The Base64 scheme encodes uppercase and lowercase English characters, numbers from 0 to 9, and two symbols. In the TAPE64 scheme, uppercase English characters, four symbols and a whitespace, were encoded with two-fold or four-fold redundancy (FIG. 3A; Table 2).

TABLE 2
TAPE64 encoding of text symbols to 3-mer barcodes.

| ID | 30mer Barcode (NNNGGA) | Text Symbol |
|---|---|---|
| 1 | AAA | A |
| 2 | AAC | B |
| 3 | AAG | C |
| 4 | AAT | D |
| 5 | ACA | E |
| 6 | ACC | F |
| 7 | ACG | G |
| 8 | ACT | H |
| 9 | AGA | I |
| 10 | AGC | J |
| 11 | AGG | K |
| 12 | AGT | L |
| 13 | ATA | M |
| 14 | ATC | N |
| 15 | ATG | O |
| 16 | ATT | P |
| 17 | CAA | Q |
| 18 | CAC | R |
| 19 | CAG | S |
| 20 | CAT | T |
| 21 | CCA | U |
| 22 | CCC | V |
| 23 | CCG | W |
| 24 | CCT | X |
| 25 | CGA | Y |
| 26 | CGC | Z |
| 27 | CGG | A |
| 28 | CGT | B |
| 29 | CTA | C |
| 30 | CTC | D |
| 31 | CTG | E |
| 32 | CTT | F |
| 33 | GAA | G |
| 34 | GAC | H |
| 35 | GAG | I |
| 36 | GAT | J |
| 37 | GCA | K |
| 38 | GCC | L |
| 39 | GCG | M |
| 40 | GCT | N |
| 41 | GGA | O |
| 42 | GGC | P |
| 43 | GGG | Q |
| 44 | GGT | R |
| 45 | GTA | S |
| 46 | GTC | T |
| 47 | GTG | U |
| 48 | GTT | V |
| 49 | TAA | W |
| 50 | TAC | X |
| 51 | TAG | Y |
| 52 | TAT | Z |
| 53 | TCA | PERIOD |
| 54 | TCC | PERIOD |
| 55 | TCG | COMMA |
| 56 | TCT | COMMA |
| 57 | TGA | QUESTION MARK |
| 58 | TGC | QUESTION MARK |
| 59 | TGG | EXCLAMATION POINT |
| 60 | TGT | EXCLAMATION POINT |
| 61 | TTA | SPACE |
| 62 | TTC | SPACE |
| 63 | TTG | SPACE |
| 64 | TTT | SPACE |

Three messages were selected to encode: (1) “WHAT HATH GOD WROUGHT?”, the first long-distance message transmitted by Morse code in 1844; (2) “MR. WATSON, COME HERE!”, the first message transmitted by telephone in 1876; and (3) “BOUND FOREVER, DNA”, a translation of a lyric from the 2017 song DNA by the K-pop music group BTS. Each message was split into sets of four characters. Plasmids encoding a given set of pegRNAs were concurrently transfected with a plasmid encoding PE2 to 5×TAPE-1(+) HEK293T cells at a ratio of 7:5:3:1, such that the ratio encoded the order of the four characters within each set (FIG. 3B). As such, each full message could be recorded by five to six consecutive transfections spaced by three-day intervals.

To recover and decode the recorded messages, populations of cells corresponding to each message were harvested, and amplified and the tape region sequenced. From the resulting reads, first all characters in the message were identified by examining NNNGGA insertions at Site-1 of 5×TAPE-1. These characters were then grouped into sets by hierarchical clustering (FIG. 9A), while also ordering these sets relative to one another, by applying the algorithm used for the previous experiment to the bigram transition matrix (FIG. 3C-3E). Finally, the four characters within each set were arranged by decreasing order of their edit score-corrected frequency, as within each set, earlier characters were encoded at a higher plasmid concentration.

For all three messages, reconstructions of the original text were reasonable but imperfect. From the first message, 17/22 characters were correctly recovered and ordered, with three deletion errors and one swap between adjacent characters to yield “WA HATH GOD WRUOGT?” (FIG. 3C). Of note, the deletion errors were due to repeated use of pegRNA barcodes ‘ACT’, ‘CAT’, and ‘GAC’ to encode multiple ‘H’ or ‘T’ characters, and as such were not expected to be recovered separately. These deletion errors are the result of the encoding scheme which used only 64 unique pegRNAs; it can be anticipated that greater information content per edit can be achieved with pegRNAs with longer barcodes, e.g., 6-bp barcodes would have allowed each instance of repeated characters to be represented by different insertions, thereby avoiding this kind of error. Consistent with the previous analysis on decoding complex event histories, this inference was robust to sequencing depth, as undersampling did not appreciably add more errors to decoded messages (FIG. 9B). From the second message, 20/22 characters were correctly recovered and ordered, with two deletions and one insertion to yield “MR. WATSON, COMI HEE!” (FIG. 3D). From the third message, 16/18 characters were correctly recovered and ordered, with a single swap between adjacent characters to yield “BOUND FOREVE,R DNA” (FIG. 3E). Despite these errors, this experiment demonstrates the potential of DNA Typewriter to digitally record the content and order of information to the genomes of populations of mammalian cells.

### Ordered Recording of Cell Lineage

Beginning with Genome Editing of Synthetic Target Arrays for Lineage Tracing (GESTALT), several approaches have been developed that leverage stochastic genome editing to generate a combinatorial diversity of mutations that irreversibly accumulate to a compact DNA barcode during in vivo development (McKenna, A. et al. Whole-organism lineage tracing by combinatorial and cumulative genome editing. Science 353, aaf7907 (2016), McKenna, A. & Gagnon, J. A. Recording development with single cell dynamic lineage tracing. Development 146, (2019)). Such stochastically evolving barcodes mark cells and enable inference of their lineage relationships based on patterns of shared mutations. However, despite their promise, GESTALT and similar recorders remain sharply limited by several technical challenges, including: (1) a failure to explicitly record the order of editing events, which renders phylogenetic reconstruction of cell lineage highly challenging; (2) a reliance on double-stranded breaks (DSBs) and nonhomologous end-joining (NHEJ) to introduce edits; DSBs frequently delete or corrupt consecutively located targets within a barcode; and (3) the number of target sites available to CRISPR-Cas9 decreases as sites are irreversibly edited, which effectively makes it impossible to sustain continuous lineage recording over long periods of time without sacrificing resolution.

The ordered manner in which edits accrue with DNA Typewriter, the use of a prime editor with a Cas9 nickase to insert one of many possible “symbols” at the type-guide, the predefined sequences and locations of potential edits, and the fact that one-and-only-one monomer is an active type-guide at any given moment, have the potential to address all of these limitations at once. To demonstrate this potential, cell lineage during the expansion of a monoclonal cell line was recorded, leveraging DNA Typewriter in combination with single cell RNA-seq (sc-RNA-seq). First, a HEK293T cell line that expresses doxycycline (Dox)-inducible PE2 (iPE2(+) HEK293T) was constructed. A lentiviral construct was designed and cloned that includes: (1) the 5×TAPE-1 sequence, associated with a random 8-bp barcode region (TargetBC) at its 5′-end; (2) a transcription cassette for the TargetBC-5×TAPE-1 with a reverse transcription capture sequence for enrichment during sc-RNA-seq; and (3) a constitutive pegRNA expression cassette that targets TAPE-1 for a 6-bp insertion (NNNGGA; referred to below as InsertBC; GGA is the key sequence for TAPE-1) (FIG. 4A). Lentiviral transduction of this construct to the cell line at a high multiplicity of infection (MOI) was followed by serial dilution to isolate a monoclonal cell line that grew from 1 cell to ˜1.2M cells via ˜20 doublings over 25 days in the presence of Dox (FIG. 4B; FIG. 10A). After harvesting, sc-RNA-seq was used to recover and sequence multiple TargetBC-5×TAPE-1 arrays from each of ˜12,000 cells.

The frequency distribution of recurrently observed TargetBCs and InsertBCs in these data suggested that the MOI for this monoclonal cell line was ˜19 (FIG. 10B-10C; Methods). However, the DNA Tapes associated with some TargetBCs were recovered more effectively than others (FIG. 10D), presumably due to site-of-integration effects on expression. To minimise complications related to missing data, the analysis was focused on cells for which tape sequences were recovered from all of the 13 most frequently observed TargetBCs, excluding one tape sequence with a corrupted type-guide (TargetBC ATAAGCGG). Although the sequencing error rate was estimated to be very low (FIG. 10D-10E), the accumulation of errors across edited sites might affect the lineage reconstruction. Therefore, it was also required that all edits to these tapes were among the 19 most frequently observed InsertBCs.

Applying these filters left 3,257 cells, for each of which intact TAPE-1 sequences were recovered for each of the 13 prioritised TargetBCs. Although nine of these TAPE-1 sequences were the expected 5 monomers in length, three were 4 monomers in length (TargetBCs TGGACGAC, TTTCGTGA, TGGTTTTG), and one was 2 monomers in length (TargetBC TTCACGTA). Because of their consistent length across the dataset, it can be inferred that these TargetBC-specific contractions are due to pre-existing heterogeneity in the TAPE-1 lentiviral library prior to integration, rather than having been caused by editing. Thus, the TAPE-1 arrays on which the analyses focused included 13 active type-guides and 59 editable sites. With 59 editable sites and 19 potential edits per site, the overall complement of assayed DNA Tape in each cell has on the order of 1075 possible states.

During the monoclonal expansion, the generation of lineage barcodes in each cell was efficient, such that the vast majority of assayed cells contained a unique editing pattern across the 59 sites (3236/3257 or 99.4%; 9 patterns recurred in two cells, and 1 in three cells). After 25 days of editing, the first sites of active TAPE-1 arrays were edited to near-saturation (mean 96.8%) while the fifth sites were only modestly edited (mean 19.7%) (FIG. 4C). Across all 13 tape arrays, the number of edits accruing per cell resembled a Poisson distribution, with the mean number of discrete events per cell (u=39.4) roughly equaling the variance (02=40.0) (FIG. 4D). Assuming 20 cell divisions, this corresponds to an average of 2 edits accruing per cell division. The mean number of pairwise differences between cells, including sites at which one cell was edited and the other unedited, was 41.9±5.3 (FIG. 4E).

Next, a cell lineage tree was constructed. In contrast with GESTALT and other CRISPR-Cas9-based lineage recording systems, edits accruing to the multi-copy DNA Tape derive from a finite set of pegRNA-specified symbols, analogous to the finite set of nucleotides or amino acids used to build conventional phylogenetic trees. However, in further contrast with GESTALT but also with conventional phylogenetics, DNA Typewriter provides explicit information regarding the order in which differences accrued. To leverage this, a 3,257-by-3,257 similarity matrix was constructed by calculating, for all possible pairs of cells, the number of shared edits across the 59 sites. However, for shared edits at any given site to be counted, it was required that all earlier sites along that DNA Tape were also identically edited (Methods). Across all 5.3M pairwise comparisons of cells, 24M out of 33M shared edits met this criterion; those that did not presumably correspond to coincident occurrences of the same edit at the same site in different cells, and as such are appropriate to discount. After converting this similarity matrix to a distance matrix, two phylogenetic trees were generated, using either the unweighted pair group method with arithmetic mean (UPGMA) or the neighbour-joining (NJ) hierarchical clustering method. Between these two methods, UPGMA resulted in a tree with a lower parsimony score of 123,625, compared to 124,997 for a tree constructed using NJ.

Reconstruction of a monophyletic cell lineage tree using DNA Typewriter. In some embodiments, a monophyletic lineage tree of the 3,257 cells with all 13 TargetBC tape arrays recovered. The unweighted pair group method with arithmetic mean (UPGMA) clustering method was used to construct the tree from a distance matrix that takes account the order of edits within the TAPE-1 arrays, by discounting matches for which earlier sites along the same tape were not also identically edited. In some embodiments, a lineage tree constructed by order-aware UPGMA for a subset of 32 cells drawn from the larger tree.

To assess robustness, first, two distantly related clades of 16 cells from the global UPGMA tree were identified, merged them into a new set of 32 cells, and then performed conventional bootstrapping, treating the sites associated with each of 13 TargetBCs as independent groups, sampling 13 TargetBC groups with replacement, and then constructing and comparing UPGMA-based trees (Methods). Across 100 resamplings, all 31 branchings were observed multiple times, 20 with bootstrap values over 50%, and a bootstrap value of 100% for the separation between the two distantly related clades. Bootstrap analysis of an additional clade of 81 cells was also performed; for this clade, all 80 branchings were observed multiple times, 38 with bootstrap values over 50%. Finally, bootstrap analysis of the entire matrix was performed, resulting in the tree in which 76% of branches were seen multiple times, and 25% with bootstrap values over 50%.

In summary, over the course of 25 days of expansion of a monoclonal cell line from 1 to ˜1.2M cells, the ordered accumulation of 39.4±6.3 edits to 59 sites located within 13 DNA Tape arrays were observed. Although the number of active type-guides at these arrays declined (13 in the founding cell→mean 8.6 active type-guides per cell after 25 days), the recording capacity of the system (only 1 of 3,257 sampled cells was edited at all 59 sites) was not exhausted. To further assess whether editing was maintained throughout the experiment, the number of pairwise differences between each cell and its “nearest neighbour” were examined within the sampled set of 3,257 cells (FIG. 4F). On average, cells were separated from their nearest neighbour by 22.8 edits (or, assuming a constant rate of ˜2 edits per generation, 11 to 12 generations). This result is interpreted as strong support for the conclusion that editing of the DNA Tapes was maintained throughout the clonal expansion.

### Editing and Recovering Longer DNA TAPE

As illustrated by this lineage tracing experiment, at least a dozen DNA Tapes can be deployed and recovered in each cell, which substantially increases information capacity. However, even with multiple DNA Tapes, the maximum potential recording duration of each DNA Tape remains directly proportional to the number of consecutive monomers on each tape. Although 5×TAPE-1 appears to be very stable within cells as well as throughout amplification and sequencing (FIG. 8B), longer tandem arrays might introduce additional technical challenges, e.g., being difficult to synthesise, clone and maintain, prone to instability during in vivo DNA replication or repair as well as during in vitro PCR, and difficult to accurately and fully sequence.

To evaluate the extent to which such issues might be limiting in practice, a synthetic minisatellite in the form of 12 or 20 repeats of the 14-bp TAPE-1 monomer was generated. 12×TAPE-1 was synthesised as single-stranded DNA (IDT) and 20×TAPE-1 as a plasmid (GenScript). PCR amplicons of each array were cloned into the piggyBAC vector via Gibson assembly. Of note, cloned constructs were used “as is”, even though it is possible that some degree of variation in repeat number was already present (FIG. 11A-11B). PiggyBAC vectors bearing ˜12×TAPE-1 or ˜20×TAPE-1 were integrated into HEK293T cells expressing both PE2 and pegRNAs targeting TAPE-1 for NNNGGA insertions (PE2(+) 3N-TAPE-1-pegRNA(+) HEK293T) in triplicate. These cell lines were cultured for 40 days before collecting genomic DNA. PCR amplification of TAPE-1 was followed by standard library construction and sequencing on the Pacific Bioscience Sequel platform to obtain circular consensus sequencing (CCS) reads. On average, 8.4±3.3 repeats of TAPE-1 monomers were recovered from 12×TAPE-1 and 12.5±4.3 repeats from 20×TAPE-1. In each case, there was a sharp drop off after the intended length of 12 or 20 monomers, suggesting that regardless of the mechanism, these longer arrays are more prone to contraction than expansion (FIG. 11C). Of note, the editing rates were the same between the constructs (4.5±1.3 edits and 4.5±1.5 edits per 12×TAPE-1 and 20×TAPE-1 arrays, respectively: FIG. 11D). This is expected, as each DNA Tape has exactly one active type-guide, and as such the rates at which they are written to should be independent of their length.

CCS reads were grouped within each replicate based on a degenerate 8-bp barcode (TargetBC), as these presumably derived from the same integration. On average, each TargetBC group had 3.1±3.4 and 3.8±5.7 reads for ˜ 12×TAPE-1 and ˜20×TAPE-1, respectively. Within TargetBC groups, shorter arrays appeared more stable, with a greater proportion matching the maximum length within that group (FIG. 11E-11F). Of representative CCS reads for 4784 and 6254 integrated arrays for 12×TAPE-1 and 20×TAPE-1, respectively, the overwhelming majority (>99.5%) exhibited clear patterns of sequential, directed editing (FIG. 11G-11H). In terms of the maximum extent to which any given tape was edited, one TargetBC was observed for which 14 distinct 3-bp insertion events were recorded along a 14-monomer tape.

This experiment illustrates that it is possible to construct and use synthetic minisatellites corresponding to at least 20 monomers as a DNA Tape, and that sequential recording of at least 14 consecutive events with DNA Typewriter is possible. Nonetheless, further experiments are required to quantify the extent to which variation in synthetic minisatellite length is due to: (1) piggyBAC vector heterogeneity, i.e., variation that existed prior to integration; (2) DNA replication and microsatellite instability in HEK293T cells; (3) DNA repair subsequent to prime editing-induced nicks; and/or (4) PCR amplification artefacts. Of note, the observed variation in array length tended to occur within the unedited portion of the tape (FIG. 11G-11H). The inventors have yet to observe any clear examples of “information erasure”, possibly because the edits themselves disrupt the tandem repeats, inhibiting processes that might otherwise lead to erasure from spreading proximal to the type-guide.

### DISCUSSION

Digital systems represent information through both the content and order of discrete symbols, with each symbol drawn from a finite set. Digital systems are ancient, and include written text, morse code, and binary data, and, of course, genomic DNA. In this proof-of-concept of DNA Typewriter, this Example demonstrates how sequential genome editing of a monomeric array constitutes an artificial digital system that is operational within living eukaryotic cells, capable of “writing” thousands of discrete symbols to DNA in an ordered fashion.

DNA Typewriter improves on existing CRISPR-based molecular recorders in important ways (Table 3).

TABLE 3
Comparison of example CRISPR-based molecular recording methods to DNA
Typewriter.

| | TRACE; | | | | | |
|---|---|---|---|---|---|---|
| | Record- | | | | | |
| | seq; | | | | | |
| | Shipman et | | HOMING; | | CAMERA; | DNA |
| | al. | GESTALT | stgRNA | CHYRON | DOMINO | Typewriter |
| DNA editing | Cas1-Cas2 | Cas9 | Cas9 | Cas9 nuclease | Base editors | Prime editors |
| modality | integrase | nuclease | nuclease | and terminal | (e.g., Cas9 | (e.g., Cas9 |
| | | | | deoxynucleotidyl | nickase with | nickase with |
| | | | | transferase | cytidine | reverse- |
| | | | | (Tdt) | deaminase) | transcriptase) |
| Information | Up to 28- | Stochastic | Stochastic | 8.4 bp insertion | Up to 2-bp | Up to 6-bp |
| content per | bp preset | NHEJ/ | NHEJ/ | on average | within the | preset |
| recording | spacer | MMEJ | MMEJ DNA | (<17 bits) | target | insertion |
| | sequence | DNA repair | repair | | editing | sequence |
| | (<56 bits) | outcome | outcome | | window (2 | (<12 bits) |
| | | | | | bits) | |
| Possible | >1 trillion | 1 | 1 | 1 | 1 | 10-1000 |
| symbols per | | | | | | |
| target | | | | | | |
| Recording | Expansion | Unordered | Accumulated | Accumulated | 5+ | 14+ |
| operation | of array | indels at | indels at self- | indels at self- | sequential | sequential |
| | during | adjacent | targeting site | targeting site | single base- | insertional |
| | spacer | targets | (del > ins) | (del > ins) | pair edits | edits w/ |
| | acquisition | (del > ins) | | | | pegRNAs |
| Limiting | Number of | Array size | Number of | Number of | Number of | Number of |
| factor in | spacer | | stgRNAs | stgRNAs | sgRNAs and | pegRNAs |
| multiplexing | precursors | | | | targets | |
| Temporal | Position | Shared edits | Position | Position within | Programmed | Position |
| inference | within the | among | within the | the stgRNA | to each | within the |
| | target array | target arrays | stgRNA | | target- | target array |
| | | | | | sgRNA | |
| | | | | | circuit | |
| Necessary | Cas1-Cas2, | Cas9, | Cas9, | CAsp, TdT, | Base editor, | Prime editor, |
| cellular | Spacer | sgRNAs, | stgRNAs | stgRNAs | sgRNAs | pegRNAs, |
| components | DNA | target arrays | | | | DNA Tape |
| | oligos | | | | | array |
| Demonstrated | Escherichia | Eukaryotic | Eukaryotic | Eukaryotic | Prokaryotic | eukaryotic |
| host range | coli | | | | and | |
| | | | | | eukaryotic | |
| Key | Portability | Inability to | Inability to | Inability to | Limited | High |
| limitations | to | capture the | form target | form target | diversity in | multiplicity |
| | eukaryotic | event order; | arrays; | arrays; reduced | editing per | of integration |
| | cells | large | reduced | activity over | target | necessary for |
| | | deletion on | activity for | time | | highly |
| | | target arrays | longer gRNA | | | diverse edits |
| References | Shipman et | McKenna et | Perli et al., | Loveless et al., | Tang and | |
| | al., 2016 | al., 2016 | 2016 | 2021 | Liu, 2018 | |
| | Schmidt et | Chan et al., | Kalhor et al., | | Farzadfard | |
| | al., 2017 | 2019 | 2017 | | et al., 2019 | |
| | Sheth et al., | Bowling et | Kalhor et al., | | | |
| | 2017 | al., 2020 | 2018 | | | |
| | | | Park at al., | | | |
| | | | 2021 | | | |

The sequential editing achieved by DNA Typewriter resembles Cas1-Cas2-based recording, which at present are limited to bacterial systems. In DOMINO and CAMERA, base editors are used to record biological signals to “pre-programmed logic circuits” composed of multiple targets for base editing. Although these methods are conceptual predecessors to DNA Typewriter, there are critical differences. In particular, with all three methods, a recording event creates a new target for further editing (i.e., the type-guide). However, with DOMINO and CAMERA, each logic circuit is designed to record a specific order. In contrast, a single DNA Typewriter construct can potentially record any order. For example, to distinguish pairwise orderings within a set of n events, DOMINO or CAMERA would require n-choose-2 recording logic circuits or a system that contains the order of n² number of unique gRNA and their targets. In contrast and as demonstrated here (FIG. 2), DNA Typewriter requires only a single target array such as 5×TAPE-1, along with n unique pegRNAs that encode different insertions but share the same target.

As described in this Example, pegRNAs are used to encode symbols (i.e., insertional barcodes), but these pegRNAs are introduced by artificial transduction or stochastic expression. However, several groups have engineered guide RNAs whose activity is dependent on the binding of specific small molecules or ligands. Also, we recently developed ENGRAM, a prime editing-based system in which biological signals of interest such as NF-κB and Wnt signals are coupled to the production of specific pegRNAs. These pegRNAs mediate the insertion of signal-specific barcodes to a DNA-based recording site, providing quantitative information with respect to the strength and/or duration of the signal(s). At least in principle, such strategies are compatible with the current implementation of DNA Typewriter, potentially enabling the temporal dynamics of multiple biological signals or other cellular events to be recorded and resolved. In this context, the use of longer and therefore more diverse insertion barcodes could enable extensive multiplexing, although this might come at the expense of recording efficiency. As described in this Example, the rate of prime editing can be on the order of days, such that DNA Typewriter may be most useful for recording information about biological processes that unfold over a time-scale of days or weeks, rather than minutes or hours.

For example, one such process is biological development, wherein the unfolding of a cell lineage tree is of fundamental interest. In a proof-of-concept experiment, as described in this Example, DNA Typewriter overcomes the major limitations of earlier editing-based lineage recorders like GESTALT, by reducing ambiguity about the order in which editing events occurred, eschewing double-stranded breaks and thereby minimising the risk of inter-target deletion, predefining the locations to which edits accrue, predefining the “symbol set” from which edits are drawn, and stabilising the rate of editing by ensuring one-and-only-one type-guide per active DNA Tape. These attributes clearly pay off in the proof-of-concept experiment, as a seemingly steady accumulation of edits can be sustained to multi-copy DNA Tape across 25 days of in vitro expansion, from a single cell to over one million cells. Although this is longer than the gestation period of a mouse, the recording capacity of the system was not exhausted. Furthermore, the resulting data are sufficiently rich and complete to build and characterise cell lineage trees from these data with conventional phylogenetic algorithms (e.g., UPGMA, NJ), with only minor modifications directed at leveraging information about the order of edits, not available in other contexts in which phylogenetics is applied. In this experiment, the number of edits accruing per cell resembled a Poisson distribution. Further experiments are needed to assess the extent to which this rate of accrual is a function of absolute time, cell cycle, or some combination thereof. However, as it has been shown that prime editing continues to take place in non-mitotic cells such as neurons (Anzalone, A. V. et al. Search-and-replace genome editing without double-strand breaks or donor DNA. Nature 576, 149-157 (2019)), it is likely to be primarily a function of time.

What are the limits of this approach? Under the assumption that a similar performance can be achieved in vivo (multiple efficiently recovered DNA Tapes per cell; steady accrual of edits over several weeks; multiple edits per lineage per cell division), the inventors can readily conceive of a technical path to Sulston-esque reconstructions (Sulston, J. E., Schierenberg, E., White, J. G. & Thomson, J. N. The embryonic cell lineage of the nematode Caenorhabditis elegans. Dev. Biol. 100, 64-119 (1983)) of the cell lineage histories of non-transparent model organisms, e.g., fly, mouse, zebrafish, macaque. Further, it ca be envisioned that a single, synthetic DNA construct that encodes a prime editing enzyme, multiple recording arrays, and a combination of stochastic and signal-specific pegRNAs, could be used to simultaneously record both lineage and biological signals in any multicellular system, i.e. a molecular “flight recorder” locus. A single locus design would be less affected by site-of-integration effects, such as the inventors have observed with multiple DNA Tape constructs integrated across the genome. Alternatively, if genomic sites with a high prime editing efficiency can be identified, such sites might be leveraged to boost information capture. A separate risk is that prime editing efficiency might vary substantially across cell types. However, any such variation could potentially be ameliorated by technical improvements to system components (Nelson, J. W. et al. Engineered pegRNAs improve prime editing efficiency. Nat. Biotechnol. (2021) doi: 10.1038/s41587-021-01039-7, Chen, P. J. et al. Enhanced prime editing systems by manipulating cellular determinants of editing outcomes. Cell 184, 5635-5652.e29 (2021)), by increasing recording capacity and/or by modelling it during tree reconstruction. Although challenging to engineer, a generic recorder locus would enable the use of DNA as an in vivo digital recording medium, e.g., not only to characterise wildtype development, but also to enable the systematic comparison of the developmental histories of wildtype and mutant individuals.

### Materials and Methods

### Plasmid Cloning

Both pegRNA and DNA Tape constructs were cloned either using Gibson assembly (Gibson Assembly Master Mix, New England Biolab) or ligation after restriction (T4 DNA Ligase, New England Biolabs). For the Gibson assembly protocol, inserts of interest, usually ordered in the form of single-stranded DNA (IDT; Ultramer, up to 200-bp, or IDT oPool, up to 350-bp), were amplified using polymerase chain reaction (PCR; KAPA HiFi polymerase) and converted into double-stranded DNA molecules. For ligation, single-stranded DNAs (IDT) were annealed to have 4 bp overhangs in both ends of double-stranded DNAs, which is a substrate for T4 DNA ligase. Cloning backbones were digested either with BsaI-HFv2 or BsmBI-v2 (NEB), gel-purified, and mixed with inserts in the Gibson Assembly reaction. A small amount (1-2 uL) of Gibson Assembly reaction mix or T4 ligation mix was added to NEB Stbl cell (C3040) for transformation and grown at 30° C. or 37° C. for the plasmid DNA preparation (Qiagen miniprep). The resulting plasmids were sequence-verified using Sanger sequencing (Genewiz). The pegRNA plasmids used in transient transfection experiments were cloned using plasmid backbone pU6-pegRNA-GG-acceptor (Addgene #132777), following the protocol outlined in Anzalone et al. (Anzalone, A. V. et al. Search-and-replace genome editing without double-strand breaks or donor DNA. Nature 576, 149-157 (2019)). The resulting pegRNA expression cassette would have a U6 promoter and poly-T terminator. For the epegRNA cloning, another fragment including the evoPreQ1 sequence was added, where each strand of oligos were purchased phosphorylated from IDT. The Lenti-TargetBC-5×TAPE-1-pegRNA-InsertBC construct was cloned based on the CROP-seq vector (Datlinger, P. et al. Pooled CRISPR screening with single-cell transcriptome readout. Nat. Methods 14, 297-301 (2017)) (CROP-seq-Guide-Puro; Addgene #86708). The vector was modified to include the GFP-TargetBC-5×TAPE-1-CaptureSequence1 sequence, and the U6-promoter downstream sequence has been modified to allow the insertion of InsertBC-pegRNA sequence. Plasmids encoding DNA Typewriter constructs (piggyBAC-5×TAPE-1-BlastR), lineage tracing constructs (Lenti-TargetBC-5×TAPE-1-pegRNA-InsertBC) and pegRNAs (pU6-CApegTAPE1) have been submitted to Addgene (ID 175808, 183790, and 175809).

### Tissue Culture, Transfection, Lentiviral Transduction, and Transgene Integration

The HEK293T cell line was purchased from ATCC and maintained by following the recommended protocol from the vendor. The primary mouse embryonic fibroblast (MEF) cells were purchased from Millipore-Sigma (PMEF-CFL; EmbryoMax Primary Mouse Embryonic Fibroblasts, Strain CF1, not treated, passage 3). Both HEK293T and MEF cells were cultured in Dulbecco's modified Eagle's medium (DMEM) with high glucose (GIBCO), supplemented with 10% fetal bovine serum (Rocky Mountain Biologicals) and 1% penicillin-streptomycin (GIBCO). The mouse embryonic stem cells (mESCs; E14tg2a) were a generous gift from Dr. Christian Schröter. mESCs were cultured in the Ndiff 227 medium (Takara) supplemented with 1% penicillin-streptomycin, 3 uM CHIR99021 (Millipore-Sigma), 1 uM STEMGENT PD0325901 (Reprocell), and 1,000 units of ESGRO Recombinant Mouse LIF protein (Sigma-Aldrich). For culturing both MEF and mESCs, wells in the culture plates were coated with 0.1% gelatin in a 37° C. incubator for 1 hour. Cells were grown with 5% CO2 at 37° C. Cell lines were used as received without an authentication or a test for mycoplasma.

For transient transfection, HEK293T cells were cultured to 70-90% confluency in a 24-well plate. For prime editing, 375 ng of Prime Editor-2 enzyme plasmid (Addgene #132776) and 125 ng of pegRNA plasmid were mixed and prepared with a transfection reagent (Lipofectamine 3000) following the recommended protocol from the vendor. Cells were cultured for four to five days after the initial transfection unless noted otherwise, and its genomic DNA was harvested following cell lysis and protease protocol from Anzalone et al. (Anzalone, A. V. et al. Search-and-replace genome editing without double-strand breaks or donor DNA. Nature 576, 149-157 (2019)).

Both MEF cells and mESCs were transfected using 4D-Nucleofector (Lonza Bioscience). For MEF cells, about 200,000 cells were resuspended in 20 uL Nucleofector buffer with supplement, mixed with 800 ng of DNA plasmids (600 ng of pCMV-PEmax-P2A-hMLH1dn and 200 ng of epegRNA plasmid), loaded onto 16-well strip cuvette, and electroporated using program CM137 in the 4D-Nucleofector. For mESCs, about 50,000 cells were resuspended in 20 uL Nucleofector buffer with supplement, mixed with 800 ng of DNA plasmids (600 ng of pCMV-PEmax-P2A-hMLH1dn and 200 ng of epegRNA plasmid), loaded onto 16-well strip cuvette, and electroporated using program CG104 in the 4D-Nucleofector. Cells were cultured for 4 more days before genomic DNA harvesting or the subsequent transfection in the case of mESCs.

For lentivirus generation, about 300,000 HEK293T cells were seeded to each well in a 6-well plate and cultured to 70-90% confluency. The lentiviral plasmid was transfected along with the ViraPower lentiviral expression system (Thermo Fisher), following the recommended protocol from the vendor. Lentivirus was harvested following the same protocol, concentrated overnight using Peg-it Virus Precipitation Solution (SBI), and used within 1-2 days to transduce HEK293T cells without a freeze-thaw cycle. To achieve high multiplicity of infection, we used Magnetofection protocol (OZ Bioscience). For the lineage-tracing experiments, transduced cells were serially diluted and seeded to 96-well plates to identify monoclonal lines. Dox concentrations were maintained by having 10 mg/L in the initial culture and replenished every five days, to account for the 24 to 48 half-life of Dox in culturing media.

For transposase integration, 500 ng of cargo plasmid and 100 ng of Super piggyBAC transposase expression vector (SBI) were mixed and prepared with a transfection reagent (Lipofectamine 3000) following the recommended protocol from the vendor and transfected to a confluent 24-wells. The monoclonal Dox-inducible PE2 cell line was generated by integrating PE2 using the piggyBAC transposase system and selecting clones by prime-editing activity, as previously described (Choi, J. et al. Precise genomic deletions using paired prime editing. Nat. Biotechnol. (2021) doi: 10.1038/s41587-021-01025-z).

### Genomic DNA Collection and Sequencing Library Preparation

The targeted region from collected genomic DNA was amplified using two-step PCR and sequenced using Illumina sequencing platform (NextSeq or MiSeq). The first PCR reaction (KAPA Robust polymerase) included 1.5 uL of cell lysate, 0.04 to 0.4 uM of forward and reverse primers in a final reaction volume of 25 μL. The first PCR reaction was programmed to be: (1) 3 minutes at 95° C., (2) 15 seconds at 95° C., (3) 10 seconds at 65° C., (4) 90 seconds at 72° C., (5) 25-28 cycles of repeating step 2 through 4, and (6) 1 minute at 72° C. Primers included sequencing adapters to their 3′-ends, appending them to both termini of PCR products that amplified genomic DNA. After the first PCR step, products were assessed on 6% TBE-gel and purified using 1.0×AMPure (Beckman Coulter) and added to the second PCR reaction that appended dual sample indexes and flow cell adapters. The second PCR reaction program was identical to the first PCR program except it ran for only 5-10 cycles. Products were again purified using AMPure and assessed on the TapeStation (Agilent) before being denatured for the sequencing run.

For appending 10-bp unique molecular identifiers (UMI), the PCR reaction was performed in three steps: First, genomic DNA was linearly amplified in the presence of 0.04 to 0.4 uM of single forward primer in two PCR cycles using KAPA Robust polymerase. Specifically, the UMI-appending linear PCR reaction was programmed to be: (1) 3 minutes and 15 seconds at 95° C., (2) 1 minute at 65° C., (3) 2 minutes at 72° C., (4) 5 cycles of repeating step 2 and 3, (5) 15 seconds at 95° C., (6) 1 minute at 65° C., (7) 2 minutes at 72° C., and (8) another 5 cycles of repeating step 6 and 7. Second, this reaction was cleaned up using 1.5×AMPure, and then to a second PCR with forward and reverse primers: (1) 3 minutes at 95° C., (2) 15 seconds at 95° C., (3) 10 seconds at 65° C., (4) 90 seconds at 72° C., (5) 25-28 cycles of repeating step 2 through 4, and (6) 1 minute at 72° C. In this case, the forward primer binds upstream of the UMI sequence and is not specific to the genomic locus. Finally, after PCR amplification, products were cleaned up using AMPure magnetic beads (1.0×, following the protocol from Beckman Coulter) and added to the third and last PCR reaction that appended dual sample indexes and flow cell adapters. The run parameters for the third PCR reaction was the same as the second PCR reaction, except only 5-10 cycles of repeating step 2 through 4 were used. TAPE construct sequences and PCR primer sequences are provided in Table 4 and Table 5, respectively.

TABLE 4
Nucleic acid sequences of experimental constructs

| | | SEQ |
|---|---|---|
| Name | Sequence | ID NO |
| 2xTAPE-1 | GGATGATGGTGAGCACGTGATGGTGAGCACGTGATGGTGAGC | 58 |
| 3xTAPE-1 | GGATGATGGTGAGCACGTGATGGTGAGCACGTGATGGTGAGCACGTGA | 59 |
| | TGGTGAGC | |
| 5xTAPE-1 | GGATGATGGTGAGCACGTGATGGTGAGCACGTGATGGTGAGCACGTGATGG | 60 |
| | TGAGCACGTGATGGTGAGCACGTGATGGTGAGC | |
| 5xTAPE-1 | AATGATACGGCGACCACCGAGATCTACACNNNNNNNNNNTCGTCGGCAGC | 61 |
| Amplicon | GTCAGATGTGTATAAGAGACAGGGCTTTATATATCTTGTGGAAAGGACGAA | |
| sequence | ACACCNNNNNNNNGGATGATGGTGAGCACGTGATGGTGAGCACGTGATGG | |
| | TGAGCACGTGATGGTGAGCACGTGATGGTGAGCACGTGATGGTGAGCGCTT | |
| | TAAGGCCGGTCCTAGCAATTTTTTTaagcttggcgtaactagatcttgagacaaAGATCGGAA | |
| | GAGCACACGTCTGAACTCCAGTCACNNNNNNNNNNATCTCGTATGCCGTCT | |
| | TCTGCTTG | |
| pegRNA- | GGATGATGGTGAGCACGTGAGTTTTAGAGCTAGAAATAGCAAGTTAAAATA | 62 |
| 2N-TAPE- | AGGCTAGTCCGTTATCAACTTGAAAAAGTGGCACCGAGTCGGTGCTCACCA | |
| 1 | TCATCCNNCGTGCTCACCATC | |
| pegRNA- | GGATGATGGTGAGCACGTGAGTTTTAGAGCTAGAAATAGCAAGTTAAAATA | 63 |
| 3N-TAPE- | AGGCTAGTCCGTTATCAACTTGAAAAAGTGGCACCGAGTCGGTGCTCACCA | |
| 1 | TCATCCNNNCGTGCTCACCATC | |
| epegRNA- | GGATGATGGTGAGCACGTGAGTTTTAGAGCTAGAAATAGCAAGTTAAAATA | 64 |
| 2N-TAPE- | AGGCTAGTCCGTTATCAACTTGAAAAAGTGGCACCGAGTCGGTGCTCACCA | |
| 1 | TCATCCNNCGTGCTCACCATCTTGACGCGGTTCTATCTAGTTACGCGTTAAA | |
| | CCAACTAGAAA | |
| epegRNA- | GGATGATGGTGAGCACGTGAGTTTTAGAGCTAGAAATAGCAAGTTAAAATA | 65 |
| 3N-TAPE- | AGGCTAGTCCGTTATCAACTTGAAAAAGTGGCACCGAGTCGGTGCTCACCA | |
| 1 | TCATCCNNNCGTGCTCACCATCTTGACGCGGTTCTATCTAGTTACGCGTTAA | |
| | ACCAACTAGAAA | |
| epegRNA- | GGATGATGGTGAGCACGTGAGTTTTAGAGCTAGAAATAGCAAGTTAAAATA | 66 |
| 4N-TAPE- | AGGCTAGTCCGTTATCAACTTGAAAAAGTGGCACCGAGTCGGTGCTCACCA | |
| 1 | TCATCCNNNNCGTGCTCACCATCTTGACGCGGTTCTATCTAGTTACGCGTTA | |
| | AACCAACTAGAAA | |
| epegRNA- | GGATGATGGTGAGCACGTGAGTTTTAGAGCTAGAAATAGCAAGTTAAAATA | 67 |
| 5N-TAPE- | AGGCTAGTCCGTTATCAACTTGAAAAAGTGGCACCGAGTCGGTGCTCACCA | |
| 1 | TCATCCNNNNNCGTGCTCACCATCTTGACGCGGTTCTATCTAGTTACGCGTT | |
| | AAACCAACTAGAAA | |
| epegRNA- | GGATGATGGTGAGCACGTGAGTTTTAGAGCTAGAAATAGCAAGTTAAAATA | 68 |
| 6N-TAPE- | AGGCTAGTCCGTTATCAACTTGAAAAAGTGGCACCGAGTCGGTGCTCACCA | |
| 1 | TCATCCNNNNNNCGTGCTCACCATCTTGACGCGGTTCTATCTAGTTACGCGT | |
| | TAAACCAACTAGAAA | |
| 2Npeg- | GGATGATGGTGAGCACGTGAGTTTTAGAGCTAGAAATAGCAAGTTAAAATA | 69 |
| 3xTAPE-1 | AGGCTAGTCCGTTATCAACTTGAAAAAGTGGCACCGAGTCGGTGCTCACCA | |
| | TCATCCNNCGTGCTCACCATCTTTTTTGGATGATGGTGAGCACGTGATGGTG | |
| | AGCACGTGATGGTGAGCACGTGATGGTGAGC | |
| 2Npeg- | GGACGATGGCACCTCCTCGAGTTTTAGAGCTAGAAATAGCAAGTTAAAATA | 70 |
| 3xTAPE-2 | AGGCTAGTCCGTTATCAACTTGAAAAAGTGGCACCGAGTCGGTGCAGGTGC | |
| | CATCGTCCNNAGGAGGTGCCATCTTTTTTGGACGATGGCACCTCCTCGATGG | |
| | CACCTCCTCGATGGCACCTCCTCGATGGCACCTGC | |
| 2Npeg- | GGTCTCTGGAGCATCTGCTCGTTTTAGAGCTAGAAATAGCAAGTTAAAATA | 71 |
| 3xTAPE-3 | AGGCTAGTCCGTTATCAACTTGAAAAAGTGGCACCGAGTCGGTGCATGCTC | |
| | CAGAGACCNNCAGATGCTCCAGATTTTTTGGTCTCTGGAGCATCTGCTCTGG | |
| | AGCATCTGCTCTGGAGCATCTGCTCTGGAGCATGC | |
| 2Npeg- | GGACCGAGGAGTGCTACCCGGTTTTAGAGCTAGAAATAGCAAGTTAAAATA | 72 |
| 3xTAPE-4 | AGGCTAGTCCGTTATCAACTTGAAAAAGTGGCACCGAGTCGGTGCACTCCT | |
| | CGGTCCNNGTAGCACTCCTCGTTTTTTGGACCGAGGAGTGCTACCCGAGGA | |
| | GTGCTACCCGAGGAGTGCTACCCGAGGAGTGC | |
| 2Npeg- | GAGACCTGGTCTGCAGCACCGTTTTAGAGCTAGAAATAGCAAGTTAAAATA | 73 |
| 3xTAPE-5 | AGGCTAGTCCGTTATCAACTTGAAAAAGTGGCACCGAGTCGGTGCAGACCA | |
| | GGTCTCNNGCTGCAGACCAGGTTTTTTGAGACCTGGTCTGCAGCACCTGGTC | |
| | TGCAGCACCTGGTCTGCAGCACCTGGTCTGC | |
| 2Npeg- | GGAGCGCGGGCTTTCCCGCGGTTTTAGAGCTAGAAATAGCAAGTTAAAATA | 74 |
| 3xTAPE-6 | AGGCTAGTCCGTTATCAACTTGAAAAAGTGGCACCGAGTCGGTGCAAAGCC | |
| | CGCGCTCCNNGGGAAAGCCCGCGTTTTTTGGAGCGCGGGCTTTCCCGCGCG | |
| | GGCTTTCCCGCGCGGGCTTTCCCGCGCGGGCTTTGC | |
| 2Npeg- | GCACGCCGGCTGGTAGGCGCGTTTTAGAGCTAGAAATAGCAAGTTAAAATA | 75 |
| 3xTAPE-7 | AGGCTAGTCCGTTATCAACTTGAAAAAGTGGCACCGAGTCGGTGCTACCAG | |
| | CCGGCGTGCNNCCTACCAGCCGGCTTTTTTGCACGCCGGCTGGTAGGCGCC | |
| | GGCTGGTAGGCGCCGGCTGGTAGGCGCCGGCTGGTAGC | |
| 2Npeg- | GGACCAGGGCCACGACGCCAGTTTTAGAGCTAGAAATAGCAAGTTAAAATA | 76 |
| 3xTAPE-8 | AGGCTAGTCCGTTATCAACTTGAAAAAGTGGCACCGAGTCGGTGCTGGCCC | |
| | TGGTCCNNCGTCGTGGCCCTGTTTTTTGGACCAGGGCCACGACGCCAGGGC | |
| | CACGACGCCAGGGCCACGACGCCAGGGCCAGC | |
| 2Npeg- | GGATGATGGCAGAGGAGTGAGTTTTAGAGCTAGAAATAGCAAGTTAAAATA | 77 |
| 3xTAPE-9 | AGGCTAGTCCGTTATCAACTTGAAAAAGTGGCACCGAGTCGGTGCCTCTGC | |
| | CATCATCCNNCTCCTCTGCCATCTTTTTTGGATGATGGCAGAGGAGTGATGG | |
| | CAGAGGAGTGATGGCAGAGGAGTGATGGCAGAGGC | |
| 2Npeg- | GGACGATGGCATTCACTCGAGTTTTAGAGCTAGAAATAGCAAGTTAAAATA | 78 |
| 3xTAPE- | AGGCTAGTCCGTTATCAACTTGAAAAAGTGGCACCGAGTCGGTGCGAATGC | |
| 10 | CATCGTCCNNAGTGAATGCCATCTTTTTTGGACGATGGCATTCACTCGATGG | |
| | CATTCACTCGATGGCATTCACTCGATGGCATTCGC | |
| 2Npeg- | GGTCTCTGGCTCCATGGCTCGTTTTAGAGCTAGAAATAGCAAGTTAAAATA | 79 |
| 3xTAPE- | AGGCTAGTCCGTTATCAACTTGAAAAAGTGGCACCGAGTCGGTGCTGGAGC | |
| 11 | CAGAGACCNNCCATGGAGCCAGATTTTTTGGTCTCTGGCTCCATGGCTCTGG | |
| | CTCCATGGCTCTGGCTCCATGGCTCTGGCTCCAGC | |
| 2Npeg- | GACCGAGGTGCGAGGCCCGGTTTTAGAGCTAGAAATAGCAAGTTAAAATAA | 80 |
| 3xTAPE- | GGCTAGTCCGTTATCAACTTGAAAAAGTGGCACCGAGTCGGTGCTCGCACC | |
| 12 | TCGGTCCNNGCCTCGCACCTCGTTTTTTGGACCGAGGTGCGAGGCCCGAGGT | |
| | GCGAGGCCCGAGGTGCGAGGCCCGAGGTGCGAGC | |
| 2Npeg- | GAGACCTGGATCGCTTCACCGTTTTAGAGCTAGAAATAGCAAGTTAAAATA | 81 |
| 3xTAPE- | AGGCTAGTCCGTTATCAACTTGAAAAAGTGGCACCGAGTCGGTGCGATCCA | |
| 13 | GGTCTCNNGAAGCGATCCAGGTTTTTTGAGACCTGGATCGCTTCACCTGGAT | |
| | CGCTTCACCTGGATCGCTTCACCTGGATCGC | |
| 2Npeg- | GGAGCGCGGCGCCGCCCGCGGTTTTAGAGCTAGAAATAGCAAGTTAAAATA | 82 |
| 3xTAPE- | AGGCTAGTCCGTTATCAACTTGAAAAAGTGGCACCGAGTCGGTGCGCGCCG | |
| 14 | CGCTCCNNGGGCGGCGCCGCGTTTTTTGGAGCGCGGCGCCGCCCGCGCGGC | |
| | GCCGCCCGCGCGGCGCCGCCCGCGCGGCGCGC | |
| 2Npeg- | GCACGCCGGCGTAGGCGCGCGTTTTAGAGCTAGAAATAGCAAGTTAAAATA | 83 |
| 3xTAPE- | AGGCTAGTCCGTTATCAACTTGAAAAAGTGGCACCGAGTCGGTGCCCTACG | |
| 15 | CCGGCGTGCNNCGCCTACGCCGGCTTTTTTGCACGCCGGCGTAGGCGCGCC | |
| | GGCGTAGGCGCGCCGGCGTAGGCGCGCCGGCGTAGGGC | |
| 2Npeg- | GGACCAGGGCTGCGGGGCCAGTTTTAGAGCTAGAAATAGCAAGTTAAAATA | 84 |
| 3xTAPE- | AGGCTAGTCCGTTATCAACTTGAAAAAGTGGCACCGAGTCGGTGCCAGCCC | |
| 16 | TGGTCCNNCCCCGCAGCCCTGTTTTTTGGACCAGGGCTGCGGGGCCAGGGC | |
| | TGCGGGGCCAGGGCTGCGGGGCCAGGGCTGGC | |
| 2Npeg- | GGACCAGGGTGAGCACGCCAGTTTTAGAGCTAGAAATAGCAAGTTAAAATA | 85 |
| 3xTAPE- | AGGCTAGTCCGTTATCAACTTGAAAAAGTGGCACCGAGTCGGTGCTCACCC | |
| 17 | TGGTCCNNCGTGCTCACCCTGTTTTTTGGACCAGGGTGAGCACGCCAGGGTG | |
| | AGCACGCCAGGGTGAGCACGCCAGGGTGAGC | |
| 2Npeg- | GGAGTTGGGCACCTCCTGTTGTTTTAGAGCTAGAAATAGCAAGTTAAAATA | 86 |
| 3xTAPE- | AGGCTAGTCCGTTATCAACTTGAAAAAGTGGCACCGAGTCGGTGCAGGTGC | |
| 18 | CCAACTCCNNAGGAGGTGCCCAATTTTTTGGAGTTGGGCACCTCCTGTTGGG | |
| | CACCTCCTGTTGGGCACCTCCTGTTGGGCACCTGC | |
| 2Npeg- | GGTCCCTGGAGCATCTGCCCGTTTTAGAGCTAGAAATAGCAAGTTAAAATA | 87 |
| 3xTAPE- | AGGCTAGTCCGTTATCAACTTGAAAAAGTGGCACCGAGTCGGTGCATGCTC | |
| 19 | CAGGGACCNNCAGATGCTCCAGGTTTTTTGGTCCCTGGAGCATCTGCCCTGG | |
| | AGCATCTGCCCTGGAGCATCTGCCCTGGAGCATGC | |
| 2Npeg- | GGAAGTTGGAGTGCTACAGTGTTTTAGAGCTAGAAATAGCAAGTTAAAATA | 88 |
| 3xTAPE- | AGGCTAGTCCGTTATCAACTTGAAAAAGTGGCACCGAGTCGGTGCACTCCA | |
| 20 | ACTTCCNNGTAGCACTCCAACTTTTTTGGAAGTTGGAGTGCTACAGTTGGAG | |
| | TGCTACAGTTGGAGTGCTACAGTTGGAGTGC | |
| 2Npeg- | GAGATCCGGTCTGCAGCATCGTTTTAGAGCTAGAAATAGCAAGTTAAAATA | 89 |
| 3xTAPE- | AGGCTAGTCCGTTATCAACTTGAAAAAGTGGCACCGAGTCGGTGCAGACCG | |
| 21 | GATCTCNNGCTGCAGACCGGATTTTTTGAGATCCGGTCTGCAGCATCCGGTC | |
| | TGCAGCATCCGGTCTGCAGCATCCGGTCTGC | |
| 2Npeg- | GGACTCTGGGCTTTCCCCTCGTTTTAGAGCTAGAAATAGCAAGTTAAAATAA | 90 |
| 3xTAPE- | GGCTAGTCCGTTATCAACTTGAAAAAGTGGCACCGAGTCGGTGCAAAGCCC | |
| 22 | AGAGTCCNNGGGAAAGCCCAGATTTTTTGGACTCTGGGCTTTCCCCTCTGGG | |
| | CTTTCCCCTCTGGGCTTTCCCCTCTGGGCTTTGC | |
| 2Npeg- | GCATGCAGGCTGGTAGGTGCGTTTTAGAGCTAGAAATAGCAAGTTAAAATA | 91 |
| 3xTAPE- | AGGCTAGTCCGTTATCAACTTGAAAAAGTGGCACCGAGTCGGTGCTACCAG | |
| 23 | CCTGCATGCNNCCTACCAGCCTGCTTTTTTGCATGCAGGCTGGTAGGTGCAG | |
| | GCTGGTAGGTGCAGGCTGGTAGGTGCAGGCTGGTAGC | |
| 2Npeg- | GGATGCTGGCCACGACGTGCGTTTTAGAGCTAGAAATAGCAAGTTAAAATA | 92 |
| 3xTAPE- | AGGCTAGTCCGTTATCAACTTGAAAAAGTGGCACCGAGTCGGTGCTGGCCA | |
| 24 | GCATCCNNCGTCGTGGCCAGCTTTTTTGGATGCTGGCCACGACGTGCTGGCC | |
| | ACGACGTGCTGGCCACGACGTGCTGGCCAGC | |
| 2Npeg- | GACTGATGGTGAGCACGTGAGTTTTAGAGCTAGAAATAGCAAGTTAAAATA | 93 |
| 3xTAPE- | AGGCTAGTCCGTTATCAACTTGAAAAAGTGGCACCGAGTCGGTGCTCACCA | |
| 25 | TCAGTCNNCGTGCTCACCATCTTTTTTGACTGATGGTGAGCACGTGATGGTG | |
| | AGCACGTGATGGTGAGCACGTGATGGTGAGC | |
| 2Npeg- | GACCGATGGCACCTCCTCGAGTTTTAGAGCTAGAAATAGCAAGTTAAAATA | 94 |
| 3xTAPE- | AGGCTAGTCCGTTATCAACTTGAAAAAGTGGCACCGAGTCGGTGCAGGTGC | |
| 26 | CATCGGTCNNAGGAGGTGCCATCTTTTTTGACCGATGGCACCTCCTCGATGG | |
| | CACCTCCTCGATGGCACCTCCTCGATGGCACCTGC | |
| 2Npeg- | GACCTCTGGAGCATCTGCTCGTTTTAGAGCTAGAAATAGCAAGTTAAAATA | 95 |
| 3xTAPE- | AGGCTAGTCCGTTATCAACTTGAAAAAGTGGCACCGAGTCGGTGCATGCTC | |
| 27 | CAGAGGTCNNCAGATGCTCCAGATTTTTTGACCTCTGGAGCATCTGCTCTGG | |
| | AGCATCTGCTCTGGAGCATCTGCTCTGGAGCATGC | |
| 2Npeg- | GATCCGAGGAGTGCTACCCGGTTTTAGAGCTAGAAATAGCAAGTTAAAATA | 96 |
| 3xTAPE- | AGGCTAGTCCGTTATCAACTTGAAAAAGTGGCACCGAGTCGGTGCACTCCT | |
| 28 | CGGATCNNGTAGCACTCCTCGTTTTTTGATCCGAGGAGTGCTACCCGAGGA | |
| | GTGCTACCCGAGGAGTGCTACCCGAGGAGTGC | |
| 2Npeg- | GTAACCTGGTCTGCAGCACCGTTTTAGAGCTAGAAATAGCAAGTTAAAATA | 97 |
| 3xTAPE- | AGGCTAGTCCGTTATCAACTTGAAAAAGTGGCACCGAGTCGGTGCAGACCA | |
| 29 | GGTTACNNGCTGCAGACCAGGTTTTTTGTAACCTGGTCTGCAGCACCTGGTC | |
| | TGCAGCACCTGGTCTGCAGCACCTGGTCTGC | |
| 2Npeg- | GATGCGCGGGCTTTCCCGCGGTTTTAGAGCTAGAAATAGCAAGTTAAAATA | 98 |
| 3xTAPE- | AGGCTAGTCCGTTATCAACTTGAAAAAGTGGCACCGAGTCGGTGCAAAGCC | |
| 30 | CGCGCATCNNGGGAAAGCCCGCGTTTTTTGATGCGCGGGCTTTCCCGCGCG | |
| | GGCTTTCCCGCGCGGGCTTTCCCGCGCGGGCTTTGC | |
| 2Npeg- | GATCGCCGGCTGGTAGGCGCGTTTTAGAGCTAGAAATAGCAAGTTAAAATA | 99 |
| 3xTAPE- | AGGCTAGTCCGTTATCAACTTGAAAAAGTGGCACCGAGTCGGTGCTACCAG | |
| 31 | CCGGCGATCNNCCTACCAGCCGGCTTTTTTGATCGCCGGCTGGTAGGCGCCG | |
| | GCTGGTAGGCGCCGGCTGGTAGGCGCCGGCTGGTAGC | |
| 2Npeg- | GTCCCAGGGCCACGACGCCAGTTTTAGAGCTAGAAATAGCAAGTTAAAATA | 100 |
| 3xTAPE- | AGGCTAGTCCGTTATCAACTTGAAAAAGTGGCACCGAGTCGGTGCTGGCCC | |
| 32 | TGGGACNNCGTCGTGGCCCTGTTTTTTGTCCCAGGGCCACGACGCCAGGGC | |
| | CACGACGCCAGGGCCACGACGCCAGGGCCAGC | |
| 2Npeg- | GATGATGGGTGAGCACCTGAGTTTTAGAGCTAGAAATAGCAAGTTAAAATA | 101 |
| 3xTAPE- | AGGCTAGTCCGTTATCAACTTGAAAAAGTGGCACCGAGTCGGTGCTCACCC | |
| 33 | ATCATCNNGGTGCTCACCCATTTTTTTGATGATGGGTGAGCACCTGATGGGT | |
| | GAGCACCTGATGGGTGAGCACCTGATGGGTGAGC | |
| 2Npeg- | GACGATGGTCACCTCCTCGAGTTTTAGAGCTAGAAATAGCAAGTTAAAATA | 102 |
| 3xTAPE- | AGGCTAGTCCGTTATCAACTTGAAAAAGTGGCACCGAGTCGGTGCAGGTGA | |
| 34 | CCATCGTCNNAGGAGGTGACCATTTTTTTGACGATGGTCACCTCCTCGATGG | |
| | TCACCTCCTCGATGGTCACCTCCTCGATGGTCACCTGC | |
| 2Npeg- | GTCTCTGGGAGCATCTGCTCGTTTTAGAGCTAGAAATAGCAAGTTAAAATA | 103 |
| 3xTAPE- | AGGCTAGTCCGTTATCAACTTGAAAAAGTGGCACCGAGTCGGTGCATGCTC | |
| 35 | CCAGAGACNNCAGATGCTCCCAGTTTTTTGTCTCTGGGAGCATCTGCTCTGG | |
| | GAGCATCTGCTCTGGGAGCATCTGCTCTGGGAGCATGC | |
| 2Npeg- | GACCGAGGGAGTGCTACCCGGTTTTAGAGCTAGAAATAGCAAGTTAAAATA | 104 |
| 3xTAPE- | AGGCTAGTCCGTTATCAACTTGAAAAAGTGGCACCGAGTCGGTGCACTCCC | |
| 36 | TCGGTCNNGTAGCACTCCCTCTTTTTTGACCGAGGGAGTGCTACCCGAGGGA | |
| | GTGCTACCCGAGGGAGTGCTACCCGAGGGAGTGC | |
| 2Npeg- | GAACCTGGGTCTGCAGCACCGTTTTAGAGCTAGAAATAGCAAGTTAAAATA | 105 |
| 3xTAPE- | AGGCTAGTCCGTTATCAACTTGAAAAAGTGGCACCGAGTCGGTGCAGACCC | |
| 37 | AGGTTCNNGCTGCAGACCCAGTTTTTTGAACCTGGGTCTGCAGCACCTGGGT | |
| | CTGCAGCACCTGGGTCTGCAGCACCTGGGTCTGC | |
| 2Npeg- | GAGCGCGGAGCTTTCCCGCGGTTTTAGAGCTAGAAATAGCAAGTTAAAATA | 106 |
| 3xTAPE- | AGGCTAGTCCGTTATCAACTTGAAAAAGTGGCACCGAGTCGGTGCAAAGCT | |
| 38 | CCGCGCTCNNGGGAAAGCTCCGCTTTTTTGAGCGCGGAGCTTTCCCGCGCG | |
| | GAGCTTTCCCGCGCGGAGCTTTCCCGCGCGGAGCTTTGC | |
| 2Npeg- | GACGCCGGACTGGTAGGCGCGTTTTAGAGCTAGAAATAGCAAGTTAAAATA | 107 |
| 3xTAPE- | AGGCTAGTCCGTTATCAACTTGAAAAAGTGGCACCGAGTCGGTGCACCAGT | |
| 39 | CCGGCGTCNNCCTACCAGTCCGGTTTTTTGACGCCGGACTGGTAGGCGCCG | |
| | GACTGGTAGGCGCCGGACTGGTAGGCGCCGGACTGGTGC | |
| 2Npeg- | GACCAGGGACCACGACGCCAGTTTTAGAGCTAGAAATAGCAAGTTAAAATA | 108 |
| 3xTAPE- | AGGCTAGTCCGTTATCAACTTGAAAAAGTGGCACCGAGTCGGTGCTGGTCC | |
| 40 | CTGGTCNNCGTCGTGGTCCCTTTTTTTGACCAGGGACCACGACGCCAGGGAC | |
| | CACGACGCCAGGGACCACGACGCCAGGGACCAGC | |
| 2Npeg- | GGACTGATGGTGAGACGTGAGTTTTAGAGCTAGAAATAGCAAGTTAAAATA | 109 |
| 3xTAPE- | AGGCTAGTCCGTTATCAACTTGAAAAAGTGGCACCGAGTCGGTGCTCTCAC | |
| 41 | CATCAGTCCNNCGTCTCACCATCATTTTTTGGACTGATGGTGAGACGTGATG | |
| | GTGAGACGTGATGGTGAGACGTGATGGTGAGAGC | |
| 2Npeg- | GGACCGATGGCACTCCTCGAGTTTTAGAGCTAGAAATAGCAAGTTAAAATA | 110 |
| 3xTAPE- | AGGCTAGTCCGTTATCAACTTGAAAAAGTGGCACCGAGTCGGTGCAGGAGT | |
| 42 | GCCATCGGTCCNNAGGAGTGCCATCGTTTTTTGGACCGATGGCACTCCTCGA | |
| | TGGCACTCCTCGATGGCACTCCTCGATGGCACTCCTGC | |
| 2Npeg- | GGATCTCTGGAGCACTGCTCGTTTTAGAGCTAGAAATAGCAAGTTAAAATA | 111 |
| 3xTAPE- | AGGCTAGTCCGTTATCAACTTGAAAAAGTGGCACCGAGTCGGTGCAGTGCT | |
| 43 | CCAGAGATCCNNCAGTGCTCCAGAGTTTTTTGGATCTCTGGAGCACTGCTCT | |
| | GGAGCACTGCTCTGGAGCACTGCTCTGGAGCACTGC | |
| 2Npeg- | GGATCCGAGGAGTGCACCCGGTTTTAGAGCTAGAAATAGCAAGTTAAAATA | 112 |
| 3xTAPE- | AGGCTAGTCCGTTATCAACTTGAAAAAGTGGCACCGAGTCGGTGCACTCCT | |
| 44 | CGGATCCNNGTGCACTCCTCGGTTTTTTGGATCCGAGGAGTGCACCCGAGG | |
| | AGTGCACCCGAGGAGTGCACCCGAGGAGTGC | |
| 2Npeg- | GCAGACCTGGTCTGCACACCGTTTTAGAGCTAGAAATAGCAAGTTAAAATA | 113 |
| 3xTAPE- | AGGCTAGTCCGTTATCAACTTGAAAAAGTGGCACCGAGTCGGTGCAGACCA | |
| 45 | GGTCTGCNNGTGCAGACCAGGTTTTTTTGCAGACCTGGTCTGCACACCTGGT | |
| | CTGCACACCTGGTCTGCACACCTGGTCTGC | |
| 2Npeg- | GGATGCGCGGGCTTTCCGCGGTTTTAGAGCTAGAAATAGCAAGTTAAAATA | 114 |
| 3xTAPE- | AGGCTAGTCCGTTATCAACTTGAAAAAGTGGCACCGAGTCGGTGCAAAGCC | |
| 46 | CGCGCATCCNNGGAAAGCCCGCGCTTTTTTGGATGCGCGGGCTTTCCGCGC | |
| | GGGCTTTCCGCGCGGGCTTTCCGCGCGGGCTTTGC | |
| 2Npeg- | GCAGCGCCGGCTGTAGGCGCGTTTTAGAGCTAGAAATAGCAAGTTAAAATA | 115 |
| 3xTAPE- | AGGCTAGTCCGTTATCAACTTGAAAAAGTGGCACCGAGTCGGTGCTACAGC | |
| 47 | CGGCGCTGCNNCCTACAGCCGGCGTTTTTTGCAGCGCCGGCTGTAGGCGCC | |
| | GGCTGTAGGCGCCGGCTGTAGGCGCCGGCTGTAGC | |
| 2Npeg- | GGACCCAGGGCACGACGCCAGTTTTAGAGCTAGAAATAGCAAGTTAAAATA | 116 |
| 3xTAPE- | AGGCTAGTCCGTTATCAACTTGAAAAAGTGGCACCGAGTCGGTGCTCGTGC | |
| 48 | CCTGGGTCCNNCGTCGTGCCCTGGTTTTTTGGACCCAGGGCACGACGCCAG | |
| | GGCACGACGCCAGGGCACGACGCCAGGGCACGAGC | |
| TargetBC- | CAAGCAGAAGACGGCATACGAGATccgaatccgaGTGACTGGAGTTCAGACGTG | 117 |
| 5xTAPE-1 | TGCTCTTCCGATCTTGATTTGGTACCATAGCAGATGATCCATGGTCAATNNN | |
| 10X | NNNNNGGATGATGGTGAGCACGNNNGGATGATGGTGAGCACGNNNGGATG | |
| amplicon | ATGGTGAGCACGNNNGGATGATGGTGAGCACGNNNGGATGATGGTGAGCA | |
| | CGNNNGGATGATGGTGAGCCAAGGAAAGGAAGCCCTGCTTCCTCCAGAGG | |
| | GCGTCGCAGGACAGCTGCTTTAAGGCCGGTCCTAGCAANNNNNNNNNNNN | |
| | NNNNNNNNNNNNNNNNCTGTCTCTTATACACATCTGACGCTGCCGACGANN | |
| | NNNNNNNNGTGTAGATCTCGGTGGTCGCCGTATCATT | |
| TargetBC- | CACCNNNNNNNNGGATGATGGTGAGCACGTGATGGTGAGCACGTGATGGT | 118 |
| 12×TAPE- | GAGCACGTGATGGTGAGCACGTGATGGTGAGCACGTGATGGTGAGCACGTG | |
| 1 | ATGGTGAGCACGTGATGGTGAGCACGTGATGGTGAGCACGTGATGGTGAGC | |
| | ACGTGATGGTGAGCACGTGATGGTGAGCACGTGATGGTGAGCGCTTTAAGG | |
| | C | |
| TargetBC- | CACCGGNNNNNNNNTGTAAAACGAcggccagtTAATACGACTCACTATAGGGA | 119 |
| 20xTAPE- | GGATGATGGTGAGCACGTGATGGTGAGCACGTGATGGTGAGCACGTGATGG | |
| 1 | TGAGCACGTGATGGTGAGCACGTGATGGTGAGCACGTGATGGTGAGCACGT | |
| | GATGGTGAGCACGTGATGGTGAGCACGTGATGGTGAGCACGTGATGGTGAG | |
| | CACGTGATGGTGAGCACGTGATGGTGAGCACGTGATGGTGAGCACGTGATG | |
| | GTGAGCACGTGATGGTGAGCACGTGATGGTGAGCACGTGATGGTGAGCACG | |
| | TGATGGTGAGCACGTGATGGTGAGCACGTGATGGTGAGCGCTTTAAGGC | |
| PiggyBAC- | Addgene website; catalog number: 175808 | |
| 5xTAPE- | | |
| 1 | | |
| pU6-CA- | Addgene website; catalog number: 175809 | |
| pegTAPE | | |
| 1 | | |
| Lenti- | Addgene website; catalog number: 183790 | |
| TargetBC- | | |
| 5xTAPE- | | |
| 1- | | |
| pegRNA- | | |
| InsertBC | | |

TABLE 5
Primer sequences used in PCR reactions

| | | SEQ |
|---|---|---|
| Name | Sequence | ID NO |
| TAPE_amp_fwd | GCGTCAGATGTGTATAAGAGACAGGGCTTTATATATCTTGTGGA | 120 |
| | AAGGACGAAACACC | |
| TAPE_amp_rev | TTCAGACGTGTGCTCTTCCGATCTttgtctcaagatctagttacgccaagc | 121 |
| Plasmid_peg_amp_fwd | GCGTCAGATGTGTATAAGAGACAGatcttgtggaaaggacgaaacacc | 122 |
| Plasmid_peg_amp_rev | TTCAGACGTGTGCTCTTCCGATCTgcctggtatctttatagtcctgtcg | 123 |
| TAPE_Lineage Tracing_ | CTGTCTCTTATACACATCTGACGCTGCCGACGA | 124 |
| fwd | | |
| TAPE_LineageTracing_ | TTCAGACGTGTGCTCTTCCGATCTTGATTTGGTACCATAGCAGA | 125 |
| rev | TGATC | |
| TAPE_Long_amp_fwd | GCGTCAGATGTGTATAAGAGACAGATACGGGGAAAAGGCCTCC | 126 |
| | AC | |
| TAPE_Long_amp_rev | TTCAGACGTGTGCTCTTCCGATCTGAGTTGAGCAAGCAGGGTCA | 127 |
| | G | |

For long-read amplicon sequencing library preparation, a two-step PCR protocol was used: the first PCR reaction (KAPA Robust polymerase) included 1.5 uL of cell lysate, 0.04 to 0.4 uM of forward and reverse primers in a final reaction volume of 25 μL. The first PCR reaction was programmed to be: (1) 3 minutes at 95° C., (2) 15 seconds at 95° C., (3) 10 seconds at 65° C., (4) 3 minutes at 72° C., (5) 25-28 cycles of repeating step 2 through 4, and (6) 1 minute at 72° C. After the first PCR step, products were assessed on 6% TBE-gel and purified using 0.6×AMPure (Beckman Coulter) and added to the second PCR reaction that appended PacBio sample indexes. The second PCR reaction program was identical to the first PCR program except it ran for only 5-10 cycles. Products were again purified using AMPure and assessed on the TapeStation (Agilent) and sequenced on Sequel (Pacific Biosciences; Laboratory of Biotechnology and Bioanalysis, Washington State University).

### Genomic DNA Amplicon Sequencing Data Processing and Analysis

Sequencing reads from Illumina MiSeq and NextSeq platforms are first demultiplexed using BCL2fastq software (Illumina). For experiments shown in FIGS. 1 and 5-7, sequencing libraries were single-end sequenced to cover the DNA Tape from one direction. For experiments shown in FIGS. 2, 3, 8, and 9, sequencing libraries were paired-end sequenced to cover the entire array from both directions. Paired reads were then merged using PEAR (Zhang, J., Kobert, K., Flouri, T. & Stamatakis, A. PEAR: a fast and accurate Illumina Paired-End reAd mergeR. Bioinformatics 30, 614-620 (2014)) with default parameters to reduce sequencing errors. Insertion sequences, in the form of NNGGA 5-mer to NNNNNNGGA 9-mer were extracted from sequencing reads of the TAPE arrays, including 2×TAPE-1, 3×TAPE-1, and 5×TAPE-1, using pattern-matching software such as Regular Expression (package REGEX) in Python. Insertions (4 to 6 bp) on 3×TAPE-1 to 3×TAPE-48 were also extracted using REGEX pattern-matching software.

For the sequential transfection epochs experiment shown in FIG. 2, 5-mer insertions were first extracted from 5×TAPE-1 sequencing reads and used a k-means clustering algorithm to filter out possible PCR/sequencing errors with low read counts. Such filtering removed all reads that have the wrong key sequence (GGA in the case of TAPE-1), leaving a set of 16 possible 5-mer sequences in the form of NNGGA. Across 5 repeats of insertion sites on 5×TAPE-1, the separate unigram frequencies in each site were calculated, which was used to build the Unigram order as shown in FIG. 8C. Bigram frequencies between the adjacent insertion sites (Site-1 and Site-2, Site-2 and Site-3, Site-3 and Site-4, and Site-4 and Site-5 pairs) were combined, normalised across row and column, and used to build the bigram transition matrices as shown in FIG. 2C-2G. For ordering the barcodes according to their transfection history, a Unigram order was first generated by sorting its relative frequency on Site-1, where barcodes were assumed to have transfected earlier if they appeared more frequently in Site-1 than other sites. Using the resulting Unigram order as the initial order, an iterative algorithm was implemented where the order was pass through, from early to late, swap the order if their bigram frequency is inconsistent with the order, and restart the pass unless there have been no swaps in a single pass.

For the short digital text encoding experiment shown in FIG. 3, 6-mer insertions were extracted, corrected the read-counts of each 6-mers by their editing efficiencies (using separately measured insertion frequency and respective plasmid abundance, similar to described in FIG. 5D-5F), used a k-means clustering algorithm to identify NNNGGA barcodes, and built the bigram transition matrix as described in the paragraph above. The bigram transition matrices were first analysed using a hierarchical clustering algorithm, with default parameters given in the R software (using Euclidean distance measure and complete-linkage clustering method, as described in FIG. 9). Putative sets of barcodes (co-transfection sets with generally 2-4 barcodes) were visually identified based on the dendrogram and used to group barcodes in the output bigram order of the algorithm used above. The order within the co-transfection sets was determined using the corrected unigram counts combined across all five sites, where more abundant barcodes were assigned to be earlier within the set. Barcodes were mapped back to the text following the encoding table (Table 2)

For the long-read sequencing experiment described in FIG. 11, 12×TAPE-1 and 20×TAPE-1 sequences were isolated from Pacific Biosciences circular consensus (CCS) reads. The number of TAPE monomers and insertions were calculated using sequential text-matching around insertions and the expected length of the array based on insertion counts. Reads without a match between expected length and observed length were filtered out. Each 12×TAPE-1 and 20×TAPE-1 construct is associated with an 8-bp degenerate barcode sequence (TargetBC). Assuming that the integration sites for each TargetBC are different, reads from any given replicate that shared the same TargetBC were grouped. Based on the observation that array collapse is more frequent than the array expansion, the read with the maximum number of TAPE-monomers from each set of reads that shared a TargetBC was selected. If multiple reads were tied by this criterion, the one (or one of the ones) with the most edits were selected for presentation in FIG. 11G-11H. Also selected for presentation in FIG. 11C-11H, the reads that have at least 3 insertions and at most 12× or 20×TAPE-1 monomers (FIG. 11C-11F) or at most 25×TAPE-1 monomers (FIG. 11G-11H).

### Single-Cell Lineage Tracing Experiment and Analysis

Monoclonal HEK293T cells containing 5×TAPE-1, iPE2, and multiple TargetBC-5×TAPE-1-pegRNA were cultured for 25 days in the presence of 10 mg/L doxycycline (Dox) concentration. Dox was replenished every five days, to account for the 24 to 48 half-life of Dox in culturing media. The initial culture in the 96-well plate was moved to 24-well, and then subsequently to 6-well, when the culture was 80-90% confluent. Once the monoclonal cell line reached confluency in 6-well (estimated to be 1.2M cells), cells were frozen and thawed for single-cell experiment in the absence of Dox. For preparation of cells for single-cell experiment, cells were dissociated, pelleted by centrifuging cells at 200 rcf for 5 minutes, and single-cell resuspended in 0.04% BSA (NEB) supplemented 1×PBS solution to 1,000 cells per uL concentration following the Cell Preparation Guide from 10× Genomics (manual part number CG00053 Rev C). Cell numbers and single-cell suspension were checked using both the manual hemocytometer and Countess II FL Cell Counter (Thermo Fisher).

Single-cell resuspended cells were directly used in the 10× Genomics experimental protocol (Chromium Next GEM Single Cell 3′ Reagent Kits v3.1 with Feature Barcoding technology for CRISPR screening; manual part number CG000205 Rev D). The protocol was strictly followed with 20,000 targeted cell recovery (10,000 per reaction) until step 2.3. The protocol is written for the CRISPR Screening library, where the Feature Barcode components including CRISPR gRNA sequences would be collected in step 2.3B, due to its smaller size compared to 3′ Gene Expression library (collected in step 2.3A). In this case, the Feature Barcode components including TargetBC-5×TAPE-1 constructs tagged with 16-nt 10×single-cell barcodes (CBC) and 12-bp unique molecular identifier (UMI) from reverse transcription are expected to be greater than 1-kb in length and therefore collected along with the 3′ Gene Expression library. Nonetheless, both components (eluates from steps 2.3A and 2.3B) were collected and detected TargetBC-5×TAPE-1 constructs from both using quantitative PCR. Detection of TargetBC-5×TAPE-1 constructs from step 2.3B is unexpected but could have resulted from non-processive reverse transcription that generated shorter cDNA products. TargetBC-5×TAPE-1 constructs were combined, and used paired-end sequencing to obtain CBC, UMI, and TargetBC-5×TAPE-1 sequences for each read, along with the 3′ Gene Expression library.

For the initial analysis, the CellRanger pipeline from 10× Genomics was used, which filtered out single-cell barcodes (CBC) and UMIs and recovered about 12,000 cells. Reads were selected that contain approved CBC and UMI sequences and extracted TargetBC-5×TAPE-1 sequences from the CellRanger output BAM file. Reads with different UMIs were collapsed based on shared CBC-TargetBC-5×TAPE-1 and removed any CBC-TargetBC-5×TAPE-1 reads that have less than 2 UMI sequences associated with them. In cases the same CBC-TargetBC pairs were observed but with different 5×TAPE-1 sequences, the consensus sequence with a larger number of associated UMIs was selected.

For the monoclonal lineage tracing experiment, the observed TargetBC was corrected if it contained a single-nucleotide mismatch to the approved list of 19 most frequent 8-bp sequences. If the TargetBC differed from the list of sequences by more than 2 nucleotides, those reads were removed from the further analysis. For detecting the 14-bp TAPE-1 sequence, a single base-pair mismatch or substitution error was corrected to the TAPE-1 sequence. The TargetBC-5×TAPE-1 arrays that include InsertBC other than the top 19 most frequent ones were filtered.

For the lineage tree reconstruction, only cells (CBC) that include the top 13 most frequent TargetBCs were selected (3,257 cells). This “top 13” list excluded the corrupt TargetBC ATAAGCGG (where the second TAPE-1 monomer appears to have been contracted by 6-bp, inactivating the type-guide). The 3,257-by-3,257 distance matrix was calculated by counting the number of shared InsertBC across 13×5=65 sites, but only if they share the same InsertBC on previous sites (out of 5 possible sites per TargetBC; unedited sites were excluded), and the subtracting the count from the maximum number of shared InsertBC (59, excluding 6 missing sites from three 4×TAPE-1 arrays and one 2×TAPE-1 array) to calculate the distance between a pair of cells. The resulting distance matrix was used as an argument in “UPGMA” and “NJ” clustering functions in the R “phangorn” package (Schliep, K., Potts, A. J., Morrison, D. A. & Grimm, G. W. Intertwining phylogenetic trees and networks. Methods Ecol. Evol. 8, 1212-1220 (2017)). Tree visualisations, bootstrapping analysis, and parsimony analysis were done using the R “ape” package (Paradis, E. & Schliep, K. ape 5.0: an environment for modern phylogenetics and evolutionary analyses in R. Bioinformatics 35, 526-528 (2019)) and included functions. Bootstrap resampling was done on blocks of sites within the same TargetBC-TAPE-1 array (i.e., resampling with replacement of the intact TAPE-1 arrays associated with the 13 TargetBCs). The same distance-matrix calculating function was used that counts the number of shared InsertBC only if they share the same InsertBC on previous sites within the TargetBC-TAPE-1 array, as described above.

### Data Availability Statement

Raw sequencing data have been uploaded on Sequencing Read Archive (SRA) with associated BioProject ID PRJNA757179.

### Code Availability Statement

Custom analysis codes for this project are available at Github (https://github.com/shendurelab/DNATickerTape) and Figshare (doi: 10.6084/m9.figshare.19607811).

### Example 2

This Example describes a new framework for multiplex transcriptional recording, which is termed ENGRAM (ENhancer-driven Genomic Recording of transcriptional Activity in Multiplex). In brief, ENGRAM relies on enzymatic release (Haurwitz, R. E., Jinek, M., Wiedenheft, B., Zhou, K. & Doudna, J. A. Sequence- and structure-specific RNA processing by a CRISPR endonuclease. Science 329, 1355-1358 (2010); Sternberg, S. H., Haurwitz, R. E. & Doudna, J. A. Mechanism of substrate selection by a highly specific CRISPR endoribonuclease. RNA 18, 661-672 (2012); Haurwitz, R. E., Sternberg, S. H. & Doudna, J. A. Csy4 relies on an unusual catalytic dyad to position and cleave CRISPR RNA. The EMBO Journal vol. 31 2824-2832 (2012); Nissim, L., Perli, S. D., Fridkin, A., Perez-Pinera, P. & Lu, T. K. Multiplexed and programmable regulation of gene networks with an integrated RNA and CRISPR/Cas toolkit in human cells. Mol. Cell 54, 698-710 (2014)) of prime editing guide RNAs (pegRNAs) (Anzalone, A. V. et al. Search-and-replace genome editing without double-strand breaks or donor DNA. Nature 576, 149-157 (2019)) from synthetic transcripts driven by cis-regulatory-element (CRE)-coupled Pol-II promoters. Each pegRNA programs the insertion of a specific barcode to a genomically-encoded recording locus (“DNA Tape”). Because each CRE is coupled to a distinct pegRNA-encoded insertion, multiple ENGRAM recorders can operate in parallel, all relying on the same prime editing enzyme and all competing to write to the same DNA Tape, which can be read out at either the DNA or RNA level. Of note, ENGRAM is the hypothetical memory storage unit in the brain. The inventors would like to use this as the memory storage in cells too.

### Results

### Development and Evaluation of ENGRAM

An ideal DNA-based transcriptional recorder would “log” the production of specific transcripts, cis-regulatory activities and/or signal transduction pathways, via specific changes to the primary sequence of a genomic “recorder locus”. In seeking to develop a DNA-based recorder for mammalian systems, the inventors were inspired by reporter assays, an established approach wherein a cis-regulatory element (CRE) of interest is positioned upstream of a minimal promoter (minP) and reporter gene (e.g., luciferase). Reporter assays are amenable to extensive multiplexing, as the reporter can include a transcribed barcode that is linked to the CRE, resulting in the MPRA (Klein, J. C. et al. A systematic evaluation of the design and context dependencies of massively parallel reporter assays. Nat. Methods 17, 1083-1091 (2020)). However, as noted above, MPRAs depend on targeted RNA-seq of the barcodes, which is destructive and static. Nonetheless, the inventors reasoned that the basic MPRA architecture, i.e., a library of synthetic or natural enhancers positioned upstream of a minimal promoter, might be coupled to the expression of a library of “writing units”, in the form of pegRNAs (FIG. 1a). Specifically in ENGRAM, each CRE is linked to a pegRNA encoding a specific insertion to a common DNA TAPE.

One challenge to this scheme is that in order to be appropriately processed, transcripts for most translated genes, including CRE-minP-driven reporter transcripts, are made by RNA polymerase II (Pol-2), whereas small untranslated RNAs, including guide RNAs, are made by RNA polymerase III (Pol-3). To address this, the inventors leveraged the CRISPR endoribonuclease Csy4 (also known as Cas6f), which recognizes and cuts at the 3′ end of 17-bp RNA hairpins (csy4) (Haurwitz, R. E., Jinek, M., Wiedenheft, B., Zhou, K. & Doudna, J. A. Sequence- and structure-specific RNA processing by a CRISPR endonuclease. Science 329, 1355-1358 (2010); Sternberg, S. H., Haurwitz, R. E. & Doudna, J. A. Mechanism of substrate selection by a highly specific CRISPR endoribonuclease. RNA 18, 661-672 (2012); Haurwitz, R. E., Sternberg, S. H. & Doudna, J. A. Csy4 relies on an unusual catalytic dyad to position and cleave CRISPR RNA. The EMBO Journal vol. 31 2824-2832 (2012); Nissim, L., Perli, S. D., Fridkin, A., Perez-Pinera, P. & Lu, T. K. Multiplexed and programmable regulation of gene networks with an integrated RNA and CRISPR/Cas toolkit in human cells. Mol. Cell 54, 698-710 (2014)). Expression of Csy4, together with CRE-activity-dependent expression of csy4-pegRNA-csy4, should result in a liberated functional pegRNA (FIG. 12A).

ENGRAM 1.0 was first developed, in which csy4-pegRNA-csy4 is embedded within the 3′ untranslated region (UTR) of a GFP transcript and the Csy4 is constitutively expressed (FIG. 13A). To benchmark the activity of pegRNAs released from Pol-2 transcripts, an ENGRAM 1.0 recorder driven by a constitutive Pol-2 promoter (PGK) was compared to a conventional, U6-driven pegRNA. In both cases, the pegRNAs target the endogenous HEK293 target 3 (HEK3) locus and are designed to insert three nucleotides (CTT) (Anzalone, A. V. et al. Search-and-replace genome editing without double-strand breaks or donor DNA. Nature 576, 149-157 (2019)). These constructs were separately transiently transfected to monoclonal HEK293T cells constitutively expressing Prime-Editor-2 (PE2) and Csy4. Five days after transfection, genomic DNA was harvested, and then PCR amplified and sequenced the HEK3 locus. Comparable, reproducible efficiencies of CTT insertion between the ENGRAM 1.0 and U6 recorders were observed (mean 5.9% and 5.3% across three replicates, respectively; FIG. 13B). Next, the constitutive PGK promoter was replaced with a CRE-minP architecture, in which thirteen 170-bp sequences with known enhancer activity in K562 cells were selected (Klein, J. C. et al. A systematic evaluation of the design and context dependencies of massively parallel reporter assays. Nat. Methods 17, 1083-1091 (2020)). The editing efficiency of the pool of enhancer-driven recorders was compared to a pool of negative controls (minP with no upstream enhancer) via their transient transfection to K562 cells constitutively expressing both PE2 and Csy4. Enhancer-activated barcode insertions were successfully recorded with a collective efficiency of 3.9%, 1.93-fold higher than the editing efficiency of pegRNAs driven by minP alone (FIG. 13C). Overall, these results suggest that ENGRAM-based recording can work. However, the signal-to-noise ratio was modest. This was likely due in part to the accumulation of background edits due to constitutive expression of Csy4.

To reduce the background accumulation of edits to the DNA Tape, a new ENGRAM architecture was designed in which the GFP ORF is replaced by Csy4 ORF, and Csy4 is no longer constitutively expressed (FIG. 12B, FIG. 13D). In this recorder design, termed ENGRAM 2.0, the expression of Csy4 and the pegRNA are both dependent on enhancer activity. To evaluate whether these modifications reduce background recording, ENGRAM 1.0 vs. 2.0 was tested in the absence of any enhancer, i.e. minP alone driving peg5N. Transiently co-transfecting these constructs into HEK293T cells with either PE2-Csy4 plasmid (for ENGRAM 1.0) or PE2 plasmid (for ENGRAM 2.0) in triplicate, a 2.8-fold reduction in background recording was observed with ENGRAM 2.0 relative to ENGRAM 1.0 (mean 1.4% for ENGRAM 1.0→0.5% for ENGRAM 2.0, 3 days post-transfection) (FIG. 13E).

Towards further reducing the background, two additional recorders were designed: 5′ ENGRAM 2.0, in which the csy4 hairpin-flanked pegRNA is embedded within the 5′ (rather than 3′) UTR of the Csy4 transcript; and 3′-FT ENGRAM 2.0, which contains an additional csy4 hairpin in its 5′ UTR to create an auto-regulatory negative feedback loop on Csy4 levels (FIG. 16B). The background recording activity was first measured by integrating them into HEK293T cells expressing PE2 (PE2(+) HEK293T) cells via PiggyBac. The 5′ ENGRAM 2.0 and 3′-FT ENGRAM 2.0 recorders respectively exhibited 12-fold and >100-fold reductions in background activity, relative to 3′ ENGRAM 2.0 (10 days post-transfection; FIG. 12C). Of note, for all three of these integrated ENGRAM 2.0 recorders, the level of background recording plateaued after several days (FIG. 12C). This suggested that the accumulation of background recording events mostly occurs shortly after transfection, potentially due to ORI-driven, plasmid-mediated transcription, rather than minP-driven transcription from integrated recorders. However, some degree of accumulation persisted with the 3′ ENGRAM 2.0 recorder, suggesting an additional component of genomically driven background activity. Their responsiveness to enhancer activation was measured by placing a NF-κB responsive element (activated by TNFα) in the upstream of the minP. All three recorders with NF-κB responsive element are integrated into PE2(+) HEK293T cells via PiggyBac. Their recording activity was measured in the absence or presence of the ligand TNFα. A 1.4, 13.3, 23.8-fold activation was observed for 3′, 5′ and 3′-FT recorders, respectively (FIG. 12D). Although the 3′-FT design exhibited the lowest background activity, and highest activation response to enhancer activation, the 5′ ENGRAM 2.0 design was selected because its organization facilitates straightforward pairing of CREs and pegRNA-mediated insertions during cloning. Unless specified, ENGRAM in the paper specifically refers to ENGRAM 2.0 5′ architecture.

From the above recording data, different efficiency for 5N barcodes was observed. To systematically analyze the editing efficiency bias, an ENGRAM recorder was cloned with pegRNA targeting HEK3 locus to install 5N degenerate insertion driven by a PGK promoter (FIG. 12E). PE2(+) was transiently transfected into HEK293T cells and measured recording efficiency at 3 days post-transfection. Overall, 1,023 of 1,024 all possible 5-bp insertions were observed at the HEK3 locus with highly reproducible frequencies (FIG. 12F, FIG. 13A-13C). After normalizing for their abundance in the plasmid pool and removing under-represented barcodes, 948 5-mers were observed with balanced insertional efficiencies, with 91% falling within a 4-fold range (FIG. 12G). It was suspected that heterogeneity in insertional efficiencies might be a consequence of the influence of the 5-mer on pegRNA secondary structure. Consistent with this, the least efficient 5-mer is predicted to pair with the spacer sequence to form a more stable secondary structure, while the most efficient 5-mer insertion does not (FIG. 13D-13E). To ask whether insertional bias could be predicted, linear lasso regression was performed with 84 binary sequence features and 1 secondary structural feature (minimum free energy (MFE), Methods). The resulting model was reasonably accurate, with MFE emerging as the most predictive feature (FIG. 12H; FIG. 13F-13G). For subsequent experiments disclosed in this Example, the bias was rigorously controlled by picking barcodes with more balanced insertion efficiency.

During the development of ENGRAM, two studies showed that engineered pegRNA (epegRNA, with tevoPreQ1 hairpin) (Nelson, J. W. et al. Engineered pegRNAs improve prime editing efficiency. Nat. Biotechnol. 40, 402-410 (2022)) and new prime editor architecture (PEmax) (Chen, P. J. et al. Enhanced prime editing systems by manipulating cellular determinants of editing outcomes. Cell 184, 5635-5652.e29 (2021)) can improve the editing efficiency. To improve ENGRAM recording efficiency, epegRNA and PEmax were tested in the context of 5′-ENGRAM. PE2(+) was transiently transfected into HEK293T cells with pegRNA and epegRNA encoding a 5N insertion, both driven by PGK promoter, and measured their recording efficiency at 3 days post-transfection. Surprisingly, a slightly lower efficiency was observed in epegRNA than pegRNA (16.6% vs 22.2% in epegRNA and pegRNA, respectively, ˜30% lower. FIG. 15A). The inventors reasoned that the csy4 hairpin might serve a similar role as tevoPreQ1 to protect pegRNA from degradation, additional hairpin to csy4 might disrupt RNA folding. PE2 or PEmax was co-transfected with PGK-5N and measured their editing efficiency at 3 days post-transfection. A 1.7-fold increase in editing efficiency was observed with PEmax (FIG. 15B). The inventors would recommend using PEmax for all future ENGRAM recording experiments. With 5′ ENGRAM, it was also tested if tRNA can be an alternative pegRNA processing architecture for ENGRAM. The csy4 hairpin was replaced with tRNA and measured their recording activity. However, no edits were observed with tRNA-ENGRAM (FIG. 15C).

Multiplex Recording of Enhancer Activity with ENGRAM

With sensitive and robust 5′-ENGRAM, this Example further discloses if ENGRAM can work as traditional MPRA. Enhancer libraries were cloned to the upstream of minP in the 5′-ENGRAM construct and integrated them into PE2+ K562 cells. The pegRNA is targeting the HEK3 locus and encoding a 5-bp or 6-bp short insertion. Thus, enhancer activity can be recorded on either endogenous DNA TAPE (genomic HEK3 locus, 2 copies) or synthetic DNA TAPE (PiggyBac integrated HEK3 locus, 10-30 copies). The abundance of barcodes in DNA TAPE is compared to the barcode abundance in pegRNA (FIG. 16A). A pair of 170-bp sequences previously shown to have either high vs. minimal enhancer activity in K562 cells (Klein, J. C. et al. A systematic evaluation of the design and context dependencies of massively parallel reporter assays. Nat. Methods 17, 1083-1091 (2020)) were first cloned upstream of minP, together with minP-only and promoter-less constructs (FIG. 2b). Each of these four constructs drove pegRNAs encoding two distinct 5-bp insertions. An equimolar mixture of these 8 recorder plasmids was introduced via PiggyBac integration into PE2+ K562 cells in triplicate. At five days post-transfection, 3.14% of endogenous HEK3 target sites were edited, but ˜90% of inserted barcodes were associated with the active enhancer (FIG. 16B). Of note, the 17.3-fold difference in recorded insertional frequency between the active and inactive enhancer roughly matched the 15-fold difference between them measured by MPRA.

To more generally evaluate whether the enhancer activities recorded by ENGRAM are quantitatively comparable to corresponding measurements made by MPRA, 300 enhancer fragments (Klein, J. C. et al. A systematic evaluation of the design and context dependencies of massively parallel reporter assays. Nat. Methods 17, 1083-1091 (2020)) to the 5′ ENGRAM construct were cloned, each driving a pegRNA encoding a unique 6-bp insertion (FIG. 16G). Five days after introducing these recorders via PiggyBac to PE2-expressing K562 in triplicate, the HEK3 locus (from DNA, both endogenous locus and synthetic locus) or the transcribed barcode itself (from RNA) was separately recovered, amplified and sequences. From DNA, an overall editing efficiency of 3.08% and 1.76% was observed for endogenous and synthetic HEK3 locus, respectively (FIG. 17A), and recovered 292 of 300 barcodes. Various depths (6,000, 12,000, 24,000, 48,000, 96,000 cells) were sampled on both endogenous and synthetic HEK3 locus and compared their recording efficiency and sensitivity. Overall, the enhancer activity recorded on endogenous and synthetic DNA TAPE are highly correlated (FIG. 17B). With 15 copies of synthetic DNA TAPE in the genome, it is possible to record 300 enhancer activity with as little as 12,000 cells with reasonable capture efficiency and reproducibility (FIG. 17C-17D). It is recommended to have at least 100 cells/enhancer for robust enhancer activity recording. The inventors reasoned that with improved recording efficiency, this number can be lower. Both RNA and DNA-based measurements were highly consistent between transfection replicates (Supplementary FIG. 2e-f). Furthermore, a strong correlation was observed between the recorded activities (ENGRAM; DNA) and the directly measured activities (MPRA; RNA), indicating that the relative transcriptional activities of enhancer reporters can be quantitatively recorded to genomic DNA (FIG. 2c).

Quantitative Recording of Signaling Pathway Activation or Small Molecule Exposure with ENGRAM

Next, this Example discloses whether ENGRAM could be used to record the intensity or duration of signaling pathway activation or small molecule exposure. For this, several signal-responsive regulatory elements were selected: the Tet Response Element (TRE; activated by doxycycline) (Gossen, M. et al. Transcriptional Activation by Tetracyclines in Mammalian Cells. Science vol. 268 1766-1769 (1995)), a NF-κB responsive element (activated by TNFα) (Zabel, U., Schreck, R. & Baeuerle, P. A. DNA binding of purified transcription factor NF-kappa B. Affinity, specificity, Zn2 dependence, and differential half-site recognition. Journal of Biological Chemistry vol. 266 252-260 (1991)), and a TCF-LEF responsive element (Wnt signaling pathway; activated by CHIR99021) (pGL4.49 [luc2P/TCF-LEF/Hygro] Vector Protocol. promega website), each previously used to drive fluorescent reporters in a signal-responsive manner (Table 6).

TABLE 6
Signal responsive elements and barcode used in this Example

| Signaling | Response element full sequence | | |
|---|---|---|---|
| pathway | (with cloning gibson overhand) | | Motif |
| TetON | GGGGATACGGGGAAAAGGCCTCCACGGC | | TCCCTATCAGTGATAGAGA |
| | CAGCTTGGCCGCGAATCGATATGTCGAGT | | (SEQ ID NO: 129) |
| | TTACTCCCTATCAGTGATAGAGAACGTAT | | |
| | GTCGAGTTTACTCCCTATCAGTGATAGAG | | |
| | AACGATGTCGAGTTTACTCCCTATCAGTG | | |
| | ATAGAGAACGTATGTCGAGTTTACTCCCT | | |
| | ATCAGTGATAGAGAACGTATGTCGAGTTT | | |
| | ACTCCCTATCAGTGATAGAGAACGTATGT | | |
| | CGAGTTTATCCCTATCAGTGATAGAGAAC | | |
| | GTATGTCGAGTTTACTCCCTATCAGTGAT | | |
| | AGAGAACGTATGTCGAGGTAGGCGTGTAC | | |
| | GGTGGGGGCACTAGAGGGTATATAATGG | | |
| | AAGCTCGACT | | |
| | (SEQ ID NO: 128) | | |
| NF-κB | GGGGATACGGGGAAAAGGCCTCCACGGC | | GGGACTTTCC |
| | CACAGACCGTTAGACGCCAGGACGGGCT | | (SEQ ID NO: 131) |
| | GTCAGGCTGGCGCCGCGGCGCCAGCCTGA | | |
| | CAGCCCGTCCTGGCGTCTAACGGTCTGAC | | |
| | ACTAGAGACGATTAAATGGACAGCATGC | | |
| | GTCTCTCTGCCATCAACCTCCGTGCTCAGT | | |
| | CTGACTGCCGTATAGGCAGCCGCCACCAT | | |
| | GGACCACTACCTCGACATTCGCTTGC | | |
| | (SEQ ID NO: 130) | | |
| TCF/LEF | GGGGATACGGGGAAAAGGCCTCCACGGC | | TAAGATCAAAGGG |
| (WNT) | CAAGATCAAAGGGTTTAAGATCAAAGGG | | (SEQ ID NO: 135) |
| | CTTAAGATCAAAGGGTATAAGATCAAAG | | |
| | GGCCTAAGATCAAAGGGACTAAGATCAA | | |
| | AGGGTTTAAGATCAAAGGGCTTAAGATCA | | |
| | AAGGGCCTACACTAGAGACGATTAAATG | | |
| | GACAGCATGCGTCTCTCTGCCATCAGCAA | | |
| | CCGTGCTCAGTCTGACTGCCGTATAGGCA | | |
| | GCCGCCACCATGGACCACTACCTCGACAT | | |
| | TCGCTTGC | | |
| | (SEQ ID NO: 134) | | |
| Signaling | | | Barcode(s) in |
| pathway | Repeat | Barcode(s) in pegRNA | genomic DNA |
| TetON | 7 | AGAAG | CTTCT |
| NF-κB | 6 | TCCTAAATAA | TAGGATTATT |
| | | (SEQ ID NO: 132) | (SEQ ID NO: 133) |
| TCF/LEF | 7 | GCAAC | GTTGC |
| (WNT) | | | |

These signal-responsive sequences were cloned upstream of minP within 5′ ENGRAM 2.0 recorders, with each driving expression of a pegRNA encoding one or two specific insertions to the endogenous HEK3 locus (FIG. 18A). The three recorders were separately integrated into the genomes of PE2(+) HEK293T cells via PiggyBac in triplicate (for the doxycycline recorder, constitutively expressed reverse tetracycline-controlled transactivator (rtTA) was integrated separately). A 2-fold dilution series of doxycycline, TNFα or CHIR99021 (for CHIR99021 we tested a concentration around 1-4 uM) was added to the media of the cell lines into which the relevant recorder had been integrated, and genomic DNA was harvested 48 hours after the onset of exposure.

For all three signal-responsive ENGRAM recorders, editing rates at the HEK3 locus exhibited a strikingly sigmoidal dependence on the log-transformed concentration of the corresponding stimulant (FIG. 18B-18D). This was particularly the case for the Wnt signaling, wherein the corresponding recorder exhibited nearly switch-like behavior across an approximately four-fold range of CHIR99021 concentration (FIG. 18D). As with previous experiments, each ENGRAM recorder exhibited minimum non-accumulating, basal recording even in the absence of signal exposure (0.1-0.2% %; FIG. 19A), potentially due to ORI-driven, plasmid-mediated transcription shortly after transfection, as discussed above. A dynamic range in editing efficiency was observed between background vs. maximal stimulation of 11.5-fold, 19.0-fold, and 22.6-fold for the Tet, NF-κB and Wnt recorders, respectively (FIG. 18E).

To explore the dependence of ENGRAM on not only the intensity of signals but also their duration, a matrix experiment was performed on the NF-κB and Wnt recorders, varying stimulant concentration as previously described but also varying the duration of exposure from 6 to 48 hours (2 recorders×8 concentrations×8 durations×3 replicates=384 conditions; FIG. 18F-18G). In this experiment, each batch of cells was harvested 24 hours after the removal of stimulants from the media. In the resulting levels of editing, the dependency of the NF-κB and Wnt recorders on both the intensity and duration of stimulation was immediately evident (FIG. 18F-18G). For both recorders, even 6 hours of stimulation was sufficient to observe signal in excess of background. However, the NF-κB recorder appeared to exhibit faster kinetics than the Wnt recorder (FIG. 19B-19C).

Multiplex Recording of Signaling Pathway Activity with ENGRAM

This Example further describes introducing multiple ENGRAM recorders for different signaling pathways into a single population of cells, to evaluate whether they could be used together, i.e., competing to write to a shared DNA Tape (FIG. 18H). In brief, constructs corresponding to the TetON, NF-κB and Wnt recorders were mixed at an equimolar ratio and co-integrated to PE2(+) HEK293T cells. Each recorder drives pegRNA(s) encoding the insertion of one or two distinct, signal-specific barcodes (Table 6). These cells were exposed to a high concentration of all possible combinations of 0 to 3 stimuli, in triplicate (8 on/off stimulus combinations×3 replicates=24 conditions). Harvesting cells after 48 hours of stimulation, PCR amplification and sequencing of the shared DNA tape was performed. As predicted, the abundances of signal-specific barcodes were highly dependent on the precise combination of stimuli applied (FIG. 18I). Put another way, minimal cross-talk was observed, consistent with the orthogonality of these signaling pathways to one another (FIG. 19D). To push this system further, a separate experiment was performed in which populations of cells bearing all three recorders were exposed to all possible combinations of low, medium, or high concentrations of each stimulus (3 concentrations\hat{ }3 stimuli×3 replicates=81 conditions). Once again harvesting cells after 48 hours and reading the DNA Tape, it was observed that signal-specific barcodes are introduced at rates correlated with the concentration of the corresponding stimulus (FIG. 18J; FIG. 19E), further supporting the conclusion that these recorders are able to capture quantitative information on separate channels despite writing to a shared DNA Tape.

Capturing the Order in which ENGRAM Recorders are Active

In the context of a multiplex signal recorder, it is obviously of interest to capture not only the intensity and duration of individual signals, but also the order in which they are active relative to one another. To this end, ENGRAM 2.0 recorders were devised such that each comprise an “operon” of multiple, csy4 hairpin-flanked pegRNAs, each designed to program insertional edits but in a manner that depends on whether other edits had (or had not) already occurred. For example, in the simplest version of this scheme, the order of two signaling events, A and B (FIG. 17D) can be mapped. For this goal, an A-responsive recorder would encode a first pegRNA that wrote an A-specific barcode to blank DNA Tape (A), but also a second pegRNA that only targeted an already B-edited DNA Tape with a different barcode (A′). Meanwhile, a B-responsive recorder would encode a first pegRNA that wrote a B-specific barcode to blank DNA Tape (B), but also a second pegRNA that only targeted an already A-edited DNA Tape with a different barcode (B′).

To test this concept, ENGRAM 2.0 recorders encoding AA′ or BB′ pegRNA operons were cloned, each driven by the constitutive PGK promoter. A series of transfection programs were performed in which either both A & B were introduced simultaneously (1 program), only A or B was introduced (2 programs), or the recorders were serially transfected (A→B or B→A) with the recovery time between transfections varying between 8 and 72 hours (8 programs) (FIG. 4e). These experiments were performed in triplicate in PE2(+) HEK293T cells, with harvesting, amplification, and sequencing of the DNA Tape five days after the first transfection (11 programs×3 transfection replicates=33 conditions) (FIG. 21A). As predicted and provided there were 24+ hours of recovery between transfections, grossly different ratios of AB′/BA′ edits for (A→B) vs. (B→A) programs (FIG. 20F; FIG. 21B) were observed, indicating that the general scheme is compatible with the recovery of information about the order in which ENGRAM 2.0 recorders are active.

TABLE 7
Comparison of different recording strategies

| | | CRISPR cut | Base editing | Prime editing |
|---|---|---|---|---|
| Writing | Editing modality | Cas9 nuclease | Base editor (dead | Prime editor (Cas9 |
| | | | Cas9 or Cas9 nickase | nickase with reverse |
| | | | with deaminase) | transcriptase) |
| | Other writing | gRNAs + targets OR | gRNAs + targets OR | pegRNAs + common |
| | components | homing | homing | target |
| | | gRNAs/stgRNAs | gRNAs/stgRNAs | |
| | Information content | pseudorandom | pseudorandom | signal specific |
| | of edit | | | |
| | Signal quantification | Signals write to | Signals write to | Signals write to |
| | | distinct sites or sets | distinct sites or sets | shared site or sets of |
| | | of sites | of sites | sites |
| Reading | Recovery efficiency | Low (target site(s) | Moderate (target | High (shared target |
| | | corresponding to | site(s) corresponding | site(s); compatible |
| | | each signal must be | to each signal must | with sc-RNA-seq) |
| | | recovered separately; | be recovered | |
| | | further challenge of | separately) | |
| | | DSB-mediated | | |
| | | deletions) | | |
| | In situ readout | Hard (2 probes/colors | Hard (1 probe/color | Yes, in principle |
| | (FISH) | per signal, limited | per signal, but | (1 probe/color per |
| | | multiplexity) | requires probe tc | signal, 5-10 bp |
| | | | detect 1-2 bp | insertion) |
| | | | mutations, high | |
| | | | noise) | |
| | RNA-seq readout | Hard (recover distinct | Moderate (recover | Yes (One shared |
| | | targets transcribed | distinct targets | DNA Tape |
| | | from different | transcribed from | transcribed from |
| | | promoters) | different promoters | single promoter) |
| | | | OR one shared DNA | |
| | | | Tape transcribed | |
| | | | from single | |
| | | | promoter) | |
| | references | (McKenna et al. | (Tang and Liu 2018; | |
| | | 2016; Perli et al. | Farzadfard et al. | |
| | | 2017; Frieda et al. | 2019) | |
| | | 2017; Chan et al. | | |
| | | 2019; Bowling et al. | | |
| | | 2020) | | |

### DISCUSSION

This Examples discloses ENGRAM, a new strategy for multiplex, DNA-based signal recording, wherein each biological signal of interest is coupled to the Pol-2-mediated transcription of a specific guide RNA, whose expression then programs the insertion of a signal-specific barcode to a genomically encoded DNA Tape. As DNA is stable, recorded signals can be read out at any subsequent point in time, e.g., by DNA sequencing or, potentially, even by DNA FISH. A key strength of ENGRAM is its multiplexibility. For example, with the 5-bp or 6-bp insertions used here, thousands of distinct biological signals can potentially be recorded within the same cell, all competing to write to a shared DNA Tape. This multiplexibility is demonstrated by showing that analogous to an MPRA, ENGRAM can be applied to concurrently and quantitatively capture the activity of hundreds of enhancers. However, unlike an MPRA, these activities are recorded in the relative abundances of the corresponding insertional barcodes in DNA, rather than being measured from active transcription.

In metazoans, a modest number of core signaling pathways are leveraged to give rise to developmental and functional complexity. To demonstrate how ENGRAM can be applied to record the activity of core signaling pathways, Wnt and NF-κB-responsive regulatory elements were used to drive pegRNAs that write to DNA Tape in a quantitative, specific, signal-responsive manner. Further, this Examples demonstrates that both the intensity and duration of pathway stimulation contribute to observed levels of recording. A recorder for Tet-On was also built and characterized, highlighting the potential of ENGRAM to be used in conjunction with heterologous signal transduction systems. In a multiplex implementation of these three recorders, there was minimal cross-talk, consistent with the expected orthogonality of these signaling pathways to one another.

This Examples demonstrates a variant of the ENGRAM method in which the recorder comprises an “operon” of multiple pegRNAs, which are designed to either program or restrict successive edits to the DNA Tape. The resulting pattern of insertional edits allows for inferring the temporal order in which the recorders were activated. Of note, in parallel to this work, a different strategy for “pseudo-processive” genome editing was developed called DNA Typewriter (Choi, J. et al. A time-resolved, multi-symbol molecular recorder via sequential genome editing. Nature (2022) doi: 10.1038/s41586-022-04922-8). In principle, ENGRAM and DNA Typewriter are compatible. For the goal of multiplex, temporally resolved recording of core signaling pathway activity over extended periods of time, the combination of ENGRAM and DNA Typewriter may be more powerful than the ENGRAM variant described here.

In summary, ENGRAM is a method for recording specific biological signals to the genome. It is general—any signal that can be converted to Pol-2 mediated transcription can be used to construct an ENGRAM recorder. It is multiplexable—by coupling specific signals to specific insertions, the number of signals that can be encoded grows exponentially with the insertion length. It is quantitative—the strength or duration of signals, and potentially both, can be recorded and recovered. Particularly if combined with DNA Typewriter, it is envisioned that ENGRAM can be applied as a means of enriching DNA-based recordings of cellular histories, across state, space, and time.

### Materials and Methods

### Cell Culture, Transient Transfections and PiggyBac Integrations

HEK293T cells (CRL-11268) and K562 cells (CCL-243) were purchased from ATCC. HEK293T cells and K562 cells were cultured in DMEM High glucose (GIBCO) and RPMI 1640 medium (GIBCO), respectively, supplemented with 10% Fetal Bovine Serum (Rocky Mountain Biologicals) and 1% penicillin—streptomycin (GIBCO). Cells were grown with 5% CO2 at 37° C.

For transient transfections, 1×10⁵ cells were seeded on a 24-well plate a day before transfection and were transfected with 500 ng plasmid using Lipofectamine 3000 (Thermo Fisher L3000015) following the manufacturer's protocol.

For integrations mediated by the PiggyBac transposon, 1×10⁵ cells were seeded on a 24-well plate a day before transfection and then transfected with 500 ng cargo plasmid and 200 ng Super PiggyBac transposase expression vector (SBI) using Lipofectamine 3000 following the manufacturer's protocol. Monoclonal lines expressing PE2 were constructed by sorting single cells into 96 wells and selected based on prime editing efficiency.

Most ENGRAM recorders tested in this study were integrated into monoclonal PE2(+) HEK293T cell line via the PiggyBac transposon method described above. Of note, for doxycycline recorders, an extra integration was performed to introduce the reverse tetracycline-controlled transactivator (rtTA), which is activated by doxycycline and binds to the tetracycline response element to activate downstream recorder expression. For recorders co-transfected with blocking pegRNA plasmid, 200 ng plasmid was added to the 500 ng cargo plasmid and 200 ng PiggyBac transposase plasmid.

For ligand recording experiments, 1×10⁵ cells were seeded on a 48-well plate 6 h prior to treatment. 1 ml medium with ligand or negative control was added to each well. For the time-series experiment, cells were washed with warm medium and were harvested 24 hours after ligand removal. Doxycycline hyclate (Dox; Sigma, D9891) was reconstituted in 1× Phosphate Buffer Solution (PBS) to the final concentration of 10 mg/mL. TNFα (R&D systems, 210-TA-020/CF) was reconstituted in 1 ml PBS to make a 20 μg/ml stock. CHIR-99021 (Selleck, S2924) was purchased as 10 mM stock (1 ml in DMSO). All ligands were stored at −20° C. Ligands were thawed immediately before experiments and diluted with the appropriate culturing medium. The same volume of DMSO or PBS was added to the medium as a negative control.

### Library Cloning

The pegRNA-5N recorder (including ENGRAM 1.0, and all three variants of ENGRAM 2.0) was cloned with two steps. First, a gene fragment containing CTT pegRNA (Addgene #132778) was PCR amplified using primer sets adding a 5-bp degenerate barcode and flanking BsmBI site for the downstream cloning steps. A carrier plasmid containing two BsmBI sites and two csy4 hairpins was ordered from Twist. Carrier plasmid and the PCR product from the last step were digested with BsmBI (NEB, buffer 3.1) at 55° C. for 1 h and were purified for ligation. The complete pegRNA with 5N degenerate barcode and csy4 hairpins was PCR amplified from the ligation product. ENGRAM plasmid and PCR product from above were digested with BsmBI (NEB, buffer 3.1) at 55° C. for 1 h and purified for ligation. Ligation products were purified and resuspended with 5 μl H₂O for electroporation. Electroporation was performed using NEB® 10-beta Electrocompetent E. coli (C3020) with the manufacturer's protocol. Transformed cells were cultured at 30° C. overnight.

The libraries of 300 enhancers or plasmids bearing signal-responsive elements were cloned in two steps. First, oligos containing enhancer/CRE, two BsmBI restriction sites, barcode, 3′ end of pegRNA and csy4 hairpin were ordered as oPools from IDT. 5′-ENGRAM 2.0 recorder was digested with Xbal and Ncol (NEB, CutSmart buffer) at 37° C. for 1 h and purified. Oligos were cloned into the 5′-ENGRAM2.0 recorder using Gibson assembly. Second, a gene fragment containing minP, csy4 hairpin, HEK3 spacer sequence and pegRNA backbone flanking with two BsmBI sites were ordered as gBlock from IDT. gBlock and construct from step 1 were digested with BsmBI (NEB, buffer 3.1) at 55° C. for 1 h to generate compatible sticky ends and were purified for ligation. Ligation products were transformed into Stable Competent E. coli (NEB C3040). Transformed cells were cultured at 30° C. overnight.

All PCR and digestion purification were purified with AMPure XP beads (0.6× for plasmids and 1.2× for fragments with size 200-300 bp) using the manufacturer's protocol unless specified. All ligation reactions were using Quick ligase (NEB) with vector:insert ratio 1:6 unless specified. All Gibson reactions were using NEBuilder (NEB) with vector:insert ratio 1:6 unless specified. All plasmid DNA was prepared using a ZymoPURE II Plasmid Kit.

### Sequencing Library Generation

Genomic DNA was extracted using the protocol as follows: Wash harvested cells with PBS, add 200 μl of freshly prepared lysis buffer (10 mM Tris-HCl, pH 7.5; 0.05% SDS; 25 μg/ml protease (Thermo Fisher)) per 0.5-1M cells directly into each well of the tissue culture plate. The genomic DNA mixture was incubated at 50° C. for 1 h, followed by an 80° C. enzyme inactivation step for 30 min.

For each reaction we used 2 μl of cell lysate, 0.25 μl 100 mM forward and reverse primer sets, 22.5 μl H₂O and 25 μl Robust HotStart ReadyMix 2×(KAPA Biosystems). PCR reactions were performed as follows: 95° C.×3 mins, 22 cycles of (98° C.×20 seconds, 65° C.×15 seconds and 72° C.×40 seconds). The resulting PCR product was then size-selected using a dual size-selection cleanup of 0.5× and 1×AMPure XP beads (Beckman Coulter) to remove genomic DNA and small fragments (<200 bp) respectively. This size-selected product was subsequently re-amplified to add the flow-cell adapter and sample index for 5 cycles. The final PCR product was cleaned with 0.9×AMPure XP beads (Beckman Coulter). The library was sequenced on an Illumina NextSeq 500 sequencer, an Illumina MiSeq sequencer, or an Illumina NextSeq 2000 sequencer following the manufacturer's protocol.

### Sequence Processing Pipeline

Sequences were first aligned to HEK3 target reference using Burrows-Wheeler Aligner software (bwa) with default settings. Aligned reads were then parsed and analyzed for insertion editing efficiencies using pattern-matching functions. For the pool of hexamer barcodes used for enhancer recording, as well as the pentamer barcodes used for signal responsive recording, barcode sequences were chosen to have a Hamming Distance of greater than 2 from all other members of the same set. After extracting barcode sequences from the aligned reads, unexpected barcodes within 1 Hamming Distance from the expected sequences were corrected for insertion counts.

### RNA Structure Prediction and Editing Score Prediction

RNA structure and minimal free energy prediction were performed using the NUPACK python package (Fornace, M. E., Porubsky, N. J. & Pierce, N. A. A Unified Dynamic Programming Framework for the Analysis of Interacting Nucleic Acid Strands: Enhanced Models, Scalability, and Speed. ACS Synth. Biol. 9, 2665-2678 (2020)) with default settings. Linear lasso regression model to predict editing score of 5 bp barcodes was trained using scikit-learn python package. 85 features to characterize the 5-bp sequence for which the insertional efficiency is being predicted were defined. These were: 1) Sequence features: 84 binary features corresponding to one-hot encoded sequence, including 20 for single nucleotide content (4 nucleotides*5 positions) and 64 for dinucleotide content (16 dinucleotides*4 positions); 2) Structure feature: rescaled minimum free energy within range (0,1). Samples were split with 724 barcodes in a training set and 300 barcodes in a test set. The model was trained with 10-fold cross-validation on the training set and then used to predict the test set.

1. A nucleic acid construct for recording an iterative nucleic acid editing event, the construct comprising a first active target domain, comprising an editable recording sequence configured to hybridize with a first prime editing guide RNA (pegRNA) and one or more inactive truncated target domains comprising a non-editable sequence configured to not hybridize with the pegRNA, wherein the first pegRNA edits the first active target domain, wherein the pegRNA edit comprises the insertion of a sequence comprising from 5′ to 3′ a barcode tag sequence and a target activation sequence, and wherein the pegRNA edit shifts the position of the recording sequence from the editable sequence to the non-editable sequence, thereby changing the editable sequence to a non-editable sequence and the inactive truncated target domain to a second active target domain comprising a second recording sequence configured to hybridize with a second pegRNA.

2-3. (canceled)

4. The nucleic acid construct of claim 1, wherein the bar code tag sequence:
(i) uniquely identifies each pegRNA and each active target domain is programmed by a different pegRNA, thereby each active target domain includes a different barcode tag sequence;
(ii) is constant for each pegRNA and each active target domain is programmed by the same pegRNA, thereby each active target domain includes the same barcode tag sequence; or
(iii) is constant or different for 2, 3, or more unique pegRNAs to alternatively target each activation target domain, thereby every alternating active domain or every 2, 3, or more alternative active domains include the same or different barcode tag sequence.

5-6. (canceled)

7. The nucleic acid construct of claim 1, wherein:
(i) the pegRNA edit inactivates the first active target domain thereby preventing a second hybridization with a second pegRNA; and
(ii) the target activation sequence extends the inactive truncated target domain thereby activating this domain and allowing hybridization with a second pegRNA.

8. The nucleic acid construct of claim 1, comprising 1, 5, 10, 15, 20, 25, 30, 35, 40, 45, 50, 60, 70, 80, 90, 100, 125, 150, 175, 200, or more inactive truncated target domains adjacent to the first active target domain.

9. The nucleic acid construct of claim 8, wherein each inactive truncated target domain comprises 1, 5, 10, 15, 20, 25, 30, 35, 40, 45, 50, 60, 70, 80, 90, 100, 125, 150, 175, 200, or more units.

10. The nucleic acid construct of claim 1, wherein the pegRNA additionally inserts a homology sequence to correct insertion errors.

11. The nucleic acid construct of any preceding claim 1, wherein:
the active target domain is 15-45 nucleotides in length and the inactive truncated target domain is 0-45 nucleotides in length.

12. The nucleic acid construct of any preceding claim 1, wherein:
(i) the first active target domain comprises from 5′ to 3′ a full length CRISPR-Cas9 target site, a protospacer adjacent motif (PAM) sequence, and a homology sequence; and
(ii) the inactive truncated target domain comprises a truncated CRISPR-Cas9 target site, a protospacer adjacent motif (PAM) sequence, and a homology sequence, wherein the pegRNA edit inserts 5′ to the truncated CRISP-Cas9 target site a sequence comprising from 5′ to 3′ the barcode tag sequence and a target activation sequence, wherein the target activation sequence extends the truncated CRISPR-Cas9 target site.

13. (canceled)

14. The nucleic acid construct of claim 1, wherein the nucleic acid construct is a double stranded DNA.

15. A vector comprising a nucleic acid sequence encoding the nucleic acid construct in claim 1 coupled to a promoter and/or a transcribed form of a RNA molecule.

16. A cell comprising the vector of claim 15.

17. The cell of claim 16, further comprising:
(i) one or more nucleic acids encoding one or more pegRNAs; and/or
(ii) a nucleic acid encoding a prime editing enzyme.

18. (canceled)

19. The cell of claim 17, wherein the prime editing enzyme comprises a nickase enzyme operatively associated with a reverse-transcriptase enzyme.

20. A system for recording iterative nucleic acid editing events, the system comprising:
the nucleic acid construct recited in claim 1, or a nucleic acid encoding the nucleic acid construct;
one or more pegRNAs or one or more nucleic acids encoding the one or more pegRNAs configured to hybridize to a first active target domain;
a prime editing enzyme, or a nucleic acid encoding the prime editing enzyme;
wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

21. (canceled)

22. A method of iteratively recording editing events, the method comprising:
contacting the nucleic acid construct recited in claim 1 with one or more pegRNAs and a prime editing enzyme;
wherein the pegRNA is configured to hybridize to the first active target domain, and with a prime editing enzyme insert a sequence 5′ to an inactive truncated target domain, wherein the inserted sequence comprises from 5′ to 3′, a barcode tag sequence and a target activation sequence, and wherein the target activation sequence inactivates the first active target domain and extends and actives the truncated target domain, shifting the position of the active target domain by one unit in the 3′ direction.

23. The method of claim 22, wherein the barcode tag sequence:
(i) uniquely identifies each pegRNA and each active target domain is programmed by a different pegRNA, thereby each active target domain includes a different barcode tag sequence;
(ii) is constant for each pegRNA and each active target domain is programmed by the same pegRNA, thereby each active target domain includes the same barcode tag sequence; or
(iii) is constant or different for 2, 3, or more unique pegRNAs to alternatively target each activation target domain, thereby every alternating active domain or every 2, 3, or more alternative active domains include the same or different barcode tag sequence.

24-26. (canceled)

27. The method of claim 22, wherein the method further comprises sequencing the nucleic acid construct following iterative editing.

28. A method for multiplexed transcription recording, the method comprising:
contacting the nucleic acid construct recited in claim 1 with a prime editing guide RNA (pegRNA) expression cassette, a prime editing enzyme, and an endonuclease,
wherein the expression cassette comprises a promoter, an endonuclease system comprising a first endonuclease target 5′ to the pegRNA and a second endonuclease target 3′ to the pegRNA, an optional nucleic acid construct encoding a functional GFP and/or an endonuclease,
wherein the transcribed region of the nucleic acid construct comprises one or more pegRNAs and expression of one or more pegRNAs is driven by activation of the promoter
releasing the one or more pegRNA by cleavage of the endonuclease target by an endonuclease;
hybridizing the one or more pegRNAs to a target domain; and
editing the target domain by inserting a barcode tag sequence.

29. An expression cassette comprising a cis-regulatory-element (CRE) coupled promoter sequence and a nucleic acid sequence encoding from 5′ to 3′ a first endonuclease target, one or more prime editing guide RNAs (pegRNA), and a second endonuclease target, wherein the nucleic acid sequence is operably linked to the CRE coupled promoter sequence, and wherein cleavage of the first endonuclease target and the second endonuclease target releases the one or more pegRNAs causing the one or more pegRNAs to hybridize to a nucleic acid target and edit the nucleic acid target by inserting a barcode tag sequence.

30. A method for multiplex transcriptional recording, the method comprising:
coupling a cis-regulatory element (CRE) coupled promoter sequence to a nucleic acid sequence encoding from 5′ to 3′ a first endonuclease target, one or more prime editing guide RNAs (pegRNAs), and a second endonuclease target,
releasing the one or more pegRNAs from a transcript by the addition of an endonuclease; and editing of a target nucleic acid sequence by inserting a barcode tag sequence.


SEQUENCE LISTING (135 sequences)

SEQ ID NO: 1 - DNA, 31 bp, synthetic construct
  source 1..31: mol_type = other DNA
ggcccagactgagcacgtgatggcagaggaa

SEQ ID NO: 2 - DNA, 31 bp, synthetic construct
  source 1..31: mol_type = other DNA
ggatgatggtgagcacgtgatggtgagcacg

SEQ ID NO: 3 - DNA, 31 bp, synthetic construct
  source 1..31: mol_type = other DNA
ggatgatggcagaggagtgatggcagaggag

SEQ ID NO: 4 - DNA, 31 bp, synthetic construct
  source 1..31: mol_type = other DNA
ggaccagggtgagcacgccagggtgagcacg

SEQ ID NO: 5 - DNA, 31 bp, synthetic construct
  source 1..31: mol_type = other DNA
gactgatggtgagcacgtgatggtgagcacg

SEQ ID NO: 6 - DNA, 32 bp, synthetic construct
  source 1..32: mol_type = other DNA
gatgatgggtgagcacctgatgggtgagcacc

SEQ ID NO: 7 - DNA, 30 bp, synthetic construct
  source 1..30: mol_type = other DNA
ggactgatggtgagacgtgatggtgagacg

SEQ ID NO: 8 - DNA, 31 bp, synthetic construct
  source 1..31: mol_type = other DNA
gttgttgttcacctcctcgatggcattcact

SEQ ID NO: 9 - DNA, 31 bp, synthetic construct
  source 1..31: mol_type = other DNA
ggacgatggcacctcctcgatggcacctcct

SEQ ID NO: 10 - DNA, 31 bp, synthetic construct
  source 1..31: mol_type = other DNA
ggacgatggcattcacgcgatggcattcacg

SEQ ID NO: 11 - DNA, 31 bp, synthetic construct
  source 1..31: mol_type = other DNA
ggagttgggcacctcctgttgggcacctcct

SEQ ID NO: 12 - DNA, 31 bp, synthetic construct
  source 1..31: mol_type = other DNA
gaccgatggcacctcctcgatggcacctcct

SEQ ID NO: 13 - DNA, 32 bp, synthetic construct
  source 1..32: mol_type = other DNA
gacgatggtcacctcctcgatggtcacctcct

SEQ ID NO: 14 - DNA, 30 bp, synthetic construct
  source 1..30: mol_type = other DNA
ggaccgatggcactcctcgatggcactggt

SEQ ID NO: 15 - DNA, 31 bp, synthetic construct
  source 1..31: mol_type = other DNA
ggtccctccagcatctgctctggctccatgg

SEQ ID NO: 16 - DNA, 31 bp, synthetic construct
  source 1..31: mol_type = other DNA
ggactctggagcatctgctctggagcatctg

SEQ ID NO: 17 - DNA, 31 bp, synthetic construct
  source 1..31: mol_type = other DNA
ggactctggctccatggctctggctccatgg

SEQ ID NO: 18 - DNA, 31 bp, synthetic construct
  source 1..31: mol_type = other DNA
ggaccctggagcatctgccctggagcatctg

SEQ ID NO: 19 - DNA, 31 bp, synthetic construct
  source 1..31: mol_type = other DNA
gacctctggagcatctgctctggagcatctg

SEQ ID NO: 20 - DNA, 32 bp, synthetic construct
  source 1..32: mol_type = other DNA
gtctctgggagcatctgctctgggagcatctg

SEQ ID NO: 21 - DNA, 30 bp, synthetic construct
  source 1..30: mol_type = other DNA
ggatctctggagcacctctctggagcacct

SEQ ID NO: 22 - DNA, 31 bp, synthetic construct
  source 1..31: mol_type = other DNA
gcgagttcaagtgctacccgaggtgcgaggc

SEQ ID NO: 23 - DNA, 31 bp, synthetic construct
  source 1..31: mol_type = other DNA
ggaccgaggagtgctacccgaggagtgctac

SEQ ID NO: 24 - DNA, 31 bp, synthetic construct
  source 1..31: mol_type = other DNA
ggaccgaggtgcgaggcccgaggtgcgaggc

SEQ ID NO: 25 - DNA, 31 bp, synthetic construct
  source 1..31: mol_type = other DNA
ggaagttggagtgctacagttggagtgctac

SEQ ID NO: 26 - DNA, 31 bp, synthetic construct
  source 1..31: mol_type = other DNA
gatccgaggagtgctacccgaggagtgctac

SEQ ID NO: 27 - DNA, 32 bp, synthetic construct
  source 1..32: mol_type = other DNA
gaccgagggagtgctacccgagggagtgctac

SEQ ID NO: 28 - DNA, 30 bp, synthetic construct
  source 1..30: mol_type = other DNA
ggatccgaggagtgcacccgaggagtgcac

SEQ ID NO: 29 - DNA, 31 bp, synthetic construct
  source 1..31: mol_type = other DNA
ggaatcccttctgcagcacctggatcgcttc

SEQ ID NO: 30 - DNA, 31 bp, synthetic construct
  source 1..31: mol_type = other DNA
gagacctggtctgcagcacctggtctgcagc

SEQ ID NO: 31 - DNA, 31 bp, synthetic construct
  source 1..31: mol_type = other DNA
gagacctggatcgcttcacctggatcgcttc

SEQ ID NO: 32 - DNA, 31 bp, synthetic construct
  source 1..31: mol_type = other DNA
gagatccggtctgcagcatccggtctgcagc

SEQ ID NO: 33 - DNA, 31 bp, synthetic construct
  source 1..31: mol_type = other DNA
gtaacctggtctgcagcacctggtctgcagc

SEQ ID NO: 34 - DNA, 32 bp, synthetic construct
  source 1..32: mol_type = other DNA
gaacctgggtctgcagcacctgggtctgcagc

SEQ ID NO: 35 - DNA, 30 bp, synthetic construct
  source 1..30: mol_type = other DNA
gcagacctggtctgcacacctggtctgcac

SEQ ID NO: 36 - DNA, 31 bp, synthetic construct
  source 1..31: mol_type = other DNA
aacctctcggctttcccgcgcggcgccgccc

SEQ ID NO: 37 - DNA, 31 bp, synthetic construct
  source 1..31: mol_type = other DNA
ggagcgcgggctttcccgcgcgggctttccc

SEQ ID NO: 38 - DNA, 31 bp, synthetic construct
  source 1..31: mol_type = other DNA
ggagcgcggcgccgcccgcgcggcgccgccc

SEQ ID NO: 39 - DNA, 31 bp, synthetic construct
  source 1..31: mol_type = other DNA
ggactctgggctttcccctctgggctttccc

SEQ ID NO: 40 - DNA, 31 bp, synthetic construct
  source 1..31: mol_type = other DNA
gatgcgcgggctttcccgcgcgggctttccc

SEQ ID NO: 41 - DNA, 32 bp, synthetic construct
  source 1..32: mol_type = other DNA
gagcgcggagctttcccgcgcggagctttccc

SEQ ID NO: 42 - DNA, 30 bp, synthetic construct
  source 1..30: mol_type = other DNA
ggatgcgcgggctttccgcgcgggctttcc

SEQ ID NO: 43 - DNA, 31 bp, synthetic construct
  source 1..31: mol_type = other DNA
gcctgcaaactggtaggcgccggcgtaggcg

SEQ ID NO: 44 - DNA, 31 bp, synthetic construct
  source 1..31: mol_type = other DNA
gcacgccggctggtaggcgccggctggtagg

SEQ ID NO: 45 - DNA, 31 bp, synthetic construct
  source 1..31: mol_type = other DNA
gcacgccggcgtaggcgcgccggcgtaggcg

SEQ ID NO: 46 - DNA, 31 bp, synthetic construct
  source 1..31: mol_type = other DNA
gcatgcaggctggtaggtgcaggctggtagg

SEQ ID NO: 47 - DNA, 31 bp, synthetic construct
  source 1..31: mol_type = other DNA
gatcgccggctggtaggcgccggctggtagg

SEQ ID NO: 48 - DNA, 32 bp, synthetic construct
  source 1..32: mol_type = other DNA
gacgccggactggtaggcgccggactggtagg

SEQ ID NO: 49 - DNA, 30 bp, synthetic construct
  source 1..30: mol_type = other DNA
gcagcgccggctgtaggcgccggctgtagg

SEQ ID NO: 50 - DNA, 31 bp, synthetic construct
  source 1..31: mol_type = other DNA
agctgctcaccacgacgccagggctgcgggg

SEQ ID NO: 51 - DNA, 31 bp, synthetic construct
  source 1..31: mol_type = other DNA
ggaccagggccacgacgccagggccacgacg

SEQ ID NO: 52 - DNA, 31 bp, synthetic construct
  source 1..31: mol_type = other DNA
ggaccagggctgcggggccagggctgcgggg

SEQ ID NO: 53 - DNA, 31 bp, synthetic construct
  source 1..31: mol_type = other DNA
ggatgctggccacgacgtgctggccacgacg

SEQ ID NO: 54 - DNA, 31 bp, synthetic construct
  source 1..31: mol_type = other DNA
gtcccagggccacgacgccagggccacgacg

SEQ ID NO: 55 - DNA, 32 bp, synthetic construct
  source 1..32: mol_type = other DNA
gaccagggaccacgacgccagggaccacgacg

SEQ ID NO: 56 - DNA, 30 bp, synthetic construct
  source 1..30: mol_type = other DNA
ggacccagggcacgacgccagggcacgacg

SEQ ID NO: 57 - DNA, 14 bp, synthetic construct
  source 1..14: mol_type = other DNA
tgatggtgagcacg

SEQ ID NO: 58 - DNA, 42 bp, synthetic construct
  source 1..42: mol_type = other DNA
ggatgatggtgagcacgtgatggtgagcacgtgatggtgagc

SEQ ID NO: 59 - DNA, 56 bp, synthetic construct
  source 1..56: mol_type = other DNA
ggatgatggtgagcacgtgatggtgagcacgtgatggtgagcacgtgatggtgagc

SEQ ID NO: 60 - DNA, 84 bp, synthetic construct
  source 1..84: mol_type = other DNA
ggatgatggtgagcacgtgatggtgagcacgtgatggtgagcacgtgatggtgagcacgtgatggtgagcacgtgatggtgagc

SEQ ID NO: 61 - DNA, 338 bp, synthetic construct
  source 1..338: mol_type = other DNA
aatgatacggcgaccaccgagatctacacnnnnnnnnnntcgtcggcagcgtcagatgtgtataagagacagggctttatatatcttgtggaaaggacgaaacaccnnnnnnnnggatgatggtgagcacgtgatggtgagcacgtgatggtgagcacgtgatggtgagcacgtgatggtgagcacgtgatggtgagcacgtgatggtgagcgctttaaggccggtcctagcaatttttttaagcttggcgtaactagatcttgagacaaagatcggaagagcacacgtctgaactccagtcacnnnnnnnnnnatctcgtatgccgtcttctgcttg

SEQ ID NO: 62 - DNA, 123 bp, synthetic construct
  source 1..123: mol_type = other DNA
ggatgatggtgagcacgtgagttttagagctagaaatagcaagttaaaataaggctagtccgttatcaacttgaaaaagtggcaccgagtcggtgctcaccatcatccnncgtgctcaccatc

SEQ ID NO: 63 - DNA, 126 bp, synthetic construct
  source 1..126: mol_type = other DNA
ggatgatggtgagcacgtgagttttagagctagaaatagcaagttaaaataaggctagtccgttatcaacttgaaaaagtgtggcaccgagtcggtgctcaccatcatccnnncgtgctcaccatc

SEQ ID NO: 64 - DNA, 165 bp, synthetic construct
  source 1..165: mol_type = other DNA
ggatgatggtgagcacgtgagttttagagctagaaatagcaagttaaaataaggctagtccgttatcaacttgaaaaagtggcaccgagtcggtgctcaccatcatccnncgtgctcaccatcttgacgcggttctatctagttacgcgttaaaccaactagaaa

SEQ ID NO: 65 - DNA, 166 bp, synthetic construct
  source 1..166: mol_type = other DNA
ggatgatggtgagcacgtgagttttagagctagaaatagcaagttaaaataaggctagtccgttatcaacttgaaaaagtggcaccgagtcggtgctcaccatcatccnnncgtgctcaccatcttgacgcggttctatctagttacgcgttaaaccaactagaaa

SEQ ID NO: 66 - DNA, 167 bp, synthetic construct
  source 1..167: mol_type = other DNA
ggatgatggtgagcacgtgagttttagagctagaaatagcaagttaaaataaggctagtccgttatcaacttgaaaaagtggcaccgagtcggtgctcaccatcatccnnnncgtgctcaccatcttgacgcggttctatctagttacgcgttaaaccaactagaaa

SEQ ID NO: 67 - DNA, 168 bp, synthetic construct
  source 1..168: mol_type = other DNA
ggatgatggtgagcacgtgagttttagagctagaaatagcaagttaaaataaggctagtccgttatcaacttgaaaaagtggcaccgagtcggtgctcaccatcatccnnnnncgtgctcaccatcttgacgcggttctatctagttacgcgttaaaccaactagaaa

SEQ ID NO: 68 - DNA, 169 bp, synthetic construct
  source 1..169: mol_type = other DNA
ggatgatggtgagcacgtgagttttagagctagaaatagcaagttaaaataaggctagtccgttatcaacttgaaaaagtggcaccgagtcggtgctcaccatcatccnnnnnncgtgctcaccatcttgacgcggttctatctagttacgcgttaaaccaactagaaa

SEQ ID NO: 69 - DNA, 185 bp, synthetic construct
  source 1..185: mol_type = other DNA
ggatgatggtgagcacgtgagttttagagctagaaatagcaagttaaaataaggctagtccgttatcaacttgaaaaagtggcaccgagtcggtgctcaccatcatccnncgtgctcaccatcttttttggatgatggtgagcacgtgatggtgagcacgtgatggtgagcacgtgatggtgagc

SEQ ID NO: 70 - DNA, 189 bp, synthetic construct
  source 1..189: mol_type = other DNA
ggacgatggcacctcctcgagttttagagctagaaatagcaagttaaaataaggctagtccgttatcaacttgaaaaagtggcaccgagtcggtgcaggtgccatcgtccnnaggaggtgccatcttttttggacgatggcacctcctcgatggcacctcctcgatggcacctcctcgatggcacctgc

SEQ ID NO: 71 - DNA, 189 bp, synthetic construct
  source 1..189: mol_type = other DNA
ggtctctggagcatctgctcgttttagagctagaaatagcaagttaaaataaggctagtccgttatcaacttgaaaaagtggcaccgagtcggtgcatgctccagagaccnncagatgctccagattttttggtctctggagcatctgctctggagcatctgctctggagcatctgctctggagcatgc

SEQ ID NO: 72 - DNA, 185 bp, synthetic construct
  source 1..185: mol_type = other DNA
ggaccgaggagtgctacccggttttagagctagaaatagcaagttaaaataaggctagtccgttatcaacttgaaaaagtggcaccgagtcggtgcactcctcggtccnngtagcactcctcgttttttggaccgaggagtgctacccgaggagtgctacccgaggagtgctacccgaggagtgc

SEQ ID NO: 73 - DNA, 185 bp, synthetic construct
  source 1..185: mol_type = other DNA
gagacctggtctgcagcaccgttttagagctagaaatagcaagttaaaataaggctagtccgttatcaacttgaaaaagtggcaccgagtcggtgcagaccaggtctcnngctgcagaccaggttttttgagacctggtctgcagcacctggtctgcagcacctggtctgcagcacctggtctgc

SEQ ID NO: 74 - DNA, 189 bp, synthetic construct
  source 1..189: mol_type = other DNA
ggagcgcgggctttcccgcggttttagagctagaaatagcaagttaaaataaggctagtccgttatcaacttgaaaaagtggcaccgagtcggtgcaaagcccgcgctccnngggaaagcccgcgttttttggagcgcgggctttcccgcgcgggctttcccgcgcgggctttcccgcgcgggctttgc

SEQ ID NO: 75 - DNA, 191 bp, synthetic construct
  source 1..191: mol_type = other DNA
gcacgccggctggtaggcgcgttttagagctagaaatagcaagttaaaataaggctagtccgttatcaacttgaaaaagtggcaccgagtcggtgctaccagccggcgtgcnncctaccagccggcttttttgcacgccggctggtaggcgccggctggtaggcgccggctggtaggcgccggctggtagc

SEQ ID NO: 76 - DNA, 185 bp, synthetic construct
  source 1..185: mol_type = other DNA
ggaccagggccacgacgccagttttagagctagaaatagcaagttaaaataaggctagtccgttatcaacttgaaaaagtggcaccgagtcggtgctggccctggtccnncgtcgtggccctgttttttggaccagggccacgacgccagggccacgacgccagggccacgacgccagggccagc

SEQ ID NO: 77 - DNA, 189 bp, synthetic construct
  source 1..189: mol_type = other DNA
ggatgatggcagaggagtgagttttagagctagaaatagcaagttaaaataaggctagtccgttatcaacttgaaaaagtggcaccgagtcggtgcctctgccatcatccnnctcctctgccatcttttttggatgatggcagaggagtgatggcagaggagtgatggcagaggagtgatggcagaggc

SEQ ID NO: 78 - DNA, 189 bp, synthetic construct
  source 1..189: mol_type = other DNA
ggacgatggcattcactcgagttttagagctagaaatagcaagttaaaataaggctagtccgttatcaacttgaaaaagtggcaccgagtcggtgcgaatgccatcgtccnnagtgaatgccatcttttttggacgatggcattcactcgatggcattcactcgatggcattcactcgatggcattcgc

SEQ ID NO: 79 - DNA, 189 bp, synthetic construct
  source 1..189: mol_type = other DNA
ggtctctggctccatggctcgttttagagctagaaatagcaagttaaaataaggctagtccgttatcaacttgaaaaagtggcaccgagtcggtgctggagccagagaccnnccatggagccagattttttggtctctggctccatggctctggctccatggctctggctccatggctctggctccagc

SEQ ID NO: 80 - DNA, 188 bp, synthetic construct
  source 1..188: mol_type = other DNA
gaccgaggtgcgaggcccggttttagagctagaaatagcaagttaaaataaggctagtccgttatcaacttgaaaaagtggcaccgagtcggtgctcgcacctcggtccnngcctcgcacctcgttttttggaccgaggtgcgaggcccgaggtgcgaggcccgaggtgcgaggcccgaggtgcgagc

SEQ ID NO: 81 - DNA, 185 bp, synthetic construct
  source 1..185: mol_type = other DNA
gagacctggatcgcttcaccgttttagagctagaaatagcaagttaaaataaggctagtccgttatcaacttgaaaaagtggcaccgagtcggtgcgatccaggtctcnngaagcgatccaggttttttgagacctggatcgcttcacctggatcgcttcacctggatcgcttcacctggatcgc

SEQ ID NO: 82 - DNA, 185 bp, synthetic construct
  source 1..185: mol_type = other DNA
ggagcgcggcgccgcccgcggttttagagctagaaatagcaagttaaaataaggctagtccgttatcaacttgaaaaagtggcaccgagtcggtgcgcgccgcgctccnngggcggcgccgcgttttttggagcgcggcgccgcccgcgcggcgccgcccgcgcggcgccgcccgcgcggcgcgc

SEQ ID NO: 83 - DNA, 191 bp, synthetic construct
  source 1..191: mol_type = other DNA
gcacgccggcgtaggcgcgcgttttagagctagaaatagcaagttaaaataaggctagtccgttatcaacttgaaaaagtggcaccgagtcggtgccctacgccggcgtgcnncgcctacgccggcttttttgcacgccggcgtaggcgcgccggcgtaggcgcgccggcgtaggcgcgccggcgtagggc

SEQ ID NO: 84 - DNA, 185 bp, synthetic construct
  source 1..185: mol_type = other DNA
ggaccagggctgcggggccagttttagagctagaaatagcaagttaaaataaggctagtccgttatcaacttgaaaaagtggcaccgagtcggtgccagccctggtccnnccccgcagccctgttttttggaccagggctgcggggccagggctgcggggccagggctgcggggccagggctggc

SEQ ID NO: 85 - DNA, 185 bp, synthetic construct
  source 1..185: mol_type = other DNA
ggaccagggtgagcacgccagttttagagctagaaatagcaagttaaaataaggctagtccgttatcaacttgaaaaagtggcaccgagtcggtgctcaccctggtccnncgtgctcaccctgttttttggaccagggtgagcacgccagggtgagcacgccagggtgagcacgccagggtgagc

SEQ ID NO: 86 - DNA, 189 bp, synthetic construct
  source 1..189: mol_type = other DNA
ggagttgggcacctcctgttgttttagagctagaaatagcaagttaaaataaggctagtccgttatcaacttgaaaaagtggcaccgagtcggtgcaggtgcccaactccnnaggaggtgcccaattttttggagttgggcacctcctgttgggcacctcctgttgggcacctcctgttgggcacctgc

SEQ ID NO: 87 - DNA, 189 bp, synthetic construct
  source 1..189: mol_type = other DNA
ggtccctggagcatctgcccgttttagagctagaaatagcaagttaaaataaggctagtccgttatcaacttgaaaaagtggcaccgagtcggtgcatgctccagggaccnncagatgctccaggttttttggtccctggagcatctgccctggagcatctgccctggagcatctgccctggagcatgc

SEQ ID NO: 88 - DNA, 185 bp, synthetic construct
  source 1..185: mol_type = other DNA
ggaagttggagtgctacagtgttttagagctagaaatagcaagttaaaataaggctagtccgttatcaacttgaaaaagtggcaccgagtcggtgcactccaacttccnngtagcactccaacttttttggaagttggagtgctacagttggagtgctacagttggagtgctacagttggagtgc

SEQ ID NO: 89 - DNA, 185 bp, synthetic construct
  source 1..185: mol_type = other DNA
gagatccggtctgcagcatcgttttagagctagaaatagcaagttaaaataaggctagtccgttatcaacttgaaaaagtggcaccgagtcggtgcagaccggatctcnngctgcagaccggattttttgagatccggtctgcagcatccggtctgcagcatccggtctgcagcatccggtctgc

SEQ ID NO: 90 - DNA, 189 bp, synthetic construct
  source 1..189: mol_type = other DNA
ggactctgggctttcccctcgttttagagctagaaatagcaagttaaaataaggctagtccgttatcaacttgaaaaagtggcaccgagtcggtgcaaagcccagagtccnngggaaagcccagattttttggactctgggctttcccctctgggctttcccctctgggctttcccctctgggctttgc

SEQ ID NO: 91 - DNA, 191 bp, synthetic construct
  source 1..191: mol_type = other DNA
gcatgcaggctggtaggtgcgttttagagctagaaatagcaagttaaaataaggctagtccgttatcaacttgaaaaagtggcaccgagtcggtgctaccagcctgcatgcnncctaccagcctgcttttttgcatgcaggctggtaggtgcaggctggtaggtgcaggctggtaggtgcaggctggtagc

SEQ ID NO: 92 - DNA, 185 bp, synthetic construct
  source 1..185: mol_type = other DNA
ggatgctggccacgacgtgcgttttagagctagaaatagcaagttaaaataaggctagtccgttatcaacttgaaaaagtggcaccgagtcggtgctggccagcatccnncgtcgtggccagcttttttggatgctggccacgacgtgctggccacgacgtgctggccacgacgtgctggccagc

SEQ ID NO: 93 - DNA, 185 bp, synthetic construct
  source 1..185: mol_type = other DNA
gactgatggtgagcacgtgagttttagagctagaaatagcaagttaaaataaggctagtccgttatcaacttgaaaaagtggcaccgagtcggtgctcaccatcagtcnncgtgctcaccatcttttttgactgatggtgagcacgtgatggtgagcacgtgatggtgagcacgtgatggtgagc

SEQ ID NO: 94 - DNA, 189 bp, synthetic construct
  source 1..189: mol_type = other DNA
gaccgatggcacctcctcgagttttagagctagaaatagcaagttaaaataaggctagtccgttatcaacttgaaaaagtggcaccgagtcggtgcaggtgccatcggtcnnaggaggtgccatcttttttgaccgatggcacctcctcgatggcacctcctcgatggcacctcctcgatggcacctgc

SEQ ID NO: 95 - DNA, 189 bp, synthetic construct
  source 1..189: mol_type = other DNA
gacctctggagcatctgctcgttttagagctagaaatagcaagttaaaataaggctagtccgttatcaacttgaaaaagtggcaccgagtcggtgcatgctccagaggtcnncagatgctccagattttttgacctctggagcatctgctctggagcatctgctctggagcatctgctctggagcatgc

SEQ ID NO: 96 - DNA, 185 bp, synthetic construct
  source 1..185: mol_type = other DNA
gatccgaggagtgctacccggttttagagctagaaatagcaagttaaaataaggctagtccgttatcaacttgaaaaagtggcaccgagtcggtgcactcctcggatcnngtagcactcctcgttttttgatccgaggagtgctacccgaggagtgctacccgaggagtgctacccgaggagtgc

SEQ ID NO: 97 - DNA, 185 bp, synthetic construct
  source 1..185: mol_type = other DNA
gtaacctggtctgcagcaccgttttagagctagaaatagcaagttaaaataaggctagtccgttatcaacttgaaaaagtggcaccgagtcggtgcagaccaggttacnngctgcagaccaggttttttgtaacctggtctgcagcacctggtctgcagcacctggtctgcagcacctggtctgc

SEQ ID NO: 98 - DNA, 189 bp, synthetic construct
  source 1..189: mol_type = other DNA
gatgcgcgggctttcccgcggttttagagctagaaatagcaagttaaaataaggctagtccgttatcaacttgaaaaagtggcaccgagtcggtgcaaagcccgcgcatcnngggaaagcccgcgttttttgatgcgcgggctttcccgcgcgggctttcccgcgcgggctttcccgcgcgggctttgc

SEQ ID NO: 99 - DNA, 191 bp, synthetic construct
  source 1..191: mol_type = other DNA
gatcgccggctggtaggcgcgttttagagctagaaatagcaagttaaaataaggctagtccgttatcaacttgaaaaagtggcaccgagtcggtgctaccagccggcgatcnncctaccagccggcttttttgatcgccggctggtaggcgccggctggtaggcgccggctggtaggcgccggctggtagc

SEQ ID NO: 100 - DNA, 185 bp, synthetic construct
  source 1..185: mol_type = other DNA
gtcccagggccacgacgccagttttagagctagaaatagcaagttaaaataaggctagtccgttatcaacttgaaaaagtggcaccgagtcggtgctggccctgggacnncgtcgtggccctgttttttgtcccagggccacgacgccagggccacgacgccagggccacgacgccagggccagc

SEQ ID NO: 101 - DNA, 188 bp, synthetic construct
  source 1..188: mol_type = other DNA
gatgatgggtgagcacctgagttttagagctagaaatagcaagttaaaataaggctagtccgttatcaacttgaaaaagtggcaccgagtcggtgctcacccatcatcnnggtgctcacccatttttttgatgatgggtgagcacctgatgggtgagcacctgatgggtgagcacctgatgggtgagc

SEQ ID NO: 102 - DNA, 192 bp, synthetic construct
  source 1..192: mol_type = other DNA
gacgatggtcacctcctcgagttttagagctagaaatagcaagttaaaataaggctagtccgttatcaacttgaaaaagtggcaccgagtcggtgcaggtgaccatcgtcnnaggaggtgaccatttttttgacgatggtcacctcctcgatggtcacctcctcgatggtcacctcctcgatggtcacctgc

SEQ ID NO: 103 - DNA, 192 bp, synthetic construct
  source 1..192: mol_type = other DNA
gtctctgggagcatctgctcgttttagagctagaaatagcaagttaaaataaggctagtccgttatcaacttgaaaaagtggcaccgagtcggtgcatgctcccagagacnncagatgctcccagttttttgtctctgggagcatctgctctgggagcatctgctctgggagcatctgctctgggagcatgc

SEQ ID NO: 104 - DNA, 188 bp, synthetic construct
  source 1..188: mol_type = other DNA
gaccgagggagtgctacccggttttagagctagaaatagcaagttaaaataaggctagtccgttatcaacttgaaaaagtggcaccgagtcggtgcactccctcggtcnngtagcactccctcttttttgaccgagggagtgctacccgagggagtgctacccgagggagtgctacccgagggagtgc

SEQ ID NO: 105 - DNA, 188 bp, synthetic construct
  source 1..188: mol_type = other DNA
gaacctgggtctgcagcaccgttttagagctagaaatagcaagttaaaataaggctagtccgttatcaacttgaaaaagtggcaccgagtcggtgcagacccaggttcnngctgcagacccagttttttgaacctgggtctgcagcacctgggtctgcagcacctgggtctgcagcacctgggtctgc

SEQ ID NO: 106 - DNA, 192 bp, synthetic construct
  source 1..192: mol_type = other DNA
gagcgcggagctttcccgcggttttagagctagaaatagcaagttaaaataaggctagtccgttatcaacttgaaaaagtggcaccgagtcggtgcaaagctccgcgctcnngggaaagctccgcttttttgagcgcggagctttcccgcgcggagctttcccgcgcggagctttcccgcgcggagctttgc

SEQ ID NO: 107 - DNA, 192 bp, synthetic construct
  source 1..192: mol_type = other DNA
gacgccggactggtaggcgcgttttagagctagaaatagcaagttaaaataaggctagtccgttatcaacttgaaaaagtggcaccgagtcggtgcaccagtccggcgtcnncctaccagtccggttttttgacgccggactggtaggcgccggactggtaggcgccggactggtaggcgccggactggtgc

SEQ ID NO: 108 - DNA, 188 bp, synthetic construct
  source 1..188: mol_type = other DNA
gaccagggaccacgacgccagttttagagctagaaatagcaagttaaaataaggctagtccgttatcaacttgaaaaagtggcaccgagtcggtgctggtccctggtcnncgtcgtggtccctttttttgaccagggaccacgacgccagggaccacgacgccagggaccacgacgccagggaccagc

SEQ ID NO: 109 - DNA, 188 bp, synthetic construct
  source 1..188: mol_type = other DNA
ggactgatggtgagacgtgagttttagagctagaaatagcaagttaaaataaggctagtccgttatcaacttgaaaaagtggcaccgagtcggtgctctcaccatcagtccnncgtctcaccatcattttttggactgatggtgagacgtgatggtgagacgtgatggtgagacgtgatggtgagagc

SEQ ID NO: 110 - DNA, 192 bp, synthetic construct
  source 1..192: mol_type = other DNA
ggaccgatggcactcctcgagttttagagctagaaatagcaagttaaaataaggctagtccgttatcaacttgaaaaagtggcaccgagtcggtgcaggagtgccatcggtccnnaggagtgccatcgttttttggaccgatggcactcctcgatggcactcctcgatggcactcctcgatggcactcctgc

SEQ ID NO: 111 - DNA, 190 bp, synthetic construct
  source 1..190: mol_type = other DNA
ggatctctggagcactgctcgttttagagctagaaatagcaagttaaaataaggctagtccgttatcaacttgaaaaagtggcaccgagtcggtgcagtgctccagagatccnncagtgctccagagttttttggatctctggagcactgctctggagcactgctctggagcactgctctggagcactgc

SEQ ID NO: 112 - DNA, 184 bp, synthetic construct
  source 1..184: mol_type = other DNA
ggatccgaggagtgcacccggttttagagctagaaatagcaagttaaaataaggctagtccgttatcaacttgaaaaagtggcaccgagtcggtgcactcctcggatccnngtgcactcctcggttttttggatccgaggagtgcacccgaggagtgcacccgaggagtgcacccgaggagtgc

SEQ ID NO: 113 - DNA, 184 bp, synthetic construct
  source 1..184: mol_type = other DNA
gcagacctggtctgcacaccgttttagagctagaaatagcaagttaaaataaggctagtccgttatcaacttgaaaaagtggcaccgagtcggtgcagaccaggtctgcnngtgcagaccaggtttttttgcagacctggtctgcacacctggtctgcacacctggtctgcacacctggtctgc

SEQ ID NO: 114 - DNA, 188 bp, synthetic construct
  source 1..188: mol_type = other DNA
ggatgcgcgggctttccgcggttttagagctagaaatagcaagttaaaataaggctagtccgttatcaacttgaaaaagtggcaccgagtcggtgcaaagcccgcgcatccnnggaaagcccgcgcttttttggatgcgcgggctttccgcgcgggctttccgcgcgggctttccgcgcgggctttgc

SEQ ID NO: 115 - DNA, 188 bp, synthetic construct
  source 1..188: mol_type = other DNA
gcagcgccggctgtaggcgcgttttagagctagaaatagcaagttaaaataaggctagtccgttatcaacttgaaaaagtggcaccgagtcggtgctacagccggcgctgcnncctacagccggcgttttttgcagcgccggctgtaggcgccggctgtaggcgccggctgtaggcgccggctgtagc

SEQ ID NO: 116 - DNA, 188 bp, synthetic construct
  source 1..188: mol_type = other DNA
ggacccagggcacgacgccagttttagagctagaaatagcaagttaaaataaggctagtccgttatcaacttgaaaaagtggcaccgagtcggtgctcgtgccctgggtccnncgtcgtgccctggttttttggacccagggcacgacgccagggcacgacgccagggcacgacgccagggcacgagc

SEQ ID NO: 117 - DNA, 394 bp, synthetic construct
  source 1..394: mol_type = other DNA
caagcagaagacggcatacgagatccgaatccgagtgactggagttcagacgtgtgctcttccgatcttgatttggtaccatagcagatgatccatggtcaatnnnnnnnnggatgatggtgagcacgnnnggatgatggtgagcacgnnnggatgatggtgagcacgnnnggatgatggtgagcacgnnnggatgatggtgagcacgnnnggatgatggtgagccaaggaaaggaagccctgcttcctccagagggcgtcgcaggacagctgctttaaggccggtcctagcaannnnnnnnnnnnnnnnnnnnnnnnnnnnctgtctcttatacacatctgacgctgccgacgannnnnnnnnngtgtagatctcggtggtcgccgtatcatt

SEQ ID NO: 118 - DNA, 204 bp, synthetic construct
  source 1..204: mol_type = other DNA
caccnnnnnnnnggatgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcgctttaaggc

SEQ ID NO: 119 - DNA, 357 bp, synthetic construct
  source 1..357: mol_type = other DNA
caccggnnnnnnnntgtaaaacgacggccagttaatacgactcactatagggaggatgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcacgtgatggtgagcgctttaaggc

SEQ ID NO: 120 - DNA, 58 bp, synthetic construct
  source 1..58: mol_type = other DNA
gcgtcagatgtgtataagagacagggctttatatatcttgtggaaaggacgaaacacc

SEQ ID NO: 121 - DNA, 51 bp, synthetic construct
  source 1..51: mol_type = other DNA
ttcagacgtgtgctcttccgatctttgtctcaagatctagttacgccaagc

SEQ ID NO: 122 - DNA, 48 bp, synthetic construct
  source 1..48: mol_type = other DNA
gcgtcagatgtgtataagagacagatcttgtggaaaggacgaaacacc

SEQ ID NO: 123 - DNA, 49 bp, synthetic construct
  source 1..49: mol_type = other DNA
ttcagacgtgtgctcttccgatctgcctggtatctttatagtcctgtcg

SEQ ID NO: 124 - DNA, 33 bp, synthetic construct
  source 1..33: mol_type = other DNA
ctgtctcttatacacatctgacgctgccgacga

SEQ ID NO: 125 - DNA, 49 bp, synthetic construct
  source 1..49: mol_type = other DNA
ttcagacgtgtgctcttccgatcttgatttggtaccatacgagatgatc

SEQ ID NO: 126 - DNA, 45 bp, synthetic construct
  source 1..45: mol_type = other DNA
gcgtcagatgtgtataagagacagatacggggaaaaggcctccac

SEQ ID NO: 127 - DNA, 45 bp, synthetic construct
  source 1..45: mol_type = other DNA
ttcagacgtgtgctcttccgatctgagttgagcaagcagggtcag

SEQ ID NO: 128 - DNA, 356 bp, synthetic construct
  source 1..356: mol_type = other DNA
ggggatacggggaaaaggcctccacggccagcttggccgcgaatcgatatgtcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggggcactagagggtatataatggaagctcgact

SEQ ID NO: 129 - DNA, 19 bp, synthetic construct
  source 1..19: mol_type = other DNA
tccctatcagtgatagaga

SEQ ID NO: 130 - DNA, 227 bp, synthetic construct
  source 1..227: mol_type = other DNA
ggggatacggggaaaaggcctccacggccacagaccgttagacgccaggacgggctgtcaggctggcgccgcggcgccagcctgacagcccgtcctggcgtctaacggtctgacactagagacgattaaatggacagcatgcgtctctctgccatcaacctccgtgctcagtctgactgccgtataggcagccgccaccatggaccactacctcgacattcgcttgc

SEQ ID NO: 131 - DNA, 10 bp, synthetic construct
  source 1..10: mol_type = other DNA
gggactttcc

SEQ ID NO: 132 - DNA, 10 bp, synthetic construct
  source 1..10: mol_type = other DNA
tcctaaataa

SEQ ID NO: 133 - DNA, 10 bp, synthetic construct
  source 1..10: mol_type = other DNA
taggattatt

SEQ ID NO: 134 - DNA, 264 bp, synthetic construct
  source 1..264: mol_type = other DNA
ggggatacggggaaaaggcctccacggccaagatcaaagggtttaagatcaaagggcttaagatcaaagggtataagatcaaagggcctaagatcaaagggactaagatcaaagggtttaagatcaaagggcttaagatcaaagggcctacactagagacgattaaatggacagcatgcgtctctctgccatcagcaaccgtgctcagtctgactgccgtataggcagccgccaccatggaccactacctcgacattcgcttgc

SEQ ID NO: 135 - DNA, 13 bp, synthetic construct
  source 1..13: mol_type = other DNA
taagatcaaaggg